SEQUENCE LISTING (52 sequences)

SEQ ID NO: 1 - DNA, 9232 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgacaggccttcagtatttatttttagcgatgatggcactcacattgtctatcctactagcacaacagccaccaccccactcgtgcctgcacagcccagcgatgtgccctacattgttgttgacttgtatagtggaagtatggattatgatatacatcctagcctgttgcgcaggaaacgtaaaaaacgcaaacgtgtttatttttcagatggccgtgtggcttccaggcccaaatagattttacttaccccctcaacctatacaacggacattgaacacagaggaatacgtgagacgcaccagtactttcctccatgctgccactgaccgtttgcttactgttggacatccattttacaatattactaatgcggatggcaaagaggtggtccctaaagtttcctctaatcagttcagggccttccgtgtccgtttcccaaatcccaatacctttgcattttgtgataagtccctttttaaccctgacaaggagcgtctggtctggggtattcgtgggattgaggtttctaggggacagcccttaggtattggtgtaacagggaaccctttttttaataagtttgatgatgctgaaaatccctacaatggtataaacaaaaataacattactgaccaaggttcagactcaaggttgagcattgcatttgaccctaagcaaacacagctgctgatagtaggtgctaaacctgcaaagggtgagtactgggacgttgctgcaacatgtgaaaaccctccactgaccaaagcagatgacaaatgtcctgctctagagcttaagtcctcatacattgaggatgcagacatgagtgacataggcctgggaaacttgaatttttctacactgcagagaaacaaatccgatgccccattagatattgtggattctatctgcaaatatcctgactacctgcaaatgatagaagaactatatggagaccacatgtttttctatgtgcggtgtgaagctctgtatgctaggcatataatgcaacacgcgggcaagatggatgctgagcaatttcccacttctctgtacatagactcctctgtagaaggtgagaaattaaattccttgcagcgcactgataggtatttcatgacacccagcggctccctggtagctactgagcagcagctgtttaacaggcccttttggctgcagagatcccagggccataacaatggcatactgtggcacaacgaggcctttgtaacattggttgacactaccaggggaactaactttaccatcagtgttcctgagggggatgcttcttcatataacaattctaagttttttgagtttttaaggcacaccgaggagtttcagcttgcctttattctacagctgtgtaaggtagaccttacccctgagaatttggcttacatacacacaatggatccatccattattgaagactggcatttagctgtcacttcacctcccaattctgtactggaggatcattataggtacatactgtccattgcaactaaatgtccctctaaggatgcagatgatacctccactgacccatacaaagatcttaagttttgggaggttgatctacgggatcgtatgacagagcaattggaccagactccccttggcaggaagtttttgtttcaaactggtatcactcagtcatcatcaaataagcgggtgtccacgcagtctactgcccttactacctacaggcggcctactaagcgccgccggaaggcttaattctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcttcatatgtctagccaccatgcttgctaggcaaagggttaaacgcgctaatcctgaacaactgtataagacatgcaaagcaacggggggcgattgtccacccgatgttattaaacgctatgagcaaactacacctgctgatagtatattaaagtatgggagtgtaggggttttctttggcggtctgggcattggcacaggacgtggtggcggtggcacagtgcttggggctggggcagttgggggacgcccgtccatatccagtggtgcaattggtccccgggatattttgccaattgaatcaggggggccttcactggcagaggaaatacctctgcttcccatggcaccccgtgtgccaaggcctacagatccctttcggccgtcagtgctggaagagccttttattataaggcctcctgaacgcccaaacattttgcatgagcagcgtttccctacagacgctgcaccatttgacaatggcaacacagaaatcacaaccattcctagccaatatgatgttagtgggggaggggttgacattcagataattgaactccctagtgtgaatgaccccggtccctcggttgttacccgcacacaatacaacaatccaacgtttgaggtggaggtgtccactgacattagtggagaaacctcatcaacggacaacattattgtaggagctgaaagcggtggcacatccgtaggtgacaatgctgaactgatacctttgctagatatatcccggggggacacaattgacacaataatacttgcccctggcgaggaggagactgcctttgtgaccagcactcctgaacgtgtgcctatacaggagcgattacctattaggccctatggcagacagtatcagcaagtgcgagttaccgaccctgaatttttagacagcgctgcagtacttgtctctttagagaatccagtgtttgatgcagacattactctcacgtttgaggatgatctgcagcaggcactacgtagtgacacagacctgcgggacgtgcgtcgcctcagtagaccttattaccagaggcgcactactggccttcgtgttagtcgcctggggcaacgtcggggtactatatccacgcgctctggtgttcaggtaggctccgctgctcattttttccaggacattagtccaatcggccaggctattgagccaattgatgcaattgaactagatgtactgggtgagcaatccggtgaggggactattgtgagaggagaccctacgccttctattgagcaagacataggactaaccgctttgggggacaacattgaaaatgaattgcaggaaatagatttattaactgcggatggtgaagaagaccaggagggcagagacctgcagttggtattttccactggcaatgatgaggtggttgatattatgactatacctatacgtgcaggcggggatgacaggccttcagtatttatttttagcgatgatggcactcacattgtctatcctactagcacaacagccaccaccccactcgtgcctgcacagcccagcgatgtgccctacattgttgttgacttgtatagtggaagtatggattatgatatacatcctagcctgttgcgcaggaaacgtaaaaaacgcaaacgtgtttatttttcagatggccgtgtggcttccaggcccaaataggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - protein, 583 aa, Nupapillomavirus 1
MTGLQYLFLAMMALTLSILLAQQPPPHSCLHSPAMCPTLLLTCIVEVWIMIYILACCAGNVKNANVFIFQMAVWLPGPNRFYLPPQPIQRTLNTEEYVRRTSTFLHAATDRLLTVGHPFYNITNADGKEVVPKVSSNQFRAFRVRFPNPNTFAFCDKSLFNPDKERLVWGIRGIEVSRGQPLGIGVTGNPFFNKFDDAENPYNGINKNNITDQGSDSRLSIAFDPKQTQLLIVGAKPAKGEYWDVAATCENPPLTKADDKCPALELKSSYIEDADMSDIGLGNLNFSTLQRNKSDAPLDIVDSICKYPDYLQMIEELYGDHMFFYVRCEALYARHIMQHAGKMDAEQFPTSLYIDSSVEGEKLNSLQRTDRYFMTPSGSLVATEQQLFNRPFWLQRSQGHNNGILWHNEAFVTLVDTTRGTNFTISVPEGDASSYNNSKFFEFLRHTEEFQLAFILQLCKVDLTPENLAYIHTMDPSIIEDWHLAVTSPPNSVLEDHYRYILSIATKCPSKDADDTSTDPYKDLKFWEVDLRDRMTEQLDQTPLGRKFLFQTGITQSSSNKRVSTQSTALTTYRRPTKRRRKA

SEQ ID NO: 3 - protein, 554 aa, Nupapillomavirus 1
MLARQRVKRANPEQLYKTCKATGGDCPPDVIKRYEQTTPADSILKYGSVGVFFGGLGIGTGRGGGGTVLGAGAVGGRPSISSGAIGPRDILPIESGGPSLAEEIPLLPMAPRVPRPTDPFRPSVLEEPFIIRPPERPNILHEQRFPTDAAPFDNGNTEITTIPSQYDVSGGGVDIQIIELPSVNDPGPSVVTRTQYNNPTFEVEVSTDISGETSSTDNIIVGAESGGTSVGDNAELIPLLDISRGDTIDTIILAPGEEETAFVTSTPERVPIQERLPIRPYGRQYQQVRVTDPEFLDSAAVLVSLENPVFDADITLTFEDDLQQALRSDTDLRDVRRLSRPYYQRRTTGLRVSRLGQRRGTISTRSGVQVGSAAHFFQDISPIGQAIEPIDAIELDVLGEQSGEGTIVRGDPTPSIEQDIGLTALGDNIENELQEIDLLTADGEEDQEGRDLQLVFSTGNDEVVDIMTIPIRAGGDDRPSVFIFSDDGTHIVYPTSTTATTPLVPAQPSDVPYIVVDLYSGSMDYDIHPSLLRRKRKKRKRVYFSDGRVASRPK

SEQ ID NO: 4 - DNA, 8920 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgtcatcattgtggttgtcaacaacgggtaaggtctatttaccaccatcaacaccagttgccagggtgcaaagcacggactcctacatacaaagaacaaacatctattatcatgctaatactgaccgcctgttaacagtaggacatccttattttgatgtgaggaaaaataatggagatcatgaagtgttagttcccaaggtgtcaggtaatcagtacagggcctttagggtacacttaccggatcctaacagatttgctctagctgacatgtcagtggtaaatcctgatagggagcgtttggtatgggctgttagaggaatggaaattggtcgtggacagccattaggtgtaggtacatcaggacatccattatttaacaaggtgaaagacacggaaaatccaaatggctataatacaggtggaaaggatgatagggtgaatacatcctttgatcccaaacaaattcaaatgtttgttttgggttgtataccctgcttgggggaacattgggacaaggccttaccttgtgtagaaaatcctcctgatcagggagcgtgtccacctctagaattaaaaaatactattattgaagatggggacatgggagacatagggtttggaaatcttaattttaaaacattatcagtcactaagtctgatgttagtctggatattgttaatgaaatttgcaagtatccagatttcttaaaaatggctaatgatgtgtatggcaatgcttgcttcttttatgccagaagagaacaatgttatgccagacatatgttttgtagaggtgggtcagtaggagacagtattccagatgatgcagttggagaagacaaccattattatttaaaggctgccagtgatcaaaacagagatacaatggcaagttccatttacactcccacagtcagtggatctttagtttctacagatgcacagattttcaataggcctttttggctgcaaagggctcaaggccataataatggtatttgctggggtaatcaaatctttctcacagtaatagataataccaggaatactaatttctgtatcagtgtctcctcaaatgatcaggcattacaggaatacaatactgcaaactttagagaatatttgagacatgtagaagagtatgaattatcctttatattacaattatgtaaagttccattagagccagaagtattagcacaaattaatgctatgaatgcagacattttagaagattggcaattaggttttgttccttctcctgacaatcccatcaatgatacatatagatacatacattcagcagccacacggtgtccagataaaactacacctaaagaaaaagcagatccctttgcaggttatcacttttgggatgttgatttgtctgaaaagttatcattagatttagatcagtattctctgggacgtaaattcttatttcaagccaacctgcaaaacaaaagagttaacagaggggttactgtaaccgggagggctacaacctcaagaggtacaaaacgaaaacgacgctgtttctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgtccacaggacgtcttcatatgtctagccaccatggcgcgcgcacgtagagtaaagcgtgattctgttacaaatatttacaggggctgtaaggcagctggcacatgcccccctgatgttattaataaagttgaacaaaaaactattgctgaccaaattttaaagtatggcagcaccgctgcgttttttggtgggttgggtattagtacaggcaaaggaactggaggcagtactggttatgtccctttgcctgaaggacctgcacctggtgttcgcgtgggtggtacaccaactgtggtgcgccccggggtcattccagaagcgattggtcctactgatataatacctttggatacagtcaaccctattgaccctgttgcaccttcagttgtccctcttacagacacaggacctgatttgttgccaggagaaattgagaccattgctgaggtacatcctgtgtcagatgtaacacctgttgacacaccagtggtgacaggtggtagaggctcgagtgcagtattagaggttgctgacccaagtcctcccactcgtgcacgtgtcagtagaacacaatatcataacccagcttttcaaataatatctgaaacaacaccaacaactggggaagcgtcgttatctgaccaaatcattgtacaatcaggttctggaggacaaaatattggtggtagtgggccttctgtggaaatagaattagaagagttccccacaagatattcatttgaaatagaagagccaacccctcctagaaaaactagtacacctgtaagaatggctcagcaggcctcacgagctttacgtagagctttatacaatcgtagattaacacaacaggtttctgtagaaaatcctctatttttacaacagccttctaaattagttacttttcaatttgataaccctgcatatgaggaggaaataacacaaatatttgagagggatttaagctccattgaagaacctccagatagacaatttatggatgttgttaaattaggtaggcctacatatgctgaaacaccagaaggttacattagagtcagtagacttgggaaacgagcaaccatcagaacacgctctggagcacaggttggcactcaagttcacttttacagagatataagcactattgacacagaaccctccattgaattgcaactgttaggggaacattctggggatgctagtattgttcaaggcccagtagaaagtacatttgttaatatggatgtacaagaaattcctactttggaggaagtgccagaattacattctgaagatgtgctattagaggaggcattagaagactttagtggagcacaattagtttttggaaattctagaagatcaaatgtaataactattcctagatttgagactccaagagagattaatatttatacaccagatttagatggatattacatatcatatccagaaacaaggaatattccagaagttatatacactgagccagacacgactccaacaataataattcatacagaggatttcagtggtgattattatttacatccaagtttgagacgaagaaaaagaaaacgagcctatttgtaagaggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - protein, 513 aa, Alphapapillomavirus 8
MSSLWLSTTGKVYLPPSTPVARVQSTDSYIQRTNIYYHANTDRLLTVGHPYFDVRKNNGDHEVLVPKVSGNQYRAFRVHLPDPNRFALADMSVVNPDRERLVWAVRGMEIGRGQPLGVGTSGHPLFNKVKDTENPNGYNTGGKDDRVNTSFDPKQIQMFVLGCIPCLGEHWDKALPCVENPPDQGACPPLELKNTIIEDGDMGDIGFGNLNFKTLSVTKSDVSLDIVNEICKYPDFLKMANDVYGNACFFYARREQCYARHMFCRGGSVGDSIPDDAVGEDNHYYLKAASDQNRDTMASSIYTPTVSGSLVSTDAQIFNRPFWLQRAQGHNNGICWGNQIFLTVIDNTRNTNFCISVSSNDQALQEYNTANFREYLRHVEEYELSFILQLCKVPLEPEVLAQINAMNADILEDWQLGFVPSPDNPINDTYRYIHSAATRCPDKTTPKEKADPFAGYHFWDVDLSEKLSLDLDQYSLGRKFLFQANLQNKRVNRGVTVTGRATTSRGTKRKRRC

SEQ ID NO: 6 - protein, 521 aa, Alphapapillomavirus 8
MARARRVKRDSVTNIYRGCKAAGTCPPDVINKVEQKTIADQILKYGSTAAFFGGLGISTGKGTGGSTGYVPLPEGPAPGVRVGGTPTVVRPGVIPEAIGPTDIIPLDTVNPIDPVAPSVVPLTDTGPDLLPGEIETIAEVHPVSDVTPVDTPVVTGGRGSSAVLEVADPSPPTRARVSRTQYHNPAFQIISETTPTTGEASLSDQIIVQSGSGGQNIGGSGPSVEIELEEFPTRYSFEIEEPTPPRKTSTPVRMAQQASRALRRALYNRRLTQQVSVENPLFLQQPSKLVTFQFDNPAYEEEITQIFERDLSSIEEPPDRQFMDVVKLGRPTYAETPEGYIRVSRLGKRATIRTRSGAQVGTQVHFYRDISTIDTEPSIELQLLGEHSGDASIVQGPVESTFVNMDVQEIPTLEEVPELHSEDVLLEEALEDFSGAQLVFGNSRRSNVITIPRFETPREINIYTPDLDGYYISYPETRNIPEVIYTEPDTTPTIIIHTEDFSGDYYLHPSLRRRKRKRAYL

SEQ ID NO: 7 - DNA, 8866 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgtataatgtttttcagatggctgtctggttaccagcgcagaataagttctatcttcctccccagcccatcactagaatcctgtccactgatgaatatgtaaccagaaccaatctcttctaccatgcaacatctgaacgtctactgctggtcggacatcctttgtttgagatctccagtaatcaaactgtaactataccaaaagtgtcaccaaatgcatttagagtttttagggtgcgttttgctgatccaaatagatttgcatttggggataaggcaatttttaatccagaaacagaaagattagtttggggcctaagagggatagagataggtagaggccagcctttaggtataggaataacgggccaccctcttttcaataagttagatgatgcagaaaatccaacaaattatattaatactcatgcaaatggagattctagacaaaatactgcttttgatgcaaaacagacacaaatgttcctcgtcggctgtactcctgcttcaggtgaacactggacaagtagtcgttgcccaggggaacaagtgaaacttggggactgccccagggtgcaaatgatagagtctgtcatagaagatggtgacatgatggatattggttttggggctatggattttgctgctttacagcaagacaagtctgatgtccctttagatgttgttcaagcaacatgcaaatatcctgattatatcagaatgaaccatgaagcctatggcaactctatgtttttttttgcacgtcgcgagcaaatgtataccaggcacttttttactcgcgggggttcggtgggtgataaggaggcagtcccacaaagcctgtatttaacagcagatgctgaaccaagaacaactttagcaacaacaaattatgtaggcacaccaagtggctctatggtttcatctgatgtccaattgtttaatagatcttactggcttcagcgatgtcaaggccagaataatggcatttgctggagaaaccagttatttattacagttggagataataccagaggaacaagtttatctatcagtatgaaaaacaatgcaagtactacatattccaatgctaattttaatgattttctaagacatactgaagaatttgatctttcttttatagttcagctttgtaaagtaaagttaactcccgaaaatctagcctacattcatacaatggaccctaatattttagaggattggcaactatctgtatctcaaccacctaccaatcctctagaagatcaatataggtttttagggtcttccttggcagcaaaatgtccagaacaggcgcctcctgagccccagactgatccttatagtcaatataaattctgggaagtcgatctcacagaaaggatgtccgaacaattagaccaatttccactaggaaggaaatttctatatcaaagtggcatgacacaacgtactgctactagttccaccacaaagcgcaaaacagtgcgtttatctacgtcagccaagcgcaggcgtaaggcttagttctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcttcatatgtctagccaccatgtatcgcctacgtagaaaacgcgctgcccccaaagatatatacccctcatgcaaaatatcaaacacctgcccacctgacattcaaaataaaattgagcatacaacaattgctgataaaatattgcaatatggcagtctgggagtttttttgggaggtttgggcattggaacagccagaggctctggaggaagaattggttatactcccctcggtgagggtggtggggttagagttgctactcgtccaactccagtaaggcctacaatacctgtggaaacagtaggccccagtgaaattttccccatagatgttgtagatcctacaggccctgctgttattcccctacaagatttaggtagagacttcccaataccaactgtgcaggttattgcagaaattcaccctatttctgacataccaaacattgttgcttcttcaacaaatgaaggagaatctgccatattagatgtgttacagggaagtgcaaccatacgcactgtttcaagaacacaatacaataacccctctttcactgttgcatctacatctaatataagtgctggagaagcatcaacatcagatattgtatttgttagcaatggttcaggtgacagggtggtgggcgaggatatccccttggtagaattaaacttaggccttgaaacagacacatcttctgttgtacaagaaacagcattttccagcagcacaccaattgctgaaagaccctcttttaggccctcaagattctataataggcgtctatatgaacaggtgcaagtacaagaccctaggttcgttgagcagccacagtcaatggtcacttttgataatccagcatttgagccagagcttgatgaggtgtctattatcttccaaagagacttagatgctcttgctcagacaccagtgcctgaatttagagatgtagtttatctgagcaagcccacattttcgcgggaaccagggggacggttaagggttagccgccttggcaaaagttcaactattcgtacacgcctgggcacagcaattggcgccagaacccactttttctatgatttaagttctattgctccagaagactcaattgaattattgcctttaggtgagcatagtcaaacaacagtcattagttccaacttaggtgacacagcatttatacaaggtgagacagcagaggatgacttagaagttatctctttagaaacaccacaattatattcagaagaagagcttttagacacaaacgaaagtgtgggcgaaaatttgcaacttactattactaactcagagggtgaggtttctatactagatttaacacaaagcagagtcaggccaccttttggcactgaagatactagcttgcatgtatattacccaaattcttctaaagggactccaataattaatcctgaagaatcatttacacctttggttattatagctcttaacaactcaacaggggattttgagttacatcctagtcttagaaagcgtcgtaaaagagcttatgtataagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - protein, 508 aa, Mupapillomavirus 1
MYNVFQMAVWLPAQNKFYLPPQPITRILSTDEYVTRTNLFYHATSERLLLVGHPLFEISSNQTVTIPKVSPNAFRVFRVRFADPNRFAFGDKAIFNPETERLVWGLRGIEIGRGQPLGIGITGHPLFNKLDDAENPTNYINTHANGDSRQNTAFDAKQTQMFLVGCTPASGEHWTSSRCPGEQVKLGDCPRVQMIESVIEDGDMMDIGFGAMDFAALQQDKSDVPLDVVQATCKYPDYIRMNHEAYGNSMFFFARREQMYTRHFFTRGGSVGDKEAVPQSLYLTADAEPRTTLATTNYVGTPSGSMVSSDVQLFNRSYWLQRCQGQNNGICWRNQLFITVGDNTRGTSLSISMKNNASTTYSNANFNDFLRHTEEFDLSFIVQLCKVKLTPENLAYIHTMDPNILEDWQLSVSQPPTNPLEDQYRFLGSSLAAKCPEQAPPEPQTDPYSQYKFWEVDLTERMSEQLDQFPLGRKFLYQSGMTQRTATSSTTKRKTVRLSTSAKRRRKA

SEQ ID NO: 9 - protein, 507 aa, Mupapillomavirus 1
MYRLRRKRAAPKDIYPSCKISNTCPPDIQNKIEHTTIADKILQYGSLGVFLGGLGIGTARGSGGRIGYTPLGEGGGVRVATRPTPVRPTIPVETVGPSEIFPIDVVDPTGPAVIPLQDLGRDFPIPTVQVIAEIHPISDIPNIVASSTNEGESAILDVLQGSATIRTVSRTQYNNPSFTVASTSNISAGEASTSDIVFVSNGSGDRVVGEDIPLVELNLGLETDTSSVVQETAFSSSTPIAERPSFRPSRFYNRRLYEQVQVQDPRFVEQPQSMVTFDNPAFEPELDEVSIIFQRDLDALAQTPVPEFRDVVYLSKPTFSREPGGRLRVSRLGKSSTIRTRLGTAIGARTHFFYDLSSIAPEDSIELLPLGEHSQTTVISSNLGDTAFIQGETAEDDLEVISLETPQLYSEEELLDTNESVGENLQLTITNSEGEVSILDLTQSRVRPPFGTEDTSLHVYYPNSSKGTPIINPEESFTPLVIIALNNSTGDFELHPSLRKRRKRAYV

SEQ ID NO: 10 - DNA, 8911 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgtgcctgtatacacgggtcctgatattacattaccatctactacctctgtatggcccattgtatcacccacggcccctgcctctacacagtatattggtatacatggtacacattattatttgtggccattatattattttattcctaagaaacgtaaacgtgttccctatttttttgcagatggctttgtggcggcctagtgacaataccgtatatcttccacctccttctgtggcaagagttgtaaataccgatgattatgtgactcgcacaagcatattttatcatgctggcagctctagattattaactgttggtaatccatattttagggttcctgcaggtggtggcaataagcaggatattcctaaggtttctgcataccaatatagagtatttagggtgcagttacctgacccaaataaatttggtttacctgatactagtatttataatcctgaaacacaacgtttagtgtgggcctgtgctggagtggaaattggccgtggtcagcctttaggtgttggccttagtgggcatccattttataataaattagatgacactgaaagttcccatgccgccacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagagcaggtactatgggtgacactgtgcctcaatccttatatattaaaggcacaggtatgcgtgcttcacctggcagctgtgtgtattctccctctccaagtggctctattgttacctctgactcccagttgtttaataaaccatattggttacataaggcacagggtcataacaatggtgtttgctggcataatcaattatttgttactgtggtagataccactcgcagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttccccccccgccaactactagtttggtggatacatatcgttttgtacaatctgttgctattacctgtcaaaaggatgctgcaccggctgaaaataaggatccctatgataagttaaagttttggaatgtggatttaaaggaaaagttttctttagacttagatcaatatccccttggacgtaaatttttggttcaggctggattgcgtcgcaagcccaccataggccctcgcaaacgttctgctccatctgccactacgtcttctaaacctgccaagcgtgtgcgtgtacgtgccaggaagtaattctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcttcatatgtctagccaccatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtccccgcctttacagtagggcctaccaacaagtgtcagtggctaaccctgagtttcttacacgtccatcctctttaattacatatgacaacccggcctttgagcctgtggacactacattaacatttgatcctcgtagtgatgttcctgattcagattttatggatattatccgtctacataggcctgctttaacatccaggcgtgggactgttcgctttagtagattaggtcaacgggcaactatgtttacccgcagcggtacacaaataggtgctagggttcacttttatcatgatataagtcctattgcaccttccccagaatatattgaactgcagcctttagtatctgccacggaggacaatgacttgtttgatatatatgcagatgacatggaccctgcagtgcctgtaccatcgcgttctactacctcctttgcattttttaaatattcgcccactatatcttctgcctcttcctatagtaatgtaacggtccctttaacctcctcttgggatgtgcctgtatacacgggtcctgatattacattaccatctactacctctgtatggcccattgtatcacccacggcccctgcctctacacagtatattggtatacatggtacacattattatttgtggccattatattattttattcctaagaaacgtaaacgtgttccctatttttttgcagatggctttgtggcggcctaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 11 - protein, 568 aa, Alphapapillomavirus 7
MCLYTRVLILHYHLLPLYGPLYHPRPLPLHSILVYMVHIIICGHYIILFLRNVNVFPIFLQMALWRPSDNTVYLPPPSVARVVNTDDYVTRTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 12 - protein, 462 aa, Alphapapillomavirus 7
MVSHRAARRKRASVTDLYKTCKQSGTCPPDVVPKVEGTTLADKILQWSSLGIFLGGLGIGTGSGTGGRTGYIPLGGRSNTVVDVGPTRPPVVIEPVGPTDPSIVTLIEDSSVVTSGAPRPTFTGTSGFDITSAGTTTPAVLDITPSSTSVSISTTNFTNPAFSDPSIIEVPQTGEVAGNVFVGTPTSGTHGYEEIPLQTFASSGTGEEPISSTPLPTVRRVAGPRLYSRAYQQVSVANPEFLTRPSSLITYDNPAFEPVDTTLTFDPRSDVPDSDFMDIIRLHRPALTSRRGTVRFSRLGQRATMFTRSGTQIGARVHFYHDISPIAPSPEYIELQPLVSATEDNDLFDIYADDMDPAVPVPSRSTTSFAFFKYSPTISSASSYSNVTVPLTSSWDVPVYTGPDITLPSTTSVWPIVSPTAPASTQYIGIHGTHYYLWPLYYFIPKKRKRVPYFFADGFVAA

SEQ ID NO: 13 - DNA, 8893 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggctgtgtgggtaccgaacaaaggacgtctgtatttgccaccacaacgacctgtggctaaagttttgtctacagatgactatattgttggaactgatttatacttccattcgagtactgaccgccttttaacagttggacatcctttctttgatgtattaagcacagaccaaaataccgttgatgtacccaaggtatctggtaatcaattcagggtatttagactaaatcttccagatcctaaccagtttgctctaattgatacatctatttataatccagaacatgaacgccttgtatggcgtctagtaggtattgaaattgatagaggtggtcctcttggtataggtagtactggtcatccactatttaacaaattgcaggatacagaaaatccttctgtatataatggattaatcagtgaccaaaaggataacaggatgaatgtagcatttgatcccaaacaaaatcaattgtttatagtaggatgtaaacctgctgttggtcaacattgggacaaagcagaaccttgccctaacacgcgcccacccccaggaagttgcccacctcttaaattggtacatagtacaattgaggatggcgacatgtctgatatcggtttaggaaatataaatttcagtgatctttctgatgataaatccagtgcacctttggaaattattaattctaagtgtaagtggcctgattttgctttaatgaccaaagatttatttggcgacagtgccttcttttttggaaggcgtgagcaactttatgctcgccaccagtggtgcagggatggccttgtgggggacgctattccagatgaacacttttattttaatcctaatggccaggatccaaagcctcctcaatatcagcttggctcttctatttactttacaattccgagtggttcgttgactagcagcgaatcaaacatatttggtagaccatattggttgcacagagctcagggtgcaaataatggtattgcatggggcaatcaattgtttgtaactttattggacaacacacacaacacaaactttactatatctgtaagtactgaatcacaaacaacatatgataaaaacaaatttaaggtttatttacgacatgcagaggaaatagaaatagaaatcgtttgtcagctctgtaaggttcctttggaagcagatatcctggcacatttatatgctatggacccatctatattagacaactggcagctagcttttgtacctgcgccaccacaaactctagaagatacttacagatatataagatctatggctactatgtgtcccgcagatgtgcctccaaaggagccagaggacccgtacaaagatttacacttttggactattaatctgactgatagatttacttcagagttggatcaaactcctttaggtaaaagatttttgtatcagatgggattacttactggaaacaaacgcttgcgaacagattatataggttctccagttgctaaacgacgaaggacagtaaaatctagtaaaagaaagaagtcttctgcaaagtaattctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcttcatatgtctagccaccatgcaagccaataaaagacgtaagcgtgctgcagtagaagatatctatgctaaaggttgtacacagccaggaggttattgtccccctgatgtaaaaaataaagtagaaggtaatacatgggctgactttttactaaaagtgtttggaagtgtggtctattttggtgggcttggcattggaacaggtaaaggtactggtggttctacgggatacacaccactaggtggcactgtaggatctagaggcaccacaaacactataaaacctacaataccactggaccctttaggtgttccagatatagttacggtagaccctattgctccagaagccgcgtccatagtacctttagctgaaggattacccgaaccaggtgttatagacacaggcacatctttccctgggttagcagcagataatgaaaatatagtaacagtgctagaccccctatcagaggtcacaggggttggtgaacacccaaatattattactggtggtactgctgatagccctgctattttagatgtacaaacctcacccccaccagctaaaaaaatattattagatccctctattagtaaaactacaactgctgtgcaaactcatgcttcccatgtagatgcaaatctgaatatatttgtagatgcacagtcttttggtactcatgtgggttatacagaagacattcccttggaagaaataaatttaaggagtgaatttgaattagaagatagtgaacccaaaactagcacaccttttgcagaaagagttttaaataaaaccaaacagctctatagtaaatatgttcaacaagtgccaacacgtcctgctgaatttgcactttatacatctaggtttgaatttgaaaatcccgcctttgaggaggacgtcactatggaatttgaaaatgatttggcagagattggggagataacaacccccgcagtttctgatgtaagaattttaaataggccaatatattctgaaactgcagacaggactgtccgcattagtagactaggtcagcgagctggaatgaaaactagaagtggacttgaaataggccaaagggtacacttttactttgacctcagtgatattcctagagaatccatagaacttaatacctatggtaattacagtcatgaaagcactatagttgatgaattgctttctagcacgtttattaatccatttgaaatgcctgttgattcagaaatatttgcagaaaatgaattgttagatcctttagaggaggactttagagattcacatatagtagttccttatttagaagatgagcagataaatattactcctacattgccaccaggcctaggtttaaaagtttacagtgatttatcggaaagagatttattaatacattaccctgtgcagcatgcagacattatggtgccagatacaccttatattcctgtgcaacctcctgatggagttctggtagatgacaatgattattatttgcaccctggtttgtattctcgaaaaagaaaacgacgtgttttgtaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - protein, 516 aa, Betapapillomavirus 2
MAVWVPNKGRLYLPPQRPVAKVLSTDDYIVGTDLYFHSSTDRLLTVGHPFFDVLSTDQNTVDVPKVSGNQFRVFRLNLPDPNQFALIDTSIYNPEHERLVWRLVGIEIDRGGPLGIGSTGHPLFNKLQDTENPSVYNGLISDQKDNRMNVAFDPKQNQLFIVGCKPAVGQHWDKAEPCPNTRPPPGSCPPLKLVHSTIEDGDMSDIGLGNINFSDLSDDKSSAPLEIINSKCKWPDFALMTKDLFGDSAFFFGRREQLYARHQWCRDGLVGDAIPDEHFYFNPNGQDPKPPQYQLGSSIYFTIPSGSLTSSESNIFGRPYWLHRAQGANNGIAWGNQLFVTLLDNTHNTNFTISVSTESQTTYDKNKFKVYLRHAEEIEIEIVCQLCKVPLEADILAHLYAMDPSILDNWQLAFVPAPPQTLEDTYRYIRSMATMCPADVPPKEPEDPYKDLHFWTINLTDRFTSELDQTPLGKRFLYQMGLLTGNKRLRTDYIGSPVAKRRRTVKSSKRKKSSAK

SEQ ID NO: 15 - protein, 509 aa, Betapapillomavirus 2
MQANKRRKRAAVEDIYAKGCTQPGGYCPPDVKNKVEGNTWADFLLKVFGSVVYFGGLGIGTGKGTGGSTGYTPLGGTVGSRGTTNTIKPTIPLDPLGVPDIVTVDPIAPEAASIVPLAEGLPEPGVIDTGTSFPGLAADNENIVTVLDPLSEVTGVGEHPNIITGGTADSPAILDVQTSPPPAKKILLDPSISKTTTAVQTHASHVDANLNIFVDAQSFGTHVGYTEDIPLEEINLRSEFELEDSEPKTSTPFAERVLNKTKQLYSKYVQQVPTRPAEFALYTSRFEFENPAFEEDVTMEFENDLAEIGEITTPAVSDVRILNRPIYSETADRTVRISRLGQRAGMKTRSGLEIGQRVHFYFDLSDIPRESIELNTYGNYSHESTIVDELLSSTFINPFEMPVDSEIFAENELLDPLEEDFRDSHIVVPYLEDEQINITPTLPPGLGLKVYSDLSERDLLIHYPVQHADIMVPDTPYIPVQPPDGVLVDDNDYYLHPGLYSRKRKRRVL

SEQ ID NO: 16 - DNA, 10827 bp, Artificial Sequence
  Description of Artificial Sequence Synthetic polynucleotide
gacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaaggctagagtacttaatacgactcactataggctagagccaccatgagcctgtggctgcccagcgaggccaccgtgtacctgccccccgtgcccgtgagcaaggtggtgagcaccgacgagtacgtggccaggaccaacatctactaccacgccggcaccagcaggctgctggccgtgggccacccctacttccccatcaagaagcccaacaacaacaagatcctggtgcccaaggtgagcggcctgcagtacagggtgttcaggatccacctgcccgaccccaacaagttcggcttccccgacaccagcttctacaaccccgacacccagaggctggtgtgggcctgcgtgggcgtggaggtgggcaggggccagcccctgggcgtgggcatcagcggccaccccctgctgaacaagctggacgacaccgagaacgccagcgcctacgccgccaacgccggcgtggacaacagggagtgcatcagcatggactacaagcagacccagctgtgcctgatcggctgcaagccccccatcggcgagcactggggcaagggcagcccctgcaccaacgtggccgtgaaccccggcgactgcccccccctggagctgatcaacaccgtgatccaggacggcgacatggtggacaccggcttcggcgccatggacttcaccaccctgcaggccaacaagagcgaggtgcccctggacatctgcaccagcatctgcaagtaccccgactacatcaagatggtgagcgagccctacggcgacagcctgttcttctacctgaggagggagcagatgttcgtgaggcacctgttcaacagggccggcgccgtgggcgagaacgtgcccgacgacctgtacatcaagggcagcggcagcaccgccaacctggccagcagcaactacttccccacccccagcggcagcatggtgaccagcgacgcccagatcttcaacaagccctactggctgcagagggcccagggccacaacaacggcatctgctggggcaaccagctgttcgtgaccgtggtggacaccaccaggagcaccaacatgagcctgtgcgccgccatcagcaccagcgagaccacctacaagaacaccaacttcaaggagtacctgaggcacggcgaggagtacgacctgcagttcatcttccagctgtgcaagatcaccctgaccgccgacgtgatgacctacatccacagcatgaacagcaccatcctggaggactggaacttcggcctgcagcccccccccggcggcaccctggaggacacctacaggttcgtgaccagccaggccatcgcctgccagaagcacaccccccccgcccccaaggaggaccccctgaagaagtacaccttctgggaggtgaacctgaaggagaagttcagcgccgacctggaccagttccccctgggcaggaagttcctgctgcaggccggcctgaaggccaagcccaagttcaccctgggcaagaggaaggccacccccaccaccagcagcaccagcaccaccgccaagaggaagaagaggaagctgtgaaagcttatcgataccgtcgacctcgacctgcagaagcttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgctctagagccaccatgaggcacaagaggagcgccaagaggaccaagagggccagcgccacccagctgtacaagacctgcaagcaggccggcacctgcccccccgacatcatccccaaggtggagggcaagaccatcgccgaccagatcctgcagtacggcagcatgggcgtgttcttcggcggcctgggcatcggcaccggcagcggcaccggcggcaggaccggctacatccccctgggcaccaggccccccaccgccaccgacaccctggcccccgtgaggccccccctgaccgtggaccccgtgggccccagcgaccccagcatcgtgagcctggtggaggagaccagcttcatcgacgccggcgcccccaccagcgtgcccagcatcccccccgacgtgagcggcttcagcatcaccaccagcaccgacaccacccccgccatcctggacatcaacaacaccgtgaccaccgtgaccacccacaacaaccccaccttcaccgaccccagcgtgctgcagccccccacccccgccgagaccggcggccacttcaccctgagcagcagcaccatcagcacccacaactacgaggagatccccatggacaccttcatcgtgagcaccaaccccaacaccgtgaccagcagcacccccatccccggcagcaggcccgtggccaggctgggcctgtacagcaggaccacccagcaggtgaaggtggtggaccccgccttcgtgaccacccccaccaagctgatcacctacgacaaccccgcctacgagggcatcgacgtggacaacaccctgtacttcagcagcaacgacaacagcatcaacatcgcccccgaccccgacttcctggacatcgtggccctgcacaggcccgccctgaccagcaggaggaccggcatcaggtacagcaggatcggcaacaagcagaccctgaggaccaggagcggcaagagcatcggcgccaaggtgcactactactacgacctgagcaccatcgaccccgccgaggagatcgagctgcagaccatcacccccagcacctacaccaccaccagccacgccgccagccccaccagcatcaacaacggcctgtacgacatctacgccgacgacttcatcaccgacaccagcaccacccccgtgcccagcgtgcccagcaccagcctgagcggctacatccccgccaacaccaccatccccttcggtggcgcctacaacatccccctggtgagcggccccgacatccccatcaacatcaccgaccaggcccccagcctgatccccatcgtgcccggcagcccccagtacaccatcatcgccgacgccggcgacttctacctgcaccccagctactacatgctgaggaagaggaggaagaggctgccctacttcttcagcgacgtgagcctggccgcctgaaagctttttgaattctttggatccactagtggatcccccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcagaattctatcaaatatttaaagaaaaaaaaattgtatcaactttctacaatctctttcagaagacagaagcagagggaatacttcctaaatcattcaactaggccagcattaccttaataccggaactagaaaatgacattacaagaaaagaaaacaacagaccaatatctctcatgaacaaagatacaaacattttcaacaaaatattagcaaaaagaatccaagaatgtatcaaaaaatatacaccacaaccaagtagaatttattccagatatgtaagggtggttcaacgtttgaaaatcaattaacgtaatttgtcccatcaacaggttaaagaagaaaatcacatggtcatattgatagacacagaaaaagcatttgacaaaatttaacacccattcatgatgcaatctctcagtaaactaggaatagaggaaaacttcctcagcttgaatgtaccttcctctcaattttgctatgaacctgaaactcctcttaaaaaataaagtttttcatttaaaaagaaaacaaaaaacatggaggagcgttgatgtatctcattttagaccaatcagctatggatagttaggcgacagcacagatagctgctgtacttctgtttctggcaatgttccagactacatttaaaaaatttttaattatagacttgtacttaatgttcaagaaaaatatgaaaatggctttgccgtgttaatgctactcttttttaaaaaaaactaaagttcaaactttatttatatttcattagttttttagctactgttctttttctgttctgggatctcattcagaatgccacattacatataattctcatgtctccttgggttcctcttagttttgacagttcctcagacttttcttatttttgatgaccttgacagttttgaggagtactggttagatatagggtaatggtttttaaagtatatttgtcatgatttatactggggtaagggtttggggaggaagcccatggggtaaagtactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttaacaaacagcttttttggggtgaacatattgactacaaatttatgtggaaagaaagtaccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtc

SEQ ID NO: 17 - protein, 505 aa, Alphapapillomavirus 9
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 18 - protein, 473 aa, Alphapapillomavirus 9
MRHKRSAKRTKRASATQLYKTCKQAGTCPPDIIPKVEGKTIADQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDLSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

SEQ ID NO: 19 - DNA, 8941 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgtgcctgtatacacgggtcctgatattacattaccatctactacctctgtatggcccattgtatcacccacggcccctgcctctacacagtatattggtatacatggtacacattattatttgtggccattatattattttattcctaagaaacgtaaacgtgttccctatttttttgcagatggctttgtggcggcctagtgacaataccgtatatcttccacctccttctgtggcaagagttgtaaataccgatgattatgtgactcccacaagcatattttatcatgctggcagctctagattattaactgttggtaatccatattttagggttcctgcaggtggtggcaataagcaggatattcctaaggtttctgcataccaatatagagtatttagggtgcagttacctgacccaaataaatttggtttacctgatactagtatttataatcctgaaacacaacgtttagtgtgggcctgtgctggagtggaaattggccgtggtcagcctttaggtgttggccttagtgggcatccattttataataaattagatgacactgaaagttcccatgccgccacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagagcaggtactatgggtgacactgtgcctcaatccttatatattaaaggcacaggtatgcctgcttcacctggcagctgtgtgtattctccctctccaagtggctctattgttacctctgactcccagttgtttaataaaccatattggttacataaggcacagggtcataacaatggtgtttgctggcataatcaattatttgttactgtggtagataccactcccagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttcccccccccccaactactagtttggtggatacatatcgttttgtacaatctgttgctattacctgtcaaaaggatgctgcaccggctgaaaataaggatccctatgataagttaaagttttggaatgtggatttaaaggaaaagttttctttagacttagatcaatatccccttggacgtaaatttttggttcaggctggattgcgtcgcaagcccaccataggccctcgcaaacgttctgctccatctgccactacgtcttctaaacctgccaagcgtgtgcgtgtacgtgccaggaagtaattctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcttcatatgtctagccaccatgcgacacaaacgttctgcaaaacgcacaaaacgtgcatcggctacccaactttataaaacatgcaaacaggcaggtacatgtccacctgacattatacctaaggttgaaggcaaaactattgctgaacaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttctgatccttctatagtttctttagtggaagaaactagttttattgatgctggtgcaccaacatctgtaccttccattcccccagatgtatcaggatttagtattactacttcaactgataccacacctgctatattagatattaataatactgttactactgttactacacataataatcccactttcactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaggttaaagttgtagaccctgcttttgtaaccactcccactaaacttattacatatgataatcctgcatatgaaggtatagatgtggataatacattatatttttctagtaatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgatttaagtactattgatcctgcagaagaaatagaattacaaactataacaccttctacatatactaccacttcacatgcagcctcacctacttctattaataatggattatatgatatttatgcagatgactttattacagatacttctacaaccccggtaccatctgtaccctctacatctttatcaggttatattcctgcaaatacaacaattccttttggtggtgcatacaatattcctttagtatcaggtcctgatatacccattaatataactgaccaagctccttcattaattcctatagttccagggtctccacaatatacaattattgctgatgcaggtgacttttatttacatcctagttattacatgttacgaaaacgacgtaaacgtttaccatattttttttcagatgtctctttggctgcctaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 20 - protein, 568 aa, Alphapapillomavirus 9
MCLYTRVLILHYHLLPLYGPLYHPRPLPLHSILVYMVHIIICGHYIILFLRNVNVFPIFLQMALWRPSDNTVYLPPPSVARVVNTDDYVTPTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMPASPGSCVYSPSPSGSIVTSDSQLFNKPYWLHKAQGHNNGVCWHNQLFVTVVDTTPSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 21 - protein, 472 aa, Alphapapillomavirus 9
MRHKRSAKRTKRASATQLYKTCKQAGTCPPDIIPKVEGKTIAEQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDLSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLA

SEQ ID NO: 22 - DNA, 8716 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgtggcggcttaatgacaacaaggtttacctgcctcctccagggcctatagcatctattgtgagcacagatgaatatgtgcaacgcaccaacttattttattatgctggcagttcacgtttgcttgcagtgggtcacccatatttcccccttaaaaattcctctggtaaaataactgtacctaaggtttctggttatcaatacagagtatttagagttaaattgcctgaccctaataaatttggcttttcagaaacaacactggttacatcagacactcagcgtttagtctggggatgcgtaggagttgaaattggtagaggacaacctttaggtgttggaataagtggccatccgtatttaaataagtatgatgacactgaaaacccgtctgggtatggcacatcgccgggacaagataacagagaaaatgtagcaatggattataaacaaacacagctgtgtattgttggctgtacacctcctatgggtgaatattggggtcagggtgtgccttgcaacgcatcaggtgttacccaaggtgattgtcctgtaatagaattaaaaagtgaagttatacaggatggtgacatggtagatacaggatttggtgcaatggattttgcttccctacaggccagtaaaagtgatgtacccttagacctggttaatactaaaagtaaatatcctgattatttgggaatggcagcagagccttatgggaatagtttgtttttttttctacgccgggaacaaatgttccttagacatttttttaataaagctggtaaaactggcgacgttgtgccttccgatatgtatattgctggctctaataccaggtccaaaattgcagatagtatatatttttctacacccagtgggtctttggttacttctgattctcaattgtttaacaaacccttatggatacaaaaggcccagggacataataatggcatttgttttgggaatcagttgtttgttacagtggtagataccactcgtagtacaaacttaacgttatgtgcctctactgaccctactgtgcccagtacatatgacaatgcaaagtttaaggaatacctgcggcatgtggaagaatatgatctgcagtttatatttcaattatgcataataacgctaaacccagaggttatgacatatattcatactatggatcccacattattagaggactggaattttggtgtgtccccacctgcctctgcttctttggaagatacttatcgctttttgtctaacaaggccattgcatgtcaaaaaaatgctcccccaaaagaacgggaggatccctataaaaagtatacattttgggatataaatcttacagaaaagttttctgcacaacttacccagtttcccttagggcgcaaatttgttatgcaggcgggtttgcgtcccaaacctaaattaaaaactgtaaagcgttctgcaccatcctcctctacgtctgcccctgcctctaaacgcaaaaaaactaagcgataattctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcttcatatgtctagccaccatggtgtctcatacacataaaaggcgcaaacgggcatcagctacacaattatatcaaacatgcaaggctgctggcacatgtccctcggatgtaattaataaggttgagcatactacaatagcagatcagatattaaaatgggcgagcatgggagtgtattttggagggttgggtattggaacaggctcaggaactggaggcagaacaggctatgtccctctaacaacaggtcgtacgggtattgtccctaaggtgactgcagagcctggagtagtgtcacgtcctcctattgttgtagaatctgttgctccaactgatccttctattgtgtccttaattgaggaatcaagcataattcagtccggggctcctattaccaatattccatcacatggtggctttgaggtaacctcctctggatcagaggttcctgcaattttagatgtttccccatctacttcagtgcatattactacatctacacatttaaatcctgcatttactgatcctactattgtacagccaacccccccagttgaggctgggggacgtattataatatctcactccactgttactgctgatagtgctgaacaaattcctatggatacgtttgttatacacagcgatcctaccactagcacacctattccaggcactgccccacgacctcgtttgggcctgtacagtaaggcattgcagcaggtggaaattgttgaccctacatttttgtcctcgccacaacgtttaattacatatgacaatcctgtatttgaggatcctaatgctacattaacatttgaacagcctacagtacatgaagctcctgattctaggtttatggatatagttactttacatagacctgcattaacatcccgacgaggtatagttagatttagtagggtgggtgcgcgcggtactatgtatactcgcagtggtatacgtattgggggtcgtgtacacttttttacagatattagttccatacccacagaggaatcaatagaattgcagcccctaggacgttcccagtcctttcctactgtttctgatactagtgatttatatgatatatatgcagatgagaatctgttaaataatgatattagttttactgacacacacgtgtccctacagaattctactaaggttgttaatacagctgtgccacttgcaactgtacctgatatttatgcacaaacggggcctgacataagctttcctactattcctattcacattccatatattcctgtgtccccatctatttcccctcagtctgtttccatacatggcactgatttttatttgcatccttcattgtggcatttgggcaaacgccgtaaacgcttttcatatttttttacagataactatgtggcggcttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 23 - protein, 503 aa, Alphapapillomavirus 8
MWRLNDNKVYLPPPGPIASIVSTDEYVQRTNLFYYAGSSRLLAVGHPYFPLKNSSGKITVPKVSGYQYRVFRVKLPDPNKFGFSETTLVTSDTQRLVWGCVGVEIGRGQPLGVGISGHPYLNKYDDTENPSGYGTSPGQDNRENVAMDYKQTQLCIVGCTPPMGEYWGQGVPCNASGVTQGDCPVIELKSEVIQDGDMVDTGFGAMDFASLQASKSDVPLDLVNTKSKYPDYLGMAAEPYGNSLFFFLRREQMFLRHFFNKAGKTGDVVPSDMYIAGSNTRSKIADSIYFSTPSGSLVTSDSQLFNKPLWIQKAQGHNNGICFGNQLFVTVVDTTRSTNLTLCASTDPTVPSTYDNAKFKEYLRHVEEYDLQFIFQLCIITLNPEVMTYIHTMDPTLLEDWNFGVSPPASASLEDTYRFLSNKAIACQKNAPPKEREDPYKKYTFWDINLTEKFSAQLTQFPLGRKFVMQAGLRPKPKLKTVKRSAPSSSTSAPASKRKKTKR

SEQ ID NO: 24 - protein, 463 aa, Alphapapillomavirus 8
MVSHTHKRRKRASATQLYQTCKAAGTCPSDVINKVEHTTIADQILKWASMGVYFGGLGIGTGSGTGGRTGYVPLTTGRTGIVPKVTAEPGVVSRPPIVVESVAPTDPSIVSLIEESSIIQSGAPITNIPSHGGFEVTSSGSEVPAILDVSPSTSVHITTSTHLNPAFTDPTIVQPTPPVEAGGRIIISHSTVTADSAEQIPMDTFVIHSDPTTSTPIPGTAPRPRLGLYSKALQQVEIVDPTFLSSPQRLITYDNPVFEDPNATLTFEQPTVHEAPDSRFMDIVTLHRPALTSRRGIVRFSRVGARGTMYTRSGIRIGGRVHFFTDISSIPTEESIELQPLGRSQSFPTVSDTSDLYDIYADENLLNNDISFTDTHVSLQNSTKVVNTAVPLATVPDIYAQTGPDISFPTIPIHIPYIPVSPSISPQSVSIHGTDFYLHPSLWHLGKRRKRFSYFFTDNYVAA

SEQ ID NO: 25 - DNA, 8755 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgtcactttggttgccgtctgaggctaccgtataccttccccctgtgcctgtgtccaaagtagtcagtacagatgagtacgtggcgaggactaatatctattatcacgcaggaacgtccagactcctcgccgtcggccacccgtatttcccgatcaaaaaacctaacaataataagattttggtccctaaggtctccggcctccaataccgggtgttccgaattcacctgccagacccaaataagttcggtttccctgatacctccttctataaccctgacacgcaaagactggtatgggcctgtgtcggtgttgaagtgggcaggggccagcccttgggagttggcatctctgggcatcctcttcttaacaagctcgatgataccgaaaacgcgagtgcgtatgccgccaatgccggggtggataatagggagtgcattagtatggattataaacaaacgcaactgtgtctgatcggatgcaagccgcctataggcgagcattgggggaaggggtccccctgtacgaatgtagcggtgaatccgggtgactgcccgcccctggagctcatcaataccgtaattcaagatggagacatggtccatacgggatttggtgccatggactttaccaccctccaggctaacaagtctgaggtaccgctggacatttgcacctccatttgtaaatacccagactatataaaaatggttagtgagccatatggtgacagcctgtttttttacctgaggagagagcagatgttcgttaggcacttgtttaatcgcgctggtactgttggggagaatgtgccagatgatctctacatcaagggaagcggatctacggcaaaccttgctagttctaattactttccaacaccgtcaggttcaatggttacaagcgacgcgcaaatttttaacaaaccgtactggcttcaaagagcccaaggccataataacggtatctgttggggaaaccagctttttgtcacagttgtagatacaacgcgatcaacgaacatgagtttgtgtgcggcgatatccactagtgaaacgacttacaaaaatactaatttcaaagaatacctccgccatggtgaggagtatgaccttcagtttatatttcaattgtgcaagattacacttacagcggacgttatgacttatattcacagcatgaactcaacaattcttgaagactggaactttgggcttcagccgccgccagggggaaccttggaagacacttacaggttcgtaacgcaggctatcgcatgtcagaaacatacccctccagctccgaaagaagacgatcccctgaaaaagtatacattctgggaggtcaacctgaaggagaaattttccgctgatctcgatcagttccctcttgggaggaaatttttgctgcaggctggactcaaggctaaaccaaagttcacactcggcaaacgaaaagccacgccaactacaagtagtacgagtacgacagccaagcgaaagaaacgcaagttgtaattctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcttcatatgtctagccaccatgcggcacaagcgatccgccaagaggactaagagagcgtctgctacccaactttataaaacctgcaaacaggcaggcacttgccctccagacatcatccccaaggtcgagggtaagaccatcgcggaacaaattttgcaatacgggtccatgggggttttttttggcggtcttggtatagggacgggcagtggaacgggcggtaggaccggttatattcctctcggaacgcgaccacccactgcaacagacacattggcacccgtgagaccacctctgactgttgacccggtaggaccatctgatccatcaattgtcagtctcgttgaagagacgagctttatcgacgctggtgctccgacaagtgttccttctatcccacccgatgtatccggttttagtattactacgagtactgacactacccctgctatacttgacatcaacaacacggtaacaactgtcactacccacaacaacccaacgtttacggaccctagcgtgctgcaacctccaacacccgccgagacaggaggacattttactttgtctagttctacaatctctacccacaactatgaggaaattccaatggacacttttatcgtaagtaccaacccaaacacagtcaccagtagcacccccatccctggcagtcgaccggtggcaagactgggtttgtactcacggacaacgcagcaagtgaaagttgtagaccctgcgttcgttaccaccccaacaaaactgattacatatgataacccagcatatgaaggtatcgatgttgataataccctctacttcagttctaatgacaattctataaatattgctcccgaccctgactttctggacatagtagccctgcatcgaccagccctcacttctcggcgaacgggtatcaggtattctcgaataggtaacaagcaaaccctccgcacacgctcagggaagtctattggagctaaagtccattattactacgatttgagcacaattgaccccgccgaggagatcgagcttcaaacgattactccaagtacttataccactacctcccatgctgcgtctcctacgagcattaataatgggctttatgatatttacgcagacgacttcatcactgatacatctactacccccgtaccgtcagtacccagcacgagtctctcaggttacatccccgccaacaccactataccgttcggaggtgcatacaatatcccgttggtcagtgggccggacattccaataaatataactgatcaagcgccgtctcttatccccattgttcccggtagtccccaatacacgataattgccgatgcgggcgatttttacttgcacccttcttactacatgctccgaaaacgcagaaagcggcttccctatttcttcagtgatgtttccctcgcggcgtaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 26 - protein, 505 aa, Alphapapillomavirus 9
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVHTGFGAMDFTTLQANKSEVPLDICTSICKYPDYIKMVSEPYGDSLFFYLRREQMFVRHLFNRAGTVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTQAIACQKHTPPAPKEDDPLKKYTFWEVNLKEKFSADLDQFPLGRKFLLQAGLKAKPKFTLGKRKATPTTSSTSTTAKRKKRKL

SEQ ID NO: 27 - protein, 473 aa, Alphapapillomavirus 9
MRHKRSAKRTKRASATQLYKTCKQAGTCPPDIIPKVEGKTIAEQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDLSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

SEQ ID NO: 28 - DNA, 8896 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggcggtttgggtccccaataaagggcgcctttaccttcctccacagagacccgtggcgaaagttttgtcaacggatgattatattgtcgggacggacttgtattttcatagctccacagaccggttgcttacggtcggacatccgttctttgacgtactgagtacggaccaaaatacagttgatgtgcctaaggtgtccggcaatcaatttagagtttttcggctgaatttgccggacccaaatcaattcgcactgatagacacgagtatttataacccggaacatgagcggttggtttggaggctcgtcggtattgaaatcgatcgcggtgggcccctgggtatagggagtactggtcaccccctctttaacaaattgcaagacactgaaaaccccagcgtgtacaacgggctcatctctgatcaaaaggataaccgcatgaacgtagctttcgatccgaagcagaaccaactcttcatagtaggctgcaagccagctgtaggccaacattgggataaggctgaaccttgcccgaataccaggccacctcctggctcttgcccgccgctgaaactcgtgcactcaactattgaagacggggatatgtctgacattgggttgggaaatataaatttttccgacttgtccgatgataagagttccgcccctctcgagattattaactcaaagtgtaagtggcccgacttcgccctcatgacaaaagatctgttcggagatagcgcctttttctttgggcgacgggagcaactttacgcgcgacaccaatggtgtcgagatggcctggtaggggacgctataccagatgagcatttctacttcaaccctaacggacaggaccctaagccgccacagtaccagcttggatcctccatatactttactatacctagcggttcccttacatctagcgaatctaatatatttggtagaccctactggctgcacagggcccagggcgccaataacgggatcgcctggggaaatcagctgttcgttacgctccttgataatacgcataacactaacttcaccatctctgtttctactgaaagccaaacgacatatgacaaaaataaatttaaagtgtaccttcgacatgctgaggagattgaaattgagatcgtctgtcaactctgcaaagtcccacttgaagcggatatattggctcatctttatgctatggacccaagcatactcgacaactggcagctcgcgtttgtcccagcgcctcctcagacgttggaggacacataccgatacatacgcagtatggcaaccatgtgcccggcggacgtgccgccaaaagaacctgaagacccctacaaggatctgcacttctggactataaacctcacggatagattcacatctgaacttgatcaaaccccgctgggtaagcggttcctgtaccaaatgggattgctgacgggtaataaaagactccgcactgactatattggcagtcctgtggctaaacgcaggcgcaccgtgaaaagcagcaaacgcaagaagtcatctgcaaagtaattctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcttcatatgtctagccaccatgcaggccaataaacggcgcaaaagagctgcggtagaagacatttacgctaaaggctgtacccagcctggaggatattgcccaccggatgtgaagaataaagtcgagggcaacacttgggcggatttccttttgaaagtttttggaagcgtcgtgtactttggcgggcttggtattggtacaggcaaaggaaccgggggctccactggttacacccccctcggtgggacggttggtagtagggggacaactaataccatcaaacctacgattcctcttgatccacttggtgtgccggatatcgtcacggtcgatcctatcgcgccggaagcggctagcattgttccgttggccgaaggcttgcctgaaccgggagtaatcgacacgggtacttcatttccggggcttgcagcggataacgaaaacatagttaccgtgctcgaccctttgagcgaagtcacgggcgtaggagagcaccccaacataatcaccggcggcactgccgattcacctgcgattttggacgttcagacatcacccccaccggcgaagaaaatactccttgatccatctatttcaaaaacgaccaccgcggttcaaactcacgcatcacacgtggatgcaaatttgaacatcttcgtagatgctcagagtttcggaacgcatgtgggctacacggaggatatacccctcgaagaaataaatctcaggtccgaatttgagttggaggactccgagcccaaaacgtccacgccctttgccgagcgagtgctcaataaaaccaaacaattgtacagtaagtacgtccagcaggtacctacgagacccgcagaatttgcgttgtacacgtctagattcgagtttgaaaatcctgcgtttgaggaggatgtaacaatggagtttgaaaacgatctggccgaaataggcgaaatcaccactccagcggttagtgacgttcgcatacttaatcggccgatttactccgagactgccgaccggacagtaagaataagcaggcttgggcagagggccggaatgaagaccagatcagggttggaaattgggcaaagagtacatttttactttgacttgtcagacattccccgcgaatcaattgaacttaacacatatgggaactattcccacgagtcaacgatagtcgatgaactgcttagctctacttttatcaacccgttcgagatgccggtcgacagtgagattttcgcagagaacgaattgcttgacccgctcgaagaagattttcgcgactcacatatagtggtcccgtacctcgaagacgaacagatcaatataactccaaccctgcctcctgggctcggattgaaggtatattccgacctctccgaacgggatctcctgatacactaccctgtgcaacacgcggacatcatggttccggacactccatacatccccgttcagccaccggatggagtattggtagatgataatgactattaccttcatcccggtctctatagtcggaagagaaaaagaagggtattgtaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 29 - protein, 516 aa, Gammapapillomavirus 16; identical to SEQ ID NO: 14

SEQ ID NO: 30 - protein, 509 aa, Gammapapillomavirus 16; identical to SEQ ID NO: 15

SEQ ID NO: 31 - DNA, 9232 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgaccggtctgcaatacctctttcttgctatgatggctctcaccctttccatactgttggcccaacaaccgccccctcatagctgtctccacagtcccgccatgtgcccgacgcttttgcttacttgtatcgttgaggtgtggataatgatctatatccttgcctgctgcgccggcaacgttaagaatgcaaatgtttttatctttcaaatggctgtatggttgccaggcccaaaccgattctacctccctccccaaccgatccaacgcaccttgaatactgaagaatatgtgagaagaacaagtacgttcctccatgcggctacagaccgacttcttacagtcggacaccctttttacaatattacaaatgctgacgggaaggaagtagttccgaaggtctcctctaaccaatttagggcatttcgagttcgcttcccgaaccccaatacttttgcattttgcgataagagtctttttaacccagataaagaaagactcgtttggggtataagaggaatcgaagtgtcacgcggccagccactcggcatcggcgtgacagggaatccattttttaacaaattcgacgacgctgaaaatccgtacaacggaattaataagaacaacatcaccgatcaagggtctgattctaggctctctatagcgtttgacccgaagcaaacacagttgctgattgtaggagccaagccggcgaaaggggaatattgggatgtcgccgcaacatgtgagaatccaccgctgacgaaggcagacgacaagtgtcccgccctcgagttgaaatcttcttacatcgaagatgcagatatgtccgacatcgggttggggaatctgaacttctctactttgcagcgcaataagtccgacgcgccgctggacattgtcgacagtatttgcaaatatcctgactatttgcagatgatagaagaactgtacggcgatcacatgtttttctacgtgcggcgggaggcgctttacgcgcggcacattatgcagcatgctggaaagatggatgcagagcaatttccaacctctctttacattgactcttccgttgaaggtgagaaacttaatagtctccaacggacagataggtatttcatgactccctcaggctcactggtcgcgacggagcagcagctgttcaaccgacccttttggcttcaacgaagccaaggtcacaataacggcatactttggcataacgaagcctttgtcacccttgttgatactactagaggtacaaacttcactatatctgtccctgaaggtgacgcctcctcatacaacaatagtaaatttttcgaatttcttagacatacggaagagttccagttggcatttatacttcaactctgcaaggttgacttgacccccgaaaatctcgcatacatacataccatggacccatctattattgaagattggcacctcgcagtcacttccccgcctaactccgtactggaggaccactatcgatatatcctcagtatagcaacaaaatgtcctagcaaggacgcggacgatacgagcacagacccatataaagatctcaagttttgggaagttgacctccgagatcgaatgaccgaacagcttgaccaaactccgcttggcagaaagtttctcttccagacgggaatcactcagagttctagtaacaagcgggtctccactcaatcaaccgcattgaccacgtatcgacgccccactaaaaggcgaaggaaggcataattctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcttcatatgtctagccaccatgctggctaggcaaagggtgaagcgggctaacccggagcagttgtataagacatgcaaagccacgggtggggattgtcctcccgatgtaataaagcggtacgaacagacaacgccggccgacagtattttgaagtacgggagtgtaggtgtcttctttggtggcctcggcattgggaccggtagaggaggtgggggcacagtccttggagccggggcagtgggaggcaggccttcaattagctcaggagcgattgggccacgggacatcctgccgatcgaatccggagggccgagcctggcggaggagattccgcttttgcctatggcgccccgagtacccagacccactgatcctttcaggccatccgtcctcgaggagccctttataatacggcctccagaacgcccaaatatcttgcatgagcaaaggttccccacggacgctgccccatttgacaatgggaacaccgaaatcacaacaattccatcacagtatgatgtctctggagggggtgttgatatccagataatcgagctgccatccgttaatgacccaggccctagcgtcgttacgcgcactcagtacaataaccccacatttgaggttgaagtcagtacagatatatctggagaaaccagtagtaccgataatattattgttggcgctgagtcagggggtacgtcagtaggagacaatgcggaactgataccattgctcgacatttctcggggtgatactatagataccacaatccttgcaccgggagaggaagagactgcgtttgtaacgagcacccccgagagggttcctatccaggagagactgccaataagaccgtacggcagacaataccagcaggtgagagtcacggaccctgaattcttggattcagctgcggttctcgttagccttgagaatccggtttttgatgctgacattactcttactttcgaggatgatcttcagcaagcactgcgatccgatacagaccttagggacgtgcggcggcttagtaggccttattatcagcgccgcacgaccggactcagagtttcccgcctcggtcagcgaagggggacaattagtaccaggtcaggtgtgcaggtgggatctgctgcccacttcttccaagacatctccccgatcggacaggcgatagaaccgattgacgcaattgagctggatgttttgggcgagcaatctggtgagggcactatcgtgcggggagatccaacgccttccattgaacaagatattggcctcacagcacttggtgacaacatcgagaacgaattgcaagagatagatcttctcacggcagacggcgaagaagatcaagagggtcgggacctgcaattggtgttctccaccggaaacgatgaggtggtggatatcatgacgataccaattcgagccggtggtgatgaccgccccagcgtatttatcttcagcgacgatggcacgcacattgtttaccccacatctacaacggcaactacgccgctcgtcccggctcaaccgagtgatgtaccatacattgtcgtagatttgtactcaggcagtatggattacgacattcacccatccctgctccgaaggaagcgaaagaaacggaaaagggtatacttctccgatggacgagttgcatcacgcccgaagtaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 32 - protein, 583 aa, Nupapillomavirus 1
MTGLQYLFLAMMALTLSILLAQQPPPHSCLHSPAMCPTLLLTCIVEVWIMIYILACCAGNVKNANVFIFQMAVWLPGPNRFYLPPQPIQRTLNTEEYVRRTSTFLHAATDRLLTVGHPFYNITNADGKEVVPKVSSNQFRAFRVRFPNPNTFAFCDKSLFNPDKERLVWGIRGIEVSRGQPLGIGVTGNPFFNKFDDAENPYNGINKNNITDQGSDSRLSIAFDPKQTQLLIVGAKPAKGEYWDVAATCENPPLTKADDKCPALELKSSYIEDADMSDIGLGNLNFSTLQRNKSDAPLDIVDSICKYPDYLQMIEELYGDHMFFYVRREALYARHIMQHAGKMDAEQFPTSLYIDSSVEGEKLNSLQRTDRYFMTPSGSLVATEQQLFNRPFWLQRSQGHNNGILWHNEAFVTLVDTTRGTNFTISVPEGDASSYNNSKFFEFLRHTEEFQLAFILQLCKVDLTPENLAYIHTMDPSIIEDWHLAVTSPPNSVLEDHYRYILSIATKCPSKDADDTSTDPYKDLKFWEVDLRDRMTEQLDQTPLGRKFLFQTGITQSSSNKRVSTQSTALTTYRRPTKRRRKA

SEQ ID NO: 33 - protein, 554 aa, Nupapillomavirus 1
MLARQRVKRANPEQLYKTCKATGGDCPPDVIKRYEQTTPADSILKYGSVGVFFGGLGIGTGRGGGGTVLGAGAVGGRPSISSGAIGPRDILPIESGGPSLAEEIPLLPMAPRVPRPTDPFRPSVLEEPFIIRPPERPNILHEQRFPTDAAPFDNGNTEITTIPSQYDVSGGGVDIQIIELPSVNDPGPSVVTRTQYNNPTFEVEVSTDISGETSSTDNIIVGAESGGTSVGDNAELIPLLDISRGDTIDTTILAPGEEETAFVTSTPERVPIQERLPIRPYGRQYQQVRVTDPEFLDSAAVLVSLENPVFDADITLTFEDDLQQALRSDTDLRDVRRLSRPYYQRRTTGLRVSRLGQRRGTISTRSGVQVGSAAHFFQDISPIGQAIEPIDAIELDVLGEQSGEGTIVRGDPTPSIEQDIGLTALGDNIENELQEIDLLTADGEEDQEGRDLQLVFSTGNDEVVDIMTIPIRAGGDDRPSVFIFSDDGTHIVYPTSTTATTPLVPAQPSDVPYIVVDLYSGSMDYDIHPSLLRRKRKKRKRVYFSDGRVASRPK

SEQ ID NO: 34 - DNA, 8728 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggcgctgtggagaccctccgacaataccgtttatctccctccaccgtcagttgctcgggttgtaaatactgacgattacgtcacacgaaccagcattttttaccacgctgggagttcacggctcctcacggtgggaaacccctattttcgagtccccgccggaggcggtaacaagcaggatatcccgaaagtgtctgcctatcagtaccgggtgtttcgagtacagctccccgacccgaataagtttgggcttccagatacatccatctacaatcctgaaacgcaacggcttgtatgggcctgtgcgggcgtggaaataggaagaggccaaccgctgggagttggactgagcggtcacccattttacaacaaattggatgatacggagagttcacacgcggcaacctcaaatgtttccgaagacgtcagggacaatgtatcagtggattacaagcaaacacaactctgcattctgggatgtgcgcctgcaatcggtgaacactgggctaaaggaacagcttgtaagtctcgaccactcagtcagggtgactgtccaccacttgaactcaaaaatactgtgctcgaggatggggacatggtggataccgggtatggtgcgatggatttttcaacactgcaagatactaagtgcgaagttccccttgacatttgtcaaagtatctgcaaatacccggattacctccagatgagcgctgacccgtacggtgactcaatgtttttttgtcttcgacgcgaacaactcttcgcccgccacttctggaatcgggctggaacgatgggtgataccgttccccaatcattgtatataaagggtacaggtatgcgcgcttcaccaggctcctgtgtgtactctccgtccccctccggttctatagtaactagtgactctcagcttttcaacaaaccatactggcttcataaggcgcaaggccataataatggagtctgctggcacaaccagttgttcgtgacagttgtggatacgacgagaagtacgaaccttactatctgtgcatcaacacagtcccctgttccgggccaatacgatgcaactaagtttaaacaatactctcgacacgtagaagagtatgatctgcaattcatatttcagttgtgcacaataacactgacggcagatgtcatgtcatacatccactcaatgaattccagcattctggaggattggaatttcggggtcccgccgcccccaaccacctctcttgtagatacataccgattcgtacaaagcgtggcaatcacatgtcaaaaagatgcggcaccagcagaaaataaagacccctatgacaaactgaagttctggaatgtggaccttaaagaaaaatttagcttggaccttgaccaataccctttgggtaggaaatttctcgtgcaagcaggcttgcgccggaaaccgaccattggaccacgcaagcgcagtgcgccgagcgcaaccacaagtagtaagcctgcgaagagggttcgcgtgcgcgccagaaagtaattctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcttcatatgtctagccaccatggtgagccatcgagcggccagacgcaaaagggcgagcgtaaccgacttgtataaaacttgcaaacaatcagggacttgtccaccggacgtggtccccaaggtggaaggcaccacactcgccgataagatactccaatggtccagccttggtatatttcttggtggcctggggatcggaaccggatctggaactggtgggcgaacgggctacattccactggggggaagaagcaacaccgttgtcgatgtaggacctacgagacctccggtagttatagagcccgttggacccaccgatccgagcattgtaacgttgatcgaggactctagcgtggtcacctcaggtgcaccacgacctacctttacaggcacatctggatttgacataaccagcgccgggaccactactccagcggtactggacataacgccaagttccacgtccgtgagcatttccactactaactttacaaatcctgccttttctgaccctagcataatagaggtgccccaaacgggtgaggttgcggggaacgtcttcgttggcacgccgacttcaggaacccatggttacgaggaaatacctcttcagacatttgcgtcatcaggcacgggcgaagagccaatatctagcacgcccctgcctactgttcgccgagtcgcagggcctaggctttattccagggcatatcaacaggtatctgttgccaatccggaatttctcacgagaccctcatcccttattacatatgacaatccagccttcgaacccgtagacacaactctgacgtttgaccccagatcagatgtcccagatagtgacttcatggatattatacggcttcatcgaccggcacttactagtagacgcggtaccgttaggttcagccgactgggccaaagggccacgatgttcacacgctctggcactcagataggcgctagggtacacttctaccacgatatctctccgattgcaccctctcccgaatatattgagctgcagccacttgtgtcagccaccgaggataatgacctgttcgacatctacgccgatgatatggacccggcagtgcccgttcctagccggagcactacctcctttgccttttttaagtacagccccactattagttctgcttctagttatagtaatgtaactgttcccctcacctcaagttgggatgtgccagtttataccggtcccgacattacccttccatcaacgacttctgtatggccgatcgtttctccaacagcaccagcgagtacgcaatacatcggcatccatggtacgcactactatctctggcccttgtattactttataccaaaaaagagaaagcgagtcccatacttcttcgcagacggcttcgttgcggcgtaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 35 - protein, 507 aa, Alphapapillomavirus 7
MALWRPSDNTVYLPPPSVARVVNTDDYVTRTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 36 - protein, 462 aa, Alphapapillomavirus 7; identical to SEQ ID NO: 12

SEQ ID NO: 37 - DNA, 8848 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggctgtctggttgccggcgcaaaacaaattttatctgccgccacaacctataactaggattctctccacggatgagtatgtcaccaggaccaatctcttctatcacgctactagcgaacgattgctgcttgttgggcatccactttttgaaataagcagcaaccaaaccgttacaattcctaaggttagcccaaatgcctttagggtctttcgcgttcgattcgcagaccctaacagatttgccttcggagataaggcgatcttcaaccctgaaacagaaaggctcgtgtggggccttcggggtatcgaaatcggtcggggccaaccactggggattggaataaccggtcacccattgcttaataaactggatgatgccgaaaatccgactaactacatcaatacgcatgcgaacggggatagtcggcagaatacggccttcgatgccaagcaaacacaaatgtttctggtggggtgcactccagctagtggcgaacactggactagctccagatgcccgggtgagcaggtcaagctgggggactgtcctcgggtacaaatgattgaatcagtaatcgaagatggcgacatgatggacattggtttcggtgcgatggattttgcggcactccaacaagataaatctgatgtaccactcgatgtagtacaagctacatgtaagtatccggattatataaggatgaatcatgaagcatatggcaactcaatgttttttttcgcaagaagggagcaaatgtatacacggcatttttttacacggggaggtagcgtaggagataaggaagcagtaccgcagtctctgtacctgacagctgatgccgagccccggactaccctggcgacgaccaactacgtcggcacaccatctgggtcaatggtatcatcagacgtccagctgttcaatcgatcctactggcttcagaggtgccagggacaaaacaatgggatatgttggcggaaccagttgtttattactgtgggtgacaatactcgaggaacgtcactgagcatatcaatgaagaataacgcctccaccacgtatagtaacgcgaattttaatgacttcctgcgacatacggaggagtttgatctttccttcatagttcaactctgtaaagtgaagctcacgccagaaaacttggcttatatccatactatggatccgaatatcctggaggattggcagctgtcagtgagtcagccccctaccaatccccttgaagatcaataccggttcctgggcagtagcctcgcggccaagtgcccggagcaagccccacccgagccacagaccgacccatactctcaatataaattctgggaagtggacctgactgaacgaatgtctgagcaacttgaccaatttcccctggggcggaagtttctgtatcagagcggcatgacgcaacgaaccgcgacatcctccaccactaaaagaaagacggttcgagtgtctacatccgcaaaacggcgcaggaaagcgtagttctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcttcatatgtctagccaccatgtatcggctgcgccgaaagagggctgcccccaaagacatatacccaagttgtaaaatttccaacacttgcccgcctgatatacaaaataagatagagcacacaaccattgcagataaaattttgcaatacggctcactgggcgtcttcttgggtggtcttgggataggtacagctaggggcagcggagggcgcatcggatatactcccctgggagaaggcggcggggttagggtagccacccgccctacgcccgtcagacctacgattcccgtggagacagtcggacctagtgaaatcttccctattgacgtggtggatccaactggccctgcagttatccccctccaagacttgggacgagactttcctataccgaccgttcaagtaatcgcagaaatacatccaatcagcgatatccctaacattgtagcgtcttcaacgaacgagggggaatccgctatcctggatgtgctccagggttctgccacgatacgcaccgtttccaggacccaatataataatccatcttttacagttgcttccacctctaacatttccgccggggaagccagcacgtcagacatcgtctttgtgtccaacggttctggtgacagagtggtaggggaagacataccgttggtagaactcaacttgggactcgaaaccgacacaagttcagtagtccaagagactgcgttctcctccagtacccctatcgccgaacggccctctttccggcccagtcggttttataaccgacgactctatgagcaagtccaggtccaggatcctcgcttcgttgaacagccacagagcatggtgactttcgataatcccgctttcgaaccggaactggatgaagtctcaattatatttcagcgcgatctcgatgcattggcccaaactccagtaccagaatttcgcgacgtggtgtacctcagtaagccaacattttccagagagcctgggggtcgactccgagtatccaggttgggcaagagctcaactatcaggaccaggcttggaaccgcaattggggctagaactcacttcttttacgatctgtccagtattgcgcctgaagattctatagaacttcttcccctcggagagcactcacaaacaacggtgatctcttccaatttgggagacacagcatttatacagggagaaactgctgaagacgaccttgaggtgattagtctggaaacaccgcaactctactccgaggaggaactgctcgacaccaatgagtctgtaggcgagaaccttcaattgactataactaacagtgaaggcgaagttagtatacttgacctcacacagtctcgcgtgcgaccaccgttcggcacagaggatacctctttgcatgtatattaccctaattcaagtaagggaactcccataattaacccagaggagtcttttactcctcttgttataatagctttgaataacagtacgggagattttgaactgcatcccagtttgcggaagcgcaggaagagagcgtatgtataagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 38 - protein, 502 aa, Mupapillomavirus 1
MAVWLPAQNKFYLPPQPITRILSTDEYVTRTNLFYHATSERLLLVGHPLFEISSNQTVTIPKVSPNAFRVFRVRFADPNRFAFGDKAIFNPETERLVWGLRGIEIGRGQPLGIGITGHPLLNKLDDAENPTNYINTHANGDSRQNTAFDAKQTQMFLVGCTPASGEHWTSSRCPGEQVKLGDCPRVQMIESVIEDGDMMDIGFGAMDFAALQQDKSDVPLDVVQATCKYPDYIRMNHEAYGNSMFFFARREQMYTRHFFTRGGSVGDKEAVPQSLYLTADAEPRTTLATTNYVGTPSGSMVSSDVQLFNRSYWLQRCQGQNNGICWRNQLFITVGDNTRGTSLSISMKNNASTTYSNANFNDFLRHTEEFDLSFIVQLCKVKLTPENLAYIHTMDPNILEDWQLSVSQPPTNPLEDQYRFLGSSLAAKCPEQAPPEPQTDPYSQYKFWEVDLTERMSEQLDQFPLGRKFLYQSGMTQRTATSSTTKRKTVRVSTSAKRRRKA

SEQ ID NO: 39 - protein, 507 aa, Mupapillomavirus 1; identical to SEQ ID NO: 9

SEQ ID NO: 40 - DNA, 8755 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgagcctgtggctgcccagcgaggccaccgtgtacctgccccccgtgcccgtgagcaaggtggtgagcaccgacgagtacgtggccaggaccaacatctactaccacgccggcaccagcaggctgctggccgtgggccacccctacttccccatcaagaagcccaacaacaacaagatcctggtgcccaaggtgagcggcctgcagtacagggtgttcaggatccacctgcccgaccccaacaagttcggcttccccgacaccagcttctacaaccccgacacccagaggctggtgtgggcctgcgtgggcgtggaggtgggcaggggccagcccctgggcgtgggcatcagcggccaccccctgctgaacaagctggacgacaccgagaacgccagcgcctacgccgccaacgccggcgtggacaacagggagtgcatcagcatggactacaagcagacccagctgtgcctgatcggctgcaagccccccatcggcgagcactggggcaagggcagcccctgcaccaacgtggccgtgaaccccggcgactgcccccccctggagctgatcaacaccgtgatccaggacggcgacatggtggacaccggcttcggcgccatggacttcaccaccctgcaggccaacaagagcgaggtgcccctggacatctgcaccagcatctgcaagtaccccgactacatcaagatggtgagcgagccctacggcgacagcctgttcttctacctgaggagggagcagatgttcgtgaggcacctgttcaacagggccggcgccgtgggcgagaacgtgcccgacgacctgtacatcaagggcagcggcagcaccgccaacctggccagcagcaactacttccccacccccagcggcagcatggtgaccagcgacgcccagatcttcaacaagccctactggctgcagagggcccagggccacaacaacggcatctgctggggcaaccagctgttcgtgaccgtggtggacaccaccaggagcaccaacatgagcctgtgcgccgccatcagcaccagcgagaccacctacaagaacaccaacttcaaggagtacctgaggcacggcgaggagtacgacctgcagttcatcttccagctgtgcaagatcaccctgaccgccgacgtgatgacctacatccacagcatgaacagcaccatcctggaggactggaacttcggcctgcagcccccccccggcggcaccctggaggacacctacaggttcgtgaccagccaggccatcgcctgccagaagcacaccccccccgcccccaaggaggaccccctgaagaagtacaccttctgggaggtgaacctgaaggagaagttcagcgccgacctggaccagttccccctgggcaggaagttcctgctgcaggccggcctgaaggccaagcccaagttcaccctgggcaagaggaaggccacccccaccaccagcagcaccagcaccaccgccaagaggaagaagaggaagctgtgattctagtgtacgtagccagcccccgattgggggcgacactccaccatagatcactcccctgtgaggaactactgtcttcacgcagaaagcgtctagccatggcgttagtatgagagtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcttcatatgtctagccaccatgaggcacaagaggagcgccaagaggaccaagagggccagcgccacccagctgtacaagacctgcaagcaggccggcacctgcccccccgacatcatccccaaggtggagggcaagaccatcgccgaccagatcctgcagtacggcagcatgggcgtgttcttcggcggcctgggcatcggcaccggcagcggcaccggcggcaggaccggctacatccccctgggcaccaggccccccaccgccaccgacaccctggcccccgtgaggccccccctgaccgtggaccccgtgggccccagcgaccccagcatcgtgagcctggtggaggagaccagcttcatcgacgccggcgcccccaccagcgtgcccagcatcccccccgacgtgagcggcttcagcatcaccaccagcaccgacaccacccccgccatcctggacatcaacaacaccgtgaccaccgtgaccacccacaacaaccccaccttcaccgaccccagcgtgctgcagccccccacccccgccgagaccggcggccacttcaccctgagcagcagcaccatcagcacccacaactacgaggagatccccatggacaccttcatcgtgagcaccaaccccaacaccgtgaccagcagcacccccatccccggcagcaggcccgtggccaggctgggcctgtacagcaggaccacccagcaggtgaaggtggtggaccccgccttcgtgaccacccccaccaagctgatcacctacgacaaccccgcctacgagggcatcgacgtggacaacaccctgtacttcagcagcaacgacaacagcatcaacatcgcccccgaccccgacttcctggacatcgtggccctgcacaggcccgccctgaccagcaggaggaccggcatcaggtacagcaggatcggcaacaagcagaccctgaggaccaggagcggcaagagcatcggcgccaaggtgcactactactacgacctgagcaccatcgaccccgccgaggagatcgagctgcagaccatcacccccagcacctacaccaccaccagccacgccgccagccccaccagcatcaacaacggcctgtacgacatctacgccgacgacttcatcaccgacaccagcaccacccccgtgcccagcgtgcccagcaccagcctgagcggctacatccccgccaacaccaccatccccttcggtggcgcctacaacatccccctggtgagcggccccgacatccccatcaacatcaccgaccaggcccccagcctgatccccatcgtgcccggcagcccccagtacaccatcatcgccgacgccggcgacttctacctgcaccccagctactacatgctgaggaagaggaggaagaggctgccctacttcttcagcgacgtgagcctggccgcctgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 41 - DNA, 9504 bp, Artificial Sequence
  Description of Artificial Sequence Synthetic polynucleotide
taatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatacattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactataagctgcgagcaacttcacttgggtatgccggcggtagcgctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagtaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggactgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgggcccgccccaactggggtaacctttgagttctctcagttggggg

SEQ ID NO: 42 - DNA, 11298 bp, Artificial Sequence
  Description of Artificial Sequence Synthetic polynucleotide
gatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaaggcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatacattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactataagctgcgagcaacttcacttgggtatgccggcggtagcgctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgtccgaagtcgagttttcccatgagtactggatgagacacgcattgactctcgcaaagagggcttgggatgaacgcgaggtgcccgtgggggcagtactcgtgcataacaatcgcgtaatcggcgaaggttggaataggccgatcggacgccacgaccccactgcacatgcggaaatcatggcccttcgacagggagggcttgtgatgcagaattatcgacttatcgatgcgacgctgtacgtcacgcttgaaccttgcgtaatgtgcgcgggagctatgattcactcccgcattggacgagttgtattcggtgcccgcgacgccaagacgggtgccgcaggttcactgatggacgtgctgcatcacccaggcatgaaccaccgggtagaaatcacagaaggcatattggcggacgaatgtgcggcgctgttgtccgacttttttcgcatgcggaggcaggagatcaaggcccagaaaaaagcacaatcctctactgactctggtggttcttctggtggttctagcggcagcgagactcccgggacctcagagtccgccacacccgaaagttctggtggttcttctggtggttcttccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcaggtctttaacgcccagaaaaaagcacaatcctctactgactctggtggttcttctggtggttctagcggcagcgagactcccgggacctcagagtccgccacacccgaaagttctggtggttcttctggtggttctgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagat

SEQ ID NO: 43 - DNA, 10938 bp, Artificial Sequence
  Description of Artificial Sequence Synthetic polynucleotide
accaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtcggaagcggataagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatacattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactataagctgcgagcaacttcacttgggtatgccggcggtagcgctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttatgtacgggccagatatacgcgtttaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggccgcggccgctaatacgactcactatagggagagccgccaccatgaaacggacagccgacggaagcgagttcgagtcaccaaagaagaagcggaaagtcagcagtgaaaccggaccagtggcagtggacccaaccctgaggagacggattgagccccatgaatttgaagtgttctttgacccaagggagctgaggaaggagacatgcctgctgtacgagatcaagtggggcacaagccacaagatctggcgccacagctccaagaacaccacaaagcacgtggaagtgaatttcatcgagaagtttacctccgagcggcacttctgcccctctaccagctgttccatcacatggtttctgtcttggagcccttgcggcgagtgttccaaggccatcaccgagttcctgtctcagcaccctaacgtgaccctggtcatctacgtggcccggctgtatcaccacatggaccagcagaacaggcagggcctgcgcgatctggtgaattctggcgtgaccatccagatcatgacagccccagagtacgactattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcaccattgctctgcagtcttgtcactatcagcggctgcctcctcatattctgtgggctacaggcctgaagtctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctaaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagc

SEQ ID NO: 44 - DNA, 9232 bp, Artificial Sequence
  Description of Artificial Sequence Synthetic polynucleotide
ccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaatccggagtatacggatccgccaccatggtgtcccacagagccgccagacggaagcgggccagcgccaccgacctgtatcggacctgtaagcagagcggcacctgcccccctgatgtggtcgacaaggtggagggcaccacactggccgacaagatcctgcagtggaccagcctgggcatcttcctgggcggcctgggcattggcaccggcacaggcaccggcggcagaaccggctacatccccctcggcggcagacccaacaccgtggtggacgtgtcccccgccagaccccccgtggtcatcgagcccgtgggccccagcgagcccagcatcgtgcagctggtcgaggacagcagcgtgatcaccagcggcacccccgtgcccaccttcaccggcaccagcggcttcgagattacctctagctccaccaccacccctgccgtgctggacatcacccccagcagcggcagcgtgcagatcacctccacctcctacaccaaccccgccttcacagacccaagcctgatcgaggtgccccagaccggcgagacaagcggcaacatcttcgtgagcacccccacctccggcacacacggatacgaggaaatccccatggaagtgttcgccacccacggcaccgggaccgagcccatcagcagcacccctacccctggcatctctcgggtggcaggacctcggctgtactctagggctcaccagcaggtccgggtgtccaacttcgacttcgtgacccaccccagcagcttcgtgaccttcgacaaccctgccttcgagcctgtggacaccaccctgacctacgaggccgccgatatcgcccccgaccccgacttcctggacatcgtgcggctgcacagacccgccctgaccagccggaagggcaccgtgcggttctctcggctcggcaagaaagccacaatggtcaccagacggggcacccagatcggcgcccaggtgcactactaccacgacatcagctctatcgcccctgccgagagcatcgagctgcagcccctggtgcacgccgagcccagcgacgcctccgacgccctgttcgacatctacgccgacgtggacaacaacacctacctggacaccgccttcaacaacacccgggacagcggcaccacctacaacaccggcagcctccccagcgtggccagcagcgccagcaccaagtacgccaacaccaccatccctttcagcaccagctggaacatgcccgtgaacaccggccctgatatcgctctgcccagcaccaccccccagctgcctctggtgcccagcggcccaatcgacacaacctacgccatcaccatccagggcagcaactactacctgctgcccctgctgtacttcttcctgaagaagcggaagagaatcccctacttcttcagcgacggctacgtggccgtgtgatagtctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctgcctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtgatatcaaagatccgccaccatggcaatgtggagaagcagcgacagcatggtgtacctgccccctcccagcgtggccaaggtggtcaacaccgacgactacgtgacccggaccggcatctactactacgccggcagctctcggctgctgaccgtgggccacccctacttcaaagtgggcatgaacggcggcagaaagcaggacatccccaaggtgtccgcctaccagtaccgggtgttcagagtgaccctgcccgaccccaacaagttcagcatccccgacgccagcctgtacaaccccgagacacagcggctggtctgggcctgcgtgggcgtggaagtgggcagaggccagcccctgggcgtgggcatcagcggccaccccctgtacaacagacaggacgacaccgagaacagccccttcagcagcaccaccaacaaggacagccgggacaacgtgtccgtggactacaagcagacccagctgtgcatcatcggctgcgtgcctgccattggcgagcactggggcaagggcaaggcctgcaagcccaacaatgtgtccaccggcgactgcccccctctggaactggtcaacacacccatcgaggacggcgacatgatcgacaccggctacggcgccatggacttcggcgccctgcaggaaaccaagagcgaggtccccctggacatctgccagagcatctgcaagtaccccgactacctgcagatgagcgccgacgtgtacggcgactccatgttcttttgcctgcggcgggagcagctgttcgcccggcacttctggaacagaggcggcatggtcggcgacgctatccctgcccagctgtatatcaagggcaccgacatcagagccaaccccggcagctccgtgtactgccccagccccagcggctccatggtcaccagcgacagccagctgttcaacaagccctactggctgcacaaggcccagggccacaacaacggcatctgctggcacaaccagctgtttctgaccgtggtggacaccaccagaagcaccaacttcaccctgagcaccagcatcgagagcagcatccccagcacctacgacccctccaagttcaaagagtacacccggcacgtcgaggaatacgacctgcagttcatcttccagctgtgtaccgtgaccctgaccaccgacgtgatgagctacatccacaccatgaacagcagcatcctggacaactggaacttcgccgtggcccctccccctagcgccagcctggtggatacctacagatacctgcagagcgccgccatcacctgccagaaggacgcccctgcccccgagaagaaggacccctacgacggcctgaagttctggaacgtggacctgcgggagaagttcagcctggaactcgaccagtttcccctgggccggaagttcctgctgcaagccagagtcagacggaggcccaccatcggccccagaaagcggcctgccgctagcacctctagcagctccgccaccaagcacaagcggaagcgggtgtccaagtgatagtctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctg

SEQ ID NO: 45 - protein, 505 aa, Alphapapillomavirus 7
MAMWRSSDSMVYLPPPSVAKVVNTDDYVTRTGIYYYAGSSRLLTVGHPYFKVGMNGGRKQDIPKVSAYQYRVFRVTLPDPNKFSIPDASLYNPETQRLVWACVGVEVGRGQPLGVGISGHPLYNRQDDTENSPFSSTTNKDSRDNVSVDYKQTQLCIIGCVPAIGEHWGKGKACKPNNVSTGDCPPLELVNTPIEDGDMIDTGYGAMDFGALQETKSEVPLDICQSICKYPDYLQMSADVYGDSMFFCLRREQLFARHFWNRGGMVGDAIPAQLYIKGTDIRANPGSSVYCPSPSGSMVTSDSQLFNKPYWLHKAQGHNNGICWHNQLFLTVVDTTRSTNFTLSTSIESSIPSTYDPSKFKEYTRHVEEYDLQFIFQLCTVTLTTDVMSYIHTMNSSILDNWNFAVAPPPSASLVDTYRYLQSAAITCQKDAPAPEKKDPYDGLKFWNVDLREKFSLELDQFPLGRKFLLQARVRRRPTIGPRKRPAASTSSSSATKHKRKRVSK

SEQ ID NO: 46 - protein, 470 aa, Alphapapillomavirus 7
MVSHRAARRKRASATDLYRTCKQSGTCPPDVVDKVEGTTLADKILQWTSLGIFLGGLGIGTGTGTGGRTGYIPLGGRPNTVVDVSPARPPVVIEPVGPSEPSIVQLVEDSSVITSGTPVPTFTGTSGFEITSSSTTTPAVLDITPSSGSVQITSTSYTNPAFTDPSLIEVPQTGETSGNIFVSTPTSGTHGYEEIPMEVFATHGTGTEPISSTPTPGISRVAGPRLYSRAHQQVRVSNFDFVTHPSSFVTFDNPAFEPVDTTLTYEAADIAPDPDFLDIVRLHRPALTSRKGTVRFSRLGKKATMVTRRGTQIGAQVHYYHDISSIAPAESIELQPLVHAEPSDASDALFDIYADVDNNTYLDTAFNNTRDSGTTYNTGSLPSVASSASTKYANTTIPFSTSWNMPVNTGPDIALPSTTPQLPLVPSGPIDTTYAITIQGSNYYLLPLLYFFLKKRKRIPYFFSDGYVAV

SEQ ID NO: 47 - DNA, 10814 bp, Artificial Sequence
  Description of Artificial Sequence Synthetic polynucleotide
aaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgggggatccactagagccaccatggccctctggagaccctccgattccaccgtgtacttgcccccccccagcgtcgcacgcgtcgtgtctaccgacgactacgtcagcaggacctcaatcttctaccacgccgggtccagtaggctgctgaccgtggggaacccctacttccgcgtcgtgcccaacggcgccggcaacaagcaagccgtccccaaagtcagtgcctaccagtaccgcgtcttccgcgtggccctgccagaccccaacaagttcggcctgcccgacagcaccatctacaaccccgagacccagaggctcgtctgggcctgcgtgggcatggagatcggcaggggccaacccctgggcatcgggttgtccgggcaccccttctacaacaagctcgacgacaccgagtccgcccacgccgccaccgccgtcatcacccaggacgtccgcgacaacgtcagcgtcgactacaaacagacccaactctgcatcctgggctgcgtgcccgccatcggcgaacattgggcaaaggggaccttgtgcaagcccgcccagctccagcccggcgattgcccccccctcgagttgaagaatacaatcatcgaggacggcgacatggtcgacaccggctacggcgccatggacttctccaccctccaagacaccaaatgtgaagtccccctggatatctgccagagtatttgcaagtaccccgactacctccagatgagcgccgacccatacggcgacagcatgttcttctgtttgaggagggagcagctcttcgcccgccacttctggaaccgcgccggcgtcatgggcgataccgtgcccaccgatttgtacatcaaggggacctcagccaacatgagggagacaccggggtcctgcgtctacagtcccagcccatccgggagcatcatcaccagcgacagccagctgttcaacaagccctactggctgcacaaagcacaggggcacaataacggcatctgctggcacaaccaactcttcgtcaccgtggtcgataccacaaggtccaccaacctgaccctgtgcgcaagcacccagaaccccgtcccctccacctacgatcccaccaagttcaaacagtactcccgccacgtcgaagagtacgacctgcagttcatcttccaactctgtaccatcaccctgaccgccgaggtcatgagctacattcactccatgaactcctccatcctggagaactggaacttcggcgtgccccccccccccaccacctccctcgtcgacacctacaggttcgtccagagcgtcgccgtcacatgccagaaggacaccaccccccccgagaaacaggacccctacgacaagctgaagttctggaccgtcgatttgaaggagaagttcagtagtgacctcgaccagtacccattgggcaggaaattcctggtccaagccggcctgaggaggcgccccacaatcggccccaggaagaggcccgccgccagtaccagcaccgccagcaccgccagccgccccgcaaagcgcgtcaggatcaggtccaagaaatgagcccggtggatcccaatcaagctttttgcaaaagcctagggctcgaggaagcttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgctctagagccaccatggtcagtcatagggccgccaggaggaagagagcaagcgccaccgatctgtaccgcacctgcaaacagagtggcacctgtccacccgacgtcatcaataaggtcgaggggaccacactggccgacaagatcctgcaatggagctcattgggcatcttcctcggcgggttggggatcggcacagggtccggcagcggcgggaggaccggatacgtgccactgggcgggcgcagcaacaccgtcgtcgacgtcgggccaacccgcccccccgtcgtcatcgagcccgtgggccccaccgaccccagcatcgtcaccctcgtggaagacagttccgtcgtcgcaagcggcgcccccgtcccaaccttcaccggcacaagcggcttcgagatcaccagcagcggcaccacaacccccgccgtcctcgatattacccccaccgtcgatagcgtcagcatcagcagcacctccttcaccaacccagccttcagcgacccaagcatcatcgaggtcccacagaccggcgaagtcagcggcaacatcttcgtcggcacccccaccagcgggtctcacggctacgaagagatcccactgcagaccttcgccagcagcggcagcggcaccgagccaatctcctccacaccattgcccaccgtcagaagagtggccggcccaaggctctactcccgcgccaaccagcaagtcagggtcagtacaagccagttcctgacccacccaagcagcctcgtcaccttcgacaaccccgcctacgagccactcgatacaaccttgagtttcgaacccacatccaacgtccccgacagtgacttcatggacatcatcaggctccaccgccccgccctgagtagccgcagggggaccgtccgcttctcccgcctcggccagcgcgccacaatgttcaccaggtccggcaagcagatcggcggccgcgtgcacttctatcacgacatctctccaatcgccgccaccgaagagatcgagctccaacccctgatctccgccaccaacgactccgatctcttcgacgtgtacgccgattttccgccacccgccagtaccaccccctcaaccatccataagagcttcacctaccccaaatacagtctcacaatgcccagcaccgccgccagtagctattccaacgtcaccgtgcccctgaccagcgcctgggacgtgcccatctacaccgggcccgatatcatcctcccgagtcacacccccatgtggccctccaccagccccacaaacgccagtacaacaacatacatcggcatccacgggacccagtactacctgtggccctggtactactacttccccaagaagaggaagaggatcccatacttcttcgccgacgggttcgtcgccgcatgagcccgggacccagctttcttgtacaaagtggttcgatctagaatggctagtggatcccccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcagaattctatcaaatatttaaagaaaaaaaaattgtatcaactttctacaatctctttcagaagacagaagcagagggaatacttcctaaatcattcaactaggccagcattaccttaataccggaactagaaaatgacattacaagaaaagaaaacaacagaccaatatctctcatgaacaaagatacaaacattttcaacaaaatattagcaaaaagaatccaagaatgtatcaaaaaatatacaccacaaccaagtagaatttattccagatatgtaagggtggttcaacgtttgaaaatcaattaacgtaatttgtcccatcaacaggttaaagaagaaaatcacatggtcatattgatagacacagaaaaagcatttgacaaaatttaacacccattcatgatgcaatctctcagtaaactaggaatagaggaaaacttcctcagcttgaatgtaccttcctctcaattttgctatgaacctgaaactcctcttaaaaaataaagtttttcatttaaaaagaaaacaaaaaacatggaggagcgttgatgtatctcattttagaccaatcagctatggatagttaggcgacagcacagatagctgctgtacttctgtttctggcaatgttccagactacatttaaaaaatttttaattatagacttgtacttaatgttcaagaaaaatatgaaaatggctttgccgtgttaatgctactcttttttaaaaaaaactaaagttcaaactttatttatatttcattagttttttagctactgttctttttctgttctgggatctcattcagaatgccacattacatataattctcatgtctccttgggttcctcttagttttgacagttcctcagacttttcttatttttgatgaccttgacagttttgaggagtactggttagatatagggtaatggtttttaaagtatatttgtcatgatttatactggggtaagggtttggggaggaagcccatggggtaaagtactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttaacaaacagcttttttggggtgaacatattgactacaaatttatgtggaaagaaagtaccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctc

SEQ ID NO: 48 - protein, 513 aa, Alphapapillomavirus 7
MALWRPSDSTVYLPPPSVARVVSTDDYVSRTSIFYHAGSSRLLTVGNPYFRVVPNGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSIITSDSQLFNKPYWLHKAQGHNNGICWHNQLFVTVVDTTRSTNLTLCASTQNPVPSTYDPTKFKQYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGLRRRPTIGPRKRPAASTSTASTASRPAKRVRIRSKK

SEQ ID NO: 49 - protein, 463 aa, Alphapapillomavirus 7
MVSHRAARRKRASATDLYRTCKQSGTCPPDVINKVEGTTLADKILQWSSLGIFLGGLGIGTGSGSGGRTGYVPLGGRSNTVVDVGPTRPPVVIEPVGPTDPSIVTLVEDSSVVASGAPVPTFTGTSGFEITSSGTTTPAVLDITPTVDSVSISSTSFTNPAFSDPSIIEVPQTGEVSGNIFVGTPTSGSHGYEEIPLQTFASSGSGTEPISSTPLPTVRRVAGPRLYSRANQQVRVSTSQFLTHPSSLVTFDNPAYEPLDTTLSFEPTSNVPDSDFMDIIRLHRPALSSRRGTVRFSRLGQRATMFTRSGKQIGGRVHFYHDISPIAATEEIELQPLISATNDSDLFDVYADFPPPASTTPSTIHKSFTYPKYSLTMPSTAASSYSNVTVPLTSAWDVPIYTGPDIILPSHTPMWPSTSPTNASTTTYIGIHGTQYYLWPWYYYFPKKRKRIPYFFADGFVAA

SEQ ID NO: 50 - DNA, 9229 bp, Artificial Sequence
  Description of Artificial Sequence Synthetic polynucleotide
gaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaatccggagtatacggatccgccaccatggtgtcccacagagccgccagacggaagcgggccagcgccaccgacctgtacaagacctgcaagcagagcggcacctgccccagcgacgtgatcaacaaggtggagggcaccacactggccgacaagatcctgcagtggaccagcctgggcatcttcctgggcggcctgggcattggcaccggcagcggcacaggcggcagagccggctacatccccctcggcggcaagcccaacaccgtggtggacgtgtcccccgccagaccccccgtggtcatcgagcccgtgggccccaccgagcccagcatcgtgcagctggtcgaggacagcagcgtgatcacctctggcacacccgtccccaccttcaccggcaccagcggcttcgagatcaccagcagctccaccaccacccctgccgtgctggacatcacccccagcagcggcagcgtgcaggtgtccagcaccagcttcaccaaccccgccttcaccgaccccaccatcatcgaggtgccccagaccggcgaggtgtccggcaacgtgttcgtgagcacccccacctccggcactcacggctatgaggaaatccccatgcaggtgttcgccacccacggcacaggcacagaacctatcagcagcacccccatccctggcgtgtctcgggtggcaggaccccggctctactctagggctcaccagcaggtccgggtgtccaacttcgacttcgtgacccacccctctagcttcgtcaccttcgacaaccctgccttcgagcctgtggacaccactctgacctatgagcccgccgatatcgcccccgaccccgacttcctggacatcgtgcggctgcacagacccgccctgaccagcagacggggcaccgtgcggttcagcagagtgggcaagaaagccaccatgttcaccaggcgggggacccagatcggcgcccaggtgcactactaccacgacatcagcaatatcacaccagccgacagcatcgagctgcagcccctggtggcccccgagcaggccgaccccatggacaacctgtacgacatctacgctcccgatactgacaacaccaccgtgctggataccgccttccacaacgccacctttaccaccagatcccacatcagcgtgcccagcctggccagcgccgccagcaccacctacacaaacaccaccatccctctgggcaccgcctggaacacccccgtgaacaccggccctgacgtggtcctgcccagcacaacaccccagctgcctctgaccccctccacccccatcgacaccaccttcgccatcaccatctacggcagcaattactacctcctgcccctgctgttcttcctgctgaagaagcggaagcacctgccctactttttcaccgacggcatcgtggccagctgatagtctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctgcctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtgatatcaaagatccgccaccatggcactgtggagagccagcgacaacatggtgtacctgccccctcccagcgtggccaaggtggtcaacaccgacgactacgtgacccggaccggcatgtactactacgccggcacctctcggctcctgaccgtgggccacccctacttcaaggtgcccatgagcggcggcagaaagcagggcatccccaaggtgtccgcctaccagtaccgggtgttcagagtgaccctgcccgaccccaacaagttcagcgtgcccgagagcaccctgtacaaccccgacacccagcggatggtctgggcctgcgtgggcgtggagatcggcagaggccagcccctgggcgtgggcctgagcggccaccccctgtacaatcggctggacgacaccgagaacagccccttcagcagcaacaagaaccccaaggacagccgggacaacgtggccgtggactgcaagcagacccagctgtgcatcatcggctgcgtgcctgccattggcgagcactgggccaagggcaagagctgcaagcccaccaacgtgcagcagggcgactgcccccctctggaactggtcaacacacccatcgaggacggcgacatgatcgacaccggctacggcgccatggacttcggcaccctgcaggaaaccaagagcgaggtccccctggacatctgccagagcgtgtgcaagtaccccgactacctgcagatgagcgccgacgtgtacggcgacagcatgttcttttgcctgcggcgggagcagctgttcgcccggcacttctggaacagaggcggcatggtcggcgacaccatccccaccgacatgtacatcaagggcaccgacatcagagagacacccagcagctacgtgtacgcccccagccccagcggcagcatggtgtccagcgacagccagctgttcaacaagccctactggctgcacaaggcccagggccacaacaacggcatctgctggcacaaccagctgtttctgaccgtggtggacaccaccagaagcaccaacttcaccctgagcaccaccaccgacagcaccgtgcccgccgtgtacgacagcaataagttcaaagaatacgtgcggcacgtggaggaatacgacctgcagttcatcttccagctgtgtaccatcaccctgtccaccgacgtgatgagctacatccacaccatgaaccccgccatcctggacgactggaacttcggcgtggcccctccccctagcgccagcctggtggatacctacagatacctgcagagcgccgccatcacctgccagaaggacgcccctgcccccgtgaagaaggacccctacgacggcctgaacttctggaatgtggacctgaaagagaagttcagcagcgagctggaccagttccccctgggccggaagttcctgctgcaagccggcgtgcggagaaggcccaccatcggccccagaaagcggaccgccaccgcagccacaacctccacctccaagcacaagcggaagcgggtgtccaagtgatagtctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatg

SEQ ID NO: 51 - protein, 509 aa, Alphapapillomavirus 7
MALWRASDNMVYLPPPSVAKVVNTDDYVTRTGMYYYAGTSRLLTVGHPYFKVPMSGGRKQGIPKVSAYQYRVFRVTLPDPNKFSVPESTLYNPDTQRMVWACVGVEIGRGQPLGVGLSGHPLYNRLDDTENSPFSSNKNPKDSRDNVAVDCKQTQLCIIGCVPAIGEHWAKGKSCKPTNVQQGDCPPLELVNTPIEDGDMIDTGYGAMDFGTLQETKSEVPLDICQSVCKYPDYLQMSADVYGDSMFFCLRREQLFARHFWNRGGMVGDTIPTDMYIKGTDIRETPSSYVYAPSPSGSMVSSDSQLFNKPYWLHKAQGHNNGICWHNQLFLTVVDTTRSTNFTLSTTTDSTVPAVYDSNKFKEYVRHVEEYDLQFIFQLCTITLSTDVMSYIHTMNPAILDDWNFGVAPPPSASLVDTYRYLQSAAITCQKDAPAPVKKDPYDGLNFWNVDLKEKFSSELDQFPLGRKFLLQAGVRRRPTIGPRKRTATAATTSTSKHKRKRVSKSSWP

SEQ ID NO: 52 - protein, 466 aa, Alphapapillomavirus 7
GSATMVSHRAARRKRASATDLYKTCKQSGTCPSDVINKVEGTTLADKILQWTSLGIFLGGLGIGTGSGTGGRAGYIPLGGKPNTVVDVSPARPPVVIEPVGPTEPSIVQLVEDSSVITSGTPVPTFTGTSGFEITSSSTTTPAVLDITPSSGSVQVSSTSFTNPAFTDPTIIEVPQTGEVSGNVFVSTPTSGTHGYEEIPMQVFATHGTGTEPISSTPIPGVSRVAGPRLYSRAHQQVRVSNFDFVTHPSSFVTFDNPAFEPVDTTLTYEPADIAPDPDFLDIVRLHRPALTSRRGTVRFSRVGKKATMFTRRGTQIGAQVHYYHDISNITPADSIELQPLVAPEQADPMDNLYDIYAPDTDNTTVLDTAFHNATFTTRSHISVPSLASAASTTYTNTTIPLGTAWNTPVNTGPDVVLPSTTPQLPLTPSTPIDTTFAITIYGSNYYLLPLLFFLLKKRKHLPYFF

(19) United States
(12) Patent Application Publication
(10) Pub. No.: US 2023/0045095 A1
(43) Pub. Date: Feb. 9, 2023

(54) Compositions, Methods and Systems for the Delivery of Gene Editing Material to Cells

(71) Applicant: Massachusetts Institute of Technology, Cambridge, MA (US)

(72) Inventors: Omar Osama Abudayyeh, Cambridge, MA (US); Jonathan S. Gootenberg, Cambridge, MA (US)

(21) Appl. No.: 17/807,405

(22) Filed: Jun. 17, 2022

Related U.S. Application Data
(60) Provisional application No. 63/214,073, filed on Jun. 23, 2021.

(51) Int. Cl. C12N 15/86; C07K 14/005; C12N 15/11; C12N 9/22; C12N 15/90

(52) U.S. Cl. CPC C12N 15/86; C07K 14/005; C12N 15/11; C12N 9/22; C12N 15/907; C12N 2710/20042; C12N 2710/20022; C12N 2800/80; C12N 2310/20; C12N 2710/20051; A61K 48/00

(57) ABSTRACT

This disclosure provides compositions, methods, and systems comprising a papillomaviral delivery vehicle for the delivery of gene editing material to cells. The papillomaviral delivery vehicle comprises a papillomavirus-derived capsid and DNA encoding a gene editing material encapsulated by the capsid. The papillomaviral delivery vehicle can be transduced into a cell under conditions conducive for the cell to synthesize the gene editing material. The cell can comprise a polynucleotide target and the gene editing material can target the polynucleotide target. The polynucleotide target can be a DNA polynucleotide target or RNA polynucleotide target.

### CROSS REFERENCE TO RELATED APPLICATIONS

This application claims the priority benefit of U.S. Provisional Application No. 63/214,073, filed Jun. 23, 2021. The entirety of the application is hereby incorporated by reference.

### BACKGROUND

Gene editing requires the delivery of gene editing materials to cells. The delivery can be achieved using a delivery vehicle that comprises the gene editing materials and couples to targeted cells. Currently available delivery vehicles have a number of disadvantages such as a small payload capacity, a limited number of cells that can be targeted, a complex and expensive production, or a limited immunogenicity.

Thus, there is a need for better delivery vehicles to deliver gene editing materials to cells.

### SUMMARY

It has been discovered that a papillomaviral-derived capsid is useful for encapsulating a nucleic acid encoding a gene editing material and delivering it to cells where the gene editing material can edit nucleic acid targets.

In one aspect, the present application is directed to a method of delivering a material for editing a polynucleotide target in a cell, which comprises transducing the papillomaviral delivery vehicle into a cell comprising a polynucleotide target under conditions conducive for the cell to synthesize the gene editing material. The method further comprises allowing the gene editing material to edit the polynucleotide target.

In one exemplary embodiment, a papillomaviral delivery vehicle comprises the papillomavirus-derived capsid and DNA encoding a gene editing material encapsulated by the capsid. In particular embodiments, the capsid is derived from a mammalian papillomavirus. In particular embodiments, the capsid is derived from a human papillomavirus (HPV). In particular embodiments, the mammalian papillomavirus is selected from the group consisting of an HPV-1, an HPV-2, an HPV-3, an HPV-4, an HPV-5, an HPV-6, an HPV-7, an HPV-8, an HPV-9, an HPV-10, an HPV-11, an HPV-12, an HPV-13, an HPV-14, an HPV-15, an HPV-16, an HPV-17, an HPV-18, an HPV-19, an HPV-20, an HPV-21, an HPV-22, an HPV-23, an HPV-24, an HPV-25, an HPV-26, an HPV-27, an HPV-28, an HPV-29, an HPV-30, an HPV-31, an HPV-32, an HPV-33, an HPV-34, an HPV-35, an HPV-36, an HPV-37, an HPV-38, an HPV-39, an HPV-40, an HPV-41, an HPV-42, an HPV-43, an HPV-44, an HPV-45, an HPV-47, an HPV-48, an HPV-49, an HPV-50, an HPV-51, an HPV-52, an HPV-53, an HPV-54, an HPV-56, an HPV-57, an HPV-58, an HPV-59, an HPV-60, an HPV-61, an HPV-62, an HPV-63, an HPV-65, an HPV-66, an HPV-67, an HPV-68, an HPV-69, an HPV-70, an HPV-71, an HPV-72, an HPV-73, an HPV-74, an HPV-75, an HPV-76, an HPV-77, an HPV-78, an HPV-80, an HPV-81, an HPV-82, an HPV-83, an HPV-84, an HPV-85, an HPV-86, an HPV-87, an HPV-88, an HPV-89, an HPV-90, an HPV-91, an HPV-92, an HPV-93, an HPV-94, an HPV-95, an HPV-96, an HPV-97, an HPV-98, an HPV-99, an HPV-100, an HPV-101, an HPV-102, an HPV-103, an HPV-104, an HPV-105, an HPV-106, an HPV-107, an HPV-108, an HPV-109, an HPV-110, an HPV-111, an HPV-112, an HPV-113, an HPV-114, an HPV-115, an HPV-116, an HPV-117, an HPV-118, an HPV-119, an HPV-120, an HPV-121, an HPV-122, an HPV-123, an HPV-124, an HPV-125, an HPV-126, an HPV-127, an HPV-128, an HPV-129, an HPV-130, an HPV-131, an HPV-132, an HPV-133, an HPV-134, an HPV-135, an HPV-136, an HPV-137, an HPV-138, an HPV-139, an HPV-140, an HPV-141, an HPV-142, an HPV-143, an HPV-144, an HPV-145, an HPV-146, an HPV-147, an HPV-148, an HPV-149, an HPV-150, an HPV-151, an HPV-152, an HPV-153, an HPV-154, an HPV-155, an HPV-156, an HPV-157, an HPV-158, an HPV-159, an HPV-160, an HPV-161, an HPV-162, an HPV-163, an HPV-164, an HPV-165, an HPV-166, an HPV-167, an HPV-168, an HPV-169, an HPV-170, an HPV-171, an HPV-172, an HPV-173, an HPV-174, an HPV-175, an HPV-176, an HPV-177, an HPV-178, an HPV-179, an HPV-180, an HPV-181, an HPV-182, an HPV-183, an HPV-184, an HPV-185, an HPV-186, an HPV-187, an HPV-188, an HPV-189, an HPV-190, an HPV-191, an HPV-192, an HPV-193, an HPV-194, an HPV-195, an HPV-196, an HPV-197, an HPV-199, an HPV-200, an HPV-201, an HPV-202, an HPV-203, an HPV-204, an HPV-205, an HPV-206, an HPV-207, an HPV-208, an HPV-209, an HPV-210, an HPV-211, an HPV-212, an HPV-213, an HPV-214, an HPV-215, an HPV-216, an HPV-219, an HPV-220, an HPV-221, an HPV-222, an HPV-223, an HPV-224, an HPV-225, a MmuPV-1, and a variant thereof. In specific embodiments, the capsid comprises a L1 capsid protein. In specific embodiments, the capsid comprises a L2 capsid protein.

In specific embodiments, the L1 capsid protein comprises an amino acid sequence selected from the group consisting of: SEQ ID NO: 2, 5, 8, 11, 14, 17, 20, 23, 26, 29, 32, 35, 38, 45, 48, and 51.

In specific embodiments, the L2 capsid protein comprises an amino acid sequence selected from the group consisting of: SEQ ID NO: 3, 6, 9, 12, 15, 18, 21, 24, 27, 30, 33, 36, 39, 46, 49, and 52.

In another embodiment, the DNA encoding the gene editing material comprises a minicircle. In specific embodiments, the minicircle does not comprise a sequence of a bacterial origin.

In some embodiments, the gene editing material is selected from the group consisting of a nuclease, a nuclease coupled to a deaminase, a deaminase, a nickase, a transcriptase, a reverse transcriptase, an integration enzyme, an epigenetic modifier, a DNA methyltransferase, a guide RNA, a homology-directed repair (HDR) template, a reporter gene, a polynucleotide linked to a sequence complementary to an integration site, a split intein, a derivative thereof, and a combination thereof. In particular embodiments, the nuclease comprises a DNA-binding nuclease, a DNA-cleaving nuclease, a meganuclease, a zinc-finger nuclease (ZFN), a transcription activator-like effector nuclease (TALEN), a derivative thereof, or a combination thereof. In particular embodiments, the DNA binding nuclease comprises a clustered regularly interspaced short palindromic repeat (CRISPR)-associated (Cas) DNA-binding nuclease. In particular embodiments, the Cas DNA-binding nuclease comprises a Cascade (type I) nuclease, type III nuclease, a Cas9 nuclease, a Cas12 nuclease, a variant thereof, or a combination thereof.

In certain embodiments, the nuclease comprises an RNA-targeting nuclease, an RNA-binding nuclease, an RNA-cleaving nuclease, a derivative thereof, or a combination thereof. In particular embodiments, the nuclease comprises a Cas13a nuclease, a Cas13b nuclease, a Cas13c nuclease, a Cas13d nuclease, a Cas13e nucleases, a Cas7-11 nuclease, a variant thereof, or a combination thereof.

In some embodiments, the guide RNA comprises a single-guide RNA (sgRNA), a dual-guide RNA (dgRNA), a prime-editing guide RNA (pegRNA), a nicking-guide RNA (ngRNA), a derivative thereof, or a combination thereof.

In other embodiments, the reporter gene encodes a fluorescent protein. In particular embodiments, the fluorescent protein comprises a green fluorescent protein (GFP), a tdTomato protein, DsRed protein, a derivative thereof, or a combination thereof.

In some embodiments, the deaminase comprises an AncBE4 deaminase, an ABE7.10 deaminase, a derivative thereof, or a combination thereof.

In some embodiments, the gene-editing material comprises a single-stranded DNA editing material, while in other embodiments, the gene-editing material comprises a double-stranded DNA editing material.

In another aspect, the disclosure provides cell comprising the papillomaviral delivery vehicle. In specific embodiments, the cell is a eukaryotic cell. In specific embodiments, the cell is a mammalian cell. In specific embodiments, the cell is a human cell. In specific embodiments, the cell is a hematopoietic stem cell, a progenitor cell, a satellite cell, a mesenchymal progenitor cell, an astrocyte cell, a T-cell, a B cell, a hepatocyte cell, a heart cell, a muscle cell, a retinal cell, a renal cell, or a colon cell.

The disclosure also provides, a method of synthesizing a papillomaviral delivery vehicle, comprising transfecting a cell with a first vector encoding a papillomavirus-derived capsid under conditions conducive for the cell to synthesize the papillomavirus-derived capsid. The method further comprises transfecting the cell with a second vector encoding a DNA encoding a gene editing material under conditions conducive for the cell to replicate the second vector, allowing the cell to assemble the papillomaviral delivery vehicle. In specific embodiments, the papillomaviral delivery vehicle is isolated from the cells.

In another aspect, the disclosure provides a method of editing a polynucleotide target in a cell, the method comprises transducing a papillomaviral delivery vehicle into the cell comprising the polynucleotide target under conditions conducive for the cell to synthesize the gene editing material. The method further comprises allowing the gene editing material to edit the polynucleotide target. In specific embodiments, the polynucleotide target is a DNA. In specific embodiments, the polynucleotide target is a RNA. In specific embodiments, the method further comprises knocking down the polynucleotide target.

The disclosure also provides use of a papillomaviral delivery vehicle to edit a polynucleotide target in a cell is disclosed. In specific embodiments, the polynucleotide target is a DNA. In specific embodiments, the polynucleotide target is a RNA.

### BRIEF DESCRIPTION OF THE DRAWINGS

The present disclosure may be more fully understood from the following description, when read together with the accompanying drawings in which:

FIG. 1 is a tabular representation of commensal viruses in human tissues;

FIG. 2 is a graphic representation of viral vectors from human tissues;

FIG. 3 is a diagrammatic representation of families of papilloma viruses;

FIG. 4 is a schematic representation of assaying viruses for production, packaging, size, and cell type specificity;

FIG. 5 is a schematic representation of an HPV helper plasmid to generate HPV viral particles that requires only two genes;

FIG. 6 is a schematic representation of HPV production and purification;

FIG. 7A is a bar chart representation of common HPV titer;

FIG. 7B is a bar chart representation of transduce HEK293FT cells;

FIG. 8 is an energy landscape representation of HPVs transduce cells with varying efficiencies;

FIG. 9 is a bar chart representation of HPV packaged with plasmids;

FIG. 10 is a diagram representation of a panel of HPVs;

FIG. 11A is a bar chart representation of the qPCR titer of a panel of viruses;

FIG. 11B is a bar char representation of the transduction of HEK293FT cells;

FIG. 12 is an energy landscape representation of virus transduction of cell lines;

FIG. 13 is a schematic representation of the testing of HPV tropism in high throughput using PRISM;

FIG. 14 is a schematic representation of the testing of HPV tropism in high throughput using PRISM;

FIG. 15A is a photographic fluorescence representation of the high efficiency transduction of primary astrocytes, wherein in the green color represents HPV16, the red color represents GFAP astrocytes, and the blue color represents the MAP2 neurons;

FIG. 15B is a photographic fluorescence representation of the high efficiency transduction of primary astrocytes, wherein the green color represents HPV26, the red color represents GFAP astrocytes, and the orange color represents MAP2 neurons;

FIG. 15C is a photographic fluorescence representation of the high efficiency transduction of primary astrocytes, wherein the red color represents GFAP astrocytes;

FIG. 15D is a photographic fluorescence representation of the high efficiency transduction of primary astrocytes, wherein the green color represents HPV26;

FIG. 16 is a bar chart representation of the transduction with luciferase reporter transgene of primary human induced pluripotent stem cells;

FIG. 17A is a bar chart representation of the transduction with luciferase reporter transgene of primary hepatocytes at day 5;

FIG. 17B is a bar chart representation of the transduction with luciferase reporter transgene of primary hepatocytes at day 7;

FIG. 18 is a bar chart representation of the transduction of primary lung basal epithelial cells;

FIG. 19 is a schematic representation of a primary lung organoid model for HPV transduction of lung epithelia;

FIG. 20A is a bar char representation of the transduction with luciferase reporter transgene of primary lung organoids for the basal side of lung organoids;

FIG. 20B is a bar char representation of the transduction with luciferase reporter transgene of primary lung organoids for the apical mucus side of lung organoids;

FIG. 21A is a schematic representation of gene editing;

FIG. 21B is a schematic representation of circular plasmids for gene editing;

FIG. 21C is a schematic representation of the production of minicircular vectors;

FIG. 21D is a schematic representation of the production of minicircular vectors;

FIG. 22 is a bar chart representation of the efficiency of minicircle transgene vectors;

FIG. 23A is a bar chart representation of the genome editing performance of HPVs with SpaCas9 and ABE7;

FIG. 23B is a bar chart representation of the genome editing performance of HPVs with SpaCas9 and ABE7;

FIG. 23C is a bar chart representation of the genome editing performance of HPVs with AncBE4max;

FIG. 24 is a bar chart representation of the genome editing with HPV39, HPV68, HPV46, and HPV 16;

FIG. 25 is a schematic representation of a single vector homology directed repair (HDR) with SpCas9 vectors;

FIG. 26A is a schematic representation of the homology directed repair (HDR) sites on the EMX1 gene;

FIG. 26B is a bar chart representation of the performance the homology directed repair (HDR) at the EMX1 gene with HPV;

FIG. 27A is a schematic representation of the editing of endogenous T-cell receptor (TCR) at T-cell receptor alpha chain (TRAC) locus vian HPV delivery of homology directed repair (HDR) template;

FIG. 27B is a schematic representation of HPV delivery of HPV vector with T-cell receptor (TCR) in vitro/ex vivo and in vivo;

FIG. 28 is a schematic representation of using Cre reporter mice to determine in vivo tropism of HPV particles;

FIG. 29A is a schematic representation of the Cre stoplight circular plasmid;

FIG. 29B is a schematic representation of the performance of Cre gene delivery to edit stoplight cells;

FIG. 30 is a schematic representation of the structure of HPV;

FIG. 31A is a schematic representation of HPV16 testing exterior facing sites for peptide insertions;

FIG. 31B is a schematic representation of HPV16 testing exterior facing sites for peptide insertions;

FIG. 31C is a table representation of the HPV16 exterior facing sites;

FIG. 32 is a bar chart representation of the testing of the exterior facing sites for peptide insertions;

FIG. 33 is a schematic representation of the directed evolution for improved HPV efficiency;

FIG. 34 is a bar chart representation of the enhanced transduction of engineered L2 C-terminus with cell penetrating peptides;

FIG. 35A is a bar chart representation of the enhanced transduction in non-dividing cell by CPP12;

FIG. 35B is a bar chart representation of the enhanced transduction in non-dividing cell by CPP12;

FIG. 36 is a bar chart representation of L2 capsid protein modified with C-terminal tag fusions;

FIG. 37A is a table representation of production cost of common viral vectors;

FIG. 37B is a table representation of the required dose, global prevalence, and total dose needed for a range of disorders;

FIG. 38 is a schematic representation of the screening for improved HPV production; and

FIG. 39 is a schematic representation of HPV production by bacterial culture.

### DETAILED DESCRIPTION

The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

### I. Definitions

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

Furthermore, “and/or” where used herein is to be taken as specific disclosure of each of the two specified features of components with or without the other. Thus, the term “and/or” as used in a phrase such as “A and/or B” herein is intended to include “A and B,” “A or B,” “A” (alone), and “B” (alone).

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of 20% or ±10%, including 5%, ±1%, and +0.1% from the specified value, as such variations are appropriate to perform the disclosed methods.

The term “comprising” encompasses the term “including.”

As used herein, the term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

Definitions of common terms and techniques in molecular biology may be found in Molecular Cloning: A Laboratory Manual, 2nd ed. (1989) (Sambrook, Fritsch, and Maniatis); Molecular Cloning: A Laboratory Manual, 4th ed. (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): and Antibodies A Laboratory Manual, 2nd ed. 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology, 2nd ed., J. Wiley & Sons (New York, N.Y. 1994); March, Advanced Organic Chemistry Reactions, Mechanisms and Structure, 4th ed., J. Wiley & Sons (New York, N.Y. 1992); and Marten H. Hofker and Jan van Deursen, Transgenic Mouse Methods and Protocols, 2nd ed. (2011), which are incorporated by reference herein in their entirety.

As used herein, the term “polypeptide” and the like refer to an amino acid sequence including a plurality of consecutive polymerized amino acid residues (e.g., at least about two consecutive polymerized amino acid residues). “Polypeptide” refers to an amino acid sequence, oligopeptide, peptide, protein, enzyme, nuclease, or portions thereof, and the terms “polypeptide,” “oligopeptide,” “peptide,” “protein,” “enzyme,” and “nuclease,” are used interchangeably. The polypeptide may be linear or branched, it may comprise modified amino acids, and it may be interrupted by non-amino acids. The polypeptide may encompass an amino acid sequence that has been modified; for example, disulfide bond formation, glycosylation, lipidation, acetylation, phosphorylation, or any other manipulation, such as conjugation with a labeling component.

Polypeptides as described herein also include polypeptides having various amino acid additions, deletions, or substitutions relative to the native amino acid sequence of a polypeptide of the present disclosure. The polypeptides that are homologs of a polypeptide of the present disclosure can contain non-conservative changes of certain amino acids relative to the native sequence of a polypeptide of the present disclosure. The polypeptides that are homologs of a polypeptide of the present disclosure can contain conservative changes of certain amino acids relative to the native sequence of a polypeptide of the present disclosure, and thus may be referred to as conservatively modified variants. A conservatively modified variant may include individual substitutions, deletions or additions to a polypeptide sequence which result in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Thomas E. Creighton, “Proteins,” W. H. Freeman & Company (1984)). A modification of an amino acid to produce a chemically similar amino acid may be referred to as an analogous amino acid.

As used herein, the term “amino acid” and the like include natural and/or unnatural or synthetic amino acids, including glycine and both the D or L optical isomers, and amino acid analogs and peptidomimetics.

As used herein, the terms “nucleic acid,” “nucleic acid sequence,” “polynucleotide,” “oligonucleotide,” and the like refer to a deoxyribonucleic or ribonucleic oligonucleotide in either single- or double-stranded form comprising a plurality of consecutive polymerized nucleic-acid bases (e.g., at least about two consecutive polymerized nucleic-acid bases). The terms encompass nucleic acids, i.e., oligonucleotides, containing known analogues of natural nucleotides. The terms also encompass nucleic-acid-like structures with synthetic backbones, (see, e.g., Eckstein, Biomed. Biochim. Acta. 1991, 50(10-11), Si14-7; Baserga et al., Genes Dev. 1992 June, 6(6), 1120-30; Milligan et al., Nucleic Acids Res., 1993 Jan. 25, 21(2), 327-33; WO 97/03211; WO 96/39154; Mata, Toxicol Appl Pharmacol., 1997 May, 144(1), 189-97; Strauss-Soukup, Biochemistry, 1997 Aug. 19, 36(33), 10026-32; and Samstag, Antisense Nucleic Acid Drug Dev., 1996 Fall, 6(3), 153-6).

As used herein, the term “variant” and the like refer to a polypeptide or polynucleotide sequence that differs from a given polypeptide or nucleotide sequence in amino acid or nucleic acid sequence by the addition (e.g., insertion), deletion, or conservative substitution of amino acids or nucleotides, but that retains some or all the biological activity of the given polypeptide (e.g., a variant nucleic acid could still encode the same or a similar amino acid sequence). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al., J. Mol. Biol., 157, 105-132 (1982), which is incorporated by reference here in its entirety). The hydropathic index of an amino acid is based on a consideration of its hydrophobicity and charge. It is known in the art that amino acids of similar hydropathic indexes can be substituted and still retain protein function. The present disclosure provides amino acids having hydropathic indexes of 2 that can be substituted. The hydrophilicity of amino acids also can be used to reveal substitutions that would result in proteins retaining some or all biological functions. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining some or all biological activities, for example immunogenicity, as is understood in the art. The present disclosure provides substitutions that can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” also can be used to describe a polypeptide or fragment thereof that has been differentially processed, such as by proteolysis, phosphorylation, or other post-translational modification, yet retains some or all its biological and/or antigen reactivities. Use of “variant” herein is intended to encompass fragments of a variant unless otherwise contradicted by context.

Alternatively, or additionally, a “variant” is to be understood as a polynucleotide or protein which differs in comparison to the polynucleotide or protein from which it is derived by one or more changes in its length or sequence. The polypeptide or polynucleotide from which a protein or nucleic acid variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence in comparison to the parent molecule. Also encompassed modified molecules such as but not limited to post-translationally modified proteins (e.g., glycosylated, biotinylated, phosphorylated, ubiquitinated, palmitoylated, or proteolytically cleaved proteins) and modified nucleic acids such as methylated DNA. Also, mixtures of different molecules such as but not limited to RNA-DNA hybrids, are encompassed by the term “variant”. Typically, a variant is constructed artificially, for example by gene-technological means whilst the parent polypeptide or polynucleotide is a wild-type protein or polynucleotide. However, also naturally occurring variants are to be understood to be encompassed by the term “variant” as used herein. Further, the variants usable in the present disclosure may also be derived from homologs, orthologs, or paralogs of the parent molecule or from artificially constructed variant, provided that the variant exhibits at least one biological activity of the parent molecule, i.e., is functionally active.

Alternatively, or additionally, a “variant” as used herein can be characterized by a certain degree of sequence identity to the parent polypeptide or parent polynucleotide from which it is derived. More precisely, a protein variant in the context of the present disclosure exhibits at least 80% sequence identity to its parent polypeptide. A polynucleotide variant in the context of the present disclosure exhibits at least 70% sequence identity to its parent polynucleotide. The term “at least 70% sequence identity” is used throughout the specification with regard to polypeptide and polynucleotide sequence comparisons. This expression can refers to a sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

The similarity of nucleotide and amino acid sequences, i.e., the percentage of sequence identity, can be determined via sequence alignments. Such alignments can be carried out with several art-known algorithms, for example with the mathematical algorithm of Karlin and Altschul (Karlin & Altschul (1993) Proc. Natl. Acad. Sci. USA 90: 5873-5877) (which is incorporated by reference herein in its entirety), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) (which is incorporated by reference herein in its entirety) available e.g., on www.ebi.ac.uk/Tools/clustalw/or on www.ebi.ac.uk/Tools/clustalw2/index.html or on npsa-pbil.ibcp.fr/cgi-bin/npsa_automat.pl?page=/NPSA/npsa_clustalw.html. The parameters used can be the default parameters as they are set on www.ebi.ac.uk/Tools/clustalw/ or www.ebi.ac.uk/Tools/clustalw2/index.html. The grade of sequence identity (sequence matching) may be calculated using e.g., BLAST, BLAT or BlastZ (or BlastX). A similar algorithm is incorporated into the BLASTN and BLASTP programs of Altschul et al. (1990) J. Mol. Biol. 215: 403-410, which is incorporated by reference herein in its entirety. To obtain gapped alignments for comparative purposes, Gapped BLAST is utilized as described in Altschul et al. (1997) Nucleic Acids Res. 25: 3389-3402, which is incorporated by reference herein in its entirety. When utilizing BLAST and Gapped BLAST programs, the default parameters of the respective programs can be used. Sequence matching analysis may be supplemented by established homology mapping techniques like Shuffle-LAGAN (see, e.g., Brudno M., Bioinformatics, 2003b, 19 Suppl. 1, I54-I62, which is incorporated by reference herein in its entirety) or Markov random fields. When percentages of sequence identity are referred to in the present application, these percentages are calculated in relation to the full length of the longer sequence, if not specifically indicated otherwise.

As used herein, the term “minicircle vector” and the like refer to a double stranded circular DNA molecule that provides for expression of a sequence of interest that is present on the vector.

As used herein, the terms “genetically modified,” “transformed,” “transfected” and the like by exogenous nucleic acid (e.g., a polynucleotide via a recombinant vector) refer to when such nucleic acid has been introduced inside a cell. The presence of the exogenous nucleic acid results in permanent or transient genetic change.

As used herein, the term “transduced” and the like refer to when nucleic acid (e.g., a polynucleotide) has been introduced inside a cell via a viral-derived particle.

As used herein, the term “cell line” and the like refer to a clone of a primary cell can stable growth in vitro for many generations.

As used herein, the term “expression” and the like refer to the process by which a polynucleotide is transcribed from a DNA template (such as into a mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the terms “protospacer-adjacent motif” and the like refer to a DNA sequence immediately following a DNA sequence targeted by a nuclease. Examples of protospacer-adjacent motif include, without limitation, NNNNGATT, NNNNGNNN, NNG, NG, NGAN, NGNG, NGAG, NGCG, NAAG, NGN, NRN, NNGRRN, NNNRRT, TTTN, TTTV, TYCV, TATV, TYCV, TATV, TTN, KYTV, TYCV, TATV, TBN, a variant thereof, and a combination thereof.

As used herein, the terms “patient,” “subject,” “individual,” and the like refer to any animal, or cells thereof whether in vitro or in situ, amenable to the compositions, methods, and systems described herein. The patient can also be a human.

As used herein, the terms “treatment” and the like refer to the application of one or more specific procedures used for the amelioration of a disease. The specific procedure can be the administration of one or more pharmaceutical agents. “Treatment” of an individual (e.g., a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition, and may include, for example, minimal changes or improvements in one or more measurable markers of the disease or condition being treated.

As used herein, the term “disease” and the like refer to a state of health of a subject wherein the subject cannot maintain homeostasis, and wherein if the disease is not ameliorated then the subject's health continues to deteriorate. In contrast, a “disorder” in a subject is a state of health in which the subject can maintain homeostasis, but in which the subject's state of health is less favorable than it would be in the absence of the disorder. Left untreated, a disorder does not necessarily cause a further decrease in the subject's state of health.

### II. Papillomaviral Delivery Vehicle

The disclosures herein provide non-naturally occurring or engineered compositions, methods, and systems comprising a papillomaviral delivery vehicle for the delivery of gene editing material to cells. The papillomaviral delivery vehicle comprises a papillomavirus-derived capsid and DNA encoding a gene editing material encapsulated by the capsid. The cells can be eukaryotic cells, mammalian cells, or human cells. The cells can be hematopoietic stem cells, progenitor cells, satellite cells, mesenchymal progenitor cells, astrocyte cells, T-cells, B-cells, hepatocyte cells, heart cells, muscle cells, retinal cells, renal cells, or colon cells.

The components of the papillomaviral delivery vehicle can be synthesized by transfection. For example, a cell can be transfected with a first vector encoding the papillomavirus-derived capsid under condition conducive for the cell to synthesize the papillomavirus-derived capsid protein and a second vector encoding the DNA encoding the gene editing material under conditions conducive for the cell to replicate the second vector. The cell is then allowed to assemble the papillomaviral delivery vehicle and the papillomaviral delivery vehicle can be isolated from the cell. The vectors and/or mRNA encoding the capsid can be delivered to the cell via transfection, transduction, and electroporation. Any cell line that is known in the art to express and/or replicate genetic material can be used. An example of cell line includes, without limitation, HEK293FT cells.

The papillomaviral delivery vehicle can be used to edit a polynucleotide target in a cell, wherein the polynucleotide target can be a DNA or a RNA. For example, the papillomaviral delivery vehicle can be transduced in a cell comprising the polynucleotide target under condition conducive for the cell to synthesize the gene editing material. The gene editing material can then be allowed to edit the polynucleotide target. The promoter to synthesize the DNA encoding the gene editing materials must be appropriate for the cell type.

### III. Papillomavirus-Derived Capsid

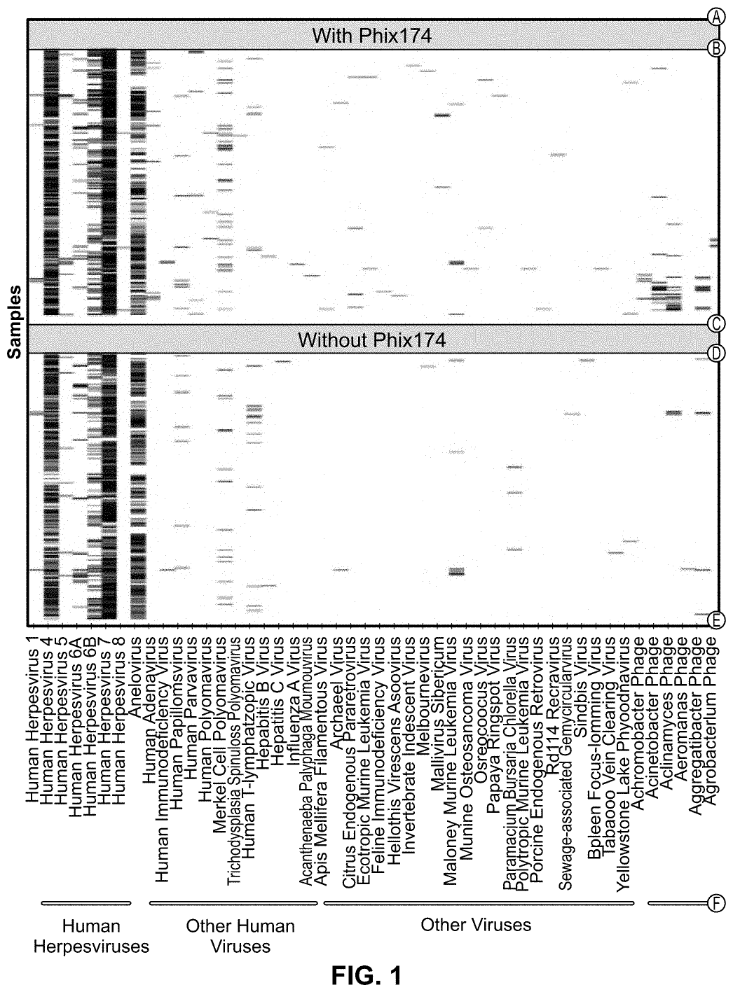

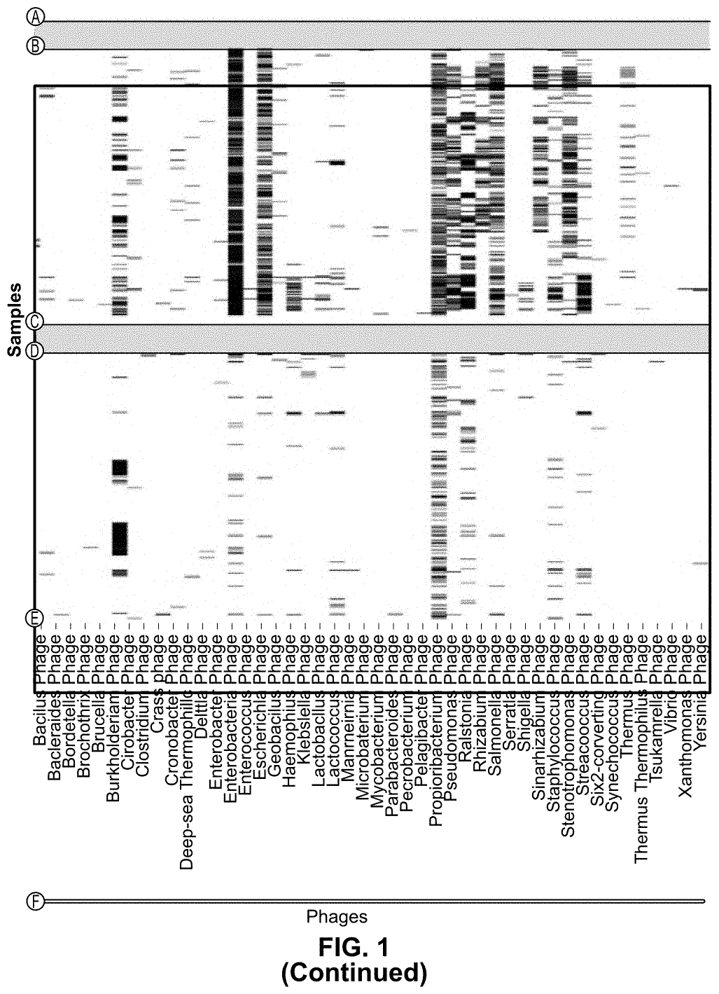

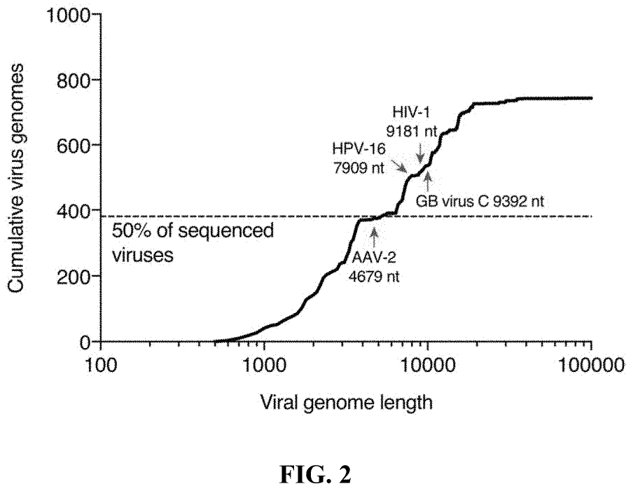

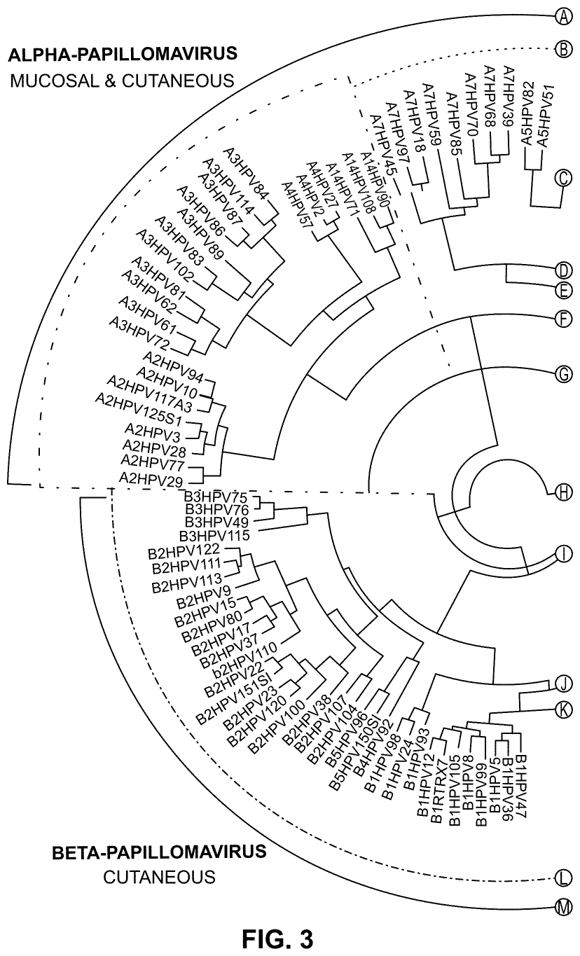

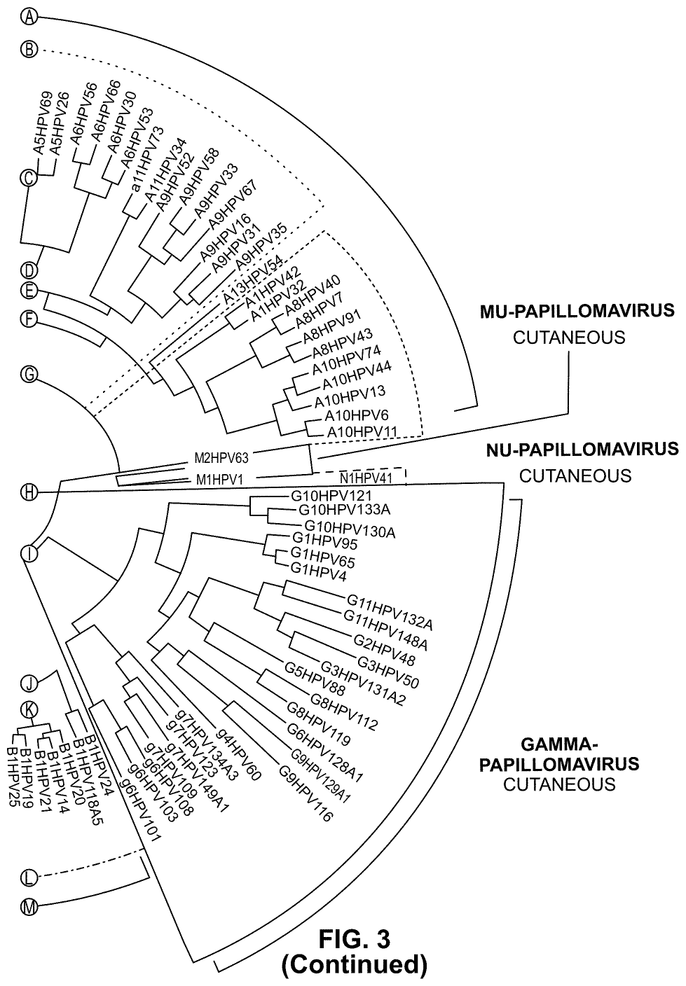

The papillomavirus-derived capsid disclosed herein is derived from a papilloma virus (FIGS. 1-3) (see, e.g., pave.niaid.nih.gov/#search/search_database). The papillomavirus-derived capsid can be derived from a mammalian papillomavirus such as for example, without limitation, a human papillomavirus (HPV). Useful mammalian papillomavirus can be an HPV-1, an HPV-2, an HPV-3, an HPV-4, an HPV-5, an HPV-6, an HPV-7, an HPV-8, an HPV-9, an HPV-10, an HPV-11, an HPV-12, an HPV-13, an HPV-14, an HPV-15, an HPV-16, an HPV-17, an HPV-18, an HPV-19, an HPV-20, an HPV-21, an HPV-22, an HPV-23, an HPV-24, an HPV-25, an HPV-26, an HPV-27, an HPV-28, an HPV-29, an HPV-30, an HPV-31, an HPV-32, an HPV-33, an HPV-34, an HPV-35, an HPV-36, an HPV-37, an HPV-38, an HPV-39, an HPV-40, an HPV-41, an HPV-42, an HPV-43, an HPV-44, an HPV-45, an HPV-47, an HPV-48, an HPV-49, an HPV-50, an HPV-51, an HPV-52, an HPV-53, an HPV-54, an HPV-56, an HPV-57, an HPV-58, an HPV-59, an HPV-60, an HPV-61, an HPV-62, an HPV-63, an HPV-65, an HPV-66, an HPV-67, an HPV-68, an HPV-69, an HPV-70, an HPV-71, an HPV-72, an HPV-73, an HPV-74, an HPV-75, an HPV-76, an HPV-77, an HPV-78, an HPV-80, an HPV-81, an HPV-82, an HPV-83, an HPV-84, an HPV-85, an HPV-86, an HPV-87, an HPV-88, an HPV-89, an HPV-90, an HPV-91, an HPV-92, an HPV-93, an HPV-94, an HPV-95, an HPV-96, an HPV-97, an HPV-98, an HPV-99, an HPV-100, an HPV-101, an HPV-102, an HPV-103, an HPV-104, an HPV-105, an HPV-106, an HPV-107, an HPV-108, an HPV-109, an HPV-110, an HPV-111, an HPV-112, an HPV-113, an HPV-114, an HPV-115, an HPV-116, an HPV-117, an HPV-118, an HPV-119, an HPV-120, an HPV-121, an HPV-122, an HPV-123, an HPV-124, an HPV-125, an HPV-126, an HPV-127, an HPV-128, an HPV-129, an HPV-130, an HPV-131, an HPV-132, an HPV-133, an HPV-134, an HPV-135, an HPV-136, an HPV-137, an HPV-138, an HPV-139, an HPV-140, an HPV-141, an HPV-142, an HPV-143, an HPV-144, an HPV-145, an HPV-146, an HPV-147, an HPV-148, an HPV-149, an HPV-150, an HPV-151, an HPV-152, an HPV-153, an HPV-154, an HPV-155, an HPV-156, an HPV-157, an HPV-158, an HPV-159, an HPV-160, an HPV-161, an HPV-162, an HPV-163, an HPV-164, an HPV-165, an HPV-166, an HPV-167, an HPV-168, an HPV-169, an HPV-170, an HPV-171, an HPV-172, an HPV-173, an HPV-174, an HPV-175, an HPV-176, an HPV-177, an HPV-178, an HPV-179, an HPV-180, an HPV-181, an HPV-182, an HPV-183, an HPV-184, an HPV-185, an HPV-186, an HPV-187, an HPV-188, an HPV-189, an HPV-190, an HPV-191, an HPV-192, an HPV-193, an HPV-194, an HPV-195, an HPV-196, an HPV-197, an HPV-199, an HPV-200, an HPV-201, an HPV-202, an HPV-203, an HPV-204, an HPV-205, an HPV-206, an HPV-207, an HPV-208, an HPV-209, an HPV-210, an HPV-211, an HPV-212, an HPV-213, an HPV-214, an HPV-215, an HPV-216, an HPV-219, an HPV-220, an HPV-221, an HPV-222, an HPV-223, an HPV-224, an HPV-225, a MmuPV-1, or a variant thereof.

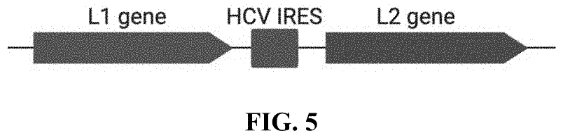

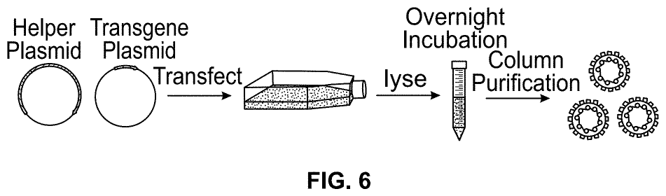

The papillomavirus-derived capsid is composed of two papillomaviral capsid proteins: L1, which is the major capsid protein, and L2, the minor capsid protein. L1 assembles into pentameric capsomers, 72 of which assemble into an icosahedron (T=7). Most of the L2 protein is located internally, but is essential for infection. L2 is also important for capsid assembly and stabilization (FIGS. 5 and 6).

The papillomavirus-derived capsid encapsulates nucleic acid, such as DNA encoding the gene editing material. The papillomavirus-derived capsid encapsulates DNA up to about 2.0 kb in length, or about 2.2 kb in length, or about 2.4 kb in length, or about 2.6 kb in length, or about 2.8 kb in length, or about 3.0 kb in length, or about 3.2 kb in length, or about 3.4 kb in length, or about 3.6 kb in length, or about 3.8 kb in length, or about 4.0 kb in length, or about 4.2 kb in length, or about 4.4 kb in length, or about 4.6 kb in length, or about 4.8 kb in length, or about 5.0 kb in length, or about 5.2 kb in length, or about 5.4 kb in length, or about 5.6 kb in length, or about 5.8 kb in length, or about 6.0 kb in length, or about 6.2 kb in length, or about 6.4 kb in length, or about 6.6 kb in length, or about 6.8 kb in length, or about 7.0 kb in length, or about 7.2 kb in length, or about 7.4 kb in length, or about 7.6 kb in length, or about 7.8 kb in length, or about 8.0 kb in length, or within a range that is made of any two or more points in the above list.

### IV. DNA Encoding the Gene Editing Material

The DNA encoding the gene editing material disclosed herein is a vector and the gene editing material can be any gene editing material that is known in the art, including Rees, H. A. et al., Nat Rev Genet 19, 770-788 (2018), doi:10.1038/s41576-018-0059-1; Anzalone, A. V., et al., Nature 576, 149-157 (2019), doi:10.1038/s41586-019-1711-4; and Villiger, L., et al., Nat Med., 2018 October, 24(10), 1519-1525, doi:10.1038/s41591-018-0209-1, which are incorporated herein by reference in their entirety).

Examples of gene editing materials include, without limitation, a nuclease, a clustered regularly interspaced short palindromic repeats (CRISPR) associated (Cas) nuclease, a miniature CRISPR nuclease, a nuclease coupled to a deaminase, a deaminase, a nickase, a transcriptase, a reverse transcriptase, an integration enzyme, an epigenetic modifier, a DNA methyltransferases, a guide RNA, a homology-directed repair (HDR) template, a reporter gene, a polynucleotide linked to a sequence complementary to an integration site, a split intein, a derivative thereof, and a combination thereof.

The nuclease disclosed herein can comprise a DNA-targeting nuclease, a DNA-binding nuclease, a DNA-cleaving nuclease, a meganuclease, a zinc-finger nuclease (ZFN), a transcription activator-like effector nuclease (TALEN), a derivative thereof, or a combination thereof. The nuclease can also comprise an RNA-targeting nuclease, an RNA-binding nuclease, an RNA-cleaving nuclease, a derivative thereof, or a combination thereof. The nuclease can also comprise any Cas nuclease orthologs and variants thereof that are known in the art such as for example, without limitation, a Cas7-11 nuclease, a Cas9 nuclease, a Cas10 nuclease, a Cas12 nuclease, a Cas13 nuclease such as a Cas13a nuclease, a Cas13b nuclease, a Cas13c nuclease, a Cas13d nuclease, and a Cas13e nuclease.

The DNA-binding nuclease disclosed herein can comprise a clustered regularly interspaced short palindromic repeat (CRISPR)-associated (Cas) DNA-binding nuclease. Such Cas DNA-binding nuclease can comprise a Cascade (type I) nuclease, type III nuclease, a Cas9 nuclease, a Cas12 nuclease, a variant thereof, or a combination thereof.

The guide RNA disclosed herein can comprise a single-guide RNA (sgRNA), a dual-guide RNA (dgRNA), a prime-editing guide RNA (pegRNA), a nicking-guide RNA (ngRNA), a derivative thereof, or a combination thereof.

Useful exemplary reporter genes disclosed herein can encode a fluorescent protein which can comprise a green fluorescent protein (GFP), a tdTomato protein, DsRed protein, a derivative thereof, or a combination thereof.

Useful exemplary deaminases disclosed herein can comprise an AncBE4 deaminase, an ABE7.10 deaminase, a derivative thereof, or a combination thereof.

The skilled person in the art will appreciate that the gene-editing material disclosed herein can comprise a single-stranded or a double-stranded DNA editing material.

### (i) Vector Encoding Gene Editing Material

The DNA encoding the gene editing material disclosed herein is in the form of a delivery vector which is discussed in more details below.

The vector can be a viral vector, such as a lenti- or baculo- or adeno-viral/adeno-associated viral vector. The viral vector may be selected from a variety of families/genera of viruses, including, but not limited to Myoviridae, Siphoviridae, Podoviridae, Corticoviridae, Lipothrixviridae, Poxviridae, Iridoviridae, Adenoviridae, Polyomaviridae, Papillomaviridae, Mimiviridae, Pandoravirusa, Salterprovirusa, Inoviridae, Microviridae, Parvoviridae, Circoviridae, Hepadnaviridae, Caulimoviridae, Retroviridae, Cystoviridae, Reoviridae, Birnaviridae, Totiviridae, Partitiviridae, Filoviridae, Orthomyxoviridae, Deltavirusa, Leviviridae, Picornaviridae, Marnaviridae, Secoviridae, Potyviridae, Caliciviridae, Hepeviridae, Astroviridae, Nodaviridae, Tetraviridae, Luteoviridae, Tombusviridae, Coronaviridae, Arteriviridae, Flaviviridae, Togaviridae, Virgaviridae, Bromoviridae, Tymoviridae, Alphaflexiviridae, Sobemovirusa, or Idaeovirusa.

A vector may mean not only a viral or yeast system, but also direct delivery of nucleic acids into a host cell. For example, baculoviruses may be used for expression in insect cells. These insect cells may, in turn be useful for producing large quantities of further vectors, such as AAV or lentivirus adapted for delivery of the present invention.

Non-viral vector delivery systems include DNA plasmids, RNA (e.g., a transcript of a vector described herein), naked nucleic acid, nucleic acid complexed with a delivery vehicle, such as a liposome, and ribonucleoprotein. Viral vector delivery systems include DNA and RNA viruses, which have either episomal or integrated genomes after delivery to the cell. For a review of gene therapy procedures, see, e.g., Anderson, Science 256:808-8313 (1992); Navel and Felgner, TIBTECH 11:211-217 (1993); Mitani and Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer and Perricaudet, British Medical Bulletin 51(1):31-44 (1995); Haddada et al., in Current Topics in Microbiology and Immunology, Doerfler and Bohm (eds.) (1995); and Yu et al., Gene Therapy 1:13-26 (1994), which are incorporated by reference herein in their entirety).

The expression of the DNA encoding the gene editing materials may be driven by a promoter. A single promoter can drive expression of a nucleic acid sequence encoding for one or more gene editing materials such as, for example, a nuclease and a guide RNA sequence. The nuclease and guide RNA sequence can be operably or not operably linked to and expressed or not expressed from the same promoter. The nuclease and guide RNA sequence can be expressed from different promoters. For example, the promoter(s) can be, but are not limited to, a UBC promoter, a PGK promoter, an EF1A promoter, a CMV promoter, an EFS promoter, a SV40 promoter, and a TRE promoter. The promoter may be a weak or a strong promoter. The promoter may be a constitutive promoter or an inducible promoter. The promoter can also be an AAV ITR, and can be advantageous for eliminating the need for an additional promoter element, which can take up space in the vector. The additional space freed up by use of an AAV ITR can be used to drive the expression of additional elements, such as guide sequences. The promoter can be a tissue specific promoter.

The DNA encoding the gene editing materials disclosed herein can be codon-optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See, e.g., Nakamura, Y., et al. “Codon usage tabulated from the international DNA sequence databases: status for the year 2000,” Nucl. Acids Res. 28:292 (2000), which is incorporated by reference herein in its entirety. Computer algorithms for codon optimizing a particular sequence for expression in a particular host cell are also available, such as Gene Forge (Aptagen; Jacobus, Pa.), are also available. One or more codons (e.g., 1, 2, 3, 4, 5, 10, 15, 20, 25, 50, or more, or all codons) in a sequence encoding a Cas protein can correspond to the most frequently used codon for a particular amino acid.

The DNA encoding the gene editing material disclosed herein may comprise a circular replicon, e.g., a minicircle. The minicircle may comprise a sequence of a bacterial origin or may not comprise a sequence of a bacterial origin.

The vector disclosed herein can comprise one or more nuclear localization sequences (NLSs), such as about or more than about one, two, three, four, five, six, seven, eight, nine, ten, or more NLSs. When more than one NLS is present, each may be selected independently of the others, such that a single NLS may be present in more than one copy and/or in combination with one or more other NLSs present in one or more copies. The NLS can be considered near the N-or C-terminus when the nearest amino acid of the NLS is within about 1, 2, 3, 4, 5, 10, 15, 20, 25, 30, 40, 50, or more amino acids along the polypeptide chain from the N- or C-terminus. Typically, an NLS consists of one or more short sequences of positively charged lysines or arginines exposed on the protein surface, bur other types of NLS are known. The NLS can be between two domains, for example between the nuclease and the viral protein. The NLS may also be between two functional domains separated or flanked by a glycine-serine linker.

The DNA encoding the gene editing material can be packaged into one or more vectors. Alternatively, or in addition, the vector encoding the gene editing material can be a targeted trans-splicing system.

### (ii) Cas Nuclease

The gene editing material disclosed herein can be a nuclease such as a Clustered Regularly Interspaced Short Palindromic Repeats (CRISPR) Associated (Cas) nuclease that is part of the Cas nuclease systems (also known as the CRISPR-Cas systems). The nuclease and related Cas nuclease systems are discussed in more details below.

In the conflict between bacterial hosts and their associated viruses, the Cas nuclease systems provide an adaptive defense mechanism that utilizes programmed immune memory. Cas nuclease systems provide their defense through three stages: adaptation, the integration of short nucleic acid sequences into the CRISPR array that serves as memory of past infections; expression, the transcription of the CRISPR array into a pre-crRNA (CRISPR RNA) transcript and processing of the pre-crRNA into functional crRNA species targeting foreign nucleic acids; and interference, the programming of CRISPR effectors by crRNA to cleave nucleic acid of foreign threats. Across all Cas nuclease systems, these fundamental stages display enormous variation, including the identity of the target nucleic acid (either RNA, DNA, or both) and the diverse domains and proteins involved in the effector ribonucleoprotein complex of the systems.

The Cas nuclease systems can be broadly split into two classes based on the architecture of the effector modules involved in pre-crRNA processing and interference. Class one systems have multi-subunit effector complexes composed of many proteins, whereas Class two systems rely on single-effector proteins with multi-domain capabilities for crRNA binding and interference; Class two effectors often provide pre-crRNA processing activity as well. Class one systems contain three types (type I, III, and IV) and 33 subtypes, including the RNA and DNA targeting type III-systems. Class two CRISPR families encompass three types (type IL, V, and VI) and 17 subtypes of systems, including the RNA-guided DNases Cas9 and Cas12 and the RNA-guided RNase Cas13. Continual sequencing of novel bacterial genomes and metagenomes uncovers new diversity of Cas nuclease systems and their evolutionary relationships, necessitating experimental work that reveals the function of these systems and develops them into new tools.

Among the currently known Cas nuclease systems or CRISPR-Cas systems, only the type III and type VI systems have been demonstrated to bind and target RNA, and these two systems have substantially different properties, the most distinguishing being their membership in Class one and Class 2, respectively. Characterized subtypes of type III, which span type III-A, B, and C systems, target both RNA and DNA species through an effector complex containing multiple Cas7 (Csm3/5 or Cmr1/4/6) RNA nuclease units in association with a single Cas10 (Csm1 or Cmr2) DNA nuclease. The RNA nuclease activity of Cas7 is mediated through acidic residues in the repeat-associated mysterious proteins (RAMP) domains, which cut at stereotyped intervals in the guide: target duplex. Type III systems also have a target restriction, and cannot efficiently target protospacers in vivo if there is extended homology between the 5′ “tag” of the crRNA and the “anti-tag” 3′ of the protospacer in the target, although this binding does not block RNA cleavage in vitro. In type III systems, pre-crRNA processing is carried out by either host factors or the associated Cas6 family protein, which can physically complex with the effector machinery.

In contrast to type III systems, type VI systems contain a single CRISPR effector Cas13 that can only effect RNA interference, mediated through basic catalytic residues of dual HEPN domains. This interference requires a protospacer flanking sequence (PFS), although the influence of the PFS varies between orthologs and families. Importantly, the RNA cleavage activity of Cas13, once triggered by crRNA: target duplex formation, is indiscriminate, and activated Cas13 enzymes will cleave other RNA species in vitro, in bacterial hosts, and mammalian cells. This activity, termed the collateral effect, has been applied to CRISPR-based nucleic acid detection technologies. In addition to the RNA interference activity, the Cas13 family members contain pre-crRNA processing activity. Just as single-effector DNA targeting systems have given rise to numerous genome editing applications, Cas13 family members have been applied to a suite of RNA-targeting technologies in both bacterial and eukaryotic cells, including RNA knockdown, RNA editing, RNA tracking, epitranscriptome editing, translational upregulation, epi-transcriptomic reading and writing via N6-Methyladenosine, and isoform modulation.

The novel type III-E system was identified from genomes of eight bacterial species and is characterized as a fusion of several Cas7 proteins and a putative Cas11 (Csm2)-like small subunit. The domain composition suggests the fusion of multiple type III effector module domains involved in crRNA binding into a single protein effector that is predicted to process pre-crRNA given its homology with Cas5 (Csm4) and conserved aspartates. The lack of other putative effector nucleases in these CRISPR loci raise the additional possibility that this fusion protein is capable of crRNA-directed RNA cleavage. If so, this system would blur the distinction of Class one and Class two systems, as it would have domains homologous to other Class one systems, but possess a single effector module characteristic of Class two systems. Beyond the single effector module present in all subtype III-E loci, a majority of type III-E family members contain a putative ancillary gene with a CHAT domain, which is a caspase family protease associated with programmed cell death (PCD), suggesting involvement of PCD-mediated antiviral strategies, as has been observed with type III and VI systems.

### Cas Nuclease for Gene Activation

The Cas nuclease disclosed here can be used with various CRISPR gene activation methods (see, e.g., Konermann S, Brigham M D, Trevino A E, Joung J, Abudayyeh O O, Barcena C, Hsu P D, Habib N, Gootenberg J S, Nishimasu H, Nureki o, Zhang F. Nature. 2015 Jan. 29; 517(7536):583-8. doi: 10.1038/nature14136. Epub 2014 Dec. 10. PMID: 25494202; PMCID: PMC4420636; David Bikard, Wenyan Jiang, Poulami Samai, Ann Hochschild, Feng Zhang, Luciano A. Marraffini, Nucleic Acids Research, Volume 41, Issue 15, 1 Aug. 2013, Pages 7429-7437, https://doi.org/10.1093/nar/gkt520; Perez-Pinera, P., Kocak, D., Vockley, C. et al. RNA-guided gene activation by CRISPR-Cas9-based transcription factors. Nat Methods 10, 973-976 (2013). https://doi.org/10.1038/nmeth.2600; Marvin E. Tanenbaum, Luke A. Gilbert, Lei S. Qi, Jonathan S. Weissman, Ronald D. Vale, Cell, vol 159, issue 3, pp. 635-646, Oct. 23, 2014, DOI: https://doi.org/10.1016/j.cell.2014.09.039; Konermann S., Brigham M. D., Trevino A. E., Joung J., Abudayyeh O. O., Barcena C., Hsu P. D., Habib N., Gootenberg J. S., Nishimasu H., Nureki O., Zhang F. Genome-scale transcriptional activation by an engineered CRISPR-Cas9 complex. Nature. 2015 Jan. 29; 517(7536):583-8. doi: 10.1038/nature14136. Epub 2014 Dec. 10. PMID: 25494202; PMCID: PMC4420636; Chavez, A., Scheiman, J., Vora, S. et al. Nat. Methods 12, 326-328 (2015). https://doi.org/10.1038/nmeth.3312; Chavez, A., Tuttle, M., Pruitt, B. et al. Nat Methods 13, 563-567 (2016). https://doi.org/10.1038/nmeth.3871; and Sajwan, S., Mannervik, M. Sci Rep 9, 18104 (2019). https://doi.org/10.1038/s41598-019-54179-x, which are incorporated herein by reference in their entirety). CRISPR gene activation methods are discussed in more details below.

Examples of CRISPR gene activation methods include, without limitation, dCas9-CBP CRISPR gene activation method, SPH CRISPR gene activation method, Synergistic Activation Mediator (SAM) CRISPR gene activation method, Sun Tag CRISPR gene activation method, VPR CRISPR gene activation method, and any alternative CRISPR gene activation methods therein. The dCas9-VP64 CRISPR gene activation method uses a nuclease lacking endonuclease ability and fused with VP64, a strong transcriptional activation domain. Guided by the nuclease, VP64 recruits transcriptional machinery to specific sequences, causing targeted gene regulation. This can be used to activate transcription during either initiation or elongation, depending on which sequence is targeted. The SAM CRISPR gene activation method uses engineered sgRNAs to increase transcription, which is done through creating a nuclease/VP64 fusion protein engineered with aptamers that bind to MS2 proteins. These MS2 proteins then recruit additional activation domains (HS1 and p65) to then activate genes. The Sun Tag CRISPR gene activation method uses, instead of a single copy of VP64 per each nuclease, a repeating peptide array to fused with multiple copies of VP64. By having multiple copies of VP64 at each loci of interest, this allows more transcriptional machinery to be recruited per targeted gene. The VPR CRISPR gene activation method uses a fused tripartite complex with a nuclease to activate transcription. This complex consists of the VP64 activator used in other CRISPR activation methods, as well as two other potent transcriptional activators (p65 and Rta). These transcriptional activators work in tandem to recruit transcription factors.

### Cas Nuclease for Base Editing

The Cas nuclease disclosed herein can be used as a base editor for base editing (see, e.g., Anzalone, A. V., et al., Nat. Biotechnol. 38, 824-844 (2020), which is incorporated herein by reference in its entirety). Cas nuclease used as a base editor for base editing is discussed in more details below.

There are generally three classes of base editors: cytosine base editors (CBEs), adenine base editors (ABEs), and dual-deaminase editor (also called SPACE, synchronous programmable adenine and cytosine editor). Base editing requires a nickase or nuclease fused or coupled to a deaminase that makes the edit, a gRNA targeting the nuclease to a specific locus, and a target base for editing within the editing window specified by the nuclease.

Cytosine base editors (CBEs) uses a cytidine deaminase coupled with an inactive nuclease. These fusions convert cytosine to uracil without cutting DNA. Uracil is then subsequently converted to thymine through DNA replication or repair. Fusing an inhibitor of uracil DNA glycosylase (UGI) to a nuclease prevents base excision repair which changes the U back to a C mutation. To increase base editing efficiency, the cell can be forced to use the deaminated DNA strand as a template by using a nuclease nickase, instead of a nuclease. The resulting editor, can nick the unmodified DNA strand so that it appears “newly synthesized” to the cell. Thus, the cell repairs the DNA using the U-containing strand as a template, copying the base edit.

Adenine base editors (ABEs) can convert adenine to inosine, resulting in an A to G change. Creating an adenine base editor requires an additional step because there are no known DNA adenine deaminases. Directed evolution can be used to create one from the RNA adenine deaminase TadA. While cytosine base editors often produce a mixed population of edits, some ABEs do not display significant A to non-G conversion at target loci. The removal of inosine from DNA is likely infrequent, thus preventing the induction of base excision repair. In terms of off-target effects, ABEs also generally compare favorably to other methods.

Suitable target nucleic acids will be readily apparent to one of skill in the art depending on the particular need or outcome. The target nucleic acid may be in, for example, a region of euchromatin (e.g., highly expressed gene), or the target nucleic acid may be in a region of heterochromatin (e.g., centromere DNA). A target nucleic acid of the present disclosure may be methylated or it may be unmethylated. The target gene can be any target gene used and/or known in the art.

### Cas Nuclease for Prime Editing

The Cas nuclease disclosed here can be used in prime editing and optionally with recombinase technology. Cas nuclease used in prime editing and optionally with recombinase technology is discussed in more details below.

Prime editing is a versatile and precise genome editing method that directly writes new genetic information into a specified DNA site. Such method is explained fully in the literature (see, e.g., Anzalone, A. V., et al. Nature 576, 149-157 (2019). Prime editing uses a catalytically-impaired Cas9 endonuclease that is fused to an engineered reverse transcriptase (RT) and programmed with a prime-editing guide RNA (pegRNA). The skilled person in the art would appreciate that the pegRNA both specifies the target site and encodes the desired edit. The catalytically-impaired Cas9 endonuclease also comprises a Cas9 nickase that is fused to the reverse transcriptase. During genetic editing, the Cas9 nickase part of the protein is guided to the DNA target site by the pegRNA. The reverse transcriptase domain then uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Afterward, the prime editor (PE) guides resolution of the heteroduplex to favor copying the edit onto the unedited strand, completing the process.

The prime editors refer to a Moloney Murine Leukemia Virus (M-MLV) reverse transcriptase (RT) fused to a Cas9 H840A nickase. Fusing the RT to the C-terminus of the Cas9 nickase may result in higher editing efficiency. Such a complex is called PE1. The Cas9(H840A) can also be linked to a non-M-MLV reverse transcriptase such as a AMV-RT or XRT (Cas9(H840A)-AMV-RT or XRT). The Cas 9(H840A) can be replaced with Cas12a/b or Cas9(D10A). A Cas9 (wild type), Cas9(H840A), Cas9(D10A) or Cas 12a/b nickase fused to a pentamutant of M-MLV RT (D200N/L603W/T330P/T306K/W313F), having up to about 45-fold higher efficiency is called PE2. The M-MLV RT can comprise one or more of the mutations Y8H, P51L, S56A, S67R, E69K, V129P, T197A, H204R, V223H, T246E, N249D, E286R, Q291L, E302K, E302R, F309N, M320L, P330E, L435G, L435R, N454K, D524A, D524G, D524N, E562Q, D583N, H594Q, E607K, D653N, and L671P. The reverse transcriptase can also be a wild-type or modified transcription xenopolymerase (RTX), avian myeloblastosis virus reverse transcriptase (AMV-RT), Feline Immunodeficiency Virus reverse transcriptase (FIV-RT), FeLV-RT (Feline leukemia virus reverse transcriptase), HIV-RT (Human Immunodeficiency Virus reverse transcriptase). PE3 involves nicking the non-edited strand, potentially causing the cell to remake that strand using the edited strand as the template to induce HR. The nicking of the non-edited strand can involve the use of a nicking guide RNA (ngRNA).

Nicking the non-edited strand can increase editing efficiency. For example, nicking the non-edited strand can increase editing efficiency by about 1.1 fold, about 1.3 fold, about 1.5 fold, about 1.7 fold, about 1.9 fold, about 2.1 fold, about 2.3 fold, about 2.5 fold, about 2.7 fold, about 2.9 fold, about 3.1 fold, about 3.3 fold, about 3.5 fold, about 3.7 fold, about 3.9 fold, 4.1 fold, about 4.3 fold, about 4.5 fold, about 4.7 fold, about 4.9 fold, or any range that is formed from any two of those values as endpoints.

Although the optimal nicking position varies depending on the genomic site, nicks positioned 3′ of the edit about 40 to about 90 bp from the pegRNA-induced nick can generally increase editing efficiency without excess indel formation. The prime editing practice allows starting with non-edited strand nicks about 50 bp from the pegRNA-mediated nick, and testing alternative nick locations if indel frequencies exceed acceptable levels.

The guide RNA can guide the insertion or deletion of one or more genes of interest or one or more nucleic acid sequences of interest into a target genome. The gRNA can also refer to a prime editing guide RNA (pegRNA), a nicking guide RNA (ngRNA), a single guide RNA (sgRNA), and the like.

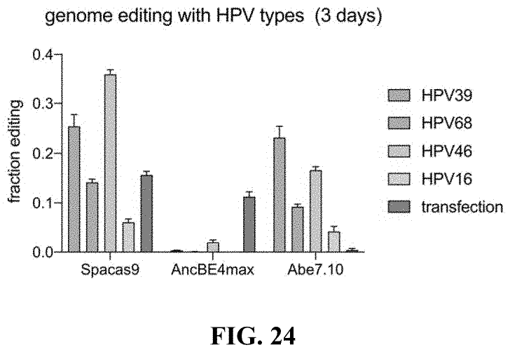

The pegRNA and the like refer to an extended sgRNA comprising a primer binding site (PBS), a reverse transcriptase (RT) template sequence, and an integration site sequence that can be recognized by recombinases, integrases, or transposases. Exemplary design parameters for pegRNA are shown in FIG. 24A. For example, the PBS can have a length of at least about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, or more nt. For example, the PBS can have a length of about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, or any range that is formed from any two of those values as endpoints. For example, the RT template sequence can have a length of at least about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, or more nt, For example, the RT template sequence can have a length of about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, or any range that is formed from any two of those values as endpoints.

The ngRNA and the like refer to an RNA sequence that can nick a strand such as an edited strand and a non-edited strand. Exemplary design parameters for ngRNA are shown in FIG. 24B. The ngRNA can induce nicks at about one or more nt away from the site of the gRNA-induced nick. For example, the ngRNA can nick at least at about 1, 3, 5, 7, 9, 11, 13, 15, 17, 19, 21, 23, 24, 25, 26, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, or more nt away from the site of the gRNA induced nick.

The gRNA can target a nuclease or a nickase such as Cas9, Cas 12a/b Cas9(H840A) or Cas9 (D10A) molecule to a target nucleic acid or sequence in a genome. The gRNA can bind to a DNA nickase bound to a reverse transcriptase domain. A “modified gRNA,” as used herein, refers to a gRNA molecule that has an improved half-life after being introduced into a cell as compared to a non-modified gRNA molecule after being introduced into a cell. The gRNA can facilitate the addition of the insertion site sequence for recognition by integrases, transposases, or recombinases.

During genome editing, the primer binding site allows the 3′ end of the nicked DNA strand to hybridize to the pegRNA, while the RT template serves as a template for the synthesis of edited genetic information. The pegRNA can for example, without limitation, (i) identify the target nucleotide sequence to be edited, and (ii) encode new genetic information that replaces the targeted sequence. The pegRNA can for example, without limitation, (i) identify the target nucleotide sequence to be edited, and (ii) encode an integration site that replaces the targeted sequence.

As used herein, the terms “reverse transcriptase,” “reverse transcriptase domain,” and the like refer to an enzyme or an enzymatically active domain that can reverse a RNA transcribe into a complementary DNA. The reverse transcriptase or reverse transcriptase domain is a RNA dependent DNA polymerase. Such reverse transcriptase domains encompass, but are not limited, to a M-MLV reverse transcriptase, or a modified reverse transcriptase such as, without limitation, Superscript® reverse transcriptase (Invitrogen; Carlsbad, Calif.), Superscript® VILO™ cDNA synthesis (Invitrogen; Carlsbad, Calif.), RTX, AMV-RT, and Quantiscript Reverse Transcriptase (Qiagen, Hilden, Germany).

The pegRNA-PE complex disclosed herein recognizes the target site in the genome and the Cas9 for example nicks a protospacer adjacent motif (PAM) strand. The primer binding site (PBS) in the pegRNA hybridizes to the PAM strand. The RT template operably linked to the PBS, containing the edit sequence, directs the reverse transcription of the RT template to DNA into the target site. Equilibration between the edited 3′ flap and the unedited 5′ flap, cellular 5′ flap cleavage and ligation, and DNA repair results in stably edited DNA. To optimize base editing, a Cas9 nickase can be used to nick the non-edited strand, thereby directing DNA repair to that strand, using the edited strand as a template.

(iii) Guide RNA

The gene editing material disclosed herein can be a guide RNA (gRNA) which is part of the Cas nuclease systems. Guide RNAs are discussed in more details below.

The gRNA can direct the Cas nuclease to a target nucleic acid sequence from a single stranded or double stranded DNA targeted by the nuclease. The gRNA can be a single-guide RNA (sgRNA) and can comprise a CRISPR RNA (crRNA), a trans-activating CRISPR RNA (tracrRNA), or a combination thereof. The crRNA and tracrRNA aid in directing the nuclease to a target nucleic acid sequence, and these RNA molecules can be specifically engineered to target specific nucleic acid sequences.

In general, the guide sequence from the gRNA is any polynucleotide sequence having sufficient complementarity with a target polynucleotide sequence to hybridize with the target sequence and direct sequence-specific binding of a target specific nuclease to the target sequence. The degree of complementarity between a guide sequence and its corresponding target sequence, when optimally aligned using a suitable alignment algorithm, can be about or more than about 50%, 52%, 54%, 56%, 58%, 60%, 62%, 64%, 66%, 68%, 70%, 72%, 74%, 76%, 78%, 80%, 82%, 84%, 86%, 88%, 90%, 91%, 92%, 93%, 94%, 95%, 96%, 97%, 98%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, ClustalX, BLAT, Novoalign (Novocraft Technologies, ELAND (Illumina, San Diego, Calif.), SOAP (available at soap.genomics.org.cn), and Maq (available at maq.sourceforge.net). The guide sequence can be about or more than about 5, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, or more nucleotides in length. The guide sequence can be less than about 75, 50, 45, 40, 35, 30, 25, 20, 15, 12, or fewer nucleotides in length. The guide RNA can have a spacer region with a sequence having a length of from about 20 to about 53 nucleotides (nt), or from about 25 to about 53 nt, or from about 29 to about 53 nt, or from about 40 to about 50 nt. The guide RNA can have a spacer region with a sequence having a length of about 20 nt, about 21 nt, about 22 nt, about 23 nt, about 24 nt, about 25 nt, about 26 nt, about 27 nt, about 28 nt, about 29 nt, about 30 nt, about 31 nt, about 32 nt, about 33 nt, about 34 nt, about 35 nt, about 36 nt, about 37 nt, about 38 nt, about 39 nt, about 40 nt, about 41 nt, about 42 nt, about 43 nt, about 44 nt, about 45 nt, about 46 nt, about 47 nt, about 48 nt, about 49 nt, about 50 nt, or within any ranges that are made of any two or more points in the above list. The guide RNA can have a direct repeat region with a sequence having a length of about 15 nt, about 16 nt, about 17 nt, about 18 nt, about 19 nt, about 20 nt, about 21 nt, about 22 nt, about 23 nt, about 24 nt, about 25 nt, about 26 nt, about 27 nt, about 28 nt, about 29 nt, about 30 nt, about 31 nt, about 32 nt, about 33 nt, about 34 nt, about 35 nt, about 36 nt, about 37 nt, about 38 nt, about 39 nt, about 40 nt, about 41 nt, about 42 nt, about 43 nt, about 44 nt, about 45 nt, about 46 nt, about 47 nt, about 48 nt, about 49 nt, about 50 nt, or within any ranges that are made of any two or more points in the above list. The guide RNA can have a tracrRNA region having a sequence with a length of about 15 nt, about 16 nt, about 17 nt, about 18 nt, about 19 nt, about 20 nt, about 21 nt, about 22 nt, about 23 nt, about 24 nt, about 25 nt, about 26 nt, about 27 nt, about 28 nt, about 29 nt, about 30 nt, about 31 nt, about 32 nt, about 33 nt, about 34 nt, about 35 nt, about 36 nt, about 37 nt, about 38 nt, about 39 nt, about 40 nt, about 41 nt, about 42 nt, about 43 nt, about 44 nt, about 45 nt, about 46 nt, about 47 nt, about 48 nt, about 49 nt, about 50 nt, or within any ranges that are made of any two or more points in the above list. The ability of a guide sequence to direct sequence-specific binding of a Cas nuclease to a target sequence may be assessed by any suitable assay.

### (iv) Zinc Finger Nuclease (ZFN)

The gene editing material disclosed herein can be a zinc finger nuclease (ZFN) which is discussed in more details below.

ZFNs are among very common DNA binding motifs found in eukaryotes. There are likely about 500 zinc finger proteins encoded by the yeast genome, and that likely 1% of all mammalian genes encode zinc finger containing proteins. These proteins are classified according to the number and position of the cysteine and histidine residues available for zinc coordination. ZFNs are useful for targeted cleavage and recombination. They are fusion proteins comprising a cleavage domain (or a cleavage half domain) and a zinc finger binding domain. A zinc finger binding domain can comprise one or more zinc fingers (e.g., two, three, four, five, six, seven, eight, nine or more zinc fingers), and can be engineered to bind to any genomic sequence. Thus, by identifying a target genomic region of interest at which cleavage or recombination is desired, using the compositions, methods, and systems disclosed herein, fusion proteins can be constructed comprising a cleavage domain (or cleavage half-domain) and a zinc finger domain engineered to recognize a target sequence in a genomic region. The presence of such a fusion protein in a cell results in binding of the fusion protein to its binding site and cleavage within or near the genomic region. Moreover, if an exogenous polynucleotide homologous to the genomic region is also present in such a cell, homologous recombination occurs at a high rate between the genomic region and the exogenous polynucleotide.

In addition to ZFNs, restriction endonucleases are also present in many species and are capable of sequence-specific binding to DNA at a recognition site and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA at five nucleotides from its recognition site on one strand and 13 nucleotides from its recognition site on the other (see, e.g., U.S. Pat. No. 5,356,802; 5,436,150 and 5,487,994; as well as Li et al. (1992) Proc. Natl Acad. Sci. USA 89:4275-4279; Li et al. (1993) Proc. Nat'l Acad. Sci. USA 90:2764-2768; Kim et al. (1994a) Proc. Natl. Acad. Sci. USA 91:883-887; Kim et al. (1994b) J. Biol. Chem. 269:31,978-31,982; and Bitinaite et al. (1998) Proc. Natl. Acad. Sci. USA 95: 10,570-10,575, which are incorporated by reference herein in their entirety). Thus, fusion proteins can comprise the cleavage domain (or cleavage half-domain) from at least one Type IIS restriction enzyme and one or more zinc finger binding domains, which may or may not be engineered. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a zinc finger binding domain and two Fok I cleavage half-domains can also be used.

In general, a cleavage domain or cleavage half-domain can be any portion of a protein that retains cleavage activity, or that retains the ability to multimerize (e.g., dimerize) to form a functional cleavage domain. A cleavage domain comprises one or more polypeptide sequences which possesses catalytic activity for DNA cleavage. A cleavage domain can be contained in a single polypeptide chain or cleavage activity can result from the association of two (or more) polypeptides. A cleavage half-domain is a polypeptide sequence which, in conjunction with a second polypeptide (either identical or different) forms a complex having cleavage activity (for example a double-strand cleavage activity).

### (v) Transcription Activator-Like Effector Nuclease (TALEN)

The gene editing material disclosed herein can be a transcription activator-like effector nuclease which is discussed in more details below.

Transcription Activator-Like Effector Nucleases (TALENs) are artificial restriction enzymes generated by fusing the TAL effector DNA binding domain to a DNA cleavage domain. These reagents enable efficient, programmable, and specific DNA cleavage and represent powerful tools for genome editing in situ. Transcription activator-like effectors (TALENs) can be quickly engineered to bind practically any DNA sequence. The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA (see, e.g., U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, which are incorporated by reference herein in their entirety).

TAL effectors are proteins secreted by Xanthomonas bacteria. The DNA binding domain contains a highly conserved about 33-34 amino acid sequence with the exception of the 12th and 13th amino acids. These two locations are highly variable (Repeat Variable Diresidue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs.

The non-specific DNA cleavage domain from the end of a FokI endonuclease can be used to construct hybrid nucleases that are active in a yeast assay. These reagents are also active in plant cells and in animal cells. Initial TALEN studies used the wild-type FokI cleavage domain, but some subsequent TALEN studies also used FokI cleavage domain variants with mutations designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. The number of amino acid residues between the TALEN DNA binding domain and the FokI cleavage domain may be modified by introduction of a spacer (distinct from the spacer sequence) between the plurality of TAL effector repeat sequences and the FokI endonuclease domain. The spacer sequence may be about 12 to 30 nucleotides.

### V. Delivery of the Papillomavirus Delivery Vehicle

The papillomaviral delivery vehicle disclosed herein can be delivered to a tissue comprising the target cell of interest by, for example, an intramuscular injection or via intravenous, transdermal, intranasal, oral, mucosal, intrathecal, intracranial or other delivery methods. Such delivery may be either via a single dose, or multiple doses. One skilled in the art understands that the actual dosage to be delivered herein may vary greatly depending upon a variety of factors, such as the vector chosen, the target cell, organism, or tissue, the general condition of the subject to be treated, the degree of transformation/modification sought, the administration route, the administration mode, the type of transformation/modification sought, etc.

The cell receiving the DNA encoding the gene editing material can be transiently or non-transiently transduced. The cell can be taken from a subject, derived from cells taken from a subject, and/or be from a cell line. Cell lines are available from a variety of sources known to those with skill in the art (see, e.g., the American Type Culture Collection (ATCC) (Manassas, Va.). The cell transduced with the DNA encoding the gene editing material can be used to establish a new cell line comprising sequences derived from the DNA encoding the gene editing material.

### VI. Kits

The present disclosure also provides kits for carrying out the method according to the disclosure. The kits can contain any one or more of the elements disclosed in the above compositions, methods, and systems. For example, the kit comprises the papillomaviral delivery vehicle disclosed herein and optionally instructions for using the kit. The kit can comprise a papillomaviral delivery vehicle comprising regulatory elements. Elements may be provided individually or in combinations, and may be provided in any suitable container, such as a vial, a bottle, or a tube. The kit can include instruction in one or more languages, for examples, in more than one language.

The kit can comprise one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer that is known in the art, including but not limited to a sodium carbonate buffer, a sodium bicarbonate buffer, a borate buffer, a Tris buffer, a MOPS buffer, a HEPES buffer, and a combination thereof. The buffer can be alkaline and have a pH from about seven to about ten

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Assaying HPV Viruses for Production, Packaging Size, and Cell Type Specificity

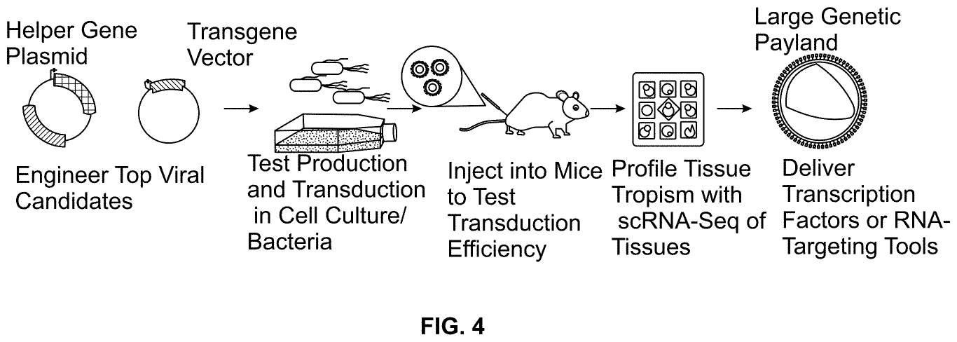

HPV viruses were assayed to assess production, packaging size, and cell type specificity (FIG. 4).

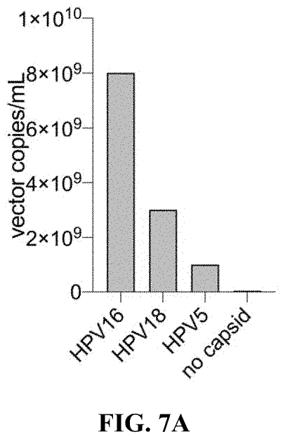

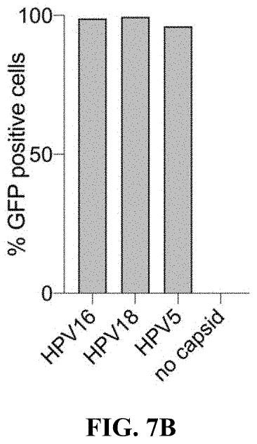

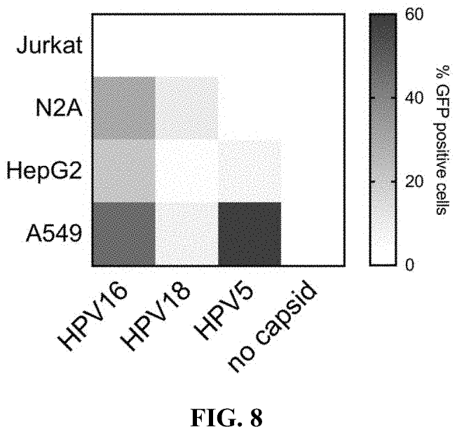

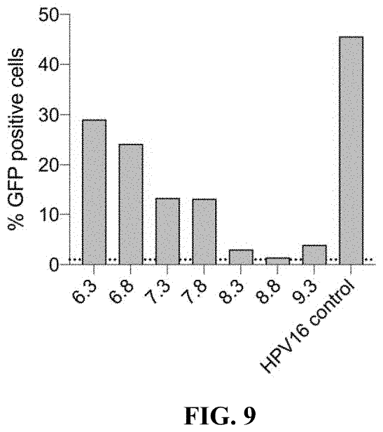

Top viral candidates were engineered using a helper gene plasmid vector comprising L1 and L2 genes and a transgene vector (FIGS. 5 and 6). The vectors were transfected and expressed using a cell culture, and the cells were then lysed, incubated, and purified by column chromatography. The number of copied vectors and the percentage of green fluorescent protein (GFP) positive in HEK293FT cells, Jurkat cells, N2A cells, HepG2 cells, and A549 cells were measured for HPV-16, HPV-18, and HPV-5 virus (FIGS. 7A, 7B, and 8). The percentage of GFP positive cells for payloads between about 6.3 kb to about 9.3 kb was also assessed (FIG. 9).

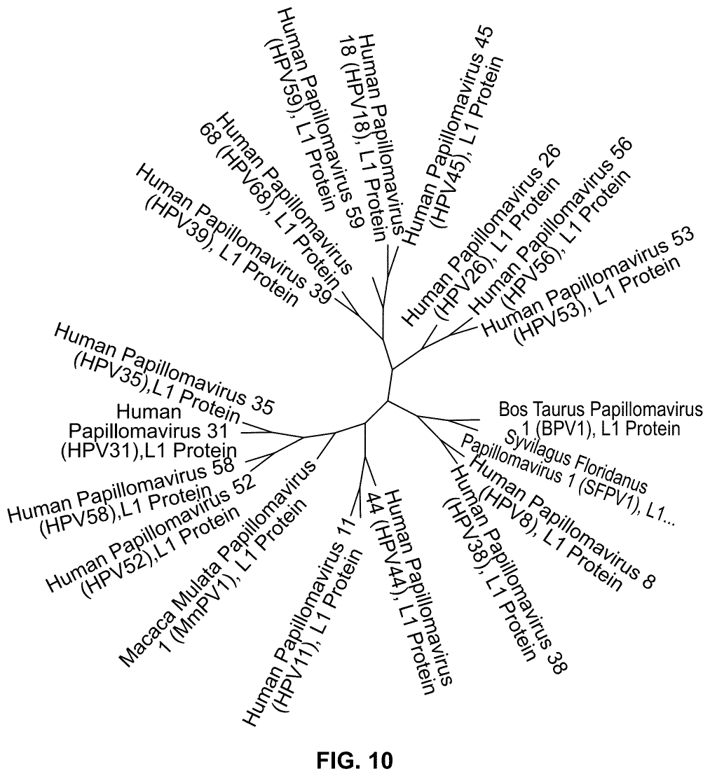

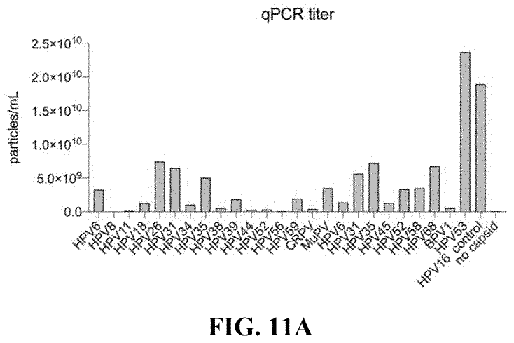

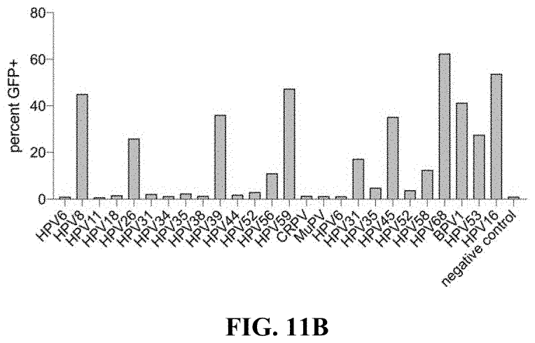

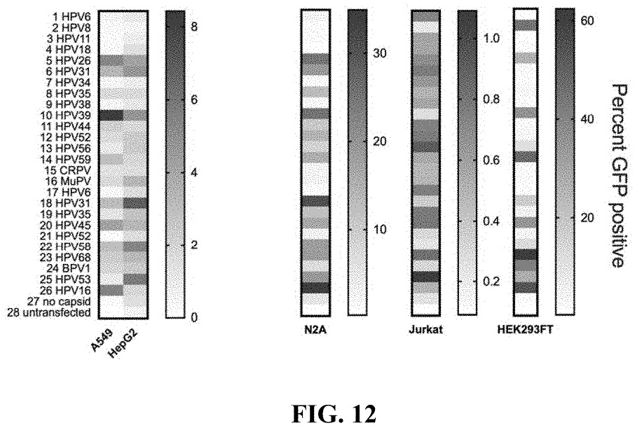

A large panel of HPVs were assayed by qPCR and transduced in HEK293FT cells, A549 cells, HepG2 cells, N2A cells, and Jurkat cells (FIGS. 10, 11A, 11B, 12).

### Example 2

### Testing HPV Tropism in High Throughput Using PRISM

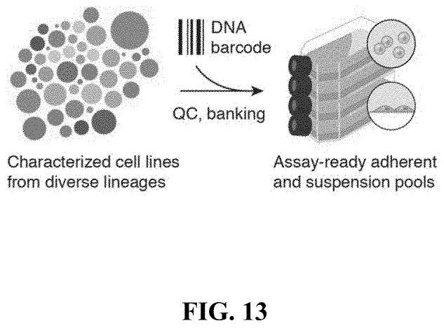

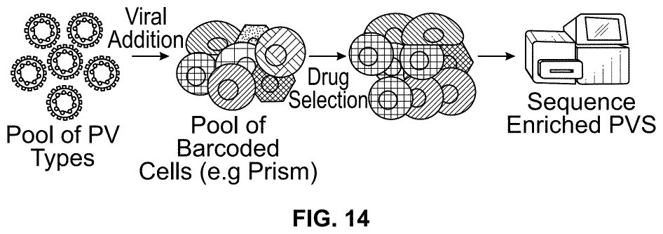

HPV tropism can be tested in high throughput using the PRISM method as illustrated in FIGS. 13 and 14 (see, e.g., Yu et al., Nat. Biotechnol, 2017, 34(4), 419-23, which is incorporated by reference herein in its entirety).

### Example 3

### Transduction of Primary Astrocytes with Labeled HPV-16, MAP2 and GFAP

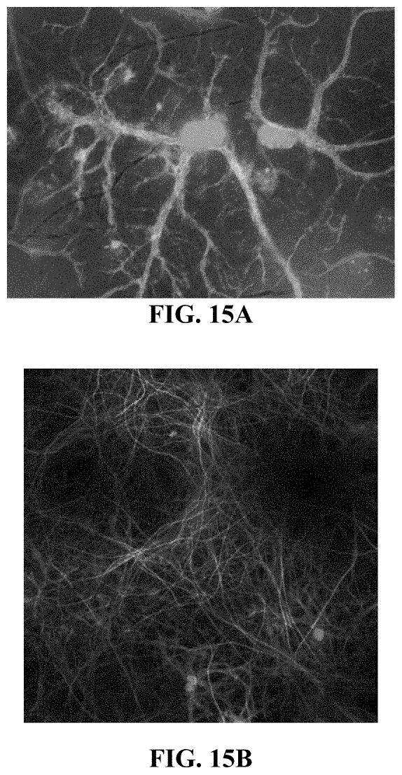

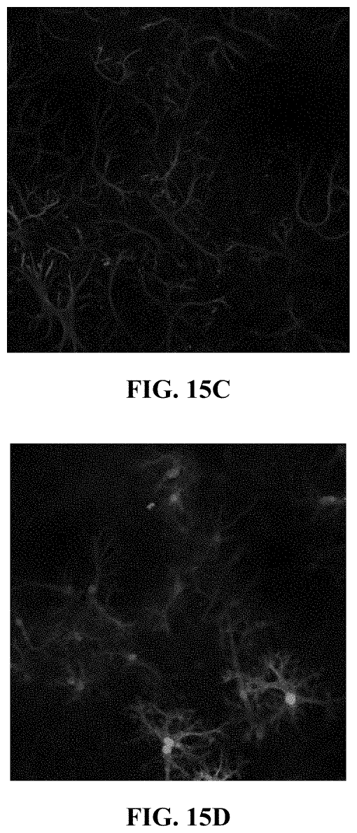

The transduction of primary astrocytes was assessed (FIGS. 15A-15D). As illustrated in FIG. 15A, HPV-16 (green label), GFAP (red label, astrocytes), and MAP2 (blue label, neurons) were transduced. As illustrated in FIG. 15B-15D, HPV-26 (green label), GFAP (red label, astrocytes), and MAP2 (orange label, neurons) were transduced.

### Example 4

### Transduction with Luciferase Reporter Transgene

Transductions with luciferase reporter transgene were assessed.

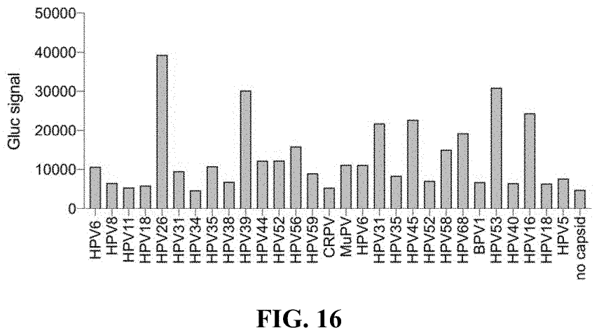

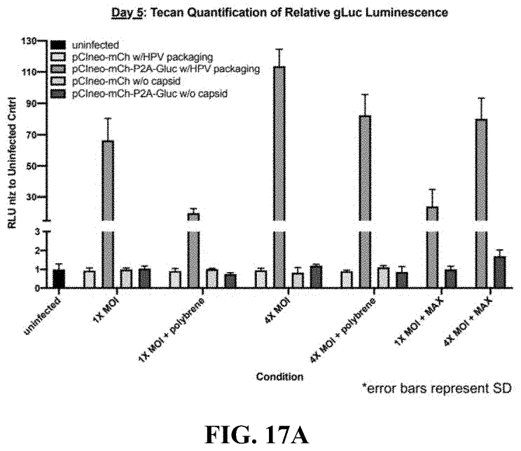

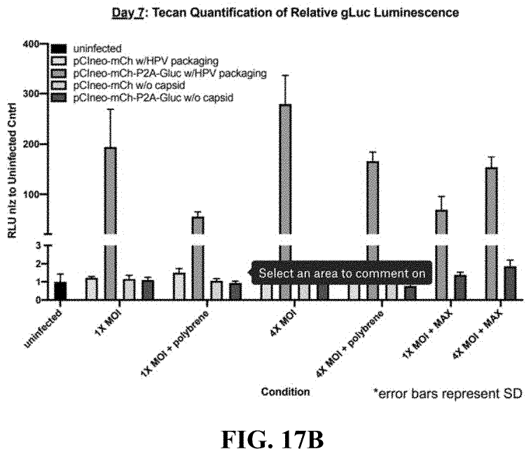

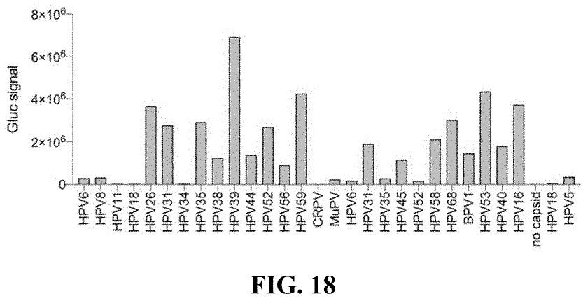

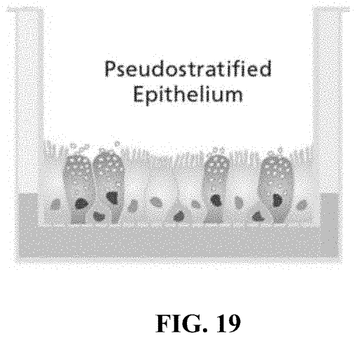

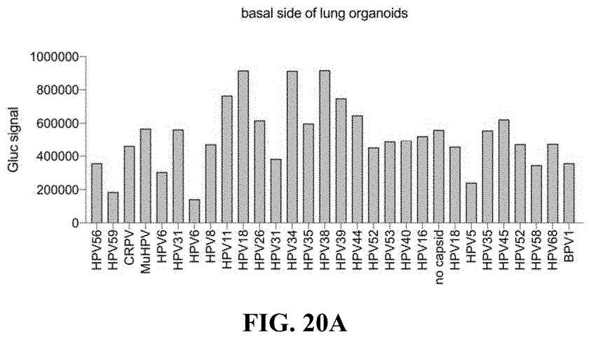

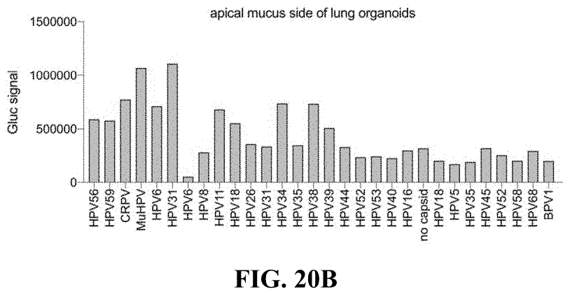

Primary human induced pluripotent stem cells, primary hepatocytes, and primary lung basal epithelial cells (from the basal and apical mucus sides of the lung organoids) were transduced with luciferase reporter transgene (FIGS. 16-20).

### Example 5

### DNA Encoding Gene Editing Material Delivered into Cells with HPV Capsid

The delivery of DNA encoding gene editing material into cells using HPV capsid was assessed.

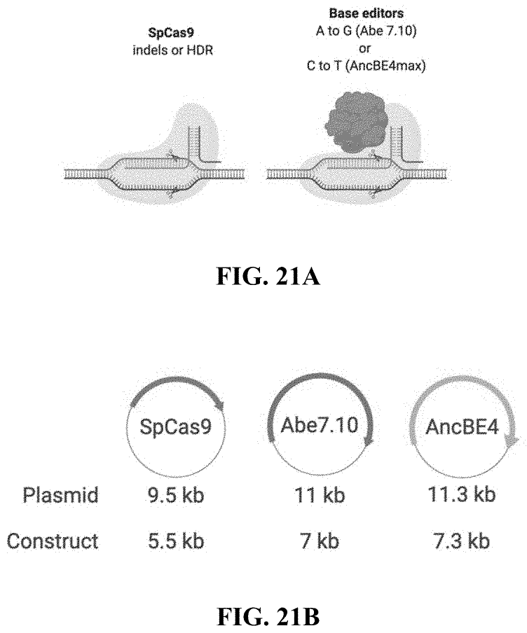

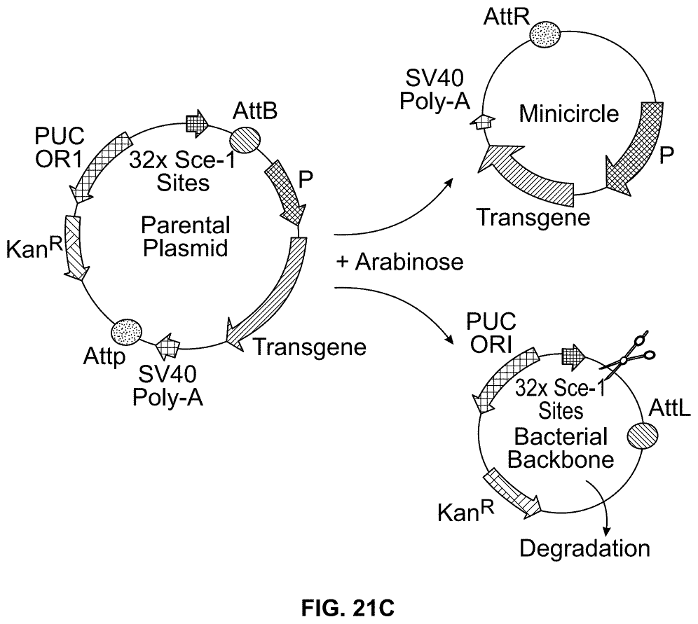

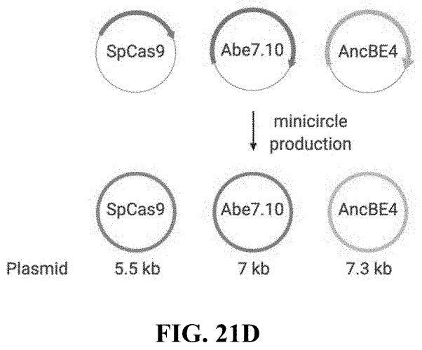

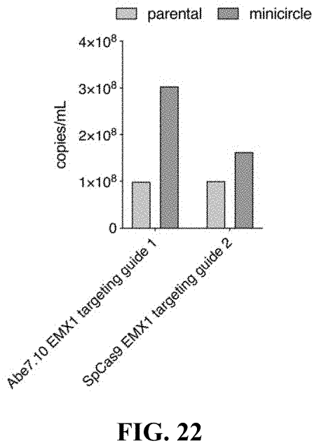

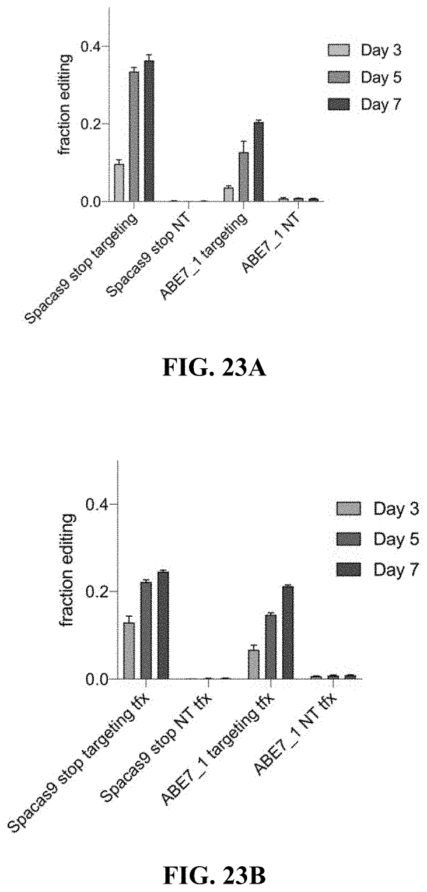

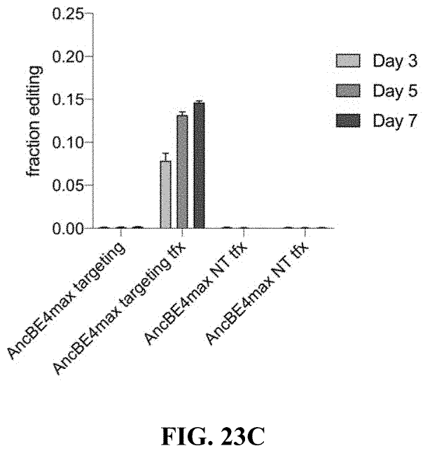

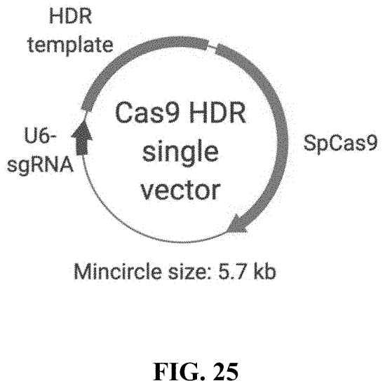

DNA encoding gene editing material, such as the Cas gene editing nuclease for indel editing, homology directed repair (HDR) editing, and/or base editing illustrated in FIG. 21A, can be delivered into cells using HPV capsids. The DNA can be a plasmid and/or a minicircle construct as illustrated in FIGS. 21B-D (see, e.g., Kay, M. et al., Nat. Biotechnol. 28, 1287-1289 (2010), doi:10.1038/nbt.1708, which is incorporated by reference herein in its entirety). The efficiency of the parental and minicircle transgene vectors (FIG. 22) and the performance of the genome editing using SpaCas9, Abe7, and AncBE4max inserts (FIGS. 23A-C) and HPV-16, -39,-46, and -68 viruses (FIG. 24) were assessed. The skilled person in the art will appreciate that a minicircle vector HDR with SpCas9 and U6-sgRNA can have a size of about 5.7 kb and can accommodate an HDR template up to about 2.0 kb in length as illustrated in FIG. 25. The template can be up to about 3.0 kb in length if the SpCas9 is switch to an SaCas9.

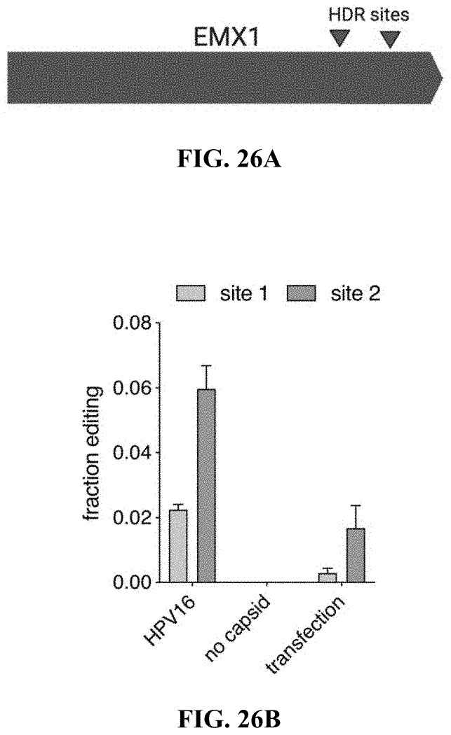

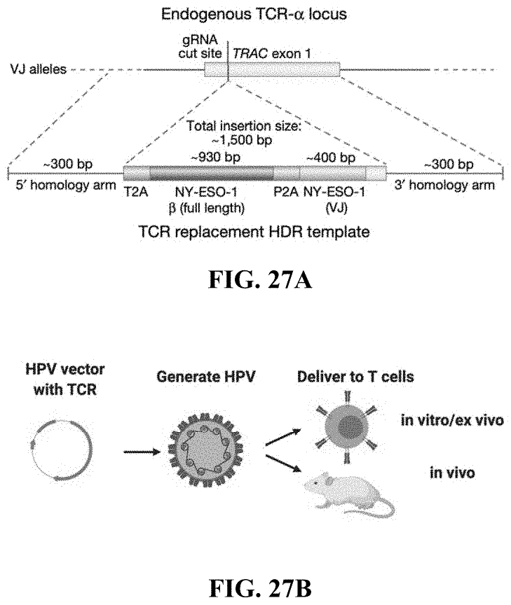

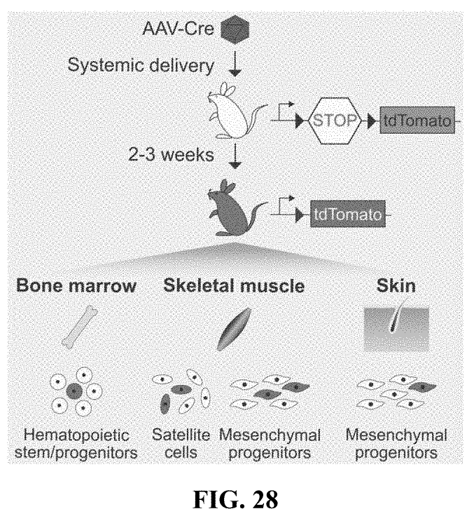

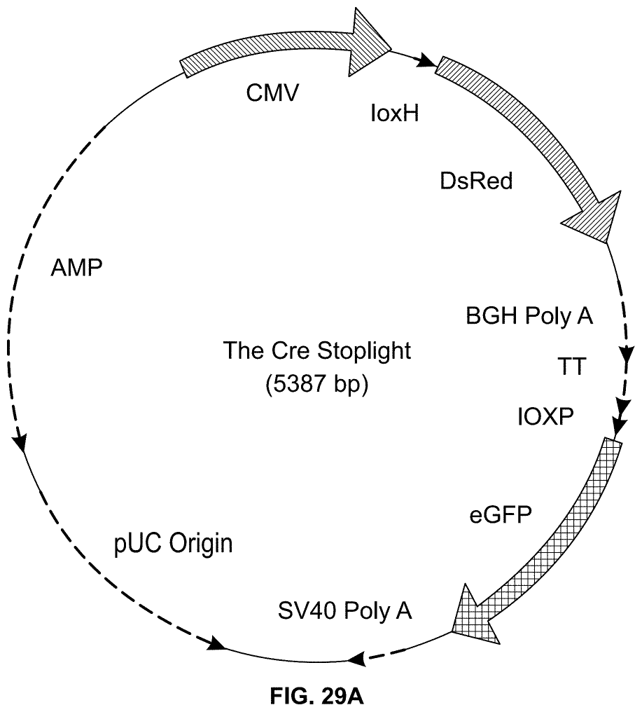

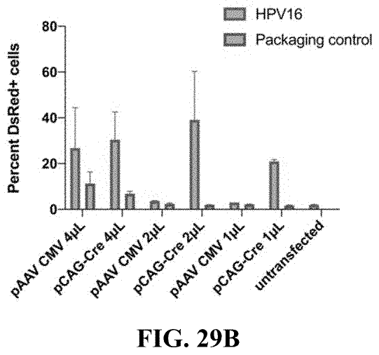

Homology directed repair (HDR) was performed at the EMX1 gene with HPV (FIGS. 26A-B). The 130 bp HDR template can insert a sequence of 10 bp with 60 bp homology arms. The editing of endogenous T-cell receptor (TCR) at T-cell receptor alpha chain (TRAC) locus vian HPV delivery of homology directed repair (HDR) template can be assessed as well as illustrated in FIGS. 27A-B. HPV vector with TCR can used to generate an HPV delivery vehicle to deliver to T-cells the gene editing material vector in vitro/ex vivo and in vivo (see, e.g., Roth et al., Nature Letter (2018), 559, 405-9, which is incorporated by reference herein in its entirety). Using Cre reporter mice, in vivo tropism of HPV particles can also be assessed as illustrated in FIG. 28 (see, e.g., Goldstein, et al., Cell Reports 2019, 27, 1254-64, which is incorporated by reference herein in its entirety). The Cre gene delivery effectively edits Stoplight cells as illustrated in FIGS. 29A-B.

### Example 6

### Directed Evolution of HPV Virus

HPV diversity and structure were assessed to find areas and sequences for directed evolution.

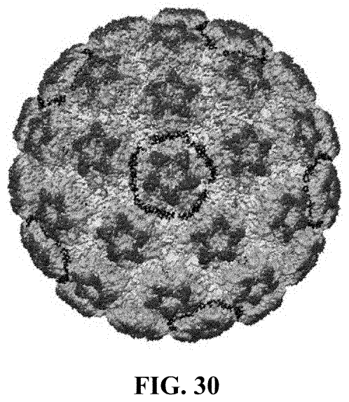

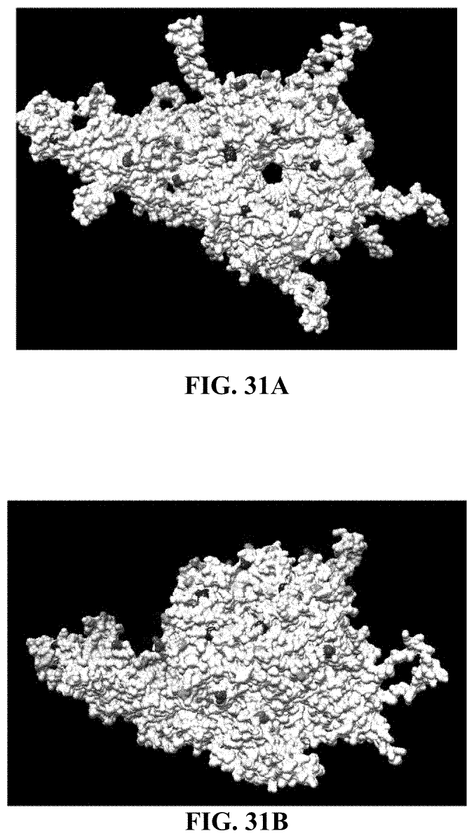

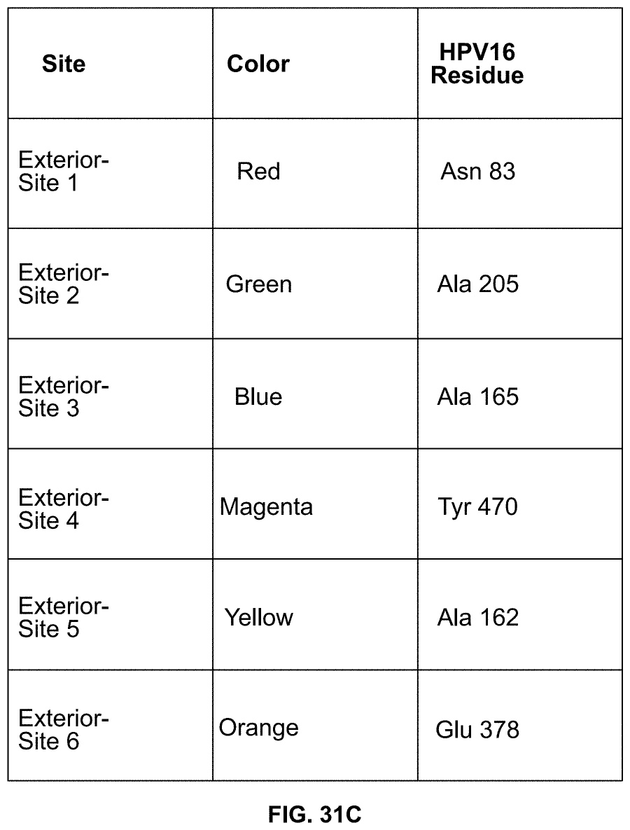

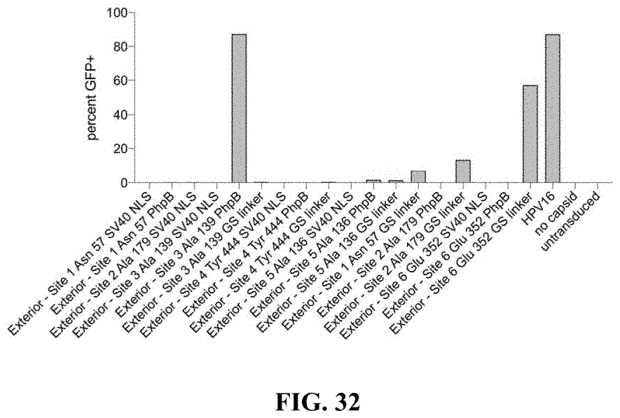

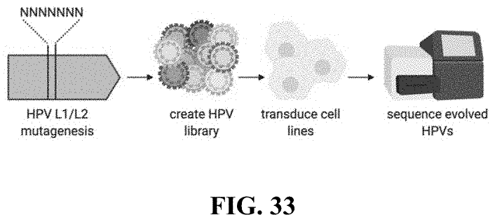

Exterior facing sites of HPV capsid were tested for peptide insertions (FIGS. 30, 31A-C, 32). Tested sites with three 7-peptides included SV40 NLS, PhpB, and GS linker. Specific peptides at sites one, two, three, and six were found to have transduction activity, which demonstrates that HPV capsids can be modified contrary to the long-held belief in the field. The directed evolution for improving HPV efficiency can be performed using HPV L1/L2 mutagenesis to create an HPV library and transduce cell lines as illustrated in FIG. 33. The resulting cell line can be analyzed by qPCR reaction. 7-mer insertion libraries designed for HPV-16 at sites one, two, three, and six were tested.

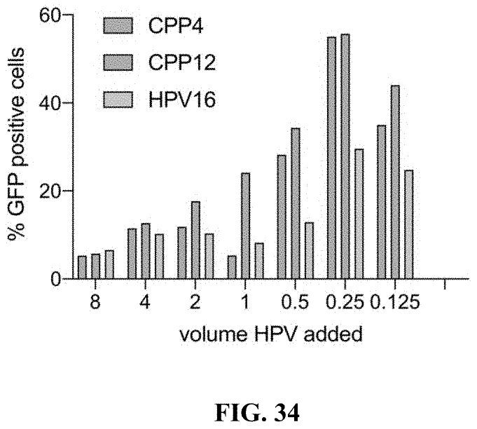

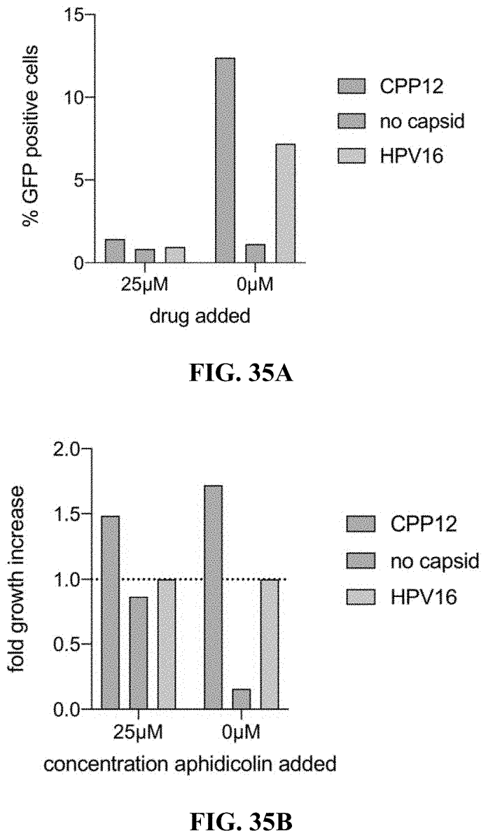

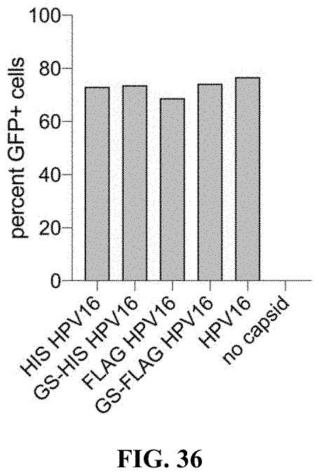

Engineering of L2 C-terminus with cell penetrating peptides using CPP4 (TAT-FWF CCP), CPP12 (TAT-FWF CPP+c-Myc NLS) was found to enhance transduction as illustrated in FIG. 34. The CCP12 was found to enhance transduction in non-dividing cells as well (FIG. 35A-B), and the L2 capsid protein was also found be modifiable with C-terminal tag fusions for easier and more pure purification (FIG. 36). All fusions were found to retain significant transduction activity, as good as the unmodified HPV-16.

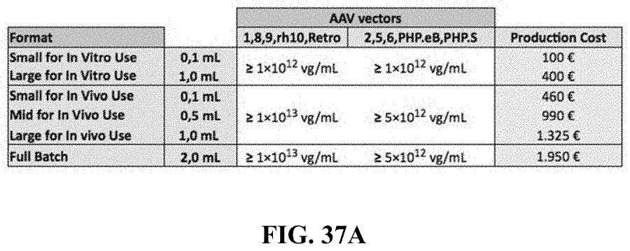

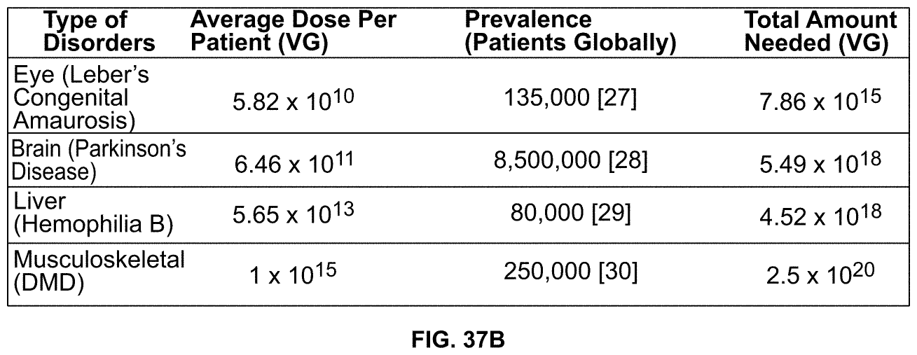

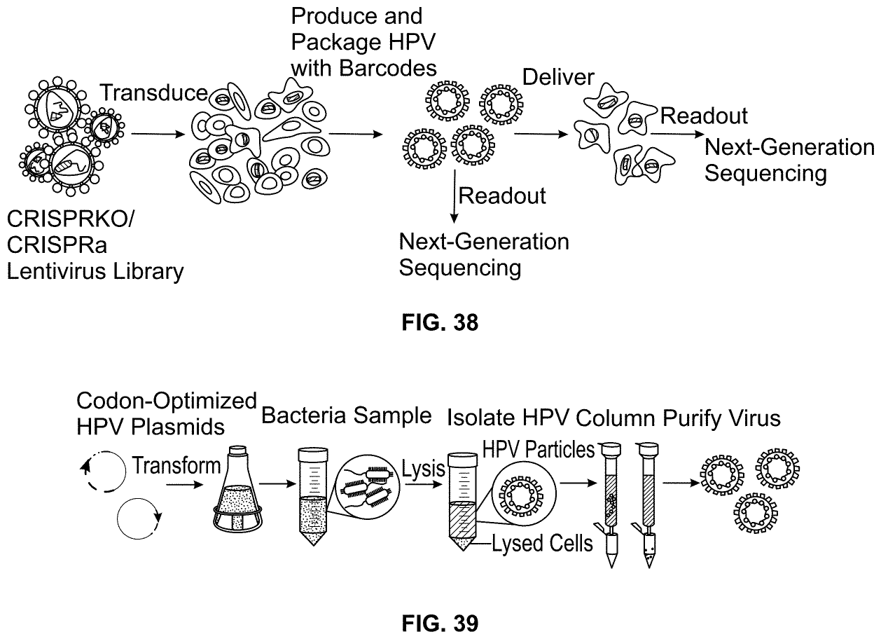

One skilled person in the art will appreciate that papillomaviral delivery vehicle can be significantly cheaper to use compared with other delivery vehicles known in the art (FIG. 37A-B) (see, e.g., Rodrigez, “Production of AAV vectors for gene therapy: a cost-effectiveness and risk assessment,” Ph.D. Thesis, M I T, 2016, which is incorporated by reference herein in its entirety), and the vehicle can be screened to improve production and thus its production cost as illustrated in FIGS. 38 and 39.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

SEQUENCE LISTING

| SEQUENCE | |
|---|---|
| ID | SEQUENCE |
| pDY0003HPV | gacggatcgggagatctcccgatcccctatggtgcactctcagtacaatctgctctgatgcc |
| 41 L1-HCV | gcatagttaagccagtatctgctccctgcttgtgtgttggaggtcgctgagtagtgcgcgag |
| IRES-L2 | caaaatttaagctacaacaaggcaaggcttgaccgacaattgcatgaagaatctgcttag |
| (seq | ggttaggcgttttgcgctgcttcgcgatgtacgggccagatatacgcgttgacattgattatt |
| 0D9LeHGo) | gactagttattaatagtaatcaattacggggtcattagttcatagcccatatatggagttccg |
| CMV | cgttacataacttacggtaaatggcccgcctggctgaccgcccaacgacccccgcccattg |
| promoter: | acgtcaataatgacgtatgttcccatagtaacgccaatagggactttccattgacgtcaatg |
| nucleotides | ggtggagtatttacggtaaactgcccacttggcagtacatcaagtgtatcatatgccaagta |
| 232 to 819 | cgccccctattgacgtcaatgacggtaaatggcccgcctggcattatgcccagtacatgac |
| T7 promoter: | cttatgggactttcctacttggcagtacatctacgtattagtcatcgctattaccatggtgatg |
| nucleotides | cggttttggcagtacatcaatgggcgtggatagcggtttgactcacggggatttccaagtct |
| 863 to 879 | ccaccccattgacgtcaatgggagtttgttttggcaccaaaatcaacgggactttccaaaat |
| HPV 41 L1 | gtcgtaacaactccgccccattgacgcaaatgggcggtaggcgtgtacggtgggaggtct |
| coding | atataagcagagctctctggctaactagagaacccactgcttactggcttatcgaaattaat |
| sequence: | acgactcactatagggagacccaagctggctagcgtttaaacttaagcttgccaccatgac |
| nucleotides | aggccttcagtatttatttttagcgatgatggcactcacattgtctatcctactagcacaaca |
| 923 to 2674 | gccaccaccccactcgtgcctgcacagcccagcgatgtgccctacattgttgttgacttgtat |
| IRES: | agtggaagtatggattatgatatacatcctagcctgttgcgcaggaaacgtaaaaaacgc |
| nucleotides | aaacgtgtttatttttcagatggccgtgtggcttccaggcccaaatagattttacttaccccct |
| 2675 to 3113 | caacctatacaacggacattgaacacagaggaatacgtgagacgcaccagtactttcctc |
| HPV 41 L2: | catgctgccactgaccgtttgcttactgttggacatccattttacaatattactaatgcggatg |
| nucleotides | gcaaagaggtggtccctaaagtttcctctaatcagttcagggccttccgtgtccgtttcccaa |
| 3114 to 4778 | atcccaatacctttgcattttgtgataagtccctttttaaccctgacaaggagcgtctggtctg |
| BGH polyA: | gggtattcgtgggattgaggtttctaggggacagcccttaggtattggtgtaacagggaac |
| nucleotides | cctttttttaataagtttgatgatgctgaaaatccctacaatggtataaacaaaaataacatt |
| 4829 to 5053 | actgaccaaggttcagactcaaggttgagcattgcatttgaccctaagcaaacacagctgc |
| | tgatagtaggtgctaaacctgcaaagggtgagtactgggacgttgctgcaacatgtgaaa |
| | accctccactgaccaaagcagatgacaaatgtcctgctctagagcttaagtcctcatacatt |
| | gaggatgcagacatgagtgacataggcctgggaaacttgaatttttctacactgcagaga |
| | aacaaatccgatgccccattagatattgtggattctatctgcaaatatcctgactacctgca |
| | aatgatagaagaactatatggagaccacatgtttttctatgtgcggTgtgaagctctgtatg |
| | ctaggcatataatgcaacacgcgggcaagatggatgctgagcaatttcccacttctctgta |
| | catagactcctctgtagaaggtgagaaattaaattccttgcagcgcactgataggtatttca |
| | tgacacccagcggctccctggtagctactgagcagcagctgtttaacaggcccttttggctg |
| | cagagatcccagggccataacaatggcatactgtggcacaacgaggcctttgtaacattg |
| | gttgacactaccaggggaactaactttaccatcagtgttcctgagggggatgcttcttcatat |
| | aacaattctaagttttttgagtttttaaggcacaccgaggagtttcagcttgcctttattctac |
| | agctgtgtaaggtagaccttacccctgagaatttggcttacatacacacaatggatccatcc |
| | attattgaagactggcatttagctgtcacttcacctcccaattctgtactggaggatcattata |
| | ggtacatactgtccattgcaactaaatgtccctctaaggatgcagatgatacctccactgac |
| | ccatacaaagatcttaagttttgggaggttgatctacgggatcgtatgacagagcaattgg |
| | accagactccccttggcaggaagtttttgtttcaaactggtatcactcagtcatcatcaaata |
| | agcgggtgtccacgcagtctactgcccttactacctacaggcggcctactaagcgccgccg |
| | gaaggcttaattctagtgtacgtagccagcccccgattgggggcgacactccaccatagat |
| | cactcccctgtgaggaactactgtcttcacgcagaaagcgtctagccatggcgttagtatga |
| | gagtcgtgcagcctccaggaccccccctcccgggagagccatagtggtctgcggaaccgg |
| | tgagtacaccggaattgccaggacgaccgggtcctttcttggatcaacccgctcaatgcct |
| | ggagatttgggcgtgcccccgcaagactgctagccgagtagtgttgggtcgcgaaaggcc |
| | ttgtggtactgcctgatagggtgcttgcgagtgccccgggaggtctcgtagaccgtgcacc |
| | atgagcacgaatcctaaacctcaaagaaaaaccaaacgtaacaccaaccgccgcccaca |
| | ggacgtcttcatatgtctagccaccatgcttgctaggcaaagggttaaacgcgctaatcctg |
| | aacaactgtataagacatgcaaagcaacggggggcgattgtccacccgatgttattaaac |
| | gctatgagcaaactacacctgctgatagtatattaaagtatgggagtgtaggggttttctttg |
| | gcggtctgggcattggcacaggacgtggtggcggtggcacagtgcttggggctggggcag |
| | ttgggggacgcccgtccatatccagtggtgcaattggtccccgggatattttgccaattgaa |
| | tcaggggggccttcactggcagaggaaatacctctgcttcccatggcaccccgtgtgccaa |
| | ggcctacagatccctttcggccgtcagtgctggaagagccttttattataaggcctcctgaa |
| | cgcccaaacattttgcatgagcagcgtttccctacagacgctgcaccatttgacaatggca |
| | acacagaaatcacaaccattcctagccaatatgatgttagtgggggaggggttgacattca |
| | gataattgaactccctagtgtgaatgaccccggtccctcggttgttacccgcacacaataca |
| | acaatccaacgtttgaggtggaggtgtccactgacattagtggagaaacctcatcaacgg |
| | acaacattattgtaggagctgaaagcggtggcacatccgtaggtgacaatgctgaactgat |
| | acctttgctagatatatcccggggggacacaattgacacaaTaatacttgcccctggcga |
| | ggaggagactgcctttgtgaccagcactcctgaacgtgtgcctatacaggagcgattacct |
| | attaggccctatggcagacagtatcagcaagtgcgagttaccgaccctgaatttttagaca |
| | gcgctgcagtacttgtctctttagagaatccagtgtttgatgcagacattactctcacgtttga |
| | ggatgatctgcagcaggcactacgtagtgacacagacctgcgggacgtgcgtcgcctcag |
| | tagaccttattaccagaggcgcactactggccttcgtgttagtcgcctggggcaacgtcggg |
| | gtactatatccacgcgctctggtgttcaggtaggctccgctgctcattttttccaggacattag |
| | tccaatcggccaggctattgagccaattgatgcaattgaactagatgtactgggtgagcaa |
| | tccggtgaggggactattgtgagaggagaccctacgccttctattgagcaagacatagga |
| | ctaaccgctttgggggacaacattgaaaatgaattgcaggaaatagatttattaactgcgg |
| | atggtgaagaagaccaggagggcagagacctgcagttggtattttccactggcaatgatg |
| | aggtggttgatattatgactatacctatacgtgcaggcggggatgacaggccttcagtattt |
| | atttttagcgatgatggcactcacattgtctatcctactagcacaacagccaccaccccact |
| | cgtgcctgcacagcccagcgatgtgccctacattgttgttgacttgtatagtggaagtatgg |
| | attatgatatacatcctagcctgttgcgcaggaaacgtaaaaaacgcaaacgtgtttattttt |
| | cagatggccgtgtggcttccaggcccaaataggcggccgctcgagtctagagggcccgttt |
| | aaacccgctgatcagcctcgactgtgccttctagttgccagccatctgttgtttgcccctccc |
| | ccgtgccttccttgaccctggaaggtgccactcccactgtcctttcctaataaaatgaggaa |
| | attgcatcgcattgtctgagtaggtgtcattctattctggggggtggggtggggcaggacag |
| | caagggggaggattgggaagacaatagcaggcatgctggggatgcggtgggctctatgg |
| | cttctgaggcggaaagaaccagctggggctctagggggtatccccacgcgccctgtagcg |
| | gcgcattaagcgcggcgggtgtggtggttacgcgcagcgtgaccgctacacttgccagcg |
| | ccctagcgcccgctcctttcgctttcttcccttcctttctcgccacgttcgccggctttccccgtc |
| | aagctctaaatcgggggctccctttagggttccgatttagtgctttacggcacctcgacccc |
| | aaaaaacttgattagggtgatggttcacgtagtgggccatcgccctgatagacggtttttcg |
| | ccctttgacgttggagtccacgttctttaatagtggactcttgttccaaactggaacaacact |
| | caaccctatctcggtctattcttttgatttataagggattttgccgatttcggcctattggttaa |
| | aaaatgagctgatttaacaaaaatttaacgcgaattaattctgtggaatgtgtgtcagttag |
| | ggtgtggaaagtccccaggctccccagcaggcagaagtatgcaaagcatgcatctcaatt |
| | agtcagcaaccaggtgtggaaagtccccaggctccccagcaggcagaagtatgcaaagc |
| | atgcatctcaattagtcagcaaccatagtcccgcccctaactccgcccatcccgcccctaac |
| | tccgcccagttccgcccattctccgccccatggctgactaattttttttatttatgcagaggcc |
| | gaggccgcctctgcctctgagctattccagaagtagtgaggaggcttttttggaggcctagg |
| | cttttgcaaaaagctcccgggagcttgtatatccattttcggatctgatcaagagacaggat |
| | gaggatcgtttcgcatgattgaacaagatggattgcacgcaggttctccggccgcttgggt |
| | ggagaggctattcggctatgactgggcacaacagacaatcggctgctctgatgccgccgt |
| | gttccggctgtcagcgcaggggcgcccggttctttttgtcaagaccgacctgtccggtgccc |
| | tgaatgaactgcaggacgaggcagcgcggctatcgtggctggccacgacgggcgttcctt |
| | gcgcagctgtgctcgacgttgtcactgaagcgggaagggactggctgctattgggcgaag |
| | tgccggggcaggatctcctgtcatctcaccttgctcctgccgagaaagtatccatcatggct |
| | gatgcaatgcggcggctgcatacgcttgatccggctacctgcccattcgaccaccaagcga |
| | aacatcgcatcgagcgagcacgtactcggatggaagccggtcttgtcgatcaggatgatct |
| | ggacgaagagcatcaggggctcgcgccagccgaactgttcgccaggctcaaggcgcgca |
| | tgcccgacggcgaggatctcgtcgtgacccatggcgatgcctgcttgccgaatatcatggt |
| | ggaaaatggccgcttttctggattcatcgactgtggccggctgggtgtggcggaccgctatc |
| | aggacatagcgttggctacccgtgatattgctgaagagcttggcggcgaatgggctgacc |
| | gcttcctcgtgctttacggtatcgccgctcccgattcgcagcgcatcgccttctatcgccttct |
| | tgacgagttcttctgagcgggactctggggttcgaaatgaccgaccaagcgacgcccaac |
| | ctgccatcacgagatttcgattccaccgccgccttctatgaaaggttgggcttcggaatcgtt |
| | ttccgggacgccggctggatgatcctccagcgcggggatctcatgctggagttcttcgccca |
| | ccccaacttgtttattgcagcttataatggttacaaataaagcaatagcatcacaaatttcac |
| | aaataaagcatttttttcactgcattctagttgtggtttgtccaaactcatcaatgtatcttatc |
| | atgtctgtataccgtcgacctctagctagagcttggcgtaatcatggtcatagctgtttcctgt |
| | gtgaaattgttatccgctcacaattccacacaacatacgagccggaagcataaagtgtaaa |
| | gcctggggtgcctaatgagtgagctaactcacattaattgcgttgcgctcactgcccgctttc |
| | cagtcgggaaacctgtcgtgccagctgcattaatgaatcggccaacgcgcggggagagg |
| | cggtttgcgtattgggcgctcttccgcttcctcgctcactgactcgctgcgctcggtcgttcgg |
| | ctgcggcgagcggtatcagctcactcaaaggcggtaatacggttatccacagaatcaggg |
| | gataacgcaggaaagaacatgtgagcaaaaggccagcaaaaggccaggaaccgtaaa |
| | aaggccgcgttgctggcgtttttccataggctccgcccccctgacgagcatcacaaaaatc |
| | gacgctcaagtcagaggtggcgaaacccgacaggactataaagataccaggcgtttcccc |
| | ctggaagctccctcgtgcgctctcctgttccgaccctgccgcttaccggatacctgtccgcct |
| | ttctcccttcgggaagcgtggcgctttctcatagctcacgctgtaggtatctcagttcggtgta |
| | ggtcgttcgctccaagctgggctgtgtgcacgaaccccccgttcagcccgaccgctgcgcc |
| | ttatccggtaactatcgtcttgagtccaacccggtaagacacgacttatcgccactggcagc |
| | agccactggtaacaggattagcagagcgaggtatgtaggcggtgctacagagttcttgaa |
| | gtggtggcctaactacggctacactagaagaacagtatttggtatctgcgctctgctgaagc |
| | cagttaccttcggaaaaagagttggtagctcttgatccggcaaacaaaccaccgctggtag |
| | cGGTggtttttttgtttgcaagcagcagattacgcgcagaaaaaaaggatctcaagaag |
| | atcctttgatcttttctacggggtctgacgctcagtggaacgaaaactcacgttaagggattt |
| | tggtcatgagattatcaaaaaggatcttcacctagatccttttaaattaaaaatgaagtttta |
| | aatcaatctaaagtatatatgagtaaacttggtctgacagttaccaatgcttaatcagtgag |
| | gcacctatctcagcgatctgtctatttcgttcatccatagttgcctgactccccgtcgtgtaga |
| | taactacgatacgggagggcttaccatctggccccagtgctgcaatgataccgcgagacc |
| | cacgctcaccggctccagatttatcagcaataaaccagccagccggaagggccgagcgc |
| | agaagtggtcctgcaactttatccgcctccatccagtctattaattgttgccgggaagctag |
| | agtaagtagttcgccagttaatagtttgcgcaacgttgttgccattgctacaggcatcgtggt |
| | gtcacgctcgtcgtttggtatggcttcattcagctccggttcccaacgatcaaggcgagttac |
| | atgatcccccatgttgtgcaaaaaagcggttagctccttcggtcctccgatcgttgtcagaa |
| | gtaagttggccgcagtgttatcactcatggttatggcagcactgcataattctcttactgtcat |
| | gccatccgtaagatgcttttctgtgactggtgagtactcaaccaagtcattctgagaatagt |
| | gtatgcggcgaccgagttgctcttgcccggcgtcaatacgggataataccgcgccacatag |
| | cagaactttaaaagtgctcatcattggaaaacgttcttcggggcgaaaactctcaaggatc |
| | ttaccgctgttgagatccagttcgatgtaacccactcgtgcacccaactgatcttcagcatct |
| | tttactttcaccagcgtttctgggtgagcaaaaacaggaaggcaaaatgccgcaaaaaag |
| | ggaataagggcgacacggaaatgttgaatactcatactcttcctttttcaatattattgaagc |
| | atttatcagggttattgtctcatgagcggatacatatttgaatgtatttagaaaaataaacaa |
| | ataggggttccgcgcacatttccccgaaaagtgccacctgacgtc (SEQ ID NO: 1) |
| HPV 41 L1 | MTGLQYLFLAMMALTLSILLAQQPPPHSCLHSPAMCPTL |
| amino acid | LLTCIVEVWIMIYILACCAGNVKNANVFIFQMAVWLPGP |
| sequence | NRFYLPPQPIQRTLNTEEYVRRTSTFLHAATDRLLTVGHP |
| | FYNITNADGKEVVPKVSSNQFRAFRVRFPNPNTFAFCDKS |
| | LFNPDKERLVWGIRGIEVSRGQPLGIGVTGNPFFNKFDDA |
| | ENPYNGINKNNITDQGSDSRLSIAFDPKQTQLLIVGAKPAK |
| | GEYWDVAATCENPPLTKADDKCPALELKSSYIEDADMSD |
| | IGLGNLNFSTLQRNKSDAPLDIVDSICKYPDYLQMIEELYG |
| | DHMFFYVRCEALYARHIMQHAGKMDAEQFPTSLYIDSSV |
| | EGEKLNSLQRTDRYFMTPSGSLVATEQQLFNRPFWLQRS |
| | QGHNNGILWHNEAFVTLVDTTRGTNFTISVPEGDASSYNN |
| | SKFFEFLRHTEEFQLAFILQLCKVDLTPENLAYIHTMDPSI |
| | IEDWHLAVTSPPNSVLEDHYRYILSIATKCPSKDADDTSTD |
| | PYKDLKFWEVDLRDRMTEQLDQTPLGRKFLFQTGITQSS |
| | SNKRVSTQSTALTTYRRPTKRRRKA (SEQ ID NO: 2) |
| HPV 41 L2 | MLARQRVKRANPEQLYKTCKATGGDCPPDVIKRYEQTT |
| amino acid | PADSILKYGSVGVFFGGLGIGTGRGGGGTVLGAGAVGGR |
| sequence | PSISSGAIGPRDILPIESGGPSLAEEIPLLPMAPRVPRPTDPF |
| | RPSVLEEPFIIRPPERPNILHEQRFPTDAAPFDNGNTEITTIP |
| | SQYDVSGGGVDIQIIELPSVNDPGPSVVTRTQYNNPTFEVE |
| | VSTDISGETSSTDNIIVGAESGGTSVGDNAELIPLLDISRGD |
| | TIDTIILAPGEEETAFVTSTPERVPIQERLPIRPYGRQYQQV |
| | RVTDPEFLDSAAVLVSLENPVFDADITLTFEDDLQQALRS |
| | DTDLRDVRRLSRPYYQRRTTGLRVSRLGQRRGTISTRSG |
| | VQVGSAAHFFQDISPIGQAIEPIDAIELDVLGEQSGEGTIVR |
| | GDPTPSIEQDIGLTALGDNIENELQEIDLLTADGEEDQEGR |
| | DLQLVFSTGNDEVVDIMTIPIRAGGDDRPSVFIFSDDGTHI |
| | VYPTSTTATTPLVPAQPSDVPYIVVDLYSGSMDYDIHPSLL |
| | RRKRKKRKRVYFSDGRVASRPK (SEQ ID NO: 3) |
| PDY0004HPV | gacggatcgggagatctcccgatcccctatggtgcactctcagtacaatctgctctgatgcc |
| 96 L1-HCV | gcatagttaagccagtatctgctccctgcttgtgtgttggaggtcgctgagtagtgcgcgag |
| IRES-L2 | caaaatttaagctacaacaaggcaaggcttgaccgacaattgcatgaagaatctgcttag |
| (seq | ggttaggcgttttgcgctgcttcgcgatgtacgggccagatatacgcgttgacattgattatt |
| WKo64IPx) | gactagttattaatagtaatcaattacggggtcattagttcatagcccatatatggagttccg |
| CMV | cgttacataacttacggtaaatggcccgcctggctgaccgcccaacgacccccgcccattg |
| promoter: | acgtcaataatgacgtatgttcccatagtaacgccaatagggactttccattgacgtcaatg |
| nucleotides | ggtggagtatttacggtaaactgcccacttggcagtacatcaagtgtatcatatgccaagta |
| 232 to 819 | cgccccctattgacgtcaatgacggtaaatggcccgcctggcattatgcccagtacatgac |
| T7 promoter: | cttatgggactttcctacttggcagtacatctacgtattagtcatcgctattaccatggtgatg |
| nucleotides | cggttttggcagtacatcaatgggcgtggatagcggtttgactcacggggatttccaagtct |
| 863 to 879 | ccaccccattgacgtcaatgggagtttgttttggcaccaaaatcaacgggactttccaaaat |
| HPV 96 L1 | gtcgtaacaactccgccccattgacgcaaatgggcggtaggcgtgtacggtgggaggtct |
| sequence: | atataagcagagctctctggctaactagagaacccactgcttactggcttatcgaaattaat |
| nucleotides | acgactcactatagggagacccaagctggctagcgtttaaacttaAGCTTGCCAC |
| 923 to 2461 | Catgtcatcattgtggttgtcaacaacgggtaaggtctatttaccaccatcaacaccagttg |
| IRES: | ccagggtgcaaagcacggactcctacatacaaagaacaaacatctattatcatgctaata |
| nucleotides | ctgaccgcctgttaacagtaggacatccttattttgatgtgaggaaaaataatggagatcat |
| 2462 to 2900 | gaagtgttagttcccaaggtgtcaggtaatcagtacagggcctttagggtacacttaccgg |
| HPV 96 L2 | atcctaacagatttgctctagctgacatgtcagtggtaaatcctgatagggagcgtttggtat |
| sequence: | gggctgttagaggaatggaaattggtcgtggacagccattaggtgtaggtacatcaggac |
| nucleotides | atccattatttaacaaggtgaaagacacggaaaatccaaatggctataatacaggtggaa |
| 2901 to 4466 | aggatgatagggtgaatacatcctttgatcccaaacaaattcaaatgtttgttttgggttgta |
| BGH polyA: | taccctgcttgggggaacattgggacaaggccttaccttgtgtagaaaatcctcctgatcag |
| nucleotides | ggagcgtgtccacctctagaattaaaaaatactattattgaagatggggacatgggagac |
| 4517 to 4741 | atagggtttggaaatcttaattttaaaacattatcagtcactaagtctgatgttagtctggat |
| | attgttaatgaaatttgcaagtatccagatttcttaaaaatggctaatgatgtgtatggcaat |
| | gcttgcttcttttatgccagaagagaacaatgttatgccagacatatgttttgtagaggtggg |
| | tcagtaggagacagtattccagatgatgcagttggagaagacaaccattattatttaaagg |
| | ctgccagtgatcaaaacagagatacaatggcaagttccatttacactcccacagtcagtgg |
| | atctttagtttctacagatgcacagattttcaataggcctttttggctgcaaagggctcaagg |
| | ccataataatggtatttgctggggtaatcaaatctttctcacagtaatagataataccagga |
| | atactaatttctgtatcagtgtctcctcaaatgatcaggcattacaggaatacaatactgca |
| | aactttagagaatatttgagacatgtagaagagtatgaattatcctttatattacaattatgt |
| | aaagttccattagagccagaagtattagcacaaattaatgctatgaatgcagacattttag |
| | aagattggcaattaggttttgttccttctcctgacaatcccatcaatgatacatatagataca |
| | tacattcagcagccacacggtgtccagataaaactacacctaaagaaaaagcagatccct |
| | ttgcaggttatcacttttgggatgttgatttgtctgaaaagttatcattagatttagatcagtat |
| | tctctgggacgtaaattcttatttcaagccaacctgcaaaacaaaagagttaacagagggg |
| | ttactgtaaccgggagggctacaacctcaagaggtacaaaacgaaaacgacgctgTttct |
| | agtgtacgtagccagcccccgattgggggcgacactccaccatagatcactcccctgtgag |
| | gaactactgtcttcacgcagaaagcgtctagccatggcgttagtatgagagtcgtgcagcc |
| | tccaggaccccccctcccgggagagccatagtggtctgcggaaccggtgagtacaccgga |
| | attgccaggacgaccgggtcctttcttggatcaacccgctcaatgcctggagatttgggcgt |
| | gcccccgcaagactgctagccgagtagtgttgggtcgcgaaaggccttgtggtactgcctg |
| | atagggtgcttgcgagtgccccgggaggtctcgtagaccgtgcaccatgagcacgaatcct |
| | aaacctcaaagaaaaaccaaacgtaacaccaaccgccgTccacaggacgtcttcatatg |
| | tctagccaccatggcgcgcgcacgtagagtaaagcgtgattctgttacaaatatttacagg |
| | ggctgtaaggcagctggcacatgcccccctgatgttattaataaagttgaacaaaaaacta |
| | ttgctgaccaaattttaaagtatggcagcaccgctgcgttttttggtgggttgggtattagta |
| | caggcaaaggaactggaggcagtactggttatgtccctttgcctgaaggacctgcacctgg |
| | tgttcgcgtgggtggtacaccaactgtggtgcgccccggggtcattccagaagcgattggt |
| | cctactgatataatacctttggatacagtcaaccctattgaccctgttgcaccttcagttgtcc |
| | ctcttacagacacaggacctgatttgttgccaggagaaattgagaccattgctgaggtaca |
| | tcctgtgtcagatgtaacacctgttgacacaccagtggtgacaggtggtagaggctcgagt |
| | gcagtattagaggttgctgacccaagtcctcccactcgtgcacgtgtcagtagaacacaat |
| | atcataacccagcttttcaaataatatctgaaacaacaccaacaactggggaagcgtcgtt |
| | atctgaccaaatcattgtacaatcaggttctggaggacaaaatattggtggtagtgggcctt |
| | ctgtggaaatagaattagaagagttccccacaagatattcatttgaaatagaagagccaa |
| | cccctcctagaaaaactagtacacctgtaagaatggctcagcaggcctcacgagctttacg |
| | tagagctttatacaatcgtagattaacacaacaggtttctgtagaaaatcctctatttttaca |
| | acagccttctaaattagttacttttcaatttgataaccctgcatatgaggaggaaataacac |
| | aaatatttgagagggatttaagctccattgaagaacctccagatagacaatttatggatgtt |
| | gttaaattaggtaggcctacatatgctgaaacaccagaaggttacattagagtcagtagac |
| | ttgggaaacgagcaaccatcagaacacgctctggagcacaggttggcactcaagttcact |
| | tttacagagatataagcactattgacacagaaccctccattgaattgcaactgttagggga |
| | acattctggggatgctagtattgttcaaggcccagtagaaagtacatttgttaatatggatgt |
| | acaagaaattcctactttggaggaagtgccagaattacattctgaagatgtgctattagag |
| | gaggcattagaagactttagtggagcacaattagtttttggaaattctagaagatcaaatgt |
| | aataactattcctagatttgagactccaagagagattaatatttatacaccagatttagatg |
| | gatattacatatcatatccagaaacaaggaatattccagaagttatatacactgagccaga |
| | cacgactccaacaataataattcatacagaggatttcagtggtgattattatttacatccaa |
| | gtttgagacgaagaaaaagaaaacgagcctatttgtaagAggccgctcgagtctagagg |
| | gcccgtttaaacccgctgatcagcctcgactgtgccttctagttgccagccatctgttgtttgc |
| | ccctcccccgtgccttccttgaccctggaaggtgccactcccactgtcctttcctaataaaat |
| | gaggaaattgcatcgcattgtctgagtaggtgtcattctattctggggggtggggtggggca |
| | ggacagcaagggggaggattgggaagacaatagcaggcatgctggggatgcggtgggc |
| | tctatggcttctgaggcggaaagaaccagctggggctctagggggtatccccacgcgccct |
| | gtagcggcgcattaagcgcggcgggtgtggtggttacgcgcagcgtgaccgctacacttg |
| | ccagcgccctagcgcccgctcctttcgctttcttcccttcctttctcgccacgttcgccggcttt |
| | ccccgtcaagctctaaatcgggggctccctttagggttccgatttagtgctttacggcacctc |
| | gaccccaaaaaacttgattagggtgatggttcacgtagtgggccatcgccctgatagacgg |
| | tttttcgccctttgacgttggagtccacgttctttaatagtggactcttgttccaaactggaac |
| | aacactcaaccctatctcggtctattcttttgatttataagggattttgccgatttcggcctatt |
| | ggttaaaaaatgagctgatttaacaaaaatttaacgcgaattaattctgtggaatgtgtgtc |
| | agttagggtgtggaaagtccccaggctccccagcaggcagaagtatgcaaagcatgcatc |
| | tcaattagtcagcaaccaggtgtggaaagtccccaggctccccagcaggcagaagtatgc |
| | aaagcatgcatctcaattagtcagcaaccatagtcccgcccctaactccgcccatcccgcc |
| | cctaactccgcccagttccgcccattctccgccccatggctgactaattttttttatttatgcag |
| | aggccgaggccgcctctgcctctgagctattccagaagtagtgaggaggcttttttggagg |
| | cctaggcttttgcaaaaagctcccgggagcttgtatatccattttcggatctgatcaagaga |
| | caggatgaggatcgtttcgcatgattgaacaagatggattgcacgcaggttctccggccgc |
| | ttgggtggagaggctattcggctatgactgggcacaacagacaatcggctgctctgatgcc |
| | gccgtgttccggctgtcagcgcaggggcgcccggttctttttgtcaagaccgacctgtccgg |
| | tgccctgaatgaactgcaggacgaggcagcgcggctatcgtggctggccacgacgggcgt |
| | tccttgcgcagctgtgctcgacgttgtcactgaagcgggaagggactggctgctattgggc |
| | gaagtgccggggcaggatctcctgtcatctcaccttgctcctgccgagaaagtatccatcat |
| | ggctgatgcaatgcggcggctgcatacgcttgatccggctacctgcccattcgaccaccaa |
| | gcgaaacatcgcatcgagcgagcacgtactcggatggaagccggtcttgtcgatcaggat |
| | gatctggacgaagagcatcaggggctcgcgccagccgaactgttcgccaggctcaaggc |
| | gcgcatgcccgacggcgaggatctcgtcgtgacccatggcgatgcctgcttgccgaatatc |
| | atggtggaaaatggccgcttttctggattcatcgactgtggccggctgggtgtggcggacc |
| | gctatcaggacatagcgttggctacccgtgatattgctgaagagcttggcggcgaatgggc |
| | tgaccgcttcctcgtgctttacggtatcgccgctcccgattcgcagcgcatcgccttctatcg |
| | ccttcttgacgagttcttctgagcgggactctggggttcgaaatgaccgaccaagcgacgc |
| | ccaacctgccatcacgagatttcgattccaccgccgccttctatgaaaggttgggcttcgga |
| | atcgttttccgggacgccggctggatgatcctccagcgcggggatctcatgctggagttctt |
| | cgcccaccccaacttgtttattgcagcttataatggttacaaataaagcaatagcatcacaa |
| | atttcacaaataaagcatttttttcactgcattctagttgtggtttgtccaaactcatcaatgta |
| | tcttatcatgtctgtataccgtcgacctctagctagagcttggcgtaatcatggtcatagctgt |
| | ttcctgtgtgaaattgttatccgctcacaattccacacaacatacgagccggaagcataaa |
| | gtgtaaagcctggggtgcctaatgagtgagctaactcacattaattgcgttgcgctcactgc |
| | ccgctttccagtcgggaaacctgtcgtgccagctgcattaatgaatcggccaacgcgcggg |
| | gagaggcggtttgcgtattgggcgctcttccgcttcctcgctcactgactcgctgcgctcggt |
| | cgttcggctgcggcgagcggtatcagctcactcaaaggcggtaatacggttatccacaga |
| | atcaggggataacgcaggaaagaacatgtgagcaaaaggccagcaaaaggccaggaa |
| | ccgtaaaaaggccgcgttgctggcgtttttccataggctccgcccccctgacgagcatcac |
| | aaaaatcgacgctcaagtcagaggtggcgaaacccgacaggactataaagataccaggc |
| | gtttccccctggaagctccctcgtgcgctctcctgttccgaccctgccgcttaccggatacct |
| | gtccgcctttctcccttcgggaagcgtggcgctttctcatagctcacgctgtaggtatctcagt |
| | tcggtgtaggtcgttcgctccaagctgggctgtgtgcacgaaccccccgttcagcccgacc |
| | gctgcgccttatccggtaactatcgtcttgagtccaacccggtaagacacgacttatcgcca |
| | ctggcagcagccactggtaacaggattagcagagcgaggtatgtaggcggtgctacaga |
| | gttcttgaagtggtggcctaactacggctacactagaagaacagtatttggtatctgcgctc |
| | tgctgaagccagttaccttcggaaaaagagttggtagctcttgatccggcaaacaaaccac |
| | cgctggtagcGGTggtttttttgtttgcaagcagcagattacgcgcagaaaaaaaggat |
| | ctcaagaagatcctttgatcttttctacggggtctgacgctcagtggaacgaaaactcacgt |
| | taagggattttggtcatgagattatcaaaaaggatcttcacctagatccttttaaattaaaaa |
| | tgaagttttaaatcaatctaaagtatatatgagtaaacttggtctgacagttaccaatgctta |
| | atcagtgaggcacctatctcagcgatctgtctatttcgttcatccatagttgcctgactccccg |
| | tcgtgtagataactacgatacgggagggcttaccatctggccccagtgctgcaatgatacc |
| | gcgagacccacgctcaccggctccagatttatcagcaataaaccagccagccggaaggg |
| | ccgagcgcagaagtggtcctgcaactttatccgcctccatccagtctattaattgttgccgg |
| | gaagctagagtaagtagttcgccagttaatagtttgcgcaacgttgttgccattgctacagg |
| | catcgtggtgtcacgctcgtcgtttggtatggcttcattcagctccggttcccaacgatcaag |
| | gcgagttacatgatcccccatgttgtgcaaaaaagcggttagctccttcggtcctccgatcg |
| | ttgtcagaagtaagttggccgcagtgttatcactcatggttatggcagcactgcataattctc |
| | ttactgtcatgccatccgtaagatgcttttctgtgactggtgagtactcaaccaagtcattctg |
| | agaatagtgtatgcggcgaccgagttgctcttgcccggcgtcaatacgggataataccgcg |
| | ccacatagcagaactttaaaagtgctcatcattggaaaacgttcttcggggcgaaaactct |
| | caaggatcttaccgctgttgagatccagttcgatgtaacccactcgtgcacccaactgatct |
| | tcagcatcttttactttcaccagcgtttctgggtgagcaaaaacaggaaggcaaaatgccg |
| | caaaaaagggaataagggcgacacggaaatgttgaatactcatactcttcctttttcaatat |
| | tattgaagcatttatcagggttattgtctcatgagcggatacatatttgaatgtatttagaaa |
| | aataaacaaataggggttccgcgcacatttccccgaaaagtgccacctgacgtc |
| | (SEQ ID NO: 4) |
| HPV 96 L1 | MSSLWLSTTGKVYLPPSTPVARVQSTDSYIQRTNIYYHAN |
| amino acid | TDRLLTVGHPYFDVRKNNGDHEVLVPKVSGNQYRAFRV |
| sequence | HLPDPNRFALADMSVVNPDRERLVWAVRGMEIGRGQPL |
| | GVGTSGHPLFNKVKDTENPNGYNTGGKDDRVNTSFDPK |
| | QIQMFVLGCIPCLGEHWDKALPCVENPPDQGACPPLELK |
| | NTIIEDGDMGDIGFGNLNFKTLSVTKSDVSLDIVNEICKYP |
| | DFLKMANDVYGNACFFYARREQCYARHMFCRGGSVGDS |
| | IPDDAVGEDNHYYLKAASDQNRDTMASSIYTPTVSGSLVS |
| | TDAQIFNRPFWLQRAQGHNNGICWGNQIFLTVIDNTRNT |
| | NFCISVSSNDQALQEYNTANFREYLRHVEEYELSFILQLC |
| | KVPLEPEVLAQINAMNADILEDWQLGFVPSPDNPINDTYR |
| | YIHSAATRCPDKTTPKEKADPFAGYHFWDVDLSEKLSLD |
| | LDQYSLGRKFLFQANLQNKRVNRGVTVTGRATTSRGTK |
| | RKRRC (SEQ ID NO: 5) |
| HPV 96 L2 | MARARRVKRDSVTNIYRGCKAAGTCPPDVINKVEQKTIA |
| amino acid | DQILKYGSTAAFFGGLGISTGKGTGGSTGYVPLPEGPAPG |
| sequence | VRVGGTPTVVRPGVIPEAIGPTDIIPLDTVNPIDPVAPSVVP |
| | LTDTGPDLLPGEIETIAEVHPVSDVTPVDTPVVTGGRGSSA |
| | VLEVADPSPPTRARVSRTQYHNPAFQIISETTPTTGEASLS |
| | DQIIVQSGSGGQNIGGSGPSVEIELEEFPTRYSFEIEEPTPP |
| | RKTSTPVRMAQQASRALRRALYNRRLTQQVSVENPLFLQ |
| | QPSKLVTFQFDNPAYEEEITQIFERDLSSIEEPPDRQFMDV |
| | VKLGRPTYAETPEGYIRVSRLGKRATIRTRSGAQVGTQV |
| | HFYRDISTIDTEPSIELQLLGEHSGDASIVQGPVESTFVNM |
| | DVQEIPTLEEVPELHSEDVLLEEALEDFSGAQLVFGNSRR |
| | SNVITIPRFETPREINIYTPDLDGYYISYPETRNIPEVIYTEP |
| | DTTPTIIIHTEDFSGDYYLHPSLRRRKRKRAYL |
| | (SEQ ID NO: 6) |
| pDY0005HPV- | gacggatcgggagatctcccgatcccctatggtgcactctcagtacaatctgctctgatgcc |
| 1a L1-HCV | gcatagttaagccagtatctgctccctgcttgtgtgttggaggtcgctgagtagtgcgcgag |
| IRES-L2 | caaaatttaagctacaacaaggcaaggcttgaccgacaattgcatgaagaatctgcttag |
| (seq | ggttaggcgttttgcgctgcttcgcgatgtacgggccagatatacgcgttgacattgattatt |
| j7815OQL) | gactagttattaatagtaatcaattacggggtcattagttcatagcccatatatggagttccg |
| CMV | cgttacataacttacggtaaatggcccgcctggctgaccgcccaacgacccccgcccattg |
| promoter: | acgtcaataatgacgtatgttcccatagtaacgccaatagggactttccattgacgtcaatg |
| nucleotides | ggtggagtatttacggtaaactgcccacttggcagtacatcaagtgtatcatatgccaagta |
| 232 to 819 | cgccccctattgacgtcaatgacggtaaatggcccgcctggcattatgcccagtacatgac |
| T7 promoter: | cttatgggactttcctacttggcagtacatctacgtattagtcatcgctattaccatggtgatg |
| nucleotides | cggttttggcagtacatcaatgggcgtggatagcggtttgactcacggggatttccaagtct |
| 863 to 879 | ccaccccattgacgtcaatgggagtttgttttggcaccaaaatcaacgggactttccaaaat |
| HPV-1a L1 | gtcgtaacaactccgccccattgacgcaaatgggcggtaggcgtgtacggtgggaggtct |
| coding | atataagcagagctctctggctaactagagaacccactgcttactggcttatcgaaattaat |
| sequence: | acgactcactatagggagacccaagctggctagcgtttaaacttaagcttgccaccatgta |
| nucleotides | taatgtttttcagatggctgtctggttaccagcgcagaataagttctatcttcctccccagccc |
| 923 to 2449 | atcactagaatcctgtccactgatgaatatgtaaccagaaccaatctcttctaccatgcaac |
| IRES: | atctgaacgtctactgctggtcggacatcctttgtttgagatctccagtaatcaaactgtaac |
| nucleotides | tataccaaaagtgtcaccaaatgcatttagagtttttagggtgcgttttgctgatccaaatag |
| 2450 to 2888 | atttgcatttggggataaggcaatttttaatccagaaacagaaagattagtttggggcctaa |
| HPV-1a L2: | gagggatagagataggtagaggccagcctttaggtataggaataacgggccaccctctttt |
| Nucleotides | caataagttagatgatgcagaaaatccaacaaattatattaatactcatgcaaatggagat |
| 2889 to 4412 | tctagacaaaatactgcttttgatgcaaaacagacacaaatgttcctcgtcggctgtactcc |
| BGH polyA: | tgcttcaggtgaacactggacaagtagtcgttgcccaggggaacaagtgaaacttgggga |
| nucleotides | ctgccccagggtgcaaatgatagagtctgtcatagaagatggtgacatgatggatattggt |
| 4463 to 4687 | tttggggctatggattttgctgctttacagcaagacaagtctgatgtccctttagatgttgttc |
| | aagcaacatgcaaatatcctgattatatcagaatgaaccatgaagcctatggcaactctat |
| | gtttttttttgcacgtcgcgagcaaatgtataccaggcacttttttactcgcgggggttcggtg |
| | ggtgataaggaggcagtcccacaaagcctgtatttaacagcagatgctgaaccaagaac |
| | aactttagcaacaacaaattatgtaggcacaccaagtggctctatggtttcatctgatgtcc |
| | aattgtttaatagatcttactggcttcagcgatgtcaaggccagaataatggcatttgctgg |
| | agaaaccagttatttattacagttggagataataccagaggaacaagtttatctatcagtat |
| | gaaaaacaatgcaagtactacatattccaatgctaattttaatgattttctaagacatactg |
| | aagaatttgatctttcttttatagttcagctttgtaaagtaaagttaactcccgaaaatctagc |
| | ctacattcatacaatggaccctaatattttagaggattggcaactatctgtatctcaaccacc |
| | taccaatcctctagaagatcaatataggtttttagggtcttccttggcagcaaaatgtccag |
| | aacaggcgcctcctgagccccagactgatccttatagtcaatataaattctgggaagtcga |
| | tctcacagaaaggatgtccgaacaattagaccaatttccactaggaaggaaatttctatat |
| | caaagtggcatgacacaacgtactgctactagttccaccacaaagcgcaaaacagtgcgt |
| | ttatctacgtcagccaagcgcaggcgtaaggcttagttctagtgtacgtagccagcccccg |
| | attgggggcgacactccaccatagatcactcccctgtgaggaactactgtcttcacgcaga |
| | aagcgtctagccatggcgttagtatgagagtcgtgcagcctccaggaccccccctcccggg |
| | agagccatagtggtctgcggaaccggtgagtacaccggaattgccaggacgaccgggtc |
| | ctttcttggatcaacccgctcaatgcctggagatttgggcgtgcccccgcaagactgctagc |
| | cgagtagtgttgggtcgcgaaaggccttgtggtactgcctgatagggtgcttgcgagtgcc |
| | ccgggaggtctcgtagaccgtgcaccatgagcacgaatcctaaacctcaaagaaaaacc |
| | aaacgtaacaccaaccgccgcccacaggacgtcttcatatgtctagccaccatgtatcgcc |
| | tacgtagaaaacgcgctgcccccaaagatatatacccctcatgcaaaatatcaaacacct |
| | gcccacctgacattcaaaataaaattgagcatacaacaattgctgataaaatattgcaata |
| | tggcagtctgggagtttttttgggaggtttgggcattggaacagccagaggctctggagga |
| | agaattggttatactcccctcggtgagggtggtggggttagagttgctactcgtccaactcc |
| | agtaaggcctacaatacctgtggaaacagtaggccccagtgaaattttccccatagatgtt |
| | gtagatcctacaggccctgctgttattcccctacaagatttaggtagagacttcccaatacc |
| | aactgtgcaggttattgcagaaattcaccctatttctgacataccaaacattgttgcttcttca |
| | acaaatgaaggagaatctgccatattagatgtgttacagggaagtgcaaccatacgcact |
| | gtttcaagaacacaatacaataacccctctttcactgttgcatctacatctaatataagtgct |
| | ggagaagcatcaacatcagatattgtatttgttagcaatggttcaggtgacagggtggtgg |
| | gcgaggatatccccttggtagaattaaacttaggccttgaaacagacacatcttctgttgta |
| | caagaaacagcattttccagcagcacaccaattgctgaaagaccctcttttaggccctcaa |
| | gattctataataggcgtctatatgaacaggtgcaagtacaagaccctaggttcgttgagca |
| | gccacagtcaatggtcacttttgataatccagcatttgagccagagcttgatgaggtgtcta |
| | ttatcttccaaagagacttagatgctcttgctcagacaccagtgcctgaatttagagatgta |
| | gtttatctgagcaagcccacattttcgcgggaaccagggggacggttaagggttagccgcc |
| | ttggcaaaagttcaactattcgtacacgcctgggcacagcaattggcgccagaacccactt |
| | tttctatgatttaagttctattgctccagaagactcaattgaattattgcctttaggtgagcat |
| | agtcaaacaacagtcattagttccaacttaggtgacacagcatttatacaaggtgagacag |
| | cagaggatgacttagaagttatctctttagaaacaccacaattatattcagaagaagagct |
| | tttagacacaaacgaaagtgtgggcgaaaatttgcaacttactattactaactcagagggt |
| | gaggtttctatactagatttaacacaaagcagagtcaggccaccttttggcactgaagata |
| | ctagcttgcatgtatattacccaaattcttctaaagggactccaataattaatcctgaagaat |
| | catttacacctttggttattatagctcttaacaactcaacaggggattttgagttacatcctag |
| | tcttagaaagcgtcgtaaaagagcttatgtataagcggccgctcgagtctagagggcccgt |
| | ttaaacccgctgatcagcctcgactgtgccttctagttgccagccatctgttgtttgcccctcc |
| | cccgtgccttccttgaccctggaaggtgccactcccactgtcctttcctaataaaatgagga |
| | aattgcatcgcattgtctgagtaggtgtcattctattctggggggtggggtggggcaggaca |
| | gcaagggggaggattgggaagacaatagcaggcatgctggggatgcggtgggctctatg |
| | gcttctgaggcggaaagaaccagctggggctctagggggtatccccacgcgccctgtagc |
| | ggcgcattaagcgcggcgggtgtggtggttacgcgcagcgtgaccgctacacttgccagc |
| | gccctagcgcccgctcctttcgctttcttcccttcctttctcgccacgttcgccggctttccccg |
| | tcaagctctaaatcgggggctccctttagggttccgatttagtgctttacggcacctcgaccc |
| | caaaaaacttgattagggtgatggttcacgtagtgggccatcgccctgatagacggtttttc |
| | gccctttgacgttggagtccacgttctttaatagtggactcttgttccaaactggaacaacac |
| | tcaaccctatctcggtctattcttttgatttataagggattttgccgatttcggcctattggtta |
| | aaaaatgagctgatttaacaaaaatttaacgcgaattaattctgtggaatgtgtgtcagtta |
| | gggtgtggaaagtccccaggctccccagcaggcagaagtatgcaaagcatgcatctcaat |
| | tagtcagcaaccaggtgtggaaagtccccaggctccccagcaggcagaagtatgcaaag |
| | catgcatctcaattagtcagcaaccatagtcccgcccctaactccgcccatcccgcccctaa |
| | ctccgcccagttccgcccattctccgccccatggctgactaattttttttatttatgcagaggc |
| | cgaggccgcctctgcctctgagctattccagaagtagtgaggaggcttttttggaggcctag |
| | gcttttgcaaaaagctcccgggagcttgtatatccattttcggatctgatcaagagacagga |
| | tgaggatcgtttcgcatgattgaacaagatggattgcacgcaggttctccggccgcttgggt |
| | ggagaggctattcggctatgactgggcacaacagacaatcggctgctctgatgccgccgt |
| | gttccggctgtcagcgcaggggcgcccggttctttttgtcaagaccgacctgtccggtgccc |
| | tgaatgaactgcaggacgaggcagcgcggctatcgtggctggccacgacgggcgttcctt |
| | gcgcagctgtgctcgacgttgtcactgaagcgggaagggactggctgctattgggcgaag |
| | tgccggggcaggatctcctgtcatctcaccttgctcctgccgagaaagtatccatcatggct |
| | gatgcaatgcggcggctgcatacgcttgatccggctacctgcccattcgaccaccaagcga |
| | aacatcgcatcgagcgagcacgtactcggatggaagccggtcttgtcgatcaggatgatct |
| | ggacgaagagcatcaggggctcgcgccagccgaactgttcgccaggctcaaggcgcgca |
| | tgcccgacggcgaggatctcgtcgtgacccatggcgatgcctgcttgccgaatatcatggt |
| | ggaaaatggccgcttttctggattcatcgactgtggccggctgggtgtggcggaccgctatc |
| | aggacatagcgttggctacccgtgatattgctgaagagcttggcggcgaatgggctgacc |
| | gcttcctcgtgctttacggtatcgccgctcccgattcgcagcgcatcgccttctatcgccttct |
| | tgacgagttcttctgagcgggactctggggttcgaaatgaccgaccaagcgacgcccaac |
| | ctgccatcacgagatttcgattccaccgccgccttctatgaaaggttgggcttcggaatcgtt |
| | ttccgggacgccggctggatgatcctccagcgcggggatctcatgctggagttcttcgccca |
| | ccccaacttgtttattgcagcttataatggttacaaataaagcaatagcatcacaaatttcac |
| | aaataaagcatttttttcactgcattctagttgtggtttgtccaaactcatcaatgtatcttatc |
| | atgtctgtataccgtcgacctctagctagagcttggcgtaatcatggtcatagctgtttcctgt |
| | gtgaaattgttatccgctcacaattccacacaacatacgagccggaagcataaagtgtaaa |
| | gcctggggtgcctaatgagtgagctaactcacattaattgcgttgcgctcactgcccgctttc |
| | cagtcgggaaacctgtcgtgccagctgcattaatgaatcggccaacgcgcggggagagg |
| | cggtttgcgtattgggcgctcttccgcttcctcgctcactgactcgctgcgctcggtcgttcgg |
| | ctgcggcgagcggtatcagctcactcaaaggcggtaatacggttatccacagaatcaggg |
| | gataacgcaggaaagaacatgtgagcaaaaggccagcaaaaggccaggaaccgtaaa |
| | aaggccgcgttgctggcgtttttccataggctccgcccccctgacgagcatcacaaaaatc |
| | gacgctcaagtcagaggtggcgaaacccgacaggactataaagataccaggcgtttcccc |
| | ctggaagctccctcgtgcgctctcctgttccgaccctgccgcttaccggatacctgtccgcct |
| | ttctcccttcgggaagcgtggcgctttctcatagctcacgctgtaggtatctcagttcggtgta |
| | ggtcgttcgctccaagctgggctgtgtgcacgaaccccccgttcagcccgaccgctgcgcc |
| | ttatccggtaactatcgtcttgagtccaacccggtaagacacgacttatcgccactggcagc |
| | agccactggtaacaggattagcagagcgaggtatgtaggcggtgctacagagttcttgaa |
| | gtggtggcctaactacggctacactagaagaacagtatttggtatctgcgctctgctgaagc |
| | cagttaccttcggaaaaagagttggtagctcttgatccggcaaacaaaccaccgctggtag |
| | cggtggtttttttgtttgcaagcagcagattacgcgcagaaaaaaaggatctcaagaagat |
| | cctttgatcttttctacggggtctgacgctcagtggaacgaaaactcacgttaagggattttg |
| | gtcatgagattatcaaaaaggatcttcacctagatccttttaaattaaaaatgaagttttaaa |
| | tcaatctaaagtatatatgagtaaacttggtctgacagttaccaatgcttaatcagtgaggc |
| | acctatctcagcgatctgtctatttcgttcatccatagttgcctgactccccgtcgtgtagata |
| | actacgatacgggagggcttaccatctggccccagtgctgcaatgataccgcgagaccca |
| | cgctcaccggctccagatttatcagcaataaaccagccagccggaagggccgagcgcag |
| | aagtggtcctgcaactttatccgcctccatccagtctattaattgttgccgggaagctagagt |
| | aagtagttcgccagttaatagtttgcgcaacgttgttgccattgctacaggcatcgtggtgtc |
| | acgctcgtcgtttggtatggcttcattcagctccggttcccaacgatcaaggcgagttacatg |
| | atcccccatgttgtgcaaaaaagcggttagctccttcggtcctccgatcgttgtcagaagta |
| | agttggccgcagtgttatcactcatggttatggcagcactgcataattctcttactgtcatgc |
| | catccgtaagatgcttttctgtgactggtgagtactcaaccaagtcattctgagaatagtgta |
| | tgcggcgaccgagttgctcttgcccggcgtcaatacgggataataccgcgccacatagca |
| | gaactttaaaagtgctcatcattggaaaacgttcttcggggcgaaaactctcaaggatctta |
| | ccgctgttgagatccagttcgatgtaacccactcgtgcacccaactgatcttcagcatctttt |
| | actttcaccagcgtttctgggtgagcaaaaacaggaaggcaaaatgccgcaaaaaaggg |
| | aataagggcgacacggaaatgttgaatactcatactcttcctttttcaatattattgaagcat |
| | ttatcagggttattgtctcatgagcggatacatatttgaatgtatttagaaaaataaacaaat |
| | aggggttccgcgcacatttccccgaaaagtgccacctgacgtc (SEQ ID NO: 7) |
| HPV-1a L1 | MYNVFQMAVWLPAQNKFYLPPQPITRILSTDEYVTRTNL |
| amino acid | FYHATSERLLLVGHPLFEISSNQTVTIPKVSPNAFRVFRVR |
| sequence | FADPNRFAFGDKAIFNPETERLVWGLRGIEIGRGQPLGIGI |
| | TGHPLFNKLDDAENPTNYINTHANGDSRQNTAFDAKQTQ |
| | MFLVGCTPASGEHWTSSRCPGEQVKLGDCPRVQMIESVI |
| | EDGDMMDIGFGAMDFAALQQDKSDVPLDVVQATCKYPD |
| | YIRMNHEAYGNSMFFFARREQMYTRHFFTRGGSVGDKE |
| | AVPQSLYLTADAEPRTTLATTNYVGTPSGSMVSSDVQLFN |
| | RSYWLQRCQGQNNGICWRNQLFITVGDNTRGTSLSISMK |
| | NNASTTYSNANFNDFLRHTEEFDLSFIVQLCKVKLTPENL |
| | AYIHTMDPNILEDWQLSVSQPPTNPLEDQYRFLGSSLAAK |
| | CPEQAPPEPQTDPYSQYKFWEVDLTERMSEQLDQFPLGR |
| | KFLYQSGMTQRTATSSTTKRKTVRLSTSAKRRRKA |
| | (SEQ ID NO: 8) |
| HPV-1a L2 | MYRLRRKRAAPKDIYPSCKISNTCPPDIQNKIEHTTIADKI |
| amino acid | LQYGSLGVFLGGLGIGTARGSGGRIGYTPLGEGGGVRVA |
| sequence | TRPTPVRPTIPVETVGPSEIFPIDVVDPTGPAVIPLQDLGRD |
| | FPIPTVQVIAEIHPISDIPNIVASSTNEGESAILDVLQGSATIR |
| | TVSRTQYNNPSFTVASTSNISAGEASTSDIVFVSNGSGDRV |
| | VGEDIPLVELNLGLETDTSSVVQETAFSSSTPIAERPSFRPS |
| | RFYNRRLYEQVQVQDPRFVEQPQSMVTFDNPAFEPELDE |
| | VSIIFQRDLDALAQTPVPEFRDVVYLSKPTFSREPGGRLRV |
| | SRLGKSSTIRTRLGTAIGARTHFFYDLSSIAPEDSIELLPLG |
| | EHSQTTVISSNLGDTAFIQGETAEDDLEVISLETPQLYSEE |
| | ELLDTNESVGENLQLTITNSEGEVSILDLTQSRVRPPFGTE |
| | DTSLHVYYPNSSKGTPIINPEESFTPLVIIALNNSTGDFELH |
| | PSLRKRRKRAYV (SEQ ID NO: 9) |
| pDY0006HPV- | gacggatcgggagatctcccgatcccctatggtgcactctcagtacaatctgctctgatgcc |
| 18 L1-HCV | gcatagttaagccagtatctgctccctgcttgtgtgttggaggtcgctgagtagtgcgcgag |
| IRES-L2 | caaaatttaagctacaacaaggcaaggcttgaccgacaattgcatgaagaatctgcttag |
| (seq | ggttaggcgttttgcgctgcttcgcgatgtacgggccagatatacgcgttgacattgattatt |
| arFWIQ9c) | gactagttattaatagtaatcaattacggggtcattagttcatagcccatatatggagttccg |
| CMV | cgttacataacttacggtaaatggcccgcctggctgaccgcccaacgacccccgcccattg |
| promoter: | acgtcaataatgacgtatgttcccatagtaacgccaatagggactttccattgacgtcaatg |
| nucleotides | ggtggagtatttacggtaaactgcccacttggcagtacatcaagtgtatcatatgccaagta |
| 232 to 819 | cgccccctattgacgtcaatgacggtaaatggcccgcctggcattatgcccagtacatgac |
| T7 promoter: | cttatgggactttcctacttggcagtacatctacgtattagtcatcgctattaccatggtgatg |
| nucleotides | cggttttggcagtacatcaatgggcgtggatagcggtttgactcacggggatttccaagtct |
| 863 to 879 | ccaccccattgacgtcaatgggagtttgttttggcaccaaaatcaacgggactttccaaaat |
| HPV-18 L1 | gtcgtaacaactccgccccattgacgcaaatgggcggtaggcgtgtacggtgggaggtct |
| coding | atataagcagagctctctggctaactagagaacccactgcttactggcttatcgaaattaat |
| sequence: | acgactcactatagggagacccaagctggctagcgtttaaacttaAGCTTGCCAC |
| nucleotides | Catgtgcctgtatacacgggtcctgatattacattaccatctactacctctgtatggcccatt |
| 923 to 2629 | gtatcacccacggcccctgcctctacacagtatattggtatacatggtacacattattatttgt |
| IRES: | ggccattatattattttattcctaagaaacgtaaacgtgttccctatttttttgcagatggcttt |
| nucleotides | gtggcggcctagtgacaataccgtatatcttccacctccttctgtggcaagagttgtaaata |
| 2630 to 3068 | ccgatgattatgtgactcGcacaagcatattttatcatgctggcagctctagattattaactg |
| HPV-18 L2 | ttggtaatccatattttagggttcctgcaggtggtggcaataagcaggatattcctaaggttt |
| coding | ctgcataccaatatagagtatttagggtgcagttacctgacccaaataaatttggtttacctg |
| sequence: | atactagtatttataatcctgaaacacaacgtttagtgtgggcctgtgctggagtggaaatt |
| nucleotides | ggccgtggtcagcctttaggtgttggccttagtgggcatccattttataataaattagatgac |
| 3069 to 4457 | actgaaagttcccatgccgccacgtctaatgtttctgaggacgttagggacaatgtgtctgt |
| BGH polyA: | agattataagcagacacagttatgtattttgggctgtgcccctgctattggggaacactggg |
| nucleotides | ctaaaggcactgcttgtaaatcgcgtcctttatcacagggcgattgcccccctttagaactta |
| 4508 to 4732 | aaaacacagttttggaagatggtgatatggtagatactggatatggtgccatggactttagt |
| | acattgcaagatactaaatgtgaggtaccattggatatttgtcagtctatttgtaaatatcct |
| | gattatttacaaatgtctgcagatccttatggggattccatgtttttttgcttacggcgtgagc |
| | agctttttgctaggcatttttggaatagagcaggtactatgggtgacactgtgcctcaatcct |
| | tatatattaaaggcacaggtatgcGtgcttcacctggcagctgtgtgtattctccctctccaa |
| | gtggctctattgttacctctgactcccagttgtttaataaaccatattggttacataaggcaca |
| | gggtcataacaatggtgtttgctggcataatcaattatttgttactgtggtagataccactcG |
| | cagtaccaatttaacaatatgtgcttctacacagtctcctgtacctgggcaatatgatgctac |
| | caaatttaagcagtatagcagacatgttgaggaatatgatttgcagtttatttttcagttgtgt |
| | actattactttaactgcagatgttatgtcctatattcatagtatgaatagcagtattttagagg |
| | attggaactttggtgttccccccccGccaactactagtttggtggatacatatcgttttgtac |
| | aatctgttgctattacctgtcaaaaggatgctgcaccggctgaaaataaggatccctatgat |
| | aagttaaagttttggaatgtggatttaaaggaaaagttttctttagacttagatcaatatccc |
| | cttggacgtaaatttttggttcaggctggattgcgtcgcaagcccaccataggccctcgcaa |
| | acgttctgctccatctgccactacgtcttctaaacctgccaagcgtgtgcgtgtacgtgccag |
| | gaagtaattctagtgtacgtagccagcccccgattgggggcgacactccaccatagatcac |
| | tcccctgtgaggaactactgtcttcacgcagaaagcgtctagccatggcgttagtatgaga |
| | gtcgtgcagcctccaggaccccccctcccgggagagccatagtggtctgcggaaccggtg |
| | agtacaccggaattgccaggacgaccgggtcctttcttggatcaacccgctcaatgcctgg |
| | agatttgggcgtgcccccgcaagactgctagccgagtagtgttgggtcgcgaaaggccttg |
| | tggtactgcctgatagggtgcttgcgagtgccccgggaggtctcgtagaccgtgcaccatg |
| | agcacgaatcctaaacctcaaagaaaaaccaaacgtaacaccaaccgccgcccacagg |
| | acgtcttcatatgtctagccaccatggtatcccaccgtgccgcacgacgcaaacgggcttc |
| | ggtaactgacttatataaaacatgtaaacaatctggtacatgtccacctgatgttgttcctaa |
| | ggtggagggcaccacgttagcagataaaatattgcaatggtcaagccttggtatatttttgg |
| | gtggacttggcataggtactggcagtggtacagggggtcgtacagggtacattccattggg |
| | tgggcgttccaatacagtggtggatgttggtcctacacgtcccccagtggttattgaacctgt |
| | gggccccacagacccatctattgttacattaatagaggactccagtgtggttacatcaggtg |
| | cacctaggcctacgtttactggcacgtctgggtttgatataacatctgcgggtacaactaca |
| | cctgcggttttggatatcacaccttcgtctacctctgtgtctatttccacaaccaattttaccaa |
| | tcctgcattttctgatccgtccattattgaagttccacaaactggggaggtggcaggtaatgt |
| | atttgttggtacccctacatctggaacacatgggtatgaggaaatacctttacaaacatttg |
| | cttcttctggtacgggggaggaacccattagtagtaccccattgcctactgtgcggcgtgta |
| | gcaggtccccgcctttacagtagggcctaccaacaagtgtcagtggctaaccctgagtttct |
| | tacacgtccatcctctttaattacatatgacaacccggcctttgagcctgtggacactacatt |
| | aacatttgatcctcgtagtgatgttcctgattcagattttatggatattatccgtctacatagg |
| | cctgctttaacatccaggcgtgggactgttcgctttagtagattaggtcaacgggcaactat |
| | gtttacccgcagcggtacacaaataggtgctagggttcacttttatcatgatataagtcctat |
| | tgcaccttccccagaatatattgaactgcagcctttagtatctgccacggaggacaatgact |
| | tgtttgatatatatgcagatgacatggaccctgcagtgcctgtaccatcgcgttctactacct |
| | cctttgcattttttaaatattcgcccactatatcttctgcctcttcctatagtaatgtaacggtcc |
| | ctttaacctcctcttgggatgtgcctgtatacacgggtcctgatattacattaccatctactac |
| | ctctgtatggcccattgtatcacccacggcccctgcctctacacagtatattggtatacatgg |
| | tacacattattatttgtggccattatattattttattcctaagaaacgtaaacgtgttccctattt |
| | ttttgcagatggctttgtggcggcctaggcggccgctcgagtctagagggcccgtttaaacc |
| | cgctgatcagcctcgactgtgccttctagttgccagccatctgttgtttgcccctcccccgtgc |
| | cttccttgaccctggaaggtgccactcccactgtcctttcctaataaaatgaggaaattgcat |
| | cgcattgtctgagtaggtgtcattctattctggggggtggggtggggcaggacagcaaggg |
| | ggaggattgggaagacaatagcaggcatgctggggatgcggtgggctctatggcttctga |
| | ggcggaaagaaccagctggggctctagggggtatccccacgcgccctgtagcggcgcatt |
| | aagcgcggcgggtgtggtggttacgcgcagcgtgaccgctacacttgccagcgccctagc |
| | gcccgctcctttcgctttcttcccttcctttctcgccacgttcgccggctttccccgtcaagctct |
| | aaatcgggggctccctttagggttccgatttagtgctttacggcacctcgaccccaaaaaac |
| | ttgattagggtgatggttcacgtagtgggccatcgccctgatagacggtttttcgccctttga |
| | cgttggagtccacgttctttaatagtggactcttgttccaaactggaacaacactcaacccta |
| | tctcggtctattcttttgatttataagggattttgccgatttcggcctattggttaaaaaatgag |
| | ctgatttaacaaaaatttaacgcgaattaattctgtggaatgtgtgtcagttagggtgtgga |
| | aagtccccaggctccccagcaggcagaagtatgcaaagcatgcatctcaattagtcagca |
| | accaggtgtggaaagtccccaggctccccagcaggcagaagtatgcaaagcatgcatctc |
| | aattagtcagcaaccatagtcccgcccctaactccgcccatcccgcccctaactccgccca |
| | gttccgcccattctccgccccatggctgactaattttttttatttatgcagaggccgaggccgc |
| | ctctgcctctgagctattccagaagtagtgaggaggcttttttggaggcctaggcttttgcaa |
| | aaagctcccgggagcttgtatatccattttcggatctgatcaagagacaggatgaggatcg |
| | tttcgcatgattgaacaagatggattgcacgcaggttctccggccgcttgggtggagaggc |
| | tattcggctatgactgggcacaacagacaatcggctgctctgatgccgccgtgttccggctg |
| | tcagcgcaggggcgcccggttctttttgtcaagaccgacctgtccggtgccctgaatgaact |
| | gcaggacgaggcagcgcggctatcgtggctggccacgacgggcgttccttgcgcagctgt |
| | gctcgacgttgtcactgaagcgggaagggactggctgctattgggcgaagtgccggggca |
| | ggatctcctgtcatctcaccttgctcctgccgagaaagtatccatcatggctgatgcaatgc |
| | ggcggctgcatacgcttgatccggctacctgcccattcgaccaccaagcgaaacatcgcat |
| | cgagcgagcacgtactcggatggaagccggtcttgtcgatcaggatgatctggacgaaga |
| | gcatcaggggctcgcgccagccgaactgttcgccaggctcaaggcgcgcatgcccgacg |
| | gcgaggatctcgtcgtgacccatggcgatgcctgcttgccgaatatcatggtggaaaatgg |
| | ccgcttttctggattcatcgactgtggccggctgggtgtggcggaccgctatcaggacatag |
| | cgttggctacccgtgatattgctgaagagcttggcggcgaatgggctgaccgcttcctcgtg |
| | ctttacggtatcgccgctcccgattcgcagcgcatcgccttctatcgccttcttgacgagttct |
| | tctgagcgggactctggggttcgaaatgaccgaccaagcgacgcccaacctgccatcacg |
| | agatttcgattccaccgccgccttctatgaaaggttgggcttcggaatcgttttccgggacgc |
| | cggctggatgatcctccagcgcggggatctcatgctggagttcttcgcccaccccaacttgt |
| | ttattgcagcttataatggttacaaataaagcaatagcatcacaaatttcacaaataaagc |
| | atttttttcactgcattctagttgtggtttgtccaaactcatcaatgtatcttatcatgtctgtat |
| | accgtcgacctctagctagagcttggcgtaatcatggtcatagctgtttcctgtgtgaaattg |
| | ttatccgctcacaattccacacaacatacgagccggaagcataaagtgtaaagcctgggg |
| | tgcctaatgagtgagctaactcacattaattgcgttgcgctcactgcccgctttccagtcggg |
| | aaacctgtcgtgccagctgcattaatgaatcggccaacgcgcggggagaggcggtttgcg |
| | tattgggcgctcttccgcttcctcgctcactgactcgctgcgctcggtcgttcggctgcggcg |
| | agcggtatcagctcactcaaaggcggtaatacggttatccacagaatcaggggataacgc |
| | aggaaagaacatgtgagcaaaaggccagcaaaaggccaggaaccgtaaaaaggccgc |
| | gttgctggcgtttttccataggctccgcccccctgacgagcatcacaaaaatcgacgctcaa |
| | gtcagaggtggcgaaacccgacaggactataaagataccaggcgtttccccctggaagct |
| | ccctcgtgcgctctcctgttccgaccctgccgcttaccggatacctgtccgcctttctcccttc |
| | gggaagcgtggcgctttctcatagctcacgctgtaggtatctcagttcggtgtaggtcgttcg |
| | ctccaagctgggctgtgtgcacgaaccccccgttcagcccgaccgctgcgccttatccggt |
| | aactatcgtcttgagtccaacccggtaagacacgacttatcgccactggcagcagccactg |
| | gtaacaggattagcagagcgaggtatgtaggcggtgctacagagttcttgaagtggtggc |
| | ctaactacggctacactagaagaacagtatttggtatctgcgctctgctgaagccagttacc |
| | ttcggaaaaagagttggtagctcttgatccggcaaacaaaccaccgctggtagcGGTg |
| | gtttttttgtttgcaagcagcagattacgcgcagaaaaaaaggatctcaagaagatcctttg |
| | atcttttctacggggtctgacgctcagtggaacgaaaactcacgttaagggattttggtcat |
| | gagattatcaaaaaggatcttcacctagatccttttaaattaaaaatgaagttttaaatcaat |
| | ctaaagtatatatgagtaaacttggtctgacagttaccaatgcttaatcagtgaggcaccta |
| | tctcagcgatctgtctatttcgttcatccatagttgcctgactccccgtcgtgtagataactac |
| | gatacgggagggcttaccatctggccccagtgctgcaatgataccgcgagacccacgctc |
| | accggctccagatttatcagcaataaaccagccagccggaagggccgagcgcagaagtg |
| | gtcctgcaactttatccgcctccatccagtctattaattgttgccgggaagctagagtaagta |
| | gttcgccagttaatagtttgcgcaacgttgttgccattgctacaggcatcgtggtgtcacgct |
| | cgtcgtttggtatggcttcattcagctccggttcccaacgatcaaggcgagttacatgatccc |
| | ccatgttgtgcaaaaaagcggttagctccttcggtcctccgatcgttgtcagaagtaagttg |
| | gccgcagtgttatcactcatggttatggcagcactgcataattctcttactgtcatgccatcc |
| | gtaagatgcttttctgtgactggtgagtactcaaccaagtcattctgagaatagtgtatgcg |
| | gcgaccgagttgctcttgcccggcgtcaatacgggataataccgcgccacatagcagaac |
| | tttaaaagtgctcatcattggaaaacgttcttcggggcgaaaactctcaaggatcttaccgc |
| | tgttgagatccagttcgatgtaacccactcgtgcacccaactgatcttcagcatcttttacttt |
| | caccagcgtttctgggtgagcaaaaacaggaaggcaaaatgccgcaaaaaagggaata |
| | agggcgacacggaaatgttgaatactcatactcttcctttttcaatattattgaagcatttatc |
| | agggttattgtctcatgagcggatacatatttgaatgtatttagaaaaataaacaaataggg |
| | gttccgcgcacatttccccgaaaagtgccacctgacgtc (SEQ ID NO: 10) |
| HPV-18 L1 | MCLYTRVLILHYHLLPLYGPLYHPRPLPLHSILVYMVHIII |
| amino acid | CGHYIILFLRNVNVFPIFLQMALWRPSDNTVYLPPPSVAR |
| sequence | VVNTDDYVTRTSIFYHAGSSRLLTVGNPYFRVPAGGGNK |
| | QDIPKVSAYQYRVFRVQLPDPNKFGLPDTSIYNPETQRLV |
| | WACAGVEIGRGQPLGVGLSGHPFYNKLDDTESSHAATSN |
| | VSEDVRDNVSVDYKQTQLCILGCAPAIGEHWAKGTACKS |
| | RPLSQGDCPPLELKNTVLEDGDMVDTGYGAMDFSTLQD |
| | TKCEVPLDICQSICKYPDYLQMSADPYGDSMFFCLRREQL |
| | FARHFWNRAGTMGDTVPQSLYIKGTGMRASPGSCVYSPS |
| | PSGSIVTSDSQLFNKPYWLHKAQGHNNGVCWHNQLFVT |
| | VVDTTRSTNLTICASTQSPVPGQYDATKFKQYSRHVEEYD |
| | LQFIFQLCTITLTADVMSYIHSMNSSILEDWNFGVPPPPTT |
| | SLVDTYRFVQSVAITCQKDAAPAENKDPYDKLKFWNVDL |
| | KEKFSLDLDQYPLGRKFLVQAGLRRKPTIGPRKRSAPSAT |
| | TSSKPAKRVRVRARK (SEQ ID NO: 11) |
| HPV-18 L2 | MVSHRAARRKRASVTDLYKTCKQSGTCPPDVVPKVEGT |
| amino acid | TLADKILQWSSLGIFLGGLGIGTGSGTGGRTGYIPLGGRS |
| sequence | NTVVDVGPTRPPVVIEPVGPTDPSIVTLIEDSSVVTSGAPRP |
| | TFTGTSGFDITSAGTTTPAVLDITPSSTSVSISTTNFTNPAFS |
| | DPSIIEVPQTGEVAGNVFVGTPTSGTHGYEEIPLQTFASSG |
| | TGEEPISSTPLPTVRRVAGPRLYSRAYQQVSVANPEFLTRP |
| | SSLITYDNPAFEPVDTTLTFDPRSDVPDSDFMDIIRLHRPAL |
| | TSRRGTVRFSRLGQRATMFTRSGTQIGARVHFYHDISPIA |
| | PSPEYIELQPLVSATEDNDLFDIYADDMDPAVPVPSRSTTS |
| | FAFFKYSPTISSASSYSNVTVPLTSSWDVPVYTGPDITLPST |
| | TSVWPIVSPTAPASTQYIGIHGTHYYLWPLYYFIPKKRKR |
| | VPYFFADGFVAA (SEQ ID NO: 12} |
| pDY0007HPV- | gacggatcgggagatctcccgatcccctatggtgcactctcagtacaatctgctctgatgcc |
| 137 L1-HCV | gcatagttaagccagtatctgctccctgcttgtgtgttggaggtcgctgagtagtgcgcgag |
| IRES-L2 | caaaatttaagctacaacaaggcaaggcttgaccgacaattgcatgaagaatctgcttag |
| (seq | ggttaggcgttttgcgctgcttcgcgatgtacgggccagatatacgcgttgacattgattatt |
| GtGsnLLL) | gactagttattaatagtaatcaattacggggtcattagttcatagcccatatatggagttccg |
| CMV | cgttacataacttacggtaaatggcccgcctggctgaccgcccaacgacccccgcccattg |
| promoter: | acgtcaataatgacgtatgttcccatagtaacgccaatagggactttccattgacgtcaatg |
| nucleotides | ggtggagtatttacggtaaactgcccacttggcagtacatcaagtgtatcatatgccaagta |
| 232 to 819 | cgccccctattgacgtcaatgacggtaaatggcccgcctggcattatgcccagtacatgac |
| T7 promoter: | cttatgggactttcctacttggcagtacatctacgtattagtcatcgctattaccatggtgatg |
| nucleotides | cggttttggcagtacatcaatgggcgtggatagcggtttgactcacggggatttccaagtct |
| 863 to 879 | ccaccccattgacgtcaatgggagtttgttttggcaccaaaatcaacgggactttccaaaat |
| HPV-137 L1 | gtcgtaacaactccgccccattgacgcaaatgggcggtaggcgtgtacggtgggaggtct |
| coding | atataagcagagctctctggctaactagagaacccactgcttactggcttatcgaaattaat |
| sequence: | acgactcactatagggagacccaagctggctagcgtttaaacttaAGCTTGCCAC |
| nucleotides | Catggctgtgtgggtaccgaacaaaggacgtctgtatttgccaccacaacgacctgtggct |
| 923 to 2473 | aaagttttgtctacagatgactatattgttggaactgatttatacttccattcgagtactgacc |
| IRES: | gccttttaacagttggacatcctttctttgatgtattaagcacagaccaaaataccgttgatg |
| nucleotides | tacccaaggtatctggtaatcaattcagggtatttagactaaatcttccagatcctaaccagt |
| 2474 to 2912 | ttgctctaattgatacatctatttataatccagaacatgaacgccttgtatggcgtctagtag |
| HPV-137 L2 | gtattgaaattgatagaggtggtcctcttggtataggtagtactggtcatccactatttaaca |
| coding | aattgcaggatacagaaaatccttctgtatataatggattaatcagtgaccaaaaggataa |
| sequence: | caggatgaatgtagcatttgatcccaaacaaaatcaattgtttatagtaggatgtaaacctg |
| nucleotides | ctgttggtcaacattgggacaaagcagaaccttgccctaacacgcgcccacccccaggaa |
| 2913 to 4442 | gttgcccacctcttaaattggtacatagtacaattgaggatggcgacatgtctgatatcggtt |
| BGH polyA: | taggaaatataaatttcagtgatctttctgatgataaatccagtgcacctttggaaattatta |
| nucleotides | attctaagtgtaagtggcctgattttgctttaatgaccaaagatttatttggcgacagtgcctt |
| 4493 to 4717 | cttttttggaaggcgtgagcaactttatgctcgccaccagtggtgcagggatggccttgtgg |
| | gggacgctattccagatgaacacttttattttaatcctaatggccaggatccaaagcctcctc |
| | aatatcagcttggctcttctatttactttacaattccgagtggttcgttgactagcagcgaatc |
| | aaacatatttggtagaccatattggttgcacagagctcagggtgcaaataatggtattgcat |
| | ggggcaatcaattgtttgtaactttattggacaacacacacaacacaaactttactatatct |
| | gtaagtactgaatcacaaacaacatatgataaaaacaaatttaaggtttatttacgacatg |
| | cagaggaaatagaaatagaaatcgtttgtcagctctgtaaggttcctttggaagcagatat |
| | cctggcacatttatatgctatggacccatctatattagacaactggcagctagcttttgtacc |
| | tgcgccaccacaaactctagaagatacttacagatatataagatctatggctactatgtgtc |
| | ccgcagatgtgcctccaaaggagccagaggacccgtacaaagatttacacttttggactat |
| | taatctgactgatagatttacttcagagttggatcaaactcctttaggtaaaagatttttgtat |
| | cagatgggattacttactggaaacaaacgcttgcgaacagattatataggttctccagttgc |
| | taaacgacgaaggacagtaaaatctagtaaaagaaagaagtcttctgcaaagtaattcta |
| | gtgtacgtagccagcccccgattgggggcgacactccaccatagatcactcccctgtgagg |
| | aactactgtcttcacgcagaaagcgtctagccatggcgttagtatgagagtcgtgcagcct |
| | ccaggaccccccctcccgggagagccatagtggtctgcggaaccggtgagtacaccgga |
| | attgccaggacgaccgggtcctttcttggatcaacccgctcaatgcctggagatttgggcgt |
| | gcccccgcaagactgctagccgagtagtgttgggtcgcgaaaggccttgtggtactgcctg |
| | atagggtgcttgcgagtgccccgggaggtctcgtagaccgtgcaccatgagcacgaatcct |
| | aaacctcaaagaaaaaccaaacgtaacaccaaccgccgcccacaggacgtcttcatatgt |
| | ctagccaccatgcaagccaataaaagacgtaagcgtgctgcagtagaagatatctatgct |
| | aaaggttgtacacagccaggaggttattgtccccctgatgtaaaaaataaagtagaaggt |
| | aatacatgggctgactttttactaaaagtgtttggaagtgtggtctattttggtgggcttggc |
| | attggaacaggtaaaggtactggtggttctacgggatacacaccactaggtggcactgtag |
| | gatctagaggcaccacaaacactataaaacctacaataccactggaccctttaggtgttcc |
| | agatatagttacggtagaccctattgctccagaagccgcgtccatagtacctttagctgaag |
| | gattacccgaaccaggtgttatagacacaggcacatctttccctgggttagcagcagataa |
| | tgaaaatatagtaacagtgctagaccccctatcagaggtcacaggggttggtgaacaccc |
| | aaatattattactggtggtactgctgatagccctgctattttagatgtacaaacctcaccccc |
| | accagctaaaaaaatattattagatccctctattagtaaaactacaactgctgtgcaaactc |
| | atgcttcccatgtagatgcaaatctgaatatatttgtagatgcacagtcttttggtactcatgt |
| | gggttatacagaagacattcccttggaagaaataaatttaaggagtgaatttgaattagaa |
| | gatagtgaacccaaaactagcacaccttttgcagaaagagttttaaataaaaccaaacag |
| | ctctatagtaaatatgttcaacaagtgccaacacgtcctgctgaatttgcactttatacatct |
| | aggtttgaatttgaaaatcccgcctttgaggaggacgtcactatggaatttgaaaatgattt |
| | ggcagagattggggagataacaacccccgcagtttctgatgtaagaattttaaataggcca |
| | atatattctgaaactgcagacaggactgtccgcattagtagactaggtcagcgagctggaa |
| | tgaaaactagaagtggacttgaaataggccaaagggtacacttttactttgacctcagtga |
| | tattcctagagaatccatagaacttaatacctatggtaattacagtcatgaaagcactatag |
| | ttgatgaattgctttctagcacgtttattaatccatttgaaatgcctgttgattcagaaatattt |
| | gcagaaaatgaattgttagatcctttagaggaggactttagagattcacatatagtagttcc |
| | ttatttagaagatgagcagataaatattactcctacattgccaccaggcctaggtttaaaag |
| | tttacagtgatttatcggaaagagatttattaatacattaccctgtgcagcatgcagacatta |
| | tggtgccagatacaccttatattcctgtgcaacctcctgatggagttctggtagatgacaatg |
| | attattatttgcaccctggtttgtattctcgaaaaagaaaacgacgtgttttgtaagcggccg |
| | ctcgagtctagagggcccgtttaaacccgctgatcagcctcgactgtgccttctagttgcca |
| | gccatctgttgtttgcccctcccccgtgccttccttgaccctggaaggtgccactcccactgtc |
| | ctttcctaataaaatgaggaaattgcatcgcattgtctgagtaggtgtcattctattctgggg |
| | ggtggggtggggcaggacagcaagggggaggattgggaagacaatagcaggcatgctg |
| | gggatgcggtgggctctatggcttctgaggcggaaagaaccagctggggctctagggggt |
| | atccccacgcgccctgtagcggcgcattaagcgcggcgggtgtggtggttacgcgcagcg |
| | tgaccgctacacttgccagcgccctagcgcccgctcctttcgctttcttcccttcctttctcgcc |
| | acgttcgccggctttccccgtcaagctctaaatcgggggctccctttagggttccgatttagt |
| | gctttacggcacctcgaccccaaaaaacttgattagggtgatggttcacgtagtgggccat |
| | cgccctgatagacggtttttcgccctttgacgttggagtccacgttctttaatagtggactctt |
| | gttccaaactggaacaacactcaaccctatctcggtctattcttttgatttataagggattttg |
| | ccgatttcggcctattggttaaaaaatgagctgatttaacaaaaatttaacgcgaattaatt |
| | ctgtggaatgtgtgtcagttagggtgtggaaagtccccaggctccccagcaggcagaagt |
| | atgcaaagcatgcatctcaattagtcagcaaccaggtgtggaaagtccccaggctcccca |
| | gcaggcagaagtatgcaaagcatgcatctcaattagtcagcaaccatagtcccgccccta |
| | actccgcccatcccgcccctaactccgcccagttccgcccattctccgccccatggctgact |
| | aattttttttatttatgcagaggccgaggccgcctctgcctctgagctattccagaagtagtg |
| | aggaggcttttttggaggcctaggcttttgcaaaaagctcccgggagcttgtatatccatttt |
| | cggatctgatcaagagacaggatgaggatcgtttcgcatgattgaacaagatggattgca |
| | cgcaggttctccggccgcttgggtggagaggctattcggctatgactgggcacaacagac |
| | aatcggctgctctgatgccgccgtgttccggctgtcagcgcaggggcgcccggttctttttgt |
| | caagaccgacctgtccggtgccctgaatgaactgcaggacgaggcagcgcggctatcgtg |
| | gctggccacgacgggcgttccttgcgcagctgtgctcgacgttgtcactgaagcgggaagg |
| | gactggctgctattgggcgaagtgccggggcaggatctcctgtcatctcaccttgctcctgc |
| | cgagaaagtatccatcatggctgatgcaatgcggcggctgcatacgcttgatccggctacc |
| | tgcccattcgaccaccaagcgaaacatcgcatcgagcgagcacgtactcggatggaagcc |
| | ggtcttgtcgatcaggatgatctggacgaagagcatcaggggctcgcgccagccgaactg |
| | ttcgccaggctcaaggcgcgcatgcccgacggcgaggatctcgtcgtgacccatggcgat |
| | gcctgcttgccgaatatcatggtggaaaatggccgcttttctggattcatcgactgtggccg |
| | gctgggtgtggcggaccgctatcaggacatagcgttggctacccgtgatattgctgaagag |
| | cttggcggcgaatgggctgaccgcttcctcgtgctttacggtatcgccgctcccgattcgca |
| | gcgcatcgccttctatcgccttcttgacgagttcttctgagcgggactctggggttcgaaatg |
| | accgaccaagcgacgcccaacctgccatcacgagatttcgattccaccgccgccttctatg |
| | aaaggttgggcttcggaatcgttttccgggacgccggctggatgatcctccagcgcgggga |
| | tctcatgctggagttcttcgcccaccccaacttgtttattgcagcttataatggttacaaataa |
| | agcaatagcatcacaaatttcacaaataaagcatttttttcactgcattctagttgtggtttgt |
| | ccaaactcatcaatgtatcttatcatgtctgtataccgtcgacctctagctagagcttggcgt |
| | aatcatggtcatagctgtttcctgtgtgaaattgttatccgctcacaattccacacaacatac |
| | gagccggaagcataaagtgtaaagcctggggtgcctaatgagtgagctaactcacattaa |
| | ttgcgttgcgctcactgcccgctttccagtcgggaaacctgtcgtgccagctgcattaatga |
| | atcggccaacgcgcggggagaggcggtttgcgtattgggcgctcttccgcttcctcgctcac |
| | tgactcgctgcgctcggtcgttcggctgcggcgagcggtatcagctcactcaaaggcggta |
| | atacggttatccacagaatcaggggataacgcaggaaagaacatgtgagcaaaaggcca |
| | gcaaaaggccaggaaccgtaaaaaggccgcgttgctggcgtttttccataggctccgccc |
| | ccctgacgagcatcacaaaaatcgacgctcaagtcagaggtggcgaaacccgacaggac |
| | tataaagataccaggcgtttccccctggaagctccctcgtgcgctctcctgttccgaccctgc |
| | cgcttaccggatacctgtccgcctttctcccttcgggaagcgtggcgctttctcatagctcac |
| | gctgtaggtatctcagttcggtgtaggtcgttcgctccaagctgggctgtgtgcacgaaccc |
| | cccgttcagcccgaccgctgcgccttatccggtaactatcgtcttgagtccaacccggtaag |
| | acacgacttatcgccactggcagcagccactggtaacaggattagcagagcgaggtatgt |
| | aggcggtgctacagagttcttgaagtggtggcctaactacggctacactagaagaacagt |
| | atttggtatctgcgctctgctgaagccagttaccttcggaaaaagagttggtagctcttgatc |
| | cggcaaacaaaccaccgctggtagcggtttttttgtttgcaagcagcagattacgcgcaga |
| | aaaaaaggatctcaagaagatcctttgatcttttctacggggtctgacgctcagtggaacg |
| | aaaactcacgttaagggattttggtcatgagattatcaaaaaggatcttcacctagatccttt |
| | taaattaaaaatgaagttttaaatcaatctaaagtatatatgagtaaacttggtctgacagtt |
| | accaatgcttaatcagtgaggcacctatctcagcgatctgtctatttcgttcatccatagttgc |
| | ctgactccccgtcgtgtagataactacgatacgggagggcttaccatctggccccagtgct |
| | gcaatgataccgcgagacccacgctcaccggctccagatttatcagcaataaaccagcca |
| | gccggaagggccgagcgcagaagtggtcctgcaactttatccgcctccatccagtctatta |
| | attgttgccgggaagctagagtaagtagttcgccagttaatagtttgcgcaacgttgttgcc |
| | attgctacaggcatcgtggtgtcacgctcgtcgtttggtatggcttcattcagctccggttccc |
| | aacgatcaaggcgagttacatgatcccccatgttgtgcaaaaaagcggttagctccttcgg |
| | tcctccgatcgttgtcagaagtaagttggccgcagtgttatcactcatggttatggcagcact |
| | gcataattctcttactgtcatgccatccgtaagatgcttttctgtgactggtgagtactcaacc |
| | aagtcattctgagaatagtgtatgcggcgaccgagttgctcttgcccggcgtcaatacggg |
| | ataataccgcgccacatagcagaactttaaaagtgctcatcattggaaaacgttcttcggg |
| | gcgaaaactctcaaggatcttaccgctgttgagatccagttcgatgtaacccactcgtgcac |
| | ccaactgatcttcagcatcttttactttcaccagcgtttctgggtgagcaaaaacaggaagg |
| | caaaatgccgcaaaaaagggaataagggcgacacggaaatgttgaatactcatactcttc |
| | ctttttcaatattattgaagcatttatcagggttattgtctcatgagcggatacatatttgaatg |
| | tatttagaaaaataaacaaataggggttccgcgcacatttccccgaaaagtgccacctgac |
| | gtc (SEQ ID NO: 13) |
| HPV-137 L1 | MAVWVPNKGRLYLPPQRPVAKVLSTDDYIVGTDLYFHSS |
| amino acid | TDRLLTVGHPFFDVLSTDQNTVDVPKVSGNQFRVFRLNL |
| sequence | PDPNQFALIDTSIYNPEHERLVWRLVGIEIDRGGPLGIGST |
| | GHPLFNKLQDTENPSVYNGLISDQKDNRMNVAFDPKQNQ |
| | LFIVGCKPAVGQHWDKAEPCPNTRPPPGSCPPLKLVHSTI |
| | EDGDMSDIGLGNINFSDLSDDKSSAPLEIINSKCKWPDFAL |
| | MTKDLFGDSAFFFGRREQLYARHQWCRDGLVGDAIPDE |
| | HFYFNPNGQDPKPPQYQLGSSIYFTIPSGSLTSSESNIFGRP |
| | YWLHRAQGANNGIAWGNQLFVTLLDNTHNTNFTISVSTE |
| | SQTTYDKNKFKVYLRHAEEIEIEIVCQLCKVPLEADILAH |
| | LYAMDPSILDNWQLAFVPAPPQTLEDTYRYIRSMATMCP |
| | ADVPPKEPEDPYKDLHFWTINLTDRFTSELDQTPLGKRFL |
| | YQMGLLTGNKRLRTDYIGSPVAKRRRTVKSSKRKKSSAK |
| | (SEQ ID NO: 14) |
| HPV-137 L2 | MQANKRRKRAAVEDIYAKGCTQPGGYCPPDVKNKVEGN |
| amino acid | TWADFLLKVFGSVVYFGGLGIGTGKGTGGSTGYTPLGGT |
| sequence | VGSRGTTNTIKPTIPLDPLGVPDIVTVDPIAPEAASIVPLAE |
| | GLPEPGVIDTGTSFPGLAADNENIVTVLDPLSEVTGVGEH |
| | PNIITGGTADSPAILDVQTSPPPAKKILLDPSISKTTTAVQT |
| | HASHVDANLNIFVDAQSFGTHVGYTEDIPLEEINLRSEFEL |
| | EDSEPKTSTPFAERVLNKTKQLYSKYVQQVPTRPAEFALY |
| | TSRFEFENPAFEEDVTMEFENDLAEIGEITTPAVSDVRILN |
| | RPIYSETADRTVRISRLGQRAGMKTRSGLEIGQRVHFYFD |
| | LSDIPRESIELNTYGNYSHESTIVDELLSSTFINPFEMPVDS |
| | EIFAENELLDPLEEDFRDSHIVVPYLEDEQINITPTLPPGLG |
| | LKVYSDLSERDLLIHYPVQHADIMVPDTPYIPVQPPDGVL |
| | VDDNDYYLHPGLYSRKRKRRVL (SEQ ID NO: 15) |
| pDY0018 | gacctctagctagagcttggcgtaatcatggtcatagctgtttcctgtgtgaaattgttatcc |
| p16sheLL | gctcacaattccacacaacatacgagccggaagcataaagtgtaaagcctggggtgccta |
| (seq | atgagtgagctaactcacattaattgcgttgcgctcactgcccgctttccagtcgggaaacc |
| LEt2NOPo) | tgtcgtgccagctgcattaatgaatcggccaacgcgcggggagaggcggtttgcgtattgg |
| CMV | gcgctcttccgcttcctcgctcactgactcgctgcgctcggtcgttcggctgcggcgagcggt |
| promoter: | atcagctcactcaaaggcggtaatacggttatccacagaatcaggggataacgcaggaa |
| nucleotides | agaacatgtgagcaaaaggccagcaaaaggccaggaaccgtaaaaaggccgcgttgct |
| 2496 to 3006 | ggcgtttttccataggctccgcccccctgacgagcatcacaaaaatcgacgctcaagtcag |
| HPV-16 L1 | aggtggcgaaacccgacaggactataaagataccaggcgtttccccctggaagctccctc |
| coding | gtgcgctctcctgttccgaccctgccgcttaccggatacctgtccgcctttctcccttcgggaa |
| sequence: | gcgtggcgctttctcatagctcacgctgtaggtatctcagttcggtgtaggtcgttcgctcca |
| nucleotides | agctgggctgtgtgcacgaaccccccgttcagcccgaccgctgcgccttatccggtaactat |
| 3207 to 4724 | cgtcttgagtccaacccggtaagacacgacttatcgccactggcagcagccactggtaaca |
| polio IRES: | ggattagcagagcgaggtatgtaggcggtgctacagagttcttgaagtggtggcctaacta |
| nucleotides | cggctacactagaagaacagtatttggtatctgcgctctgctgaagccagttaccttcggaa |
| 4764 to 5389 | aaagagttggtagctcttgatccggcaaacaaaccaccgctggtagcggtttttttgtttgc |
| HPV-16 L2 | aagcagcagattacgcgcagaaaaaaaggatctcaagaagatcctttgatcttttctacgg |
| coding | ggtctgacgctcagtggaacgaaaactcacgttaagggattttggtcatgagattatcaaa |
| sequence: | aaggatcttcacctagatccttttaaattaaaaatgaagttttaaatcaatctaaagtatata |
| nucleotides | tgagtaaacttggtctgacagttaccaatgcttaatcagtgaggcacctatctcagcgatct |
| 5409 to 6830 | gtctatttcgttcatccatagttgcctgactccccgtcgtgtagataactacgatacgggagg |
| WPRE: | gcttaccatctggccccagtgctgcaatgataccgcgagacccacgctcaccggctccaga |
| nucleotides | tttatcagcaataaaccagccagccggaagggccgagcgcagaagtggtcctgcaacttt |
| 6903 to 7491 | atccgcctccatccagtctattaattgttgccgggaagctagagtaagtagttcgccagtta |
| BGH polyA: | atagtttgcgcaacgttgttgccattgctacaggcatcgtggtgtcacgctcgtcgtttggtat |
| nucleotides | ggcttcattcagctccggttcccaacgatcaaggcgagttacatgatcccccatgttgtgca |
| 7518 to 7741 | aaaaagcggttagctccttcggtcctccgatcgttgtcagaagtaagttggccgcagtgtta |
| | tcactcatggttatggcagcactgcataattctcttactgtcatgccatccgtaagatgctttt |
| | ctgtgactggtgagtactcaaccaagtcattctgagaatagtgtatgcggcgaccgagttg |
| | ctcttgcccggcgtcaatacgggataataccgcgccacatagcagaactttaaaagtgctc |
| | atcattggaaaacgttcttcggggcgaaaactctcaaggatcttaccgctgttgagatccag |
| | ttcgatgtaacccactcgtgcacccaactgatcttcagcatcttttactttcaccagcgtttct |
| | gggtgagcaaaaacaggaaggcaaaatgccgcaaaaaagggaataagggcgacacgg |
| | aaatgttgaatactcatactcttcctttttcaatattattgaagcatttatcagggttattgtctc |
| | atgagcggatacatatttgaatgtatttagaaaaataaacaaataggggttccgcgcacat |
| | ttccccgaaaagtgccacctgacgtcgacggatcgggagatctcccgatcccctatggtgc |
| | actctcagtacaatctgctctgatgccgcatagttaagccagtatctgctccctgcttgtgtgt |
| | tggaggtcgctgagtagtgcgcgagcaaaatttaagctacaacaaggcaaggcttgaccg |
| | acaattgcatgaagaatctgcttagggttaggcgttttgcgctgcttcgcgatgtacgggcc |
| | agatatacgcgttgacattgattattgactagttattaatagtaatcaattacggggtcatta |
| | gttcatagcccatatatggagttccgcgttacataacttacggtaaatggcccgcctggctg |
| | accgcccaacgacccccgcccattgacgtcaataatgacgtatgttcccatagtaacgcca |
| | atagggactttccattgacgtcaatgggtggagtatttacggtaaactgcccacttggcagt |
| | acatcaagtgtatcatatgccaagtacgccccctattgacgtcaatgacggtaaatggccc |
| | gcctggcattatgcccagtacatgaccttatgggactttcctacttggcagtacatctacgta |
| | ttagtcatcgctattaccatggtgatgcggttttggcagtacatcaatgggcgtggatagcg |
| | gtttgactcacggggatttccaagtctccaccccattgacgtcaatgggagtttgttttggaa |
| | ccaaaatcaacgggactttccaaaatgtcgtaacaactccgccccattgacgcaaatggg |
| | cggtaggcgtgtacggtgggaggtctatataagcagagctctccctatcagtgatagagat |
| | ctccctatcagtgatagagatcgtcgacgagctcgtttagtgaaccgtcagatcgcctgga |
| | gacgccatccacgctgttttgacctccatagaagacaccgggaccgatccagcctccggac |
| | tctagcgtttaaacttaaggctagagtacttaatacgactcactataggctagagccaccat |
| | gagcctgtggctgcccagcgaggccaccgtgtacctgccccccgtgcccgtgagcaaggt |
| | ggtgagcaccgacgagtacgtggccaggaccaacatctactaccacgccggcaccagca |
| | ggctgctggccgtgggccacccctacttccccatcaagaagcccaacaacaacaagatcc |
| | tggtgcccaaggtgagcggcctgcagtacagggtgttcaggatccacctgcccgacccca |
| | acaagttcggcttccccgacaccagcttctacaaccccgacacccagaggctggtgtgggc |
| | ctgcgtgggcgtggaggtgggcaggggccagcccctgggcgtgggcatcagcggccacc |
| | ccctgctgaacaagctggacgacaccgagaacgccagcgcctacgccgccaacgccggc |
| | gtggacaacagggagtgcatcagcatggactacaagcagacccagctgtgcctgatcgg |
| | ctgcaagccccccatcggcgagcactggggcaagggcagcccctgcaccaacgtggccg |
| | tgaaccccggcgactgcccccccctggagctgatcaacaccgtgatccaggacggcgaca |
| | tggtggacaccggcttcggcgccatggacttcaccaccctgcaggccaacaagagcgagg |
| | tgcccctggacatctgcaccagcatctgcaagtaccccgactacatcaagatggtgagcga |
| | gccctacggcgacagcctgttcttctacctgaggagggagcagatgttcgtgaggcacctg |
| | ttcaacagggccggcgccgtgggcgagaacgtgcccgacgacctgtacatcaagggcag |
| | cggcagcaccgccaacctggccagcagcaactacttccccacccccagcggcagcatggt |
| | gaccagcgacgcccagatcttcaacaagccctactggctgcagagggcccagggccaca |
| | acaacggcatctgctggggcaaccagctgttcgtgaccgtggtggacaccaccaggagca |
| | ccaacatgagcctgtgcgccgccatcagcaccagcgagaccacctacaagaacaccaac |
| | ttcaaggagtacctgaggcacggcgaggagtacgacctgcagttcatcttccagctgtgca |
| | agatcaccctgaccgccgacgtgatgacctacatccacagcatgaacagcaccatcctgg |
| | aggactggaacttcggcctgcagcccccccccggcggcaccctggaggacacctacaggt |
| | tcgtgaccagccaggccatcgcctgccagaagcacaccccccccgcccccaaggaggac |
| | cccctgaagaagtacaccttctgggaggtgaacctgaaggagaagttcagcgccgacctg |
| | gaccagttccccctgggcaggaagttcctgctgcaggccggcctgaaggccaagcccaag |
| | ttcaccctgggcaagaggaaggccacccccaccaccagcagcaccagcaccaccgccaa |
| | gaggaagaagaggaagctgtgaaagcttatcgataccgtcgacctcgacctgcagaagc |
| | ttaaaacagctctggggttgtacccaccccagaggcccacgtggcggctagtactccggta |
| | ttgcggtacccttgtacgcctgttttatactcccttcccgtaacttagacgcacaaaaccaag |
| | ttcaatagaagggggtacaaaccagtaccaccacgaacaagcacttctgtttccccggtga |
| | tgtcgtatagactgcttgcgtggttgaaagcgacggatccgttatccgcttatgtacttcgag |
| | aagcccagtaccacctcggaatcttcgatgcgttgcgctcagcactcaaccccagagtgta |
| | gcttaggctgatgagtctggacatccctcaccggtgacggtggtccaggctgcgttggcgg |
| | cctacctatggctaacgccatgggacgctagttgtgaacaaggtgtgaagagcctattgag |
| | ctacataagaatcctccggcccctgaatgcggctaatcccaacctcggagcaggtggtcac |
| | aaaccagtgattggcctgtcgtaacgcgcaagtccgtggcggaaccgactactttgggtgt |
| | ccgtgtttccttttattttattgtggctgcttatggtgacaatcacagattgttatcataaagcg |
| | aattggattgcggccgctctagagccaccatgaggcacaagaggagcgccaagaggacc |
| | aagagggccagcgccacccagctgtacaagacctgcaagcaggccggcacctgcccccc |
| | cgacatcatccccaaggtggagggcaagaccatcgccgaccagatcctgcagtacggca |
| | gcatgggcgtgttcttcggcggcctgggcatcggcaccggcagcggcaccggcggcagg |
| | accggctacatccccctgggcaccaggccccccaccgccaccgacaccctggcccccgtg |
| | aggccccccctgaccgtggaccccgtgggccccagcgaccccagcatcgtgagcctggtg |
| | gaggagaccagcttcatcgacgccggcgcccccaccagcgtgcccagcatcccccccgac |
| | gtgagcggcttcagcatcaccaccagcaccgacaccacccccgccatcctggacatcaac |
| | aacaccgtgaccaccgtgaccacccacaacaaccccaccttcaccgaccccagcgtgctg |
| | cagccccccacccccgccgagaccggcggccacttcaccctgagcagcagcaccatcag |
| | cacccacaactacgaggagatccccatggacaccttcatcgtgagcaccaaccccaacac |
| | cgtgaccagcagcacccccatccccggcagcaggcccgtggccaggctgggcctgtaca |
| | gcaggaccacccagcaggtgaaggtggtggaccccgccttcgtgaccacccccaccaag |
| | ctgatcacctacgacaaccccgcctacgagggcatcgacgtggacaacaccctgtacttca |
| | gcagcaacgacaacagcatcaacatcgcccccgaccccgacttcctggacatcgtggccc |
| | tgcacaggcccgccctgaccagcaggaggaccggcatcaggtacagcaggatcggcaac |
| | aagcagaccctgaggaccaggagcggcaagagcatcggcgccaaggtgcactactacta |
| | cgacctgagcaccatcgaccccgccgaggagatcgagctgcagaccatcacccccagca |
| | cctacaccaccaccagccacgccgccagccccaccagcatcaacaacggcctgtacgac |
| | atctacgccgacgacttcatcaccgacaccagcaccacccccgtgcccagcgtgcccagc |
| | accagcctgagcggctacatccccgccaacaccaccatccccttcggtggcgcctacaac |
| | atccccctggtgagcggccccgacatccccatcaacatcaccgaccaggcccccagcctg |
| | atccccatcgtgcccggcagcccccagtacaccatcatcgccgacgccggcgacttctacc |
| | tgcaccccagctactacatgctgaggaagaggaggaagaggctgccctacttcttcagcg |
| | acgtgagcctggccgcctgaaagctttttgaattctttggatccactagtggatcccccggg |
| | ctgcaggaattcgatatcaagcttatcgataatcaacctctggattacaaaatttgtgaaag |
| | attgactggtattcttaactatgttgctccttttacgctatgtggatacgctgctttaatgccttt |
| | gtatcatgctattgcttcccgtatggctttcattttctcctccttgtataaatcctggttgctgtc |
| | tctttatgaggagttgtggcccgttgtcaggcaacgtggcgtggtgtgcactgtgtttgctgac |
| | gcaacccccactggttggggcattgccaccacctgtcagctcctttccgggactttcgctttc |
| | cccctccctattgccacggcggaactcatcgccgcctgccttgcccgctgctggacagggg |
| | ctcggctgttgggcactgacaattccgtggtgttgtcggggaaatcatcgtcctttccttggc |
| | tgctcgcctgtgttgccacctggattctgcgcgggacgtccttctgctacgtcccttcggccct |
| | caatccagcggaccttccttcccgcggcctgctgccggctctgcggcctcttccgcgtcttcg |
| | ccttcgccctcagacgagtcggatctccctttgggccgcctccccgcatcgataccgtcggc |
| | ccgtttaaacccgctgatcagcctcgactgtgccttctagttgccagccatctgttgtttgccc |
| | ctcccccgtgccttccttgaccctggaaggtgccactcccactgtcctttcctaataaaatga |
| | ggaaattgcatcgcattgtctgagtaggtgtcattctattctggggggtggggtggggcagg |
| | acagcaagggggaggattgggaagacaatagcaggcatgctggggatgcggtgggctct |
| | atggcttctgaggcggaaagaaccagctggggctctagggggtatccccacgcgccctgt |
| | agcggcgcattaagcgcggcgggtgtggtggttacgcgcagcgtgaccgctacacttgcc |
| | agcgccctagcgcccgctcctttcgctttcttcccttcctttctcgccacgttcgccggctttcc |
| | ccgtcaagctctaaatcgggggctccctttagggttccgatttagtgctttacggcacctcga |
| | ccccaaaaaacttgattagggtgatggttcacgtagtgggccatcgccctgatagacggttt |
| | ttcgccctttgacgttggagtccacgttctttaatagtggactcttgttccaaactggaacaa |
| | cactcaaccctatctcggtctattcttttgatttataagggattttgccgatttcggcctattgg |
| | ttaaaaaatgagctgatttaacaaaaatttaacgcgaattaattctgtggaatgtgtgtcag |
| | ttagggtgtggaaagtccccaggctccccagcaggcagaagtatgcaaagcatgcagaat |
| | tctatcaaatatttaaagaaaaaaaaattgtatcaactttctacaatctctttcagaagaca |
| | gaagcagagggaatacttcctaaatcattcaactaggccagcattaccttaataccggaac |
| | tagaaaatgacattacaagaaaagaaaacaacagaccaatatctctcatgaacaaagat |
| | acaaacattttcaacaaaatattagcaaaaagaatccaagaatgtatcaaaaaatataca |
| | ccacaaccaagtagaatttattccagatatgtaagggtggttcaacgtttgaaaatcaatta |
| | acgtaatttgtcccatcaacaggttaaagaagaaaatcacatggtcatattgatagacaca |
| | gaaaaagcatttgacaaaatttaacacccattcatgatgcaatctctcagtaaactaggaa |
| | tagaggaaaacttcctcagcttgaatgtaccttcctctcaattttgctatgaacctgaaactc |
| | ctcttaaaaaataaagtttttcatttaaaaagaaaacaaaaaacatggaggagcgttgatg |
| | tatctcattttagaccaatcagctatggatagttaggcgacagcacagatagctgctgtact |
| | tctgtttctggcaatgttccagactacatttaaaaaatttttaattatagacttgtacttaatgt |
| | tcaagaaaaatatgaaaatggctttgccgtgttaatgctactcttttttaaaaaaaactaaa |
| | gttcaaactttatttatatttcattagttttttagctactgttctttttctgttctgggatctcatt |
| | cagaatgccacattacatataattctcatgtctccttgggttcctcttagttttgacagttcctca |
| | gacttttcttatttttgatgaccttgacagttttgaggagtactggttagatatagggtaatgg |
| | tttttaaagtatatttgtcatgatttatactggggtaagggtttggggaggaagcccatgggg |
| | taaagtactgttctcatcacatcatatcaaggttatataccatcaatattgccacagatgtta |
| | cttagccttttaatatttctctaatttagtgtatatgcaatgatagttctctgatttctgagattg |
| | agtttctcatgtgtaatgattatttagagtttctctttcatctgttcaaatttttgtctagttttat |
| | tttttactgatttgtaagacttctttttataatctgcatattacaattctctttactggggtgttgc |
| | aaatattttctgtcattctatggcctgacttttcttaatggttttttaattttaaaaataagtctta |
| | atattcatgcaatctaattaacaatcttttctttgtggttaggactttgagtcataagaaatttt |
| | tctctacactgaagtcatgatggcatgcttctatattattttctaaaagatttaaagttttgcct |
| | tctccatttagacttataattcactggaatttttttgtgtgtatggtatgacatatgggttccctt |
| | ttattttttacatataaatatatttccctgtttttctaaaaaagaaaaagatcatcattttccca |
| | ttgtaaaatgccatatttttttcataggtcacttacatatatcaatgggtctgtttctgagctct |
| | actctattttatcagcctcactgtctatccccacacatctcatgctttgctctaaatcttgatatt |
| | tagtggaacattctttcccattttgttctacaagaatatttttgttattgtcttttgggcttctata |
| | tacattttagaatgaggttggcaagttaacaaacagcttttttggggtgaacatattgactac |
| | aaatttatgtggaaagaaagtaccaagttgaccagtgccgttccggtgctcaccgcgcgcg |
| | acgtcgccggagcggtcgagttctggaccgaccggctcgggttctcccgggacttcgtgga |
| | ggacgacttcgccggtgtggtccgggacgacgtgaccctgttcatcagcgcggtccagga |
| | ccaggtggtgccggacaacaccctggcctgggtgtgggtgcgcggcctggacgagctgta |
| | cgccgagtggtcggaggtcgtgtccacgaacttccgggacgcctccgggccggccatgac |
| | cgagatcggcgagcagccgtgggggcgggagttcgccctgcgcgacccggccggcaact |
| | gcgtgcacttcgtggccgaggagcaggactgacacgtgctacgagatttcgattccaccgc |
| | cgccttctatgaaaggttgggcttcggaatcgttttccgggacgccggctggatgatcctcc |
| | agcgcggggatctcatgctggagttcttcgcccaccccaacttgtttattgcagcttataatg |
| | gttacaaataaagcaatagcatcacaaatttcacaaataaagcatttttttcactgcattcta |
| | gttgtggtttgtccaaactcatcaatgtatcttatcatgtctgtataccgtc |
| | (SEQ ID NO: 16) |
| HPV-16 L1 | MSLWLPSEATVYLPPVPVSKVVSTDEYVARTNIYYHAGTS |
| amino acid | RLLAVGHPYFPIKKPNNNKILVPKVSGLQYRVFRIHLPDP |
| sequence | NKFGFPDTSFYNPDTQRLVWACVGVEVGRGQPLGVGISG |
| | HPLLNKLDDTENASAYAANAGVDNRECISMDYKQTQLCL |
| | IGCKPPIGEHWGKGSPCTNVAVNPGDCPPLELINTVIQDG |
| | DMVDTGFGAMDFTTLQANKSEVPLDICTSICKYPDYIKM |
| | VSEPYGDSLFFYLRREQMFVRHLFNRAGAVGENVPDDLY |
| | IKGSGSTANLASSNYFPTPSGSMVTSDAQIFNKPYWLQRA |
| | QGHNNGICWGNQLFVTVVDTTRSTNMSLCAAISTSETTY |
| | KNTNFKEYLRHGEEYDLQFIFQLCKITLTADVMTYIHSM |
| | NSTILEDWNFGLQPPPGGTLEDTYRFVTSQAIACQKHTPP |
| | APKEDPLKKYTFWEVNLKEKFSADLDQFPLGRKFLLQAG |
| | LKAKPKFTLGKRKATPTTSSTSTTAKRKKRKL |
| | (SEQ ID NO: 17) |
| HPV-16 L2 | MRHKRSAKRTKRASATQLYKTCKQAGTCPPDIIPKVEGK |
| amino acid | TIADQILQYGSMGVFFGGLGIGTGSGTGGRTGYIPLGTRP |
| sequence | PTATDTLAPVRPPLTVDPVGPSDPSIVSLVEETSFIDAGAPT |
| | SVPSIPPDVSGFSITTSTDTTPAILDINNTVTTVTTHNNPTFT |
| | DPSVLQPPTPAETGGHFTLSSSTISTHNYEEIPMDTFIVSTN |
| | PNTVTSSTPIPGSRPVARLGLYSRTTQQVKVVDPAFVTTPT |
| | KLITYDNPAYEGIDVDNTLYFSSNDNSINIAPDPDFLDIVAL |
| | HRPALTSRRTGIRYSRIGNKQTLRTRSGKSIGAKVHYYYD |
| | LSTIDPAEEIELQTITPSTYTTTSHAASPTSINNGLYDIYAD |
| | DFITDTSTTPVPSVPSTSLSGYIPANTTIPFGGAYNIPLVSGP |
| | DIPINITDQAPSLIPIVPGSPQYTIIADAGDFYLHPSYYMLR |
| | KRRKRLPYFFSDVSLAA (SEQ ID NO: 18) |
| pDY0022 | gacggatcgggagatctcccgatcccctatggtgcactctcagtacaatctgctctgatgcc |
| HPV-16 L1- | gcatagttaagccagtatctgctccctgcttgtgtgttggaggtcgctgagtagtgcgcgag |
| HCV IRES-L2 | caaaatttaagctacaacaaggcaaggcttgaccgacaattgcatgaagaatctgcttag |
| (seq | ggttaggcgttttgcgctgcttcgcgatgtacgggccagatatacgcgttgacattgattatt |
| eOHVgmwC) | gactagttattaatagtaatcaattacggggtcattagttcatagcccatatatggagttccg |
| CMV | cgttacataacttacggtaaatggcccgcctggctgaccgcccaacgacccccgcccattg |
| promoter: | acgtcaataatgacgtatgttcccatagtaacgccaatagggactttccattgacgtcaatg |
| nucleotides | ggtggagtatttacggtaaactgcccacttggcagtacatcaagtgtatcatatgccaagta |
| 232 to 819 | cgccccctattgacgtcaatgacggtaaatggcccgcctggcattatgcccagtacatgac |
| T7 promoter: | cttatgggactttcctacttggcagtacatctacgtattagtcatcgctattaccatggtgatg |
| nucleotides | cggttttggcagtacatcaatgggcgtggatagcggtttgactcacggggatttccaagtct |
| 863 to 879 | ccaccccattgacgtcaatgggagtttgttttggcaccaaaatcaacgggactttccaaaat |
| HPV-16 L1 | gtcgtaacaactccgccccattgacgcaaatgggcggtaggcgtgtacggtgggaggtct |
| coding | atataagcagagctctctggctaactagagaacccactgcttactggcttatcgaaattaat |
| sequence: | acgactcactatagggagacccaagctggctagcgtttaaacttaagcttgccaccatgtg |
| nucleotides | cctgtatacacgggtcctgatattacattaccatctactacctctgtatggcccattgtatcac |
| 923 to 2629 | ccacggcccctgcctctacacagtatattggtatacatggtacacattattatttgtggccatt |
| IRES: | atattattttattcctaagaaacgtaaacgtgttccctatttttttgcagatggctttgtggcgg |
| nucleotides | cctagtgacaataccgtatatcttccacctccttctgtggcaagagttgtaaataccgatgat |
| 2630 to 3068 | tatgtgactcccacaagcatattttatcatgctggcagctctagattattaactgttggtaatc |
| HPV-16 L2 | catattttagggttcctgcaggtggtggcaataagcaggatattcctaaggtttctgcatacc |
| coding | aatatagagtatttagggtgcagttacctgacccaaataaatttggtttacctgatactagta |
| sequence: | tttataatcctgaaacacaacgtttagtgtgggcctgtgctggagtggaaattggccgtggt |
| nucleotides | cagcctttaggtgttggccttagtgggcatccattttataataaattagatgacactgaaagt |
| 3069 to 4485 | tcccatgccgccacgtctaatgtttctgaggacgttagggacaatgtgtctgtagattataa |
| BGH polyA: | gcagacacagttatgtattttgggctgtgcccctgctattggggaacactgggctaaaggc |
| nucleotides | actgcttgtaaatcgcgtcctttatcacagggcgattgcccccctttagaacttaaaaacac |
| 4541 to 4765 | agttttggaagatggtgatatggtagatactggatatggtgccatggactttagtacattgc |
| | aagatactaaatgtgaggtaccattggatatttgtcagtctatttgtaaatatcctgattattt |
| | acaaatgtctgcagatccttatggggattccatgtttttttgcttacggcgtgagcagcttttt |
| | gctaggcatttttggaatagagcaggtactatgggtgacactgtgcctcaatccttatatatt |
| | aaaggcacaggtatgcctgcttcacctggcagctgtgtgtattctccctctccaagtggctct |
| | attgttacctctgactcccagttgtttaataaaccatattggttacataaggcacagggtcat |
| | aacaatggtgtttgctggcataatcaattatttgttactgtggtagataccactcccagtacc |
| | aatttaacaatatgtgcttctacacagtctcctgtacctgggcaatatgatgctaccaaattt |
| | aagcagtatagcagacatgttgaggaatatgatttgcagtttatttttcagttgtgtactatta |
| | ctttaactgcagatgttatgtcctatattcatagtatgaatagcagtattttagaggattgga |
| | actttggtgttcccccccccccaactactagtttggtggatacatatcgttttgtacaatctgtt |
| | gctattacctgtcaaaaggatgctgcaccggctgaaaataaggatccctatgataagttaa |
| | agttttggaatgtggatttaaaggaaaagttttctttagacttagatcaatatccccttggac |
| | gtaaatttttggttcaggctggattgcgtcgcaagcccaccataggccctcgcaaacgttct |
| | gctccatctgccactacgtcttctaaacctgccaagcgtgtgcgtgtacgtgccaggaagta |
| | attctagtgtacgtagccagcccccgattgggggcgacactccaccatagatcactcccct |
| | gtgaggaactactgtcttcacgcagaaagcgtctagccatggcgttagtatgagagtcgtg |
| | cagcctccaggaccccccctcccgggagagccatagtggtctgcggaaccggtgagtaca |
| | ccggaattgccaggacgaccgggtcctttcttggatcaacccgctcaatgcctggagatttg |
| | ggcgtgcccccgcaagactgctagccgagtagtgttgggtcgcgaaaggccttgtggtact |
| | gcctgatagggtgcttgcgagtgccccgggaggtctcgtagaccgtgcaccatgagcacg |
| | aatcctaaacctcaaagaaaaaccaaacgtaacaccaaccgccgcccacaggacgtctt |
| | catatgtctagccaccatgcgacacaaacgttctgcaaaacgcacaaaacgtgcatcggc |
| | tacccaactttataaaacatgcaaacaggcaggtacatgtccacctgacattatacctaag |
| | gttgaaggcaaaactattgctgaacaaatattacaatatggaagtatgggtgtattttttggt |
| | gggttaggaattggaacagggtcgggtacaggcggacgcactgggtatattccattggga |
| | acaaggcctcccacagctacagatacacttgctcctgtaagaccccctttaacagtagatc |
| | ctgtgggcccttctgatccttctatagtttctttagtggaagaaactagttttattgatgctggt |
| | gcaccaacatctgtaccttccattcccccagatgtatcaggatttagtattactacttcaact |
| | gataccacacctgctatattagatattaataatactgttactactgttactacacataataat |
| | cccactttcactgacccatctgtattgcagcctccaacacctgcagaaactggagggcattt |
| | tacactttcatcatccactattagtacacataattatgaagaaattcctatggatacatttatt |
| | gttagcacaaaccctaacacagtaactagtagcacacccataccagggtctcgcccagtg |
| | gcacgcctaggattatatagtcgcacaacacaacaggttaaagttgtagaccctgcttttgt |
| | aaccactcccactaaacttattacatatgataatcctgcatatgaaggtatagatgtggata |
| | atacattatatttttctagtaatgataatagtattaatatagctccagatcctgactttttggat |
| | atagttgctttacataggccagcattaacctctaggcgtactggcattaggtacagtagaat |
| | tggtaataaacaaacactacgtactcgtagtggaaaatctataggtgctaaggtacattatt |
| | attatgatttaagtactattgatcctgcagaagaaatagaattacaaactataacaccttct |
| | acatatactaccacttcacatgcagcctcacctacttctattaataatggattatatgatattt |
| | atgcagatgactttattacagatacttctacaaccccggtaccatctgtaccctctacatcttt |
| | atcaggttatattcctgcaaatacaacaattccttttggtggtgcatacaatattcctttagta |
| | tcaggtcctgatatacccattaatataactgaccaagctccttcattaattcctatagttccag |
| | ggtctccacaatatacaattattgctgatgcaggtgacttttatttacatcctagttattacat |
| | gttacgaaaacgacgtaaacgtttaccatattttttttcagatgtctctttggctgcctaggcg |
| | gccgctcgagtctagagggcccgtttaaacccgctgatcagcctcgactgtgccttctagtt |
| | gccagccatctgttgtttgcccctcccccgtgccttccttgaccctggaaggtgccactccca |
| | ctgtcctttcctaataaaatgaggaaattgcatcgcattgtctgagtaggtgtcattctattct |
| | ggggggtggggtggggcaggacagcaagggggaggattgggaagacaatagcaggca |
| | tgctggggatgcggtgggctctatggcttctgaggcggaaagaaccagctggggctctag |
| | ggggtatccccacgcgccctgtagcggcgcattaagcgcggcgggtgtggtggttacgcg |
| | cagcgtgaccgctacacttgccagcgccctagcgcccgctcctttcgctttcttcccttccttt |
| | ctcgccacgttcgccggctttccccgtcaagctctaaatcgggggctccctttagggttccga |
| | tttagtgctttacggcacctcgaccccaaaaaacttgattagggtgatggttcacgtagtgg |
| | gccatcgccctgatagacggtttttcgccctttgacgttggagtccacgttctttaatagtgg |
| | actcttgttccaaactggaacaacactcaaccctatctcggtctattcttttgatttataaggg |
| | attttgccgatttcggcctattggttaaaaaatgagctgatttaacaaaaatttaacgcgaat |
| | taattctgtggaatgtgtgtcagttagggtgtggaaagtccccaggctccccagcaggcag |
| | aagtatgcaaagcatgcatctcaattagtcagcaaccaggtgtggaaagtccccaggctc |
| | cccagcaggcagaagtatgcaaagcatgcatctcaattagtcagcaaccatagtcccgcc |
| | cctaactccgcccatcccgcccctaactccgcccagttccgcccattctccgccccatggct |
| | gactaattttttttatttatgcagaggccgaggccgcctctgcctctgagctattccagaagt |
| | agtgaggaggcttttttggaggcctaggcttttgcaaaaagctcccgggagcttgtatatcc |
| | attttcggatctgatcaagagacaggatgaggatcgtttcgcatgattgaacaagatggatt |
| | gcacgcaggttctccggccgcttgggtggagaggctattcggctatgactgggcacaaca |
| | gacaatcggctgctctgatgccgccgtgttccggctgtcagcgcaggggcgcccggttcttt |
| | ttgtcaagaccgacctgtccggtgccctgaatgaactgcaggacgaggcagcgcggctat |
| | cgtggctggccacgacgggcgttccttgcgcagctgtgctcgacgttgtcactgaagcggg |
| | aagggactggctgctattgggcgaagtgccggggcaggatctcctgtcatctcaccttgctc |
| | ctgccgagaaagtatccatcatggctgatgcaatgcggcggctgcatacgcttgatccggc |
| | tacctgcccattcgaccaccaagcgaaacatcgcatcgagcgagcacgtactcggatgga |
| | agccggtcttgtcgatcaggatgatctggacgaagagcatcaggggctcgcgccagccga |
| | actgttcgccaggctcaaggcgcgcatgcccgacggcgaggatctcgtcgtgacccatgg |
| | cgatgcctgcttgccgaatatcatggtggaaaatggccgcttttctggattcatcgactgtgg |
| | ccggctgggtgtggcggaccgctatcaggacatagcgttggctacccgtgatattgctgaa |
| | gagcttggcggcgaatgggctgaccgcttcctcgtgctttacggtatcgccgctcccgattc |
| | gcagcgcatcgccttctatcgccttcttgacgagttcttctgagcgggactctggggttcgaa |
| | atgaccgaccaagcgacgcccaacctgccatcacgagatttcgattccaccgccgccttct |
| | atgaaaggttgggcttcggaatcgttttccgggacgccggctggatgatcctccagcgcgg |
| | ggatctcatgctggagttcttcgcccaccccaacttgtttattgcagcttataatggttacaa |
| | ataaagcaatagcatcacaaatttcacaaataaagcatttttttcactgcattctagttgtgg |
| | tttgtccaaactcatcaatgtatcttatcatgtctgtataccgtcgacctctagctagagcttg |
| | gcgtaatcatggtcatagctgtttcctgtgtgaaattgttatccgctcacaattccacacaac |
| | atacgagccggaagcataaagtgtaaagcctggggtgcctaatgagtgagctaactcaca |
| | ttaattgcgttgcgctcactgcccgctttccagtcgggaaacctgtcgtgccagctgcattaa |
| | tgaatcggccaacgcgcggggagaggcggtttgcgtattgggcgctcttccgcttcctcgct |
| | cactgactcgctgcgctcggtcgttcggctgcggcgagcggtatcagctcactcaaaggcg |
| | gtaatacggttatccacagaatcaggggataacgcaggaaagaacatgtgagcaaaagg |
| | ccagcaaaaggccaggaaccgtaaaaaggccgcgttgctggcgtttttccataggctccg |
| | cccccctgacgagcatcacaaaaatcgacgctcaagtcagaggtggcgaaacccgacag |
| | gactataaagataccaggcgtttccccctggaagctccctcgtgcgctctcctgttccgacc |
| | ctgccgcttaccggatacctgtccgcctttctcccttcgggaagcgtggcgctttctcatagct |
| | cacgctgtaggtatctcagttcggtgtaggtcgttcgctccaagctgggctgtgtgcacgaa |
| | ccccccgttcagcccgaccgctgcgccttatccggtaactatcgtcttgagtccaacccggt |
| | aagacacgacttatcgccactggcagcagccactggtaacaggattagcagagcgaggt |
| | atgtaggcggtgctacagagttcttgaagtggtggcctaactacggctacactagaagaac |
| | agtatttggtatctgcgctctgctgaagccagttaccttcggaaaaagagttggtagctcttg |
| | atccggcaaacaaaccaccgctggtagcggtttttttgtttgcaagcagcagattacgcgc |
| | agaaaaaaaggatctcaagaagatcctttgatcttttctacggggtctgacgctcagtgga |
| | acgaaaactcacgttaagggattttggtcatgagattatcaaaaaggatcttcacctagatc |
| | cttttaaattaaaaatgaagttttaaatcaatctaaagtatatatgagtaaacttggtctgac |
| | agttaccaatgcttaatcagtgaggcacctatctcagcgatctgtctatttcgttcatccata |
| | gttgcctgactccccgtcgtgtagataactacgatacgggagggcttaccatctggcccca |
| | gtgctgcaatgataccgcgagacccacgctcaccggctccagatttatcagcaataaacca |
| | gccagccggaagggccgagcgcagaagtggtcctgcaactttatccgcctccatccagtct |
| | attaattgttgccgggaagctagagtaagtagttcgccagttaatagtttgcgcaacgttgtt |
| | gccattgctacaggcatcgtggtgtcacgctcgtcgtttggtatggcttcattcagctccggtt |
| | cccaacgatcaaggcgagttacatgatcccccatgttgtgcaaaaaagcggttagctcctt |
| | cggtcctccgatcgttgtcagaagtaagttggccgcagtgttatcactcatggttatggcag |
| | cactgcataattctcttactgtcatgccatccgtaagatgcttttctgtgactggtgagtactc |
| | aaccaagtcattctgagaatagtgtatgcggcgaccgagttgctcttgcccggcgtcaata |
| | cgggataataccgcgccacatagcagaactttaaaagtgctcatcattggaaaacgttctt |
| | cggggcgaaaactctcaaggatcttaccgctgttgagatccagttcgatgtaacccactcgt |
| | gcacccaactgatcttcagcatcttttactttcaccagcgtttctgggtgagcaaaaacagg |
| | aaggcaaaatgccgcaaaaaagggaataagggcgacacggaaatgttgaatactcata |
| | ctcttcctttttcaatattattgaagcatttatcagggttattgtctcatgagcggatacatattt |
| | gaatgtatttagaaaaataaacaaataggggttccgcgcacatttccccgaaaagtgcca |
| | cctgacgtc (SEQ ID NO: 19) |
| HPV-16 L1 | MCLYTRVLILHYHLLPLYGPLYHPRPLPLHSILVYMVHIII |
| amino acid | CGHYIILFLRNVNVFPIFLQMALWRPSDNTVYLPPPSVAR |
| sequence | VVNTDDYVTPTSIFYHAGSSRLLTVGNPYFRVPAGGGNK |
| | QDIPKVSAYQYRVFRVQLPDPNKFGLPDTSIYNPETQRLV |
| | WACAGVEIGRGQPLGVGLSGHPFYNKLDDTESSHAATSN |
| | VSEDVRDNVSVDYKQTQLCILGCAPAIGEHWAKGTACKS |
| | RPLSQGDCPPLELKNTVLEDGDMVDTGYGAMDFSTLQD |
| | TKCEVPLDICQSICKYPDYLQMSADPYGDSMFFCLRREQL |
| | FARHFWNRAGTMGDTVPQSLYIKGTGMPASPGSCVYSPS |
| | PSGSIVTSDSQLFNKPYWLHKAQGHNNGVCWHNQLFVT |
| | VVDTTPSTNLTICASTQSPVPGQYDATKFKQYSRHVEEYD |
| | LQFIFQLCTITLTADVMSYIHSMNSSILEDWNFGVPPPPTT |
| | SLVDTYRFVQSVAITCQKDAAPAENKDPYDKLKFWNVDL |
| | KEKFSLDLDQYPLGRKFLVQAGLRRKPTIGPRKRSAPSAT |
| | TSSKPAKRVRVRARK (SEQ ID NO: 20) |
| HPV-16 L2 | MRHKRSAKRTKRASATQLYKTCKQAGTCPPDIIPKVEGK |
| amino acid | TIAEQILQYGSMGVFFGGLGIGTGSGTGGRTGYIPLGTRP |
| sequence | PTATDTLAPVRPPLTVDPVGPSDPSIVSLVEETSFIDAGAPT |
| | SVPSIPPDVSGFSITTSTDTTPAILDINNTVTTVTTHNNPTFT |
| | DPSVLQPPTPAETGGHFTLSSSTISTHNYEEIPMDTFIVSTN |
| | PNTVTSSTPIPGSRPVARLGLYSRTTQQVKVVDPAFVTTPT |
| | KLITYDNPAYEGIDVDNTLYFSSNDNSINIAPDPDFLDIVAL |
| | HRPALTSRRTGIRYSRIGNKQTLRTRSGKSIGAKVHYYYD |
| | LSTIDPAEEIELQTITPSTYTTTSHAASPTSINNGLYDIYAD |
| | DFITDTSTTPVPSVPSTSLSGYIPANTTIPFGGAYNIPLVSGP |
| | DIPINITDQAPSLIPIVPGSPQYTIIADAGDFYLHPSYYMLR |
| | KRRKRLPYFFSDVSLA (SEQ ID NO: 21) |
| pDY0023 | gacggatcgggagatctcccgatcccctatggtgcactctcagtacaatctgctctgatgcc |
| HPV-43 L1- | gcatagttaagccagtatctgctccctgcttgtgtgttggaggtcgctgagtagtgcgcgag |
| HCV IRES-L2 | caaaatttaagctacaacaaggcaaggcttgaccgacaattgcatgaagaatctgcttag |
| (seq | ggttaggcgttttgcgctgcttcgcgatgtacgggccagatatacgcgttgacattgattatt |
| GKgnevQk) | gactagttattaatagtaatcaattacggggtcattagttcatagcccatatatggagttccg |
| CMV | cgttacataacttacggtaaatggcccgcctggctgaccgcccaacgacccccgcccattg |
| promoter: | acgtcaataatgacgtatgttcccatagtaacgccaatagggactttccattgacgtcaatg |
| nucleotides | ggtggagtatttacggtaaactgcccacttggcagtacatcaagtgtatcatatgccaagta |
| 232 to 819 | cgccccctattgacgtcaatgacggtaaatggcccgcctggcattatgcccagtacatgac |
| T7 promoter: | cttatgggactttcctacttggcagtacatctacgtattagtcatcgctattaccatggtgatg |
| nucleotides | cggttttggcagtacatcaatgggcgtggatagcggtttgactcacggggatttccaagtct |
| 863 to 879 | ccaccccattgacgtcaatgggagtttgttttggcaccaaaatcaacgggactttccaaaat |
| HPV-43 L1 | gtcgtaacaactccgccccattgacgcaaatgggcggtaggcgtgtacggtgggaggtct |
| coding | atataagcagagctctctggctaactagagaacccactgcttactggcttatcgaaattaat |
| sequence: | acgactcactatagggagacccaagctggctagcgtttaaacttaagcttgccaccatgtg |
| nucleotides | gcggcttaatgacaacaaggtttacctgcctcctccagggcctatagcatctattgtgagca |
| 923 to 2434 | cagatgaatatgtgcaacgcaccaacttattttattatgctggcagttcacgtttgcttgcag |
| IRES: | tgggtcacccatatttcccccttaaaaattcctctggtaaaataactgtacctaaggtttctg |
| nucleotides | gttatcaatacagagtatttagagttaaattgcctgaccctaataaatttggcttttcagaaa |
| 2435 to 2873 | caacactggttacatcagacactcagcgtttagtctggggatgcgtaggagttgaaattggt |
| HPV-43 L2 | agaggacaacctttaggtgttggaataagtggccatccgtatttaaataagtatgatgaca |
| coding | ctgaaaacccgtctgggtatggcacatcgccgggacaagataacagagaaaatgtagca |
| sequence: | atggattataaacaaacacagctgtgtattgttggctgtacacctcctatgggtgaatattg |
| nucleotides | gggtcagggtgtgccttgcaacgcatcaggtgttacccaaggtgattgtcctgtaatagaat |
| 2874 to 4265 | taaaaagtgaagttatacaggatggtgacatggtagatacaggatttggtgcaatggattt |
| BGH polyA: | tgcttccctacaggccagtaaaagtgatgtacccttagacctggttaatactaaaagtaaat |
| nucleotides | atcctgattatttgggaatggcagcagagccttatgggaatagtttgtttttttttctacgccg |
| 4316 to 4540 | ggaacaaatgttccttagacatttttttaataaagctggtaaaactggcgacgttgtgccttc |
| | cgatatgtatattgctggctctaataccaggtccaaaattgcagatagtatatatttttctaca |
| | cccagtgggtctttggttacttctgattctcaattgtttaacaaacccttatggatacaaaag |
| | gcccagggacataataatggcatttgttttgggaatcagttgtttgttacagtggtagatacc |
| | actcgtagtacaaacttaacgttatgtgcctctactgaccctactgtgcccagtacatatgac |
| | aatgcaaagtttaaggaatacctgcggcatgtggaagaatatgatctgcagtttatatttca |
| | attatgcataataacgctaaacccagaggttatgacatatattcatactatggatcccacat |
| | tattagaggactggaattttggtgtgtccccacctgcctctgcttctttggaagatacttatcg |
| | ctttttgtctaacaaggccattgcatgtcaaaaaaatgctcccccaaaagaacgggaggat |
| | ccctataaaaagtatacattttgggatataaatcttacagaaaagttttctgcacaacttacc |
| | cagtttcccttagggcgcaaatttgttatgcaggcgggtttgcgtcccaaacctaaattaaa |
| | aactgtaaagcgttctgcaccatcctcctctacgtctgcccctgcctctaaacgcaaaaaaa |
| | ctaagcgataattctagtgtacgtagccagcccccgattgggggcgacactccaccataga |
| | tcactcccctgtgaggaactactgtcttcacgcagaaagcgtctagccatggcgttagtatg |
| | agagtcgtgcagcctccaggaccccccctcccgggagagccatagtggtctgcggaaccg |
| | gtgagtacaccggaattgccaggacgaccgggtcctttcttggatcaacccgctcaatgcc |
| | tggagatttgggcgtgcccccgcaagactgctagccgagtagtgttgggtcgcgaaaggc |
| | cttgtggtactgcctgatagggtgcttgcgagtgccccgggaggtctcgtagaccgtgcacc |
| | atgagcacgaatcctaaacctcaaagaaaaaccaaacgtaacaccaaccgccgcccaca |
| | ggacgtcttcatatgtctagccaccatggtgtctcatacacataaaaggcgcaaacgggca |
| | tcagctacacaattatatcaaacatgcaaggctgctggcacatgtccctcggatgtaattaa |
| | taaggttgagcatactacaatagcagatcagatattaaaatgggcgagcatgggagtgta |
| | ttttggagggttgggtattggaacaggctcaggaactggaggcagaacaggctatgtccct |
| | ctaacaacaggtcgtacgggtattgtccctaaggtgactgcagagcctggagtagtgtcac |
| | gtcctcctattgttgtagaatctgttgctccaactgatccttctattgtgtccttaattgaggaa |
| | tcaagcataattcagtccggggctcctattaccaatattccatcacatggtggctttgaggta |
| | acctcctctggatcagaggttcctgcaattttagatgtttccccatctacttcagtgcatatta |
| | ctacatctacacatttaaatcctgcatttactgatcctactattgtacagccaacccccccag |
| | ttgaggctgggggacgtattataatatctcactccactgttactgctgatagtgctgaacaa |
| | attcctatggatacgtttgttatacacagcgatcctaccactagcacacctattccaggcact |
| | gccccacgacctcgtttgggcctgtacagtaaggcattgcagcaggtggaaattgttgacc |
| | ctacatttttgtcctcgccacaacgtttaattacatatgacaatcctgtatttgaggatcctaa |
| | tgctacattaacatttgaacagcctacagtacatgaagctcctgattctaggtttatggatat |
| | agttactttacatagacctgcattaacatcccgacgaggtatagttagatttagtagggtgg |
| | gtgcgcgcggtactatgtatactcgcagtggtatacgtattgggggtcgtgtacactttttta |
| | cagatattagttccatacccacagaggaatcaatagaattgcagcccctaggacgttccca |
| | gtcctttcctactgtttctgatactagtgatttatatgatatatatgcagatgagaatctgttaa |
| | ataatgatattagttttactgacacacacgtgtccctacagaattctactaaggttgttaata |
| | cagctgtgccacttgcaactgtacctgatatttatgcacaaacggggcctgacataagcttt |
| | cctactattcctattcacattccatatattcctgtgtccccatctatttcccctcagtctgtttcc |
| | atacatggcactgatttttatttgcatccttcattgtggcatttgggcaaacgccgtaaacgct |
| | tttcatatttttttacagataactatgtggcggcttaagcggccgctcgagtctagagggccc |
| | gtttaaacccgctgatcagcctcgactgtgccttctagttgccagccatctgttgtttgcccct |
| | cccccgtgccttccttgaccctggaaggtgccactcccactgtcctttcctaataaaatgagg |
| | aaattgcatcgcattgtctgagtaggtgtcattctattctggggggtggggtggggcaggac |
| | agcaagggggaggattgggaagacaatagcaggcatgctggggatgcggtgggctctat |
| | ggcttctgaggcggaaagaaccagctggggctctagggggtatccccacgcgccctgtag |
| | cggcgcattaagcgcggcgggtgtggtggttacgcgcagcgtgaccgctacacttgccag |
| | cgccctagcgcccgctcctttcgctttcttcccttcctttctcgccacgttcgccggctttcccc |
| | gtcaagctctaaatcgggggctccctttagggttccgatttagtgctttacggcacctcgacc |
| | ccaaaaaacttgattagggtgatggttcacgtagtgggccatcgccctgatagacggttttt |
| | cgccctttgacgttggagtccacgttctttaatagtggactcttgttccaaactggaacaaca |
| | ctcaaccctatctcggtctattcttttgatttataagggattttgccgatttcggcctattggtta |
| | aaaaatgagctgatttaacaaaaatttaacgcgaattaattctgtggaatgtgtgtcagtta |
| | gggtgtggaaagtccccaggctccccagcaggcagaagtatgcaaagcatgcatctcaat |
| | tagtcagcaaccaggtgtggaaagtccccaggctccccagcaggcagaagtatgcaaag |
| | catgcatctcaattagtcagcaaccatagtcccgcccctaactccgcccatcccgcccctaa |
| | ctccgcccagttccgcccattctccgccccatggctgactaattttttttatttatgcagaggc |
| | cgaggccgcctctgcctctgagctattccagaagtagtgaggaggcttttttggaggcctag |
| | gcttttgcaaaaagctcccgggagcttgtatatccattttcggatctgatcaagagacagga |
| | tgaggatcgtttcgcatgattgaacaagatggattgcacgcaggttctccggccgcttgggt |
| | ggagaggctattcggctatgactgggcacaacagacaatcggctgctctgatgccgccgt |
| | gttccggctgtcagcgcaggggcgcccggttctttttgtcaagaccgacctgtccggtgccc |
| | tgaatgaactgcaggacgaggcagcgcggctatcgtggctggccacgacgggcgttcctt |
| | gcgcagctgtgctcgacgttgtcactgaagcgggaagggactggctgctattgggcgaag |
| | tgccggggcaggatctcctgtcatctcaccttgctcctgccgagaaagtatccatcatggct |
| | gatgcaatgcggcggctgcatacgcttgatccggctacctgcccattcgaccaccaagcga |
| | aacatcgcatcgagcgagcacgtactcggatggaagccggtcttgtcgatcaggatgatct |
| | ggacgaagagcatcaggggctcgcgccagccgaactgttcgccaggctcaaggcgcgca |
| | tgcccgacggcgaggatctcgtcgtgacccatggcgatgcctgcttgccgaatatcatggt |
| | ggaaaatggccgcttttctggattcatcgactgtggccggctgggtgtggcggaccgctatc |
| | aggacatagcgttggctacccgtgatattgctgaagagcttggcggcgaatgggctgacc |
| | gcttcctcgtgctttacggtatcgccgctcccgattcgcagcgcatcgccttctatcgccttct |
| | tgacgagttcttctgagcgggactctggggttcgaaatgaccgaccaagcgacgcccaac |
| | ctgccatcacgagatttcgattccaccgccgccttctatgaaaggttgggcttcggaatcgtt |
| | ttccgggacgccggctggatgatcctccagcgcggggatctcatgctggagttcttcgccca |
| | ccccaacttgtttattgcagcttataatggttacaaataaagcaatagcatcacaaatttcac |
| | aaataaagcatttttttcactgcattctagttgtggtttgtccaaactcatcaatgtatcttatc |
| | atgtctgtataccgtcgacctctagctagagcttggcgtaatcatggtcatagctgtttcctgt |
| | gtgaaattgttatccgctcacaattccacacaacatacgagccggaagcataaagtgtaaa |
| | gcctggggtgcctaatgagtgagctaactcacattaattgcgttgcgctcactgcccgctttc |
| | cagtcgggaaacctgtcgtgccagctgcattaatgaatcggccaacgcgcggggagagg |
| | cggtttgcgtattgggcgctcttccgcttcctcgctcactgactcgctgcgctcggtcgttcgg |
| | ctgcggcgagcggtatcagctcactcaaaggcggtaatacggttatccacagaatcaggg |
| | gataacgcaggaaagaacatgtgagcaaaaggccagcaaaaggccaggaaccgtaaa |
| | aaggccgcgttgctggcgtttttccataggctccgcccccctgacgagcatcacaaaaatc |
| | gacgctcaagtcagaggtggcgaaacccgacaggactataaagataccaggcgtttcccc |
| | ctggaagctccctcgtgcgctctcctgttccgaccctgccgcttaccggatacctgtccgcct |
| | ttctcccttcgggaagcgtggcgctttctcatagctcacgctgtaggtatctcagttcggtgta |
| | ggtcgttcgctccaagctgggctgtgtgcacgaaccccccgttcagcccgaccgctgcgcc |
| | ttatccggtaactatcgtcttgagtccaacccggtaagacacgacttatcgccactggcagc |
| | agccactggtaacaggattagcagagcgaggtatgtaggcggtgctacagagttcttgaa |
| | gtggtggcctaactacggctacactagaagaacagtatttggtatctgcgctctgctgaagc |
| | cagttaccttcggaaaaagagttggtagctcttgatccggcaaacaaaccaccgctggtag |
| | cggtttttttgtttgcaagcagcagattacgcgcagaaaaaaaggatctcaagaagatcctt |
| | tgatcttttctacggggtctgacgctcagtggaacgaaaactcacgttaagggattttggtc |
| | atgagattatcaaaaaggatcttcacctagatccttttaaattaaaaatgaagttttaaatca |
| | atctaaagtatatatgagtaaacttggtctgacagttaccaatgcttaatcagtgaggcacc |
| | tatctcagcgatctgtctatttcgttcatccatagttgcctgactccccgtcgtgtagataact |
| | acgatacgggagggcttaccatctggccccagtgctgcaatgataccgcgagacccacgc |
| | tcaccggctccagatttatcagcaataaaccagccagccggaagggccgagcgcagaag |
| | tggtcctgcaactttatccgcctccatccagtctattaattgttgccgggaagctagagtaag |
| | tagttcgccagttaatagtttgcgcaacgttgttgccattgctacaggcatcgtggtgtcacg |
| | ctcgtcgtttggtatggcttcattcagctccggttcccaacgatcaaggcgagttacatgatc |
| | ccccatgttgtgcaaaaaagcggttagctccttcggtcctccgatcgttgtcagaagtaagt |
| | tggccgcagtgttatcactcatggttatggcagcactgcataattctcttactgtcatgccatc |
| | cgtaagatgcttttctgtgactggtgagtactcaaccaagtcattctgagaatagtgtatgcg |
| | gcgaccgagttgctcttgcccggcgtcaatacgggataataccgcgccacatagcagaac |
| | tttaaaagtgctcatcattggaaaacgttcttcggggcgaaaactctcaaggatcttaccgc |
| | tgttgagatccagttcgatgtaacccactcgtgcacccaactgatcttcagcatcttttacttt |
| | caccagcgtttctgggtgagcaaaaacaggaaggcaaaatgccgcaaaaaagggaata |
| | agggcgacacggaaatgttgaatactcatactcttcctttttcaatattattgaagcatttatc |
| | agggttattgtctcatgagcggatacatatttgaatgtatttagaaaaataaacaaataggg |
| | gttccgcgcacatttccccgaaaagtgccacctgacgtc (SEQ ID NO: 22) |
| HPV-43 L1 | MWRLNDNKVYLPPPGPIASIVSTDEYVQRTNLFYYAGSSR |
| amino acid | LLAVGHPYFPLKNSSGKITVPKVSGYQYRVFRVKLPDPNK |
| sequence | FGFSETTLVTSDTQRLVWGCVGVEIGRGQPLGVGISGHP |
| | YLNKYDDTENPSGYGTSPGQDNRENVAMDYKQTQLCIV |
| | GCTPPMGEYWGQGVPCNASGVTQGDCPVIELKSEVIQDG |
| | DMVDTGFGAMDFASLQASKSDVPLDLVNTKSKYPDYLG |
| | MAAEPYGNSLFFFLRREQMFLRHFFNKAGKTGDVVPSD |
| | MYIAGSNTRSKIADSIYFSTPSGSLVTSDSQLFNKPLWIQK |
| | AQGHNNGICFGNQLFVTVVDTTRSTNLTLCASTDPTVPST |
| | YDNAKFKEYLRHVEEYDLQFIFQLCIITLNPEVMTYIHTM |
| | DPTLLEDWNFGVSPPASASLEDTYRFLSNKAIACQKNAPP |
| | KEREDPYKKYTFWDINLTEKFSAQLTQFPLGRKFVMQAG |
| | LRPKPKLKTVKRSAPSSSTSAPASKRKKTKR |
| | (SEQ ID NO: 23) |
| HPV-43 L2 | MVSHTHKRRKRASATQLYQTCKAAGTCPSDVINKVEHTT |
| amino acid | IADQILKWASMGVYFGGLGIGTGSGTGGRTGYVPLTTGR |
| sequence | TGIVPKVTAEPGVVSRPPIVVESVAPTDPSIVSLIEESSIIQS |
| | GAPITNIPSHGGFEVTSSGSEVPAILDVSPSTSVHITTSTHL |
| | NPAFTDPTIVQPTPPVEAGGRIIISHSTVTADSAEQIPMDTF |
| | VIHSDPTTSTPIPGTAPRPRLGLYSKALQQVEIVDPTFLSSP |
| | QRLITYDNPVFEDPNATLTFEQPTVHEAPDSRFMDIVTLH |
| | RPALTSRRGIVRFSRVGARGTMYTRSGIRIGGRVHFFTDIS |
| | SIPTEESIELQPLGRSQSFPTVSDTSDLYDIYADENLLNNDI |
| | SFTDTHVSLQNSTKVVNTAVPLATVPDIYAQTGPDISFPTI |
| | PIHIPYIPVSPSISPQSVSIHGTDFYLHPSLWHLGKRRKRFS |
| | YFFTDNYVAA (SEQ ID NO: 24) |
| pDY0037HPV16 | gacggatcgggagatctcccgatcccctatggtgcactctcagtacaatctgctctgatgcc |
| L1-HCV | gcatagttaagccagtatctgctccctgcttgtgtgttggaggtcgctgagtagtgcgcgag |
| IRES-L2 | caaaatttaagctacaacaaggcaaggcttgaccgacaattgcatgaagaatctgcttag |
| (seq | ggttaggcgttttgcgctgcttcgcgatgtacgggccagatatacgcgttgacattgattatt |
| upE23e6b) | gactagttattaatagtaatcaattacggggtcattagttcatagcccatatatggagttccg |
| CMV | cgttacataacttacggtaaatggcccgcctggctgaccgcccaacgacccccgcccattg |
| promoter: | acgtcaataatgacgtatgttcccatagtaacgccaatagggactttccattgacgtcaatg |
| nucleotides | ggtggagtatttacggtaaactgcccacttggcagtacatcaagtgtatcatatgccaagta |
| 232 to 819 | cgccccctattgacgtcaatgacggtaaatggcccgcctggcattatgcccagtacatgac |
| T7 promoter: | cttatgggactttcctacttggcagtacatctacgtattagtcatcgctattaccatggtgatg |
| nucleotides | cggttttggcagtacatcaatgggcgtggatagcggtttgactcacggggatttccaagtct |
| 863 to 879 | ccaccccattgacgtcaatgggagtttgttttggcaccaaaatcaacgggactttccaaaat |
| HPV-16 L1 | gtcgtaacaactccgccccattgacgcaaatgggcggtaggcgtgtacggtgggaggtct |
| coding | atataagcagagctctctggctaactagagaacccactgcttactggcttatcgaaattaat |
| sequence: | acgactcactatagggagacccaagctggctagcgtttaaacttaagcttgccaccatgtc |
| nucleotides | actttggttgccgtctgaggctaccgtataccttccccctgtgcctgtgtccaaagtagtcag |
| 923 to 2440 | tacagatgagtacgtggcgaggactaatatctattatcacgcaggaacgtccagactcctc |
| IRES: | gccgtcggccacccgtatttcccgatcaaaaaacctaacaataataagattttggtccctaa |
| nucleotides | ggtctccggcctccaataccgggtgttccgaattcacctgccagacccaaataagttcggtt |
| 2441 to 2879 | tccctgatacctccttctataaccctgacacgcaaagactggtatgggcctgtgtcggtgttg |
| HPV-16 L2 | aagtgggcaggggccagcccttgggagttggcatctctgggcatcctcttcttaacaagctc |
| coding | gatgataccgaaaacgcgagtgcgtatgccgccaatgccggggtggataatagggagtg |
| sequence: | cattagtatggattataaacaaacgcaactgtgtctgatcggatgcaagccgcctataggc |
| nucleotides | gagcattgggggaaggggtccccctgtacgaatgtagcggtgaatccgggtgactgcccg |
| 2880 to 4301 | cccctggagctcatcaataccgtaattcaagatggagacatggtccatacgggatttggtg |
| BGH polyA: | ccatggactttaccaccctccaggctaacaagtctgaggtaccgctggacatttgcacctcc |
| nucleotides | atttgtaaatacccagactatataaaaatggttagtgagccatatggtgacagcctgtttttt |
| 4352 to 4576 | tacctgaggagagagcagatgttcgttaggcacttgtttaatcgcgctggtactgttgggga |
| | gaatgtgccagatgatctctacatcaagggaagcggatctacggcaaaccttgctagttct |
| | aattactttccaacaccgtcaggttcaatggttacaagcgacgcgcaaatttttaacaaacc |
| | gtactggcttcaaagagcccaaggccataataacggtatctgttggggaaaccagcttttt |
| | gtcacagttgtagatacaacgcgatcaacgaacatgagtttgtgtgcggcgatatccacta |
| | gtgaaacgacttacaaaaatactaatttcaaagaatacctccgccatggtgaggagtatga |
| | ccttcagtttatatttcaattgtgcaagattacacttacagcggacgttatgacttatattcac |
| | agcatgaactcaacaattcttgaagactggaactttgggcttcagccgccgccaggggga |
| | accttggaagacacttacaggttcgtaacgcaggctatcgcatgtcagaaacatacccctc |
| | cagctccgaaagaagacgatcccctgaaaaagtatacattctgggaggtcaacctgaagg |
| | agaaattttccgctgatctcgatcagttccctcttgggaggaaatttttgctgcaggctggac |
| | tcaaggctaaaccaaagttcacactcggcaaacgaaaagccacgccaactacaagtagt |
| | acgagtacgacagccaagcgaaagaaacgcaagttgtaattctagtgtacgtagccagcc |
| | cccgattgggggcgacactccaccatagatcactcccctgtgaggaactactgtcttcacg |
| | cagaaagcgtctagccatggcgttagtatgagagtcgtgcagcctccaggaccccccctcc |
| | cgggagagccatagtggtctgcggaaccggtgagtacaccggaattgccaggacgaccg |
| | ggtcctttcttggatcaacccgctcaatgcctggagatttgggcgtgcccccgcaagactgc |
| | tagccgagtagtgttgggtcgcgaaaggccttgtggtactgcctgatagggtgcttgcgagt |
| | gccccgggaggtctcgtagaccgtgcaccatgagcacgaatcctaaacctcaaagaaaa |
| | accaaacgtaacaccaaccgccgcccacaggacgtcttcatatgtctagccaccatgcgg |
| | cacaagcgatccgccaagaggactaagagagcgtctgctacccaactttataaaacctgc |
| | aaacaggcaggcacttgccctccagacatcatccccaaggtcgagggtaagaccatcgcg |
| | gaacaaattttgcaatacgggtccatgggggttttttttggcggtcttggtatagggacggg |
| | cagtggaacgggcggtaggaccggttatattcctctcggaacgcgaccacccactgcaac |
| | agacacattggcacccgtgagaccacctctgactgttgacccggtaggaccatctgatcca |
| | tcaattgtcagtctcgttgaagagacgagctttatcgacgctggtgctccgacaagtgttcct |
| | tctatcccacccgatgtatccggttttagtattactacgagtactgacactacccctgctatac |
| | ttgacatcaacaacacggtaacaactgtcactacccacaacaacccaacgtttacggacc |
| | ctagcgtgctgcaacctccaacacccgccgagacaggaggacattttactttgtctagttct |
| | acaatctctacccacaactatgaggaaattccaatggacacttttatcgtaagtaccaaccc |
| | aaacacagtcaccagtagcacccccatccctggcagtcgaccggtggcaagactgggttt |
| | gtactcacggacaacgcagcaagtgaaagttgtagaccctgcgttcgttaccaccccaac |
| | aaaactgattacatatgataacccagcatatgaaggtatcgatgttgataataccctctact |
| | tcagttctaatgacaattctataaatattgctcccgaccctgactttctggacatagtagccct |
| | gcatcgaccagccctcacttctcggcgaacgggtatcaggtattctcgaataggtaacaag |
| | caaaccctccgcacacgctcagggaagtctattggagctaaagtccattattactacgattt |
| | gagcacaattgaccccgccgaggagatcgagcttcaaacgattactccaagtacttatacc |
| | actacctcccatgctgcgtctcctacgagcattaataatgggctttatgatatttacgcagac |
| | gacttcatcactgatacatctactacccccgtaccgtcagtacccagcacgagtctctcagg |
| | ttacatccccgccaacaccactataccgttcggaggtgcatacaatatcccgttggtcagtg |
| | ggccggacattccaataaatataactgatcaagcgccgtctcttatccccattgttcccggt |
| | agtccccaatacacgataattgccgatgcgggcgatttttacttgcacccttcttactacatg |
| | ctccgaaaacgcagaaagcggcttccctatttcttcagtgatgtttccctcgcggcgtaggc |
| | ggccgctcgagtctagagggcccgtttaaacccgctgatcagcctcgactgtgccttctagt |
| | tgccagccatctgttgtttgcccctcccccgtgccttccttgaccctggaaggtgccactccc |
| | actgtcctttcctaataaaatgaggaaattgcatcgcattgtctgagtaggtgtcattctattc |
| | tggggggtggggtggggcaggacagcaagggggaggattgggaagacaatagcaggc |
| | atgctggggatgcggtgggctctatggcttctgaggcggaaagaaccagctggggctcta |
| | gggggtatccccacgcgccctgtagcggcgcattaagcgcggcgggtgtggtggttacgc |
| | gcagcgtgaccgctacacttgccagcgccctagcgcccgctcctttcgctttcttcccttcctt |
| | tctcgccacgttcgccggctttccccgtcaagctctaaatcgggggctccctttagggttccg |
| | atttagtgctttacggcacctcgaccccaaaaaacttgattagggtgatggttcacgtagtg |
| | ggccatcgccctgatagacggtttttcgccctttgacgttggagtccacgttctttaatagtg |
| | gactcttgttccaaactggaacaacactcaaccctatctcggtctattcttttgatttataagg |
| | gattttgccgatttcggcctattggttaaaaaatgagctgatttaacaaaaatttaacgcga |
| | attaattctgtggaatgtgtgtcagttagggtgtggaaagtccccaggctccccagcaggc |
| | agaagtatgcaaagcatgcatctcaattagtcagcaaccaggtgtggaaagtccccaggc |
| | tccccagcaggcagaagtatgcaaagcatgcatctcaattagtcagcaaccatagtcccg |
| | cccctaactccgcccatcccgcccctaactccgcccagttccgcccattctccgccccatgg |
| | ctgactaattttttttatttatgcagaggccgaggccgcctctgcctctgagctattccagaa |
| | gtagtgaggaggcttttttggaggcctaggcttttgcaaaaagctcccgggagcttgtatat |
| | ccattttcggatctgatcaagagacaggatgaggatcgtttcgcatgattgaacaagatgg |
| | attgcacgcaggttctccggccgcttgggtggagaggctattcggctatgactgggcacaa |
| | cagacaatcggctgctctgatgccgccgtgttccggctgtcagcgcaggggcgcccggttc |
| | tttttgtcaagaccgacctgtccggtgccctgaatgaactgcaggacgaggcagcgcggct |
| | atcgtggctggccacgacgggcgttccttgcgcagctgtgctcgacgttgtcactgaagcg |
| | ggaagggactggctgctattgggcgaagtgccggggcaggatctcctgtcatctcaccttg |
| | ctcctgccgagaaagtatccatcatggctgatgcaatgcggcggctgcatacgcttgatcc |
| | ggctacctgcccattcgaccaccaagcgaaacatcgcatcgagcgagcacgtactcggat |
| | ggaagccggtcttgtcgatcaggatgatctggacgaagagcatcaggggctcgcgccagc |
| | cgaactgttcgccaggctcaaggcgcgcatgcccgacggcgaggatctcgtcgtgaccca |
| | tggcgatgcctgcttgccgaatatcatggtggaaaatggccgcttttctggattcatcgactg |
| | tggccggctgggtgtggcggaccgctatcaggacatagcgttggctacccgtgatattgct |
| | gaagagcttggcggcgaatgggctgaccgcttcctcgtgctttacggtatcgccgctcccg |
| | attcgcagcgcatcgccttctatcgccttcttgacgagttcttctgagcgggactctggggtt |
| | cgaaatgaccgaccaagcgacgcccaacctgccatcacgagatttcgattccaccgccgc |
| | cttctatgaaaggttgggcttcggaatcgttttccgggacgccggctggatgatcctccagc |
| | gcggggatctcatgctggagttcttcgcccaccccaacttgtttattgcagcttataatggtt |
| | acaaataaagcaatagcatcacaaatttcacaaataaagcatttttttcactgcattctagtt |
| | gtggtttgtccaaactcatcaatgtatcttatcatgtctgtataccgtcgacctctagctagag |
| | cttggcgtaatcatggtcatagctgtttcctgtgtgaaattgttatccgctcacaattccacac |
| | aacatacgagccggaagcataaagtgtaaagcctggggtgcctaatgagtgagctaactc |
| | acattaattgcgttgcgctcactgcccgctttccagtcgggaaacctgtcgtgccagctgcat |
| | taatgaatcggccaacgcgcggggagaggcggtttgcgtattgggcgctcttccgcttcctc |
| | gctcactgactcgctgcgctcggtcgttcggctgcggcgagcggtatcagctcactcaaag |
| | gcggtaatacggttatccacagaatcaggggataacgcaggaaagaacatgtgagcaaa |
| | aggccagcaaaaggccaggaaccgtaaaaaggccgcgttgctggcgtttttccataggct |
| | ccgcccccctgacgagcatcacaaaaatcgacgctcaagtcagaggtggcgaaacccga |
| | caggactataaagataccaggcgtttccccctggaagctccctcgtgcgctctcctgttccg |
| | accctgccgcttaccggatacctgtccgcctttctcccttcgggaagcgtggcgctttctcat |
| | agctcacgctgtaggtatctcagttcggtgtaggtcgttcgctccaagctgggctgtgtgca |
| | cgaaccccccgttcagcccgaccgctgcgccttatccggtaactatcgtcttgagtccaacc |
| | cggtaagacacgacttatcgccactggcagcagccactggtaacaggattagcagagcg |
| | aggtatgtaggcggtgctacagagttcttgaagtggtggcctaactacggctacactagaa |
| | gaacagtatttggtatctgcgctctgctgaagccagttaccttcggaaaaagagttggtagc |
| | tcttgatccggcaaacaaaccaccgctggtagcggtggtttttttgtttgcaagcagcagatt |
| | acgcgcagaaaaaaaggatctcaagaagatcctttgatcttttctacggggtctgacgctc |
| | agtggaacgaaaactcacgttaagggattttggtcatgagattatcaaaaaggatcttcac |
| | ctagatccttttaaattaaaaatgaagttttaaatcaatctaaagtatatatgagtaaacttg |
| | gtctgacagttaccaatgcttaatcagtgaggcacctatctcagcgatctgtctatttcgttc |
| | atccatagttgcctgactccccgtcgtgtagataactacgatacgggagggcttaccatctg |
| | gccccagtgctgcaatgataccgcgagacccacgctcaccggctccagatttatcagcaat |
| | aaaccagccagccggaagggccgagcgcagaagtggtcctgcaactttatccgcctccat |
| | ccagtctattaattgttgccgggaagctagagtaagtagttcgccagttaatagtttgcgca |
| | acgttgttgccattgctacaggcatcgtggtgtcacgctcgtcgtttggtatggcttcattcag |
| | ctccggttcccaacgatcaaggcgagttacatgatcccccatgttgtgcaaaaaagcggtt |
| | agctccttcggtcctccgatcgttgtcagaagtaagttggccgcagtgttatcactcatggtt |
| | atggcagcactgcataattctcttactgtcatgccatccgtaagatgcttttctgtgactggtg |
| | agtactcaaccaagtcattctgagaatagtgtatgcggcgaccgagttgctcttgcccggc |
| | gtcaatacgggataataccgcgccacatagcagaactttaaaagtgctcatcattggaaa |
| | acgttcttcggggcgaaaactctcaaggatcttaccgctgttgagatccagttcgatgtaac |
| | ccactcgtgcacccaactgatcttcagcatcttttactttcaccagcgtttctgggtgagcaa |
| | aaacaggaaggcaaaatgccgcaaaaaagggaataagggcgacacggaaatgttgaat |
| | actcatactcttcctttttcaatattattgaagcatttatcagggttattgtctcatgagcggat |
| | acatatttgaatgtatttagaaaaataaacaaataggggttccgcgcacatttccccgaaa |
| | agtgccacctgacgtc (SEQ ID NO: 25) |
| HPV-16 L1 | MSLWLPSEATVYLPPVPVSKVVSTDEYVARTNIYYHAGTS |
| amino acid | RLLAVGHPYFPIKKPNNNKILVPKVSGLQYRVFRIHLPDP |
| sequence | NKFGFPDTSFYNPDTQRLVWACVGVEVGRGQPLGVGISG |
| | HPLLNKLDDTENASAYAANAGVDNRECISMDYKQTQLCL |
| | IGCKPPIGEHWGKGSPCTNVAVNPGDCPPLELINTVIQDG |
| | DMVHTGFGAMDFTTLQANKSEVPLDICTSICKYPDYIKM |
| | VSEPYGDSLFFYLRREQMFVRHLFNRAGTVGENVPDDLY |
| | IKGSGSTANLASSNYFPTPSGSMVTSDAQIFNKPYWLQRA |
| | QGHNNGICWGNQLFVTVVDTTRSTNMSLCAAISTSETTY |
| | KNTNFKEYLRHGEEYDLQFIFQLCKITLTADVMTYIHSM |
| | NSTILEDWNFGLQPPPGGTLEDTYRFVTQAIACQKHTPPA |
| | PKEDDPLKKYTFWEVNLKEKFSADLDQFPLGRKFLLQAG |
| | LKAKPKFTLGKRKATPTTSSTSTTAKRKKRKL |
| | (SEQ ID NO: 26) |
| HPV-16 L2 | MRHKRSAKRTKRASATQLYKTCKQAGTCPPDIIPKVEGK |
| amino acid | TIAEQILQYGSMGVFFGGLGIGTGSGTGGRTGYIPLGTRP |
| sequence | PTATDTLAPVRPPLTVDPVGPSDPSIVSLVEETSFIDAGAPT |
| | SVPSIPPDVSGFSITTSTDTTPAILDINNTVTTVTTHNNPTFT |
| | DPSVLQPPTPAETGGHFTLSSSTISTHNYEEIPMDTFIVSTN |
| | PNTVTSSTPIPGSRPVARLGLYSRTTQQVKVVDPAFVTTPT |
| | KLITYDNPAYEGIDVDNTLYFSSNDNSINIAPDPDFLDIVAL |
| | HRPALTSRRTGIRYSRIGNKQTLRTRSGKSIGAKVHYYYD |
| | LSTIDPAEEIELQTITPSTYTTTSHAASPTSINNGLYDIYAD |
| | DFITDTSTTPVPSVPSTSLSGYIPANTTIPFGGAYNIPLVSGP |
| | DIPINITDQAPSLIPIVPGSPQYTIIADAGDFYLHPSYYMLR |
| | KRRKRLPYFFSDVSLAA (SEQ ID NO: 27) |
| pDY0038 | gacggatcgggagatctcccgatcccctatggtgcactctcagtacaatctgctctgatgcc |
| HPV137 | gcatagttaagccagtatctgctccctgcttgtgtgttggaggtcgctgagtagtgcgcgag |
| L1-HCV | caaaatttaagctacaacaaggcaaggcttgaccgacaattgcatgaagaatctgcttag |
| IRES-L2 | ggttaggcgttttgcgctgcttcgcgatgtacgggccagatatacgcgttgacattgattatt |
| (seq | gactagttattaatagtaatcaattacggggtcattagttcatagcccatatatggagttccg |
| 3upaGXw2) | cgttacataacttacggtaaatggcccgcctggctgaccgcccaacgacccccgcccattg |
| CMV | acgtcaataatgacgtatgttcccatagtaacgccaatagggactttccattgacgtcaatg |
| promoter: | ggtggagtatttacggtaaactgcccacttggcagtacatcaagtgtatcatatgccaagta |
| nucleotides | cgccccctattgacgtcaatgacggtaaatggcccgcctggcattatgcccagtacatgac |
| 232 to 819 | cttatgggactttcctacttggcagtacatctacgtattagtcatcgctattaccatggtgatg |
| T7 promoter: | cggttttggcagtacatcaatgggcgtggatagcggtttgactcacggggatttccaagtct |
| nucleotides | ccaccccattgacgtcaatgggagtttgttttggcaccaaaatcaacgggactttccaaaat |
| 863 to 879 | gtcgtaacaactccgccccattgacgcaaatgggcggtaggcgtgtacggtgggaggtct |
| HPV-137 L1 | atataagcagagctctctggctaactagagaacccactgcttactggcttatcgaaattaat |
| coding | acgactcactatagggagacccaagctggctagcgtttaaacttaagcttgccaccatggc |
| sequence: | ggtttgggtccccaataaagggcgcctttaccttcctccacagagacccgtggcgaaagttt |
| nucleotides | tgtcaacggatgattatattgtcgggacggacttgtattttcatagctccacagaccggttgc |
| 923 to 2473 | ttacggtcggacatccgttctttgacgtactgagtacggaccaaaatacagttgatgtgcct |
| IRES: | aaggtgtccggcaatcaatttagagtttttcggctgaatttgccggacccaaatcaattcgc |
| nucleotides | actgatagacacgagtatttataacccggaacatgagcggttggtttggaggctcgtcggt |
| 2474 to 2912 | attgaaatcgatcgcggtgggcccctgggtatagggagtactggtcaccccctctttaaca |
| HPV-137 L2 | aattgcaagacactgaaaaccccagcgtgtacaacgggctcatctctgatcaaaaggata |
| coding | accgcatgaacgtagctttcgatccgaagcagaaccaactcttcatagtaggctgcaagcc |
| sequence: | agctgtaggccaacattgggataaggctgaaccttgcccgaataccaggccacctcctgg |
| nucleotides | ctcttgcccgccgctgaaactcgtgcactcaactattgaagacggggatatgtctgacattg |
| 2913 to 4442 | ggttgggaaatataaatttttccgacttgtccgatgataagagttccgcccctctcgagatta |
| BGH polyA: | ttaactcaaagtgtaagtggcccgacttcgccctcatgacaaaagatctgttcggagatag |
| nucleotides | cgcctttttctttgggcgacgggagcaactttacgcgcgacaccaatggtgtcgagatggcc |
| 4493 to 4717 | tggtaggggacgctataccagatgagcatttctacttcaaccctaacggacaggaccctaa |
| | gccgccacagtaccagcttggatcctccatatactttactatacctagcggttcccttacatc |
| | tagcgaatctaatatatttggtagaccctactggctgcacagggcccagggcgccaataac |
| | gggatcgcctggggaaatcagctgttcgttacgctccttgataatacgcataacactaactt |
| | caccatctctgtttctactgaaagccaaacgacatatgacaaaaataaatttaaagtgtacc |
| | ttcgacatgctgaggagattgaaattgagatcgtctgtcaactctgcaaagtcccacttgaa |
| | gcggatatattggctcatctttatgctatggacccaagcatactcgacaactggcagctcgc |
| | gtttgtcccagcgcctcctcagacgttggaggacacataccgatacatacgcagtatggca |
| | accatgtgcccggcggacgtgccgccaaaagaacctgaagacccctacaaggatctgca |
| | cttctggactataaacctcacggatagattcacatctgaacttgatcaaaccccgctgggta |
| | agcggttcctgtaccaaatgggattgctgacgggtaataaaagactccgcactgactatat |
| | tggcagtcctgtggctaaacgcaggcgcaccgtgaaaagcagcaaacgcaagaagtcat |
| | ctgcaaagtaattctagtgtacgtagccagcccccgattgggggcgacactccaccataga |
| | tcactcccctgtgaggaactactgtcttcacgcagaaagcgtctagccatggcgttagtatg |
| | agagtcgtgcagcctccaggaccccccctcccgggagagccatagtggtctgcggaaccg |
| | gtgagtacaccggaattgccaggacgaccgggtcctttcttggatcaacccgctcaatgcc |
| | tggagatttgggcgtgcccccgcaagactgctagccgagtagtgttgggtcgcgaaaggc |
| | cttgtggtactgcctgatagggtgcttgcgagtgccccgggaggtctcgtagaccgtgcacc |
| | atgagcacgaatcctaaacctcaaagaaaaaccaaacgtaacaccaaccgccgcccaca |
| | ggacgtcttcatatgtctagccaccatgcaggccaataaacggcgcaaaagagctgcggt |
| | agaagacatttacgctaaaggctgtacccagcctggaggatattgcccaccggatgtgaa |
| | gaataaagtcgagggcaacacttgggcggatttccttttgaaagtttttggaagcgtcgtgt |
| | actttggcgggcttggtattggtacaggcaaaggaaccgggggctccactggttacacccc |
| | cctcggtgggacggttggtagtagggggacaactaataccatcaaacctacgattcctctt |
| | gatccacttggtgtgccggatatcgtcacggtcgatcctatcgcgccggaagcggctagca |
| | ttgttccgttggccgaaggcttgcctgaaccgggagtaatcgacacgggtacttcatttccg |
| | gggcttgcagcggataacgaaaacatagttaccgtgctcgaccctttgagcgaagtcacg |
| | ggcgtaggagagcaccccaacataatcaccggcggcactgccgattcacctgcgattttg |
| | gacgttcagacatcacccccaccggcgaagaaaatactccttgatccatctatttcaaaaa |
| | cgaccaccgcggttcaaactcacgcatcacacgtggatgcaaatttgaacatcttcgtaga |
| | tgctcagagtttcggaacgcatgtgggctacacggaggatatacccctcgaagaaataaa |
| | tctcaggtccgaatttgagttggaggactccgagcccaaaacgtccacgccctttgccgag |
| | cgagtgctcaataaaaccaaacaattgtacagtaagtacgtccagcaggtacctacgaga |
| | cccgcagaatttgcgttgtacacgtctagattcgagtttgaaaatcctgcgtttgaggagga |
| | tgtaacaatggagtttgaaaacgatctggccgaaataggcgaaatcaccactccagcggt |
| | tagtgacgttcgcatacttaatcggccgatttactccgagactgccgaccggacagtaaga |
| | ataagcaggcttgggcagagggccggaatgaagaccagatcagggttggaaattgggca |
| | aagagtacatttttactttgacttgtcagacattccccgcgaatcaattgaacttaacacata |
| | tgggaactattcccacgagtcaacgatagtcgatgaactgcttagctctacttttatcaaccc |
| | gttcgagatgccggtcgacagtgagattttcgcagagaacgaattgcttgacccgctcgaa |
| | gaagattttcgcgactcacatatagtggtcccgtacctcgaagacgaacagatcaatataa |
| | ctccaaccctgcctcctgggctcggattgaaggtatattccgacctctccgaacgggatctc |
| | ctgatacactaccctgtgcaacacgcggacatcatggttccggacactccatacatccccgt |
| | tcagccaccggatggagtattggtagatgataatgactattaccttcatcccggtctctatag |
| | tcggaagagaaaaagaagggtattgtaagcggccgctcgagtctagagggcccgtttaa |
| | acccgctgatcagcctcgactgtgccttctagttgccagccatctgttgtttgcccctcccccg |
| | tgccttccttgaccctggaaggtgccactcccactgtcctttcctaataaaatgaggaaattg |
| | catcgcattgtctgagtaggtgtcattctattctggggggtggggtggggcaggacagcaa |
| | gggggaggattgggaagacaatagcaggcatgctggggatgcggtgggctctatggcttc |
| | tgaggcggaaagaaccagctggggctctagggggtatccccacgcgccctgtagcggcg |
| | cattaagcgcggcgggtgtggtggttacgcgcagcgtgaccgctacacttgccagcgccct |
| | agcgcccgctcctttcgctttcttcccttcctttctcgccacgttcgccggctttccccgtcaag |
| | ctctaaatcgggggctccctttagggttccgatttagtgctttacggcacctcgaccccaaa |
| | aaacttgattagggtgatggttcacgtagtgggccatcgccctgatagacggtttttcgccct |
| | ttgacgttggagtccacgttctttaatagtggactcttgttccaaactggaacaacactcaac |
| | cctatctcggtctattcttttgatttataagggattttgccgatttcggcctattggttaaaaaa |
| | tgagctgatttaacaaaaatttaacgcgaattaattctgtggaatgtgtgtcagttagggtgt |
| | ggaaagtccccaggctccccagcaggcagaagtatgcaaagcatgcatctcaattagtca |
| | gcaaccaggtgtggaaagtccccaggctccccagcaggcagaagtatgcaaagcatgca |
| | tctcaattagtcagcaaccatagtcccgcccctaactccgcccatcccgcccctaactccgc |
| | ccagttccgcccattctccgccccatggctgactaattttttttatttatgcagaggccgaggc |
| | cgcctctgcctctgagctattccagaagtagtgaggaggcttttttggaggcctaggcttttg |
| | caaaaagctcccgggagcttgtatatccattttcggatctgatcaagagacaggatgagga |
| | tcgtttcgcatgattgaacaagatggattgcacgcaggttctccggccgcttgggtggaga |
| | ggctattcggctatgactgggcacaacagacaatcggctgctctgatgccgccgtgttccg |
| | gctgtcagcgcaggggcgcccggttctttttgtcaagaccgacctgtccggtgccctgaatg |
| | aactgcaggacgaggcagcgcggctatcgtggctggccacgacgggcgttccttgcgcag |
| | ctgtgctcgacgttgtcactgaagcgggaagggactggctgctattgggcgaagtgccgg |
| | ggcaggatctcctgtcatctcaccttgctcctgccgagaaagtatccatcatggctgatgca |
| | atgcggcggctgcatacgcttgatccggctacctgcccattcgaccaccaagcgaaacatc |
| | gcatcgagcgagcacgtactcggatggaagccggtcttgtcgatcaggatgatctggacg |
| | aagagcatcaggggctcgcgccagccgaactgttcgccaggctcaaggcgcgcatgccc |
| | gacggcgaggatctcgtcgtgacccatggcgatgcctgcttgccgaatatcatggtggaaa |
| | atggccgcttttctggattcatcgactgtggccggctgggtgtggcggaccgctatcaggac |
| | atagcgttggctacccgtgatattgctgaagagcttggcggcgaatgggctgaccgcttcct |
| | cgtgctttacggtatcgccgctcccgattcgcagcgcatcgccttctatcgccttcttgacga |
| | gttcttctgagcgggactctggggttcgaaatgaccgaccaagcgacgcccaacctgccat |
| | cacgagatttcgattccaccgccgccttctatgaaaggttgggcttcggaatcgttttccggg |
| | acgccggctggatgatcctccagcgcggggatctcatgctggagttcttcgcccaccccaa |
| | cttgtttattgcagcttataatggttacaaataaagcaatagcatcacaaatttcacaaata |
| | aagcatttttttcactgcattctagttgtggtttgtccaaactcatcaatgtatcttatcatgtct |
| | gtataccgtcgacctctagctagagcttggcgtaatcatggtcatagctgtttcctgtgtgaa |
| | attgttatccgctcacaattccacacaacatacgagccggaagcataaagtgtaaagcctg |
| | gggtgcctaatgagtgagctaactcacattaattgcgttgcgctcactgcccgctttccagtc |
| | gggaaacctgtcgtgccagctgcattaatgaatcggccaacgcgcggggagaggcggttt |
| | gcgtattgggcgctcttccgcttcctcgctcactgactcgctgcgctcggtcgttcggctgcg |
| | gcgagcggtatcagctcactcaaaggcggtaatacggttatccacagaatcaggggataa |
| | cgcaggaaagaacatgtgagcaaaaggccagcaaaaggccaggaaccgtaaaaaggc |
| | cgcgttgctggcgtttttccataggctccgcccccctgacgagcatcacaaaaatcgacgct |
| | caagtcagaggtggcgaaacccgacaggactataaagataccaggcgtttccccctgga |
| | agctccctcgtgcgctctcctgttccgaccctgccgcttaccggatacctgtccgcctttctcc |
| | cttcgggaagcgtggcgctttctcatagctcacgctgtaggtatctcagttcggtgtaggtcg |
| | ttcgctccaagctgggctgtgtgcacgaaccccccgttcagcccgaccgctgcgccttatcc |
| | ggtaactatcgtcttgagtccaacccggtaagacacgacttatcgccactggcagcagcca |
| | ctggtaacaggattagcagagcgaggtatgtaggcggtgctacagagttcttgaagtggtg |
| | gcctaactacggctacactagaagaacagtatttggtatctgcgctctgctgaagccagtta |
| | ccttcggaaaaagagttggtagctcttgatccggcaaacaaaccaccgctggtagcggtg |
| | gtttttttgtttgcaagcagcagattacgcgcagaaaaaaaggatctcaagaagatcctttg |
| | atcttttctacggggtctgacgctcagtggaacgaaaactcacgttaagggattttggtcat |
| | gagattatcaaaaaggatcttcacctagatccttttaaattaaaaatgaagttttaaatcaat |
| | ctaaagtatatatgagtaaacttggtctgacagttaccaatgcttaatcagtgaggcaccta |
| | tctcagcgatctgtctatttcgttcatccatagttgcctgactccccgtcgtgtagataactac |
| | gatacgggagggcttaccatctggccccagtgctgcaatgataccgcgagacccacgctc |
| | accggctccagatttatcagcaataaaccagccagccggaagggccgagcgcagaagtg |
| | gtcctgcaactttatccgcctccatccagtctattaattgttgccgggaagctagagtaagta |
| | gttcgccagttaatagtttgcgcaacgttgttgccattgctacaggcatcgtggtgtcacgct |
| | cgtcgtttggtatggcttcattcagctccggttcccaacgatcaaggcgagttacatgatccc |
| | ccatgttgtgcaaaaaagcggttagctccttcggtcctccgatcgttgtcagaagtaagttg |
| | gccgcagtgttatcactcatggttatggcagcactgcataattctcttactgtcatgccatcc |
| | gtaagatgcttttctgtgactggtgagtactcaaccaagtcattctgagaatagtgtatgcg |
| | gcgaccgagttgctcttgcccggcgtcaatacgggataataccgcgccacatagcagaac |
| | tttaaaagtgctcatcattggaaaacgttcttcggggcgaaaactctcaaggatcttaccgc |
| | tgttgagatccagttcgatgtaacccactcgtgcacccaactgatcttcagcatcttttacttt |
| | caccagcgtttctgggtgagcaaaaacaggaaggcaaaatgccgcaaaaaagggaata |
| | agggcgacacggaaatgttgaatactcatactcttcctttttcaatattattgaagcatttatc |
| | agggttattgtctcatgagcggatacatatttgaatgtatttagaaaaataaacaaataggg |
| | gttccgcgcacatttccccgaaaagtgccacctgacgtc (SEQ ID NO: 28) |
| HPV-137 L1 | MAVWVPNKGRLYLPPQRPVAKVLSTDDYIVGTDLYFHSS |
| amino acid | TDRLLTVGHPFFDVLSTDQNTVDVPKVSGNQFRVFRLNL |
| | PDPNQFALIDTSIYNPEHERLVWRLVGIEIDRGGPLGIGST |
| | GHPLFNKLQDTENPSVYNGLISDQKDNRMNVAFDPKQNQ |
| | LFIVGCKPAVGQHWDKAEPCPNTRPPPGSCPPLKLVHSTI |
| | EDGDMSDIGLGNINFSDLSDDKSSAPLEIINSKCKWPDFAL |
| | MTKDLFGDSAFFFGRREQLYARHQWCRDGLVGDAIPDE |
| | HFYFNPNGQDPKPPQYQLGSSIYFTIPSGSLTSSESNIFGRP |
| | YWLHRAQGANNGIAWGNQLFVTLLDNTHNTNFTISVSTE |
| | SQTTYDKNKFKVYLRHAEEIEIEIVCQLCKVPLEADILAH |
| | LYAMDPSILDNWQLAFVPAPPQTLEDTYRYIRSMATMCP |
| | ADVPPKEPEDPYKDLHFWTINLTDRFTSELDQTPLGKRFL |
| | YQMGLLTGNKRLRTDYIGSPVAKRRRTVKSSKRKKSSAK |
| | (SEQ ID NO: 29) |
| HPV-137 L2 | MQANKRRKRAAVEDIYAKGCTQPGGYCPPDVKNKVEGN |
| amino acid | TWADFLLKVFGSVVYFGGLGIGTGKGTGGSTGYTPLGGT |
| | VGSRGTTNTIKPTIPLDPLGVPDIVTVDPIAPEAASIVPLAE |
| | GLPEPGVIDTGTSFPGLAADNENIVTVLDPLSEVTGVGEH |
| | PNIITGGTADSPAILDVQTSPPPAKKILLDPSISKTTTAVQT |
| | HASHVDANLNIFVDAQSFGTHVGYTEDIPLEEINLRSEFEL |
| | EDSEPKTSTPFAERVLNKTKQLYSKYVQQVPTRPAEFALY |
| | TSRFEFENPAFEEDVTMEFENDLAEIGEITTPAVSDVRILN |
| | RPIYSETADRTVRISRLGQRAGMKTRSGLEIGQRVHFYFD |
| | LSDIPRESIELNTYGNYSHESTIVDELLSSTFINPFEMPVDS |
| | EIFAENELLDPLEEDFRDSHIVVPYLEDEQINITPTLPPGLG |
| | LKVYSDLSERDLLIHYPVQHADIMVPDTPYIPVQPPDGVL |
| | VDDNDYYLHPGLYSRKRKRRVL (SEQ ID NO: 30) |
| pDY0039HPV41 | gacggatcgggagatctcccgatcccctatggtgcactctcagtacaatctgctctgatgcc |
| L1-HCV | gcatagttaagccagtatctgctccctgcttgtgtgttggaggtcgctgagtagtgcgcgag |
| IRES-L2 | caaaatttaagctacaacaaggcaaggcttgaccgacaattgcatgaagaatctgcttag |
| (seq | ggttaggcgttttgcgctgcttcgcgatgtacgggccagatatacgcgttgacattgattatt |
| Qd1R5EPu) | gactagttattaatagtaatcaattacggggtcattagttcatagcccatatatggagttccg |
| CMV | cgttacataacttacggtaaatggcccgcctggctgaccgcccaacgacccccgcccattg |
| promoter: | acgtcaataatgacgtatgttcccatagtaacgccaatagggactttccattgacgtcaatg |
| nucleotides | ggtggagtatttacggtaaactgcccacttggcagtacatcaagtgtatcatatgccaagta |
| 232 to 819 | cgccccctattgacgtcaatgacggtaaatggcccgcctggcattatgcccagtacatgac |
| T7 promoter: | cttatgggactttcctacttggcagtacatctacgtattagtcatcgctattaccatggtgatg |
| nucleotides | cggttttggcagtacatcaatgggcgtggatagcggtttgactcacggggatttccaagtct |
| 863 to 879 | ccaccccattgacgtcaatgggagtttgttttggcaccaaaatcaacgggactttccaaaat |
| HPV-41 L1 | gtcgtaacaactccgccccattgacgcaaatgggcggtaggcgtgtacggtgggaggtct |
| coding | atataagcagagctctctggctaactagagaacccactgcttactggcttatcgaaattaat |
| sequence: | acgactcactatagggagacccaagctggctagcgtttaaacttaagcttgccaccatgac |
| nucleotides | cggtctgcaatacctctttcttgctatgatggctctcaccctttccatactgttggcccaacaa |
| 923 to 2674 | ccgccccctcatagctgtctccacagtcccgccatgtgcccgacgcttttgcttacttgtatcg |
| IRES: | ttgaggtgtggataatgatctatatccttgcctgctgcgccggcaacgttaagaatgcaaat |
| nucleotides | gtttttatctttcaaatggctgtatggttgccaggcccaaaccgattctacctccctccccaac |
| 2675 to 3113 | cgatccaacgcaccttgaatactgaagaatatgtgagaagaacaagtacgttcctccatgc |
| HPV-41 L2 | ggctacagaccgacttcttacagtcggacaccctttttacaatattacaaatgctgacggga |
| coding | aggaagtagttccgaaggtctcctctaaccaatttagggcatttcgagttcgcttcccgaac |
| sequence: | cccaatacttttgcattttgcgataagagtctttttaacccagataaagaaagactcgtttgg |
| nucleotides | ggtataagaggaatcgaagtgtcacgcggccagccactcggcatcggcgtgacagggaa |
| 3114 to 4778 | tccattttttaacaaattcgacgacgctgaaaatccgtacaacggaattaataagaacaac |
| BGH polyA: | atcaccgatcaagggtctgattctaggctctctatagcgtttgacccgaagcaaacacagtt |
| nucleotides | gctgattgtaggagccaagccggcgaaaggggaatattgggatgtcgccgcaacatgtga |
| 4829 to 5053 | gaatccaccgctgacgaaggcagacgacaagtgtcccgccctcgagttgaaatcttcttac |
| | atcgaagatgcagatatgtccgacatcgggttggggaatctgaacttctctactttgcagcg |
| | caataagtccgacgcgccgctggacattgtcgacagtatttgcaaatatcctgactatttgc |
| | agatgatagaagaactgtacggcgatcacatgtttttctacgtgcggcgggaggcgcttta |
| | cgcgcggcacattatgcagcatgctggaaagatggatgcagagcaatttccaacctctctt |
| | tacattgactcttccgttgaaggtgagaaacttaatagtctccaacggacagataggtattt |
| | catgactccctcaggctcactggtcgcgacggagcagcagctgttcaaccgacccttttgg |
| | cttcaacgaagccaaggtcacaataacggcatactttggcataacgaagcctttgtcaccc |
| | ttgttgatactactagaggtacaaacttcactatatctgtccctgaaggtgacgcctcctcat |
| | acaacaatagtaaatttttcgaatttcttagacatacggaagagttccagttggcatttatac |
| | ttcaactctgcaaggttgacttgacccccgaaaatctcgcatacatacataccatggaccca |
| | tctattattgaagattggcacctcgcagtcacttccccgcctaactccgtactggaggacca |
| | ctatcgatatatcctcagtatagcaacaaaatgtcctagcaaggacgcggacgatacgag |
| | cacagacccatataaagatctcaagttttgggaagttgacctccgagatcgaatgaccgaa |
| | cagcttgaccaaactccgcttggcagaaagtttctcttccagacgggaatcactcagagttc |
| | tagtaacaagcgggtctccactcaatcaaccgcattgaccacgtatcgacgccccactaaa |
| | aggcgaaggaaggcataattctagtgtacgtagccagcccccgattgggggcgacactcc |
| | accatagatcactcccctgtgaggaactactgtcttcacgcagaaagcgtctagccatggc |
| | gttagtatgagagtcgtgcagcctccaggaccccccctcccgggagagccatagtggtctg |
| | cggaaccggtgagtacaccggaattgccaggacgaccgggtcctttcttggatcaacccg |
| | ctcaatgcctggagatttgggcgtgcccccgcaagactgctagccgagtagtgttgggtcg |
| | cgaaaggccttgtggtactgcctgatagggtgcttgcgagtgccccgggaggtctcgtaga |
| | ccgtgcaccatgagcacgaatcctaaacctcaaagaaaaaccaaacgtaacaccaaccg |
| | ccgcccacaggacgtcttcatatgtctagccaccatgctggctaggcaaagggtgaagcg |
| | ggctaacccggagcagttgtataagacatgcaaagccacgggtggggattgtcctcccga |
| | tgtaataaagcggtacgaacagacaacgccggccgacagtattttgaagtacgggagtgt |
| | aggtgtcttctttggtggcctcggcattgggaccggtagaggaggtgggggcacagtcctt |
| | ggagccggggcagtgggaggcaggccttcaattagctcaggagcgattgggccacggga |
| | catcctgccgatcgaatccggagggccgagcctggcggaggagattccgcttttgcctatg |
| | gcgccccgagtacccagacccactgatcctttcaggccatccgtcctcgaggagccctttat |
| | aatacggcctccagaacgcccaaatatcttgcatgagcaaaggttccccacggacgctgc |
| | cccatttgacaatgggaacaccgaaatcacaacaattccatcacagtatgatgtctctgga |
| | gggggtgttgatatccagataatcgagctgccatccgttaatgacccaggccctagcgtcg |
| | ttacgcgcactcagtacaataaccccacatttgaggttgaagtcagtacagatatatctgga |
| | gaaaccagtagtaccgataatattattgttggcgctgagtcagggggtacgtcagtaggag |
| | acaatgcggaactgataccattgctcgacatttctcggggtgatactatagataccacaatc |
| | cttgcaccgggagaggaagagactgcgtttgtaacgagcacccccgagagggttcctatc |
| | caggagagactgccaataagaccgtacggcagacaataccagcaggtgagagtcacgg |
| | accctgaattcttggattcagctgcggttctcgttagccttgagaatccggtttttgatgctga |
| | cattactcttactttcgaggatgatcttcagcaagcactgcgatccgatacagaccttaggg |
| | acgtgcggcggcttagtaggccttattatcagcgccgcacgaccggactcagagtttcccg |
| | cctcggtcagcgaagggggacaattagtaccaggtcaggtgtgcaggtgggatctgctgc |
| | ccacttcttccaagacatctccccgatcggacaggcgatagaaccgattgacgcaattgag |
| | ctggatgttttgggcgagcaatctggtgagggcactatcgtgcggggagatccaacgcctt |
| | ccattgaacaagatattggcctcacagcacttggtgacaacatcgagaacgaattgcaag |
| | agatagatcttctcacggcagacggcgaagaagatcaagagggtcgggacctgcaattg |
| | gtgttctccaccggaaacgatgaggtggtggatatcatgacgataccaattcgagccggtg |
| | gtgatgaccgccccagcgtatttatcttcagcgacgatggcacgcacattgtttaccccaca |
| | tctacaacggcaactacgccgctcgtcccggctcaaccgagtgatgtaccatacattgtcgt |
| | agatttgtactcaggcagtatggattacgacattcacccatccctgctccgaaggaagcga |
| | aagaaacggaaaagggtatacttctccgatggacgagttgcatcacgcccgaagtaggc |
| | ggccgctcgagtctagagggcccgtttaaacccgctgatcagcctcgactgtgccttctagt |
| | tgccagccatctgttgtttgcccctcccccgtgccttccttgaccctggaaggtgccactccc |
| | actgtcctttcctaataaaatgaggaaattgcatcgcattgtctgagtaggtgtcattctattc |
| | tggggggtggggtggggcaggacagcaagggggaggattgggaagacaatagcaggc |
| | atgctggggatgcggtgggctctatggcttctgaggcggaaagaaccagctggggctcta |
| | gggggtatccccacgcgccctgtagcggcgcattaagcgcggcgggtgtggtggttacgc |
| | gcagcgtgaccgctacacttgccagcgccctagcgcccgctcctttcgctttcttcccttcctt |
| | tctcgccacgttcgccggctttccccgtcaagctctaaatcgggggctccctttagggttccg |
| | atttagtgctttacggcacctcgaccccaaaaaacttgattagggtgatggttcacgtagtg |
| | ggccatcgccctgatagacggtttttcgccctttgacgttggagtccacgttctttaatagtg |
| | gactcttgttccaaactggaacaacactcaaccctatctcggtctattcttttgatttataagg |
| | gattttgccgatttcggcctattggttaaaaaatgagctgatttaacaaaaatttaacgcga |
| | attaattctgtggaatgtgtgtcagttagggtgtggaaagtccccaggctccccagcaggc |
| | agaagtatgcaaagcatgcatctcaattagtcagcaaccaggtgtggaaagtccccaggc |
| | tccccagcaggcagaagtatgcaaagcatgcatctcaattagtcagcaaccatagtcccg |
| | cccctaactccgcccatcccgcccctaactccgcccagttccgcccattctccgccccatgg |
| | ctgactaattttttttatttatgcagaggccgaggccgcctctgcctctgagctattccagaa |
| | gtagtgaggaggcttttttggaggcctaggcttttgcaaaaagctcccgggagcttgtatat |
| | ccattttcggatctgatcaagagacaggatgaggatcgtttcgcatgattgaacaagatgg |
| | attgcacgcaggttctccggccgcttgggtggagaggctattcggctatgactgggcacaa |
| | cagacaatcggctgctctgatgccgccgtgttccggctgtcagcgcaggggcgcccggttc |
| | tttttgtcaagaccgacctgtccggtgccctgaatgaactgcaggacgaggcagcgcggct |
| | atcgtggctggccacgacgggcgttccttgcgcagctgtgctcgacgttgtcactgaagcg |
| | ggaagggactggctgctattgggcgaagtgccggggcaggatctcctgtcatctcaccttg |
| | ctcctgccgagaaagtatccatcatggctgatgcaatgcggcggctgcatacgcttgatcc |
| | ggctacctgcccattcgaccaccaagcgaaacatcgcatcgagcgagcacgtactcggat |
| | ggaagccggtcttgtcgatcaggatgatctggacgaagagcatcaggggctcgcgccagc |
| | cgaactgttcgccaggctcaaggcgcgcatgcccgacggcgaggatctcgtcgtgaccca |
| | tggcgatgcctgcttgccgaatatcatggtggaaaatggccgcttttctggattcatcgactg |
| | tggccggctgggtgtggcggaccgctatcaggacatagcgttggctacccgtgatattgct |
| | gaagagcttggcggcgaatgggctgaccgcttcctcgtgctttacggtatcgccgctcccg |
| | attcgcagcgcatcgccttctatcgccttcttgacgagttcttctgagcgggactctggggtt |
| | cgaaatgaccgaccaagcgacgcccaacctgccatcacgagatttcgattccaccgccgc |
| | cttctatgaaaggttgggcttcggaatcgttttccgggacgccggctggatgatcctccagc |
| | gcggggatctcatgctggagttcttcgcccaccccaacttgtttattgcagcttataatggtt |
| | acaaataaagcaatagcatcacaaatttcacaaataaagcatttttttcactgcattctagtt |
| | gtggtttgtccaaactcatcaatgtatcttatcatgtctgtataccgtcgacctctagctagag |
| | cttggcgtaatcatggtcatagctgtttcctgtgtgaaattgttatccgctcacaattccacac |
| | aacatacgagccggaagcataaagtgtaaagcctggggtgcctaatgagtgagctaactc |
| | acattaattgcgttgcgctcactgcccgctttccagtcgggaaacctgtcgtgccagctgcat |
| | taatgaatcggccaacgcgcggggagaggcggtttgcgtattgggcgctcttccgcttcctc |
| | gctcactgactcgctgcgctcggtcgttcggctgcggcgagcggtatcagctcactcaaag |
| | gcggtaatacggttatccacagaatcaggggataacgcaggaaagaacatgtgagcaaa |
| | aggccagcaaaaggccaggaaccgtaaaaaggccgcgttgctggcgtttttccataggct |
| | ccgcccccctgacgagcatcacaaaaatcgacgctcaagtcagaggtggcgaaacccga |
| | caggactataaagataccaggcgtttccccctggaagctccctcgtgcgctctcctgttccg |
| | accctgccgcttaccggatacctgtccgcctttctcccttcgggaagcgtggcgctttctcat |
| | agctcacgctgtaggtatctcagttcggtgtaggtcgttcgctccaagctgggctgtgtgca |
| | cgaaccccccgttcagcccgaccgctgcgccttatccggtaactatcgtcttgagtccaacc |
| | cggtaagacacgacttatcgccactggcagcagccactggtaacaggattagcagagcg |
| | aggtatgtaggcggtgctacagagttcttgaagtggtggcctaactacggctacactagaa |
| | gaacagtatttggtatctgcgctctgctgaagccagttaccttcggaaaaagagttggtagc |
| | tcttgatccggcaaacaaaccaccgctggtagcggtggtttttttgtttgcaagcagcagatt |
| | acgcgcagaaaaaaaggatctcaagaagatcctttgatcttttctacggggtctgacgctc |
| | agtggaacgaaaactcacgttaagggattttggtcatgagattatcaaaaaggatcttcac |
| | ctagatccttttaaattaaaaatgaagttttaaatcaatctaaagtatatatgagtaaacttg |
| | gtctgacagttaccaatgcttaatcagtgaggcacctatctcagcgatctgtctatttcgttc |
| | atccatagttgcctgactccccgtcgtgtagataactacgatacgggagggcttaccatctg |
| | gccccagtgctgcaatgataccgcgagacccacgctcaccggctccagatttatcagcaat |
| | aaaccagccagccggaagggccgagcgcagaagtggtcctgcaactttatccgcctccat |
| | ccagtctattaattgttgccgggaagctagagtaagtagttcgccagttaatagtttgcgca |
| | acgttgttgccattgctacaggcatcgtggtgtcacgctcgtcgtttggtatggcttcattcag |
| | ctccggttcccaacgatcaaggcgagttacatgatcccccatgttgtgcaaaaaagcggtt |
| | agctccttcggtcctccgatcgttgtcagaagtaagttggccgcagtgttatcactcatggtt |
| | atggcagcactgcataattctcttactgtcatgccatccgtaagatgcttttctgtgactggtg |
| | agtactcaaccaagtcattctgagaatagtgtatgcggcgaccgagttgctcttgcccggc |
| | gtcaatacgggataataccgcgccacatagcagaactttaaaagtgctcatcattggaaa |
| | acgttcttcggggcgaaaactctcaaggatcttaccgctgttgagatccagttcgatgtaac |
| | ccactcgtgcacccaactgatcttcagcatcttttactttcaccagcgtttctgggtgagcaa |
| | aaacaggaaggcaaaatgccgcaaaaaagggaataagggcgacacggaaatgttgaat |
| | actcatactcttcctttttcaatattattgaagcatttatcagggttattgtctcatgagcggat |
| | acatatttgaatgtatttagaaaaataaacaaataggggttccgcgcacatttccccgaaa |
| | agtgccacctgacgtc (SEQ ID NO: 31) |
| HPV-41 L1 | MTGLQYLFLAMMALTLSILLAQQPPPHSCLHSPAMCPTL |
| amino acid | LLTCIVEVWIMIYILACCAGNVKNANVFIFQMAVWLPGP |
| | NRFYLPPQPIQRTLNTEEYVRRTSTFLHAATDRLLTVGHP |
| | FYNITNADGKEVVPKVSSNQFRAFRVRFPNPNTFAFCDKS |
| | LFNPDKERLVWGIRGIEVSRGQPLGIGVTGNPFFNKFDDA |
| | ENPYNGINKNNITDQGSDSRLSIAFDPKQTQLLIVGAKPAK |
| | GEYWDVAATCENPPLTKADDKCPALELKSSYIEDADMSD |
| | IGLGNLNFSTLQRNKSDAPLDIVDSICKYPDYLQMIEELYG |
| | DHMFFYVRREALYARHIMQHAGKMDAEQFPTSLYIDSSV |
| | EGEKLNSLQRTDRYFMTPSGSLVATEQQLFNRPFWLQRS |
| | QGHNNGILWHNEAFVTLVDTTRGTNFTISVPEGDASSYNN |
| | SKFFEFLRHTEEFQLAFILQLCKVDLTPENLAYIHTMDPSI |
| | IEDWHLAVTSPPNSVLEDHYRYILSIATKCPSKDADDTSTD |
| | PYKDLKFWEVDLRDRMTEQLDQTPLGRKFLFQTGITQSS |
| | SNKRVSTQSTALTTYRRPTKRRRKA (SEQ ID NO: 32) |
| HPV-41 L2 | MLARQRVKRANPEQLYKTCKATGGDCPPDVIKRYEQTT |
| amino acid | PADSILKYGSVGVFFGGLGIGTGRGGGGTVLGAGAVGGR |
| | PSISSGAIGPRDILPIESGGPSLAEEIPLLPMAPRVPRPTDPF |
| | RPSVLEEPFIIRPPERPNILHEQRFPTDAAPFDNGNTEITTIP |
| | SQYDVSGGGVDIQIIELPSVNDPGPSVVTRTQYNNPTFEVE |
| | VSTDISGETSSTDNIIVGAESGGTSVGDNAELIPLLDISRGD |
| | TIDTTILAPGEEETAFVTSTPERVPIQERLPIRPYGRQYQQ |
| | VRVTDPEFLDSAAVLVSLENPVFDADITLTFEDDLQQALR |
| | SDTDLRDVRRLSRPYYQRRTTGLRVSRLGQRRGTISTRSG |
| | VQVGSAAHFFQDISPIGQAIEPIDAIELDVLGEQSGEGTIVR |
| | GDPTPSIEQDIGLTALGDNIENELQEIDLLTADGEEDQEGR |
| | DLQLVFSTGNDEVVDIMTIPIRAGGDDRPSVFIFSDDGTHI |
| | VYPTSTTATTPLVPAQPSDVPYIVVDLYSGSMDYDIHPSLL |
| | RRKRKKRKRVYFSDGRVASRPK (SEQ ID NO: 33) |
| pDY0040HPV18 | gacggatcgggagatctcccgatcccctatggtgcactctcagtacaatctgctctgatgcc |
| L1-HCV | gcatagttaagccagtatctgctccctgcttgtgtgttggaggtcgctgagtagtgcgcgag |
| IRES-L2 | caaaatttaagctacaacaaggcaaggcttgaccgacaattgcatgaagaatctgcttag |
| (seq | ggttaggcgttttgcgctgcttcgcgatgtacgggccagatatacgcgttgacattgattatt |
| 7nckqLaW) | gactagttattaatagtaatcaattacggggtcattagttcatagcccatatatggagttccg |
| CMV | cgttacataacttacggtaaatggcccgcctggctgaccgcccaacgacccccgcccattg |
| promoter: | acgtcaataatgacgtatgttcccatagtaacgccaatagggactttccattgacgtcaatg |
| nucleotides | ggtggagtatttacggtaaactgcccacttggcagtacatcaagtgtatcatatgccaagta |
| 232 to 819 | cgccccctattgacgtcaatgacggtaaatggcccgcctggcattatgcccagtacatgac |
| T7 promoter: | cttatgggactttcctacttggcagtacatctacgtattagtcatcgctattaccatggtgatg |
| nucleotides | cggttttggcagtacatcaatgggcgtggatagcggtttgactcacggggatttccaagtct |
| 863 to 879 | ccaccccattgacgtcaatgggagtttgttttggcaccaaaatcaacgggactttccaaaat |
| HPV-18 L1 | gtcgtaacaactccgccccattgacgcaaatgggcggtaggcgtgtacggtgggaggtct |
| coding | atataagcagagctctctggctaactagagaacccactgcttactggcttatcgaaattaat |
| sequence: | acgactcactatagggagacccaagctggctagcgtttaaacttaagcttgccaccatggc |
| nucleotides | gctgtggagaccctccgacaataccgtttatctccctccaccgtcagttgctcgggttgtaaa |
| 923 to 2446 | tactgacgattacgtcacacgaaccagcattttttaccacgctgggagttcacggctcctca |
| IRES: | cggtgggaaacccctattttcgagtccccgccggaggcggtaacaagcaggatatcccga |
| nucleotides | aagtgtctgcctatcagtaccgggtgtttcgagtacagctccccgacccgaataagtttggg |
| 2447 to 2885 | cttccagatacatccatctacaatcctgaaacgcaacggcttgtatgggcctgtgcgggcgt |
| HPV-18 L2 | ggaaataggaagaggccaaccgctgggagttggactgagcggtcacccattttacaaca |
| coding | aattggatgatacggagagttcacacgcggcaacctcaaatgtttccgaagacgtcaggg |
| sequence: | acaatgtatcagtggattacaagcaaacacaactctgcattctgggatgtgcgcctgcaat |
| nucleotides | cggtgaacactgggctaaaggaacagcttgtaagtctcgaccactcagtcagggtgactgt |
| 2886 to 4274 | ccaccacttgaactcaaaaatactgtgctcgaggatggggacatggtggataccgggtat |
| BGH polyA: | ggtgcgatggatttttcaacactgcaagatactaagtgcgaagttccccttgacatttgtca |
| nucleotides | aagtatctgcaaatacccggattacctccagatgagcgctgacccgtacggtgactcaatg |
| 4325 to 4549 | tttttttgtcttcgacgcgaacaactcttcgcccgccacttctggaatcgggctggaacgatg |
| | ggtgataccgttccccaatcattgtatataaagggtacaggtatgcgcgcttcaccaggctc |
| | ctgtgtgtactctccgtccccctccggttctatagtaactagtgactctcagcttttcaacaaa |
| | ccatactggcttcataaggcgcaaggccataataatggagtctgctggcacaaccagttgt |
| | tcgtgacagttgtggatacgacgagaagtacgaaccttactatctgtgcatcaacacagtc |
| | ccctgttccgggccaatacgatgcaactaagtttaaacaatactctcgacacgtagaagag |
| | tatgatctgcaattcatatttcagttgtgcacaataacactgacggcagatgtcatgtcatac |
| | atccactcaatgaattccagcattctggaggattggaatttcggggtcccgccgcccccaac |
| | cacctctcttgtagatacataccgattcgtacaaagcgtggcaatcacatgtcaaaaagat |
| | gcggcaccagcagaaaataaagacccctatgacaaactgaagttctggaatgtggacctt |
| | aaagaaaaatttagcttggaccttgaccaataccctttgggtaggaaatttctcgtgcaagc |
| | aggcttgcgccggaaaccgaccattggaccacgcaagcgcagtgcgccgagcgcaacca |
| | caagtagtaagcctgcgaagagggttcgcgtgcgcgccagaaagtaattctagtgtacgt |
| | agccagcccccgattgggggcgacactccaccatagatcactcccctgtgaggaactact |
| | gtcttcacgcagaaagcgtctagccatggcgttagtatgagagtcgtgcagcctccaggac |
| | cccccctcccgggagagccatagtggtctgcggaaccggtgagtacaccggaattgccag |
| | gacgaccgggtcctttcttggatcaacccgctcaatgcctggagatttgggcgtgcccccgc |
| | aagactgctagccgagtagtgttgggtcgcgaaaggccttgtggtactgcctgatagggtg |
| | cttgcgagtgccccgggaggtctcgtagaccgtgcaccatgagcacgaatcctaaacctca |
| | aagaaaaaccaaacgtaacaccaaccgccgcccacaggacgtcttcatatgtctagccac |
| | catggtgagccatcgagcggccagacgcaaaagggcgagcgtaaccgacttgtataaaa |
| | cttgcaaacaatcagggacttgtccaccggacgtggtccccaaggtggaaggcaccacac |
| | tcgccgataagatactccaatggtccagccttggtatatttcttggtggcctggggatcgga |
| | accggatctggaactggtgggcgaacgggctacattccactggggggaagaagcaacac |
| | cgttgtcgatgtaggacctacgagacctccggtagttatagagcccgttggacccaccgat |
| | ccgagcattgtaacgttgatcgaggactctagcgtggtcacctcaggtgcaccacgaccta |
| | cctttacaggcacatctggatttgacataaccagcgccgggaccactactccagcggtact |
| | ggacataacgccaagttccacgtccgtgagcatttccactactaactttacaaatcctgcctt |
| | ttctgaccctagcataatagaggtgccccaaacgggtgaggttgcggggaacgtcttcgtt |
| | ggcacgccgacttcaggaacccatggttacgaggaaatacctcttcagacatttgcgtcat |
| | caggcacgggcgaagagccaatatctagcacgcccctgcctactgttcgccgagtcgcag |
| | ggcctaggctttattccagggcatatcaacaggtatctgttgccaatccggaatttctcacg |
| | agaccctcatcccttattacatatgacaatccagccttcgaacccgtagacacaactctgac |
| | gtttgaccccagatcagatgtcccagatagtgacttcatggatattatacggcttcatcgac |
| | cggcacttactagtagacgcggtaccgttaggttcagccgactgggccaaagggccacga |
| | tgttcacacgctctggcactcagataggcgctagggtacacttctaccacgatatctctccg |
| | attgcaccctctcccgaatatattgagctgcagccacttgtgtcagccaccgaggataatga |
| | cctgttcgacatctacgccgatgatatggacccggcagtgcccgttcctagccggagcact |
| | acctcctttgccttttttaagtacagccccactattagttctgcttctagttatagtaatgtaac |
| | tgttcccctcacctcaagttgggatgtgccagtttataccggtcccgacattacccttccatc |
| | aacgacttctgtatggccgatcgtttctccaacagcaccagcgagtacgcaatacatcggc |
| | atccatggtacgcactactatctctggcccttgtattactttataccaaaaaagagaaagcg |
| | agtcccatacttcttcgcagacggcttcgttgcggcgtaggcggccgctcgagtctagagg |
| | gcccgtttaaacccgctgatcagcctcgactgtgccttctagttgccagccatctgttgtttgc |
| | ccctcccccgtgccttccttgaccctggaaggtgccactcccactgtcctttcctaataaaat |
| | gaggaaattgcatcgcattgtctgagtaggtgtcattctattctggggggtggggtggggca |
| | ggacagcaagggggaggattgggaagacaatagcaggcatgctggggatgcggtgggc |
| | tctatggcttctgaggcggaaagaaccagctggggctctagggggtatccccacgcgccct |
| | gtagcggcgcattaagcgcggcgggtgtggtggttacgcgcagcgtgaccgctacacttg |
| | ccagcgccctagcgcccgctcctttcgctttcttcccttcctttctcgccacgttcgccggcttt |
| | ccccgtcaagctctaaatcgggggctccctttagggttccgatttagtgctttacggcacctc |
| | gaccccaaaaaacttgattagggtgatggttcacgtagtgggccatcgccctgatagacgg |
| | tttttcgccctttgacgttggagtccacgttctttaatagtggactcttgttccaaactggaac |
| | aacactcaaccctatctcggtctattcttttgatttataagggattttgccgatttcggcctatt |
| | ggttaaaaaatgagctgatttaacaaaaatttaacgcgaattaattctgtggaatgtgtgtc |
| | agttagggtgtggaaagtccccaggctccccagcaggcagaagtatgcaaagcatgcatc |
| | tcaattagtcagcaaccaggtgtggaaagtccccaggctccccagcaggcagaagtatgc |
| | aaagcatgcatctcaattagtcagcaaccatagtcccgcccctaactccgcccatcccgcc |
| | cctaactccgcccagttccgcccattctccgccccatggctgactaattttttttatttatgcag |
| | aggccgaggccgcctctgcctctgagctattccagaagtagtgaggaggcttttttggagg |
| | cctaggcttttgcaaaaagctcccgggagcttgtatatccattttcggatctgatcaagaga |
| | caggatgaggatcgtttcgcatgattgaacaagatggattgcacgcaggttctccggccgc |
| | ttgggtggagaggctattcggctatgactgggcacaacagacaatcggctgctctgatgcc |
| | gccgtgttccggctgtcagcgcaggggcgcccggttctttttgtcaagaccgacctgtccgg |
| | tgccctgaatgaactgcaggacgaggcagcgcggctatcgtggctggccacgacgggcgt |
| | tccttgcgcagctgtgctcgacgttgtcactgaagcgggaagggactggctgctattgggc |
| | gaagtgccggggcaggatctcctgtcatctcaccttgctcctgccgagaaagtatccatcat |
| | ggctgatgcaatgcggcggctgcatacgcttgatccggctacctgcccattcgaccaccaa |
| | gcgaaacatcgcatcgagcgagcacgtactcggatggaagccggtcttgtcgatcaggat |
| | gatctggacgaagagcatcaggggctcgcgccagccgaactgttcgccaggctcaaggc |
| | gcgcatgcccgacggcgaggatctcgtcgtgacccatggcgatgcctgcttgccgaatatc |
| | atggtggaaaatggccgcttttctggattcatcgactgtggccggctgggtgtggcggacc |
| | gctatcaggacatagcgttggctacccgtgatattgctgaagagcttggcggcgaatgggc |
| | tgaccgcttcctcgtgctttacggtatcgccgctcccgattcgcagcgcatcgccttctatcg |
| | ccttcttgacgagttcttctgagcgggactctggggttcgaaatgaccgaccaagcgacgc |
| | ccaacctgccatcacgagatttcgattccaccgccgccttctatgaaaggttgggcttcgga |
| | atcgttttccgggacgccggctggatgatcctccagcgcggggatctcatgctggagttctt |
| | cgcccaccccaacttgtttattgcagcttataatggttacaaataaagcaatagcatcacaa |
| | atttcacaaataaagcatttttttcactgcattctagttgtggtttgtccaaactcatcaatgta |
| | tcttatcatgtctgtataccgtcgacctctagctagagcttggcgtaatcatggtcatagctgt |
| | ttcctgtgtgaaattgttatccgctcacaattccacacaacatacgagccggaagcataaa |
| | gtgtaaagcctggggtgcctaatgagtgagctaactcacattaattgcgttgcgctcactgc |
| | ccgctttccagtcgggaaacctgtcgtgccagctgcattaatgaatcggccaacgcgcggg |
| | gagaggcggtttgcgtattgggcgctcttccgcttcctcgctcactgactcgctgcgctcggt |
| | cgttcggctgcggcgagcggtatcagctcactcaaaggcggtaatacggttatccacaga |
| | atcaggggataacgcaggaaagaacatgtgagcaaaaggccagcaaaaggccaggaa |
| | ccgtaaaaaggccgcgttgctggcgtttttccataggctccgcccccctgacgagcatcac |
| | aaaaatcgacgctcaagtcagaggtggcgaaacccgacaggactataaagataccaggc |
| | gtttccccctggaagctccctcgtgcgctctcctgttccgaccctgccgcttaccggatacct |
| | gtccgcctttctcccttcgggaagcgtggcgctttctcatagctcacgctgtaggtatctcagt |
| | tcggtgtaggtcgttcgctccaagctgggctgtgtgcacgaaccccccgttcagcccgacc |
| | gctgcgccttatccggtaactatcgtcttgagtccaacccggtaagacacgacttatcgcca |
| | ctggcagcagccactggtaacaggattagcagagcgaggtatgtaggcggtgctacaga |
| | gttcttgaagtggtggcctaactacggctacactagaagaacagtatttggtatctgcgctc |
| | tgctgaagccagttaccttcggaaaaagagttggtagctcttgatccggcaaacaaaccac |
| | cgctggtagcggtggtttttttgtttgcaagcagcagattacgcgcagaaaaaaaggatctc |
| | aagaagatcctttgatcttttctacggggtctgacgctcagtggaacgaaaactcacgttaa |
| | gggattttggtcatgagattatcaaaaaggatcttcacctagatccttttaaattaaaaatga |
| | agttttaaatcaatctaaagtatatatgagtaaacttggtctgacagttaccaatgcttaatc |
| | agtgaggcacctatctcagcgatctgtctatttcgttcatccatagttgcctgactccccgtcg |
| | tgtagataactacgatacgggagggcttaccatctggccccagtgctgcaatgataccgcg |
| | agacccacgctcaccggctccagatttatcagcaataaaccagccagccggaagggccg |
| | agcgcagaagtggtcctgcaactttatccgcctccatccagtctattaattgttgccgggaa |
| | gctagagtaagtagttcgccagttaatagtttgcgcaacgttgttgccattgctacaggcat |
| | cgtggtgtcacgctcgtcgtttggtatggcttcattcagctccggttcccaacgatcaaggcg |
| | agttacatgatcccccatgttgtgcaaaaaagcggttagctccttcggtcctccgatcgttgt |
| | cagaagtaagttggccgcagtgttatcactcatggttatggcagcactgcataattctcttac |
| | tgtcatgccatccgtaagatgcttttctgtgactggtgagtactcaaccaagtcattctgaga |
| | atagtgtatgcggcgaccgagttgctcttgcccggcgtcaatacgggataataccgcgcca |
| | catagcagaactttaaaagtgctcatcattggaaaacgttcttcggggcgaaaactctcaa |
| | ggatcttaccgctgttgagatccagttcgatgtaacccactcgtgcacccaactgatcttca |
| | gcatcttttactttcaccagcgtttctgggtgagcaaaaacaggaaggcaaaatgccgcaa |
| | aaaagggaataagggcgacacggaaatgttgaatactcatactcttcctttttcaatattat |
| | tgaagcatttatcagggttattgtctcatgagcggatacatatttgaatgtatttagaaaaat |
| | aaacaaataggggttccgcgcacatttccccgaaaagtgccacctgacgtc |
| | (SEQ ID NO: 34) |
| HPV-18 L1 | MALWRPSDNTVYLPPPSVARVVNTDDYVTRTSIFYHAGSS |
| amino acid | RLLTVGNPYFRVPAGGGNKQDIPKVSAYQYRVFRVQLPD |
| | PNKFGLPDTSIYNPETQRLVWACAGVEIGRGQPLGVGLS |
| | GHPFYNKLDDTESSHAATSNVSEDVRDNVSVDYKQTQLCI |
| | LGCAPAIGEHWAKGTACKSRPLSQGDCPPLELKNTVLED |
| | GDMVDTGYGAMDFSTLQDTKCEVPLDICQSICKYPDYLQ |
| | MSADPYGDSMFFCLRREQLFARHFWNRAGTMGDTVPQS |
| | LYIKGTGMRASPGSCVYSPSPSGSIVTSDSQLFNKPYWLH |
| | KAQGHNNGVCWHNQLFVTVVDTTRSTNLTICASTQSPVP |
| | GQYDATKFKQYSRHVEEYDLQFIFQLCTITLTADVMSYIH |
| | SMNSSILEDWNFGVPPPPTTSLVDTYRFVQSVAITCQKDA |
| | APAENKDPYDKLKFWNVDLKEKFSLDLDQYPLGRKFLV |
| | QAGLRRKPTIGPRKRSAPSATTSSKPAKRVRVRARK |
| | (SEQ ID NO: 35) |
| HPV-18 L2 | MVSHRAARRKRASVTDLYKTCKQSGTCPPDVVPKVEGT |
| amino acid | TLADKILQWSSLGIFLGGLGIGTGSGTGGRTGYIPLGGRS |
| | NTVVDVGPTRPPVVIEPVGPTDPSIVTLIEDSSVVTSGAPRP |
| | TFTGTSGFDITSAGTTTPAVLDITPSSTSVSISTTNFTNPAFS |
| | DPSIIEVPQTGEVAGNVFVGTPTSGTHGYEEIPLQTFASSG |
| | TGEEPISSTPLPTVRRVAGPRLYSRAYQQVSVANPEFLTRP |
| | SSLITYDNPAFEPVDTTLTFDPRSDVPDSDFMDIIRLHRPAL |
| | TSRRGTVRFSRLGQRATMFTRSGTQIGARVHFYHDISPIA |
| | PSPEYIELQPLVSATEDNDLFDIYADDMDPAVPVPSRSTTS |
| | FAFFKYSPTISSASSYSNVTVPLTSSWDVPVYTGPDITLPST |
| | TSVWPIVSPTAPASTQYIGIHGTHYYLWPLYYFIPKKRKR |
| | VPYFFADGFVAA (SEQ ID NO: 36) |
| pDY0041HPV1a | gacggatcgggagatctcccgatcccctatggtgcactctcagtacaatctgctctgatgcc |
| L1-HCV | gcatagttaagccagtatctgctccctgcttgtgtgttggaggtcgctgagtagtgcgcgag |
| IRES-L2 | caaaatttaagctacaacaaggcaaggcttgaccgacaattgcatgaagaatctgcttag |
| (seq | ggttaggcgttttgcgctgcttcgcgatgtacgggccagatatacgcgttgacattgattatt |
| dX2CDjFG) | gactagttattaatagtaatcaattacggggtcattagttcatagcccatatatggagttccg |
| CMV | cgttacataacttacggtaaatggcccgcctggctgaccgcccaacgacccccgcccattg |
| promoter: | acgtcaataatgacgtatgttcccatagtaacgccaatagggactttccattgacgtcaatg |
| nucleotides | ggtggagtatttacggtaaactgcccacttggcagtacatcaagtgtatcatatgccaagta |
| 232 to 819 | cgccccctattgacgtcaatgacggtaaatggcccgcctggcattatgcccagtacatgac |
| T7 promoter: | cttatgggactttcctacttggcagtacatctacgtattagtcatcgctattaccatggtgatg |
| nucleotides | cggttttggcagtacatcaatgggcgtggatagcggtttgactcacggggatttccaagtct |
| 863 to 879 | ccaccccattgacgtcaatgggagtttgttttggcaccaaaatcaacgggactttccaaaat |
| HPV-1a L1 | gtcgtaacaactccgccccattgacgcaaatgggcggtaggcgtgtacggtgggaggtct |
| coding | atataagcagagctctctggctaactagagaacccactgcttactggcttatcgaaattaat |
| sequence: | acgactcactatagggagacccaagctggctagcgtttaaacttaagcttgccaccatggc |
| nucleotides | tgtctggttgccggcgcaaaacaaattttatctgccgccacaacctataactaggattctctc |
| 923 to 2431 | cacggatgagtatgtcaccaggaccaatctcttctatcacgctactagcgaacgattgctgc |
| IRES: | ttgttgggcatccactttttgaaataagcagcaaccaaaccgttacaattcctaaggttagc |
| nucleotides | ccaaatgcctttagggtctttcgcgttcgattcgcagaccctaacagatttgccttcggagat |
| 2432 to 2870 | aaggcgatcttcaaccctgaaacagaaaggctcgtgtggggccttcggggtatcgaaatc |
| HPV-1a L2 | ggtcggggccaaccactggggattggaataaccggtcacccattgcttaataaactggatg |
| coding | atgccgaaaatccgactaactacatcaatacgcatgcgaacggggatagtcggcagaata |
| sequence: | cggccttcgatgccaagcaaacacaaatgtttctggtggggtgcactccagctagtggcga |
| nucleotides | acactggactagctccagatgcccgggtgagcaggtcaagctgggggactgtcctcgggt |
| 2871 to 4394 | acaaatgattgaatcagtaatcgaagatggcgacatgatggacattggtttcggtgcgatg |
| BGH polyA: | gattttgcggcactccaacaagataaatctgatgtaccactcgatgtagtacaagctacatg |
| nucleotides | taagtatccggattatataaggatgaatcatgaagcatatggcaactcaatgttttttttcgc |
| 4445 to 4669 | aagaagggagcaaatgtatacacggcatttttttacacggggaggtagcgtaggagataa |
| | ggaagcagtaccgcagtctctgtacctgacagctgatgccgagccccggactaccctggc |
| | gacgaccaactacgtcggcacaccatctgggtcaatggtatcatcagacgtccagctgttc |
| | aatcgatcctactggcttcagaggtgccagggacaaaacaatgggatatgttggcggaac |
| | cagttgtttattactgtgggtgacaatactcgaggaacgtcactgagcatatcaatgaaga |
| | ataacgcctccaccacgtatagtaacgcgaattttaatgacttcctgcgacatacggagga |
| | gtttgatctttccttcatagttcaactctgtaaagtgaagctcacgccagaaaacttggcttat |
| | atccatactatggatccgaatatcctggaggattggcagctgtcagtgagtcagccccctac |
| | caatccccttgaagatcaataccggttcctgggcagtagcctcgcggccaagtgcccggag |
| | caagccccacccgagccacagaccgacccatactctcaatataaattctgggaagtggac |
| | ctgactgaacgaatgtctgagcaacttgaccaatttcccctggggcggaagtttctgtatca |
| | gagcggcatgacgcaacgaaccgcgacatcctccaccactaaaagaaagacggttcgag |
| | tgtctacatccgcaaaacggcgcaggaaagcgtagttctagtgtacgtagccagcccccg |
| | attgggggcgacactccaccatagatcactcccctgtgaggaactactgtcttcacgcaga |
| | aagcgtctagccatggcgttagtatgagagtcgtgcagcctccaggaccccccctcccggg |
| | agagccatagtggtctgcggaaccggtgagtacaccggaattgccaggacgaccgggtc |
| | ctttcttggatcaacccgctcaatgcctggagatttgggcgtgcccccgcaagactgctagc |
| | cgagtagtgttgggtcgcgaaaggccttgtggtactgcctgatagggtgcttgcgagtgcc |
| | ccgggaggtctcgtagaccgtgcaccatgagcacgaatcctaaacctcaaagaaaaacc |
| | aaacgtaacaccaaccgccgcccacaggacgtcttcatatgtctagccaccatgtatcggc |
| | tgcgccgaaagagggctgcccccaaagacatatacccaagttgtaaaatttccaacacttg |
| | cccgcctgatatacaaaataagatagagcacacaaccattgcagataaaattttgcaatac |
| | ggctcactgggcgtcttcttgggtggtcttgggataggtacagctaggggcagcggagggc |
| | gcatcggatatactcccctgggagaaggcggcggggttagggtagccacccgccctacgc |
| | ccgtcagacctacgattcccgtggagacagtcggacctagtgaaatcttccctattgacgtg |
| | gtggatccaactggccctgcagttatccccctccaagacttgggacgagactttcctatacc |
| | gaccgttcaagtaatcgcagaaatacatccaatcagcgatatccctaacattgtagcgtctt |
| | caacgaacgagggggaatccgctatcctggatgtgctccagggttctgccacgatacgca |
| | ccgtttccaggacccaatataataatccatcttttacagttgcttccacctctaacatttccgc |
| | cggggaagccagcacgtcagacatcgtctttgtgtccaacggttctggtgacagagtggta |
| | ggggaagacataccgttggtagaactcaacttgggactcgaaaccgacacaagttcagta |
| | gtccaagagactgcgttctcctccagtacccctatcgccgaacggccctctttccggcccag |
| | tcggttttataaccgacgactctatgagcaagtccaggtccaggatcctcgcttcgttgaac |
| | agccacagagcatggtgactttcgataatcccgctttcgaaccggaactggatgaagtctc |
| | aattatatttcagcgcgatctcgatgcattggcccaaactccagtaccagaatttcgcgacg |
| | tggtgtacctcagtaagccaacattttccagagagcctgggggtcgactccgagtatccag |
| | gttgggcaagagctcaactatcaggaccaggcttggaaccgcaattggggctagaactca |
| | cttcttttacgatctgtccagtattgcgcctgaagattctatagaacttcttcccctcggagag |
| | cactcacaaacaacggtgatctcttccaatttgggagacacagcatttatacagggagaaa |
| | ctgctgaagacgaccttgaggtgattagtctggaaacaccgcaactctactccgaggagg |
| | aactgctcgacaccaatgagtctgtaggcgagaaccttcaattgactataactaacagtga |
| | aggcgaagttagtatacttgacctcacacagtctcgcgtgcgaccaccgttcggcacagag |
| | gatacctctttgcatgtatattaccctaattcaagtaagggaactcccataattaacccaga |
| | ggagtcttttactcctcttgttataatagctttgaataacagtacgggagattttgaactgcat |
| | cccagtttgcggaagcgcaggaagagagcgtatgtataagcggccgctcgagtctagag |
| | ggcccgtttaaacccgctgatcagcctcgactgtgccttctagttgccagccatctgttgtttg |
| | cccctcccccgtgccttccttgaccctggaaggtgccactcccactgtcctttcctaataaaa |
| | tgaggaaattgcatcgcattgtctgagtaggtgtcattctattctggggggtggggtggggc |
| | aggacagcaagggggaggattgggaagacaatagcaggcatgctggggatgcggtggg |
| | ctctatggcttctgaggcggaaagaaccagctggggctctagggggtatccccacgcgcc |
| | ctgtagcggcgcattaagcgcggcgggtgtggtggttacgcgcagcgtgaccgctacactt |
| | gccagcgccctagcgcccgctcctttcgctttcttcccttcctttctcgccacgttcgccggctt |
| | tccccgtcaagctctaaatcgggggctccctttagggttccgatttagtgctttacggcacct |
| | cgaccccaaaaaacttgattagggtgatggttcacgtagtgggccatcgccctgatagacg |
| | gtttttcgccctttgacgttggagtccacgttctttaatagtggactcttgttccaaactggaa |
| | caacactcaaccctatctcggtctattcttttgatttataagggattttgccgatttcggcctat |
| | tggttaaaaaatgagctgatttaacaaaaatttaacgcgaattaattctgtggaatgtgtgt |
| | cagttagggtgtggaaagtccccaggctccccagcaggcagaagtatgcaaagcatgcat |
| | ctcaattagtcagcaaccaggtgtggaaagtccccaggctccccagcaggcagaagtatg |
| | caaagcatgcatctcaattagtcagcaaccatagtcccgcccctaactccgcccatcccgc |
| | ccctaactccgcccagttccgcccattctccgccccatggctgactaattttttttatttatgca |
| | gaggccgaggccgcctctgcctctgagctattccagaagtagtgaggaggcttttttggag |
| | gcctaggcttttgcaaaaagctcccgggagcttgtatatccattttcggatctgatcaagag |
| | acaggatgaggatcgtttcgcatgattgaacaagatggattgcacgcaggttctccggccg |
| | cttgggtggagaggctattcggctatgactgggcacaacagacaatcggctgctctgatgc |
| | cgccgtgttccggctgtcagcgcaggggcgcccggttctttttgtcaagaccgacctgtccg |
| | gtgccctgaatgaactgcaggacgaggcagcgcggctatcgtggctggccacgacgggc |
| | gttccttgcgcagctgtgctcgacgttgtcactgaagcgggaagggactggctgctattggg |
| | cgaagtgccggggcaggatctcctgtcatctcaccttgctcctgccgagaaagtatccatca |
| | tggctgatgcaatgcggcggctgcatacgcttgatccggctacctgcccattcgaccacca |
| | agcgaaacatcgcatcgagcgagcacgtactcggatggaagccggtcttgtcgatcagga |
| | tgatctggacgaagagcatcaggggctcgcgccagccgaactgttcgccaggctcaaggc |
| | gcgcatgcccgacggcgaggatctcgtcgtgacccatggcgatgcctgcttgccgaatatc |
| | atggtggaaaatggccgcttttctggattcatcgactgtggccggctgggtgtggcggacc |
| | gctatcaggacatagcgttggctacccgtgatattgctgaagagcttggcggcgaatgggc |
| | tgaccgcttcctcgtgctttacggtatcgccgctcccgattcgcagcgcatcgccttctatcg |
| | ccttcttgacgagttcttctgagcgggactctggggttcgaaatgaccgaccaagcgacgc |
| | ccaacctgccatcacgagatttcgattccaccgccgccttctatgaaaggttgggcttcgga |
| | atcgttttccgggacgccggctggatgatcctccagcgcggggatctcatgctggagttctt |
| | cgcccaccccaacttgtttattgcagcttataatggttacaaataaagcaatagcatcacaa |
| | atttcacaaataaagcatttttttcactgcattctagttgtggtttgtccaaactcatcaatgta |
| | tcttatcatgtctgtataccgtcgacctctagctagagcttggcgtaatcatggtcatagctgt |
| | ttcctgtgtgaaattgttatccgctcacaattccacacaacatacgagccggaagcataaa |
| | gtgtaaagcctggggtgcctaatgagtgagctaactcacattaattgcgttgcgctcactgc |
| | ccgctttccagtcgggaaacctgtcgtgccagctgcattaatgaatcggccaacgcgcggg |
| | gagaggcggtttgcgtattgggcgctcttccgcttcctcgctcactgactcgctgcgctcggt |
| | cgttcggctgcggcgagcggtatcagctcactcaaaggcggtaatacggttatccacaga |
| | atcaggggataacgcaggaaagaacatgtgagcaaaaggccagcaaaaggccaggaa |
| | ccgtaaaaaggccgcgttgctggcgtttttccataggctccgcccccctgacgagcatcac |
| | aaaaatcgacgctcaagtcagaggtggcgaaacccgacaggactataaagataccaggc |
| | gtttccccctggaagctccctcgtgcgctctcctgttccgaccctgccgcttaccggatacct |
| | gtccgcctttctcccttcgggaagcgtggcgctttctcatagctcacgctgtaggtatctcagt |
| | tcggtgtaggtcgttcgctccaagctgggctgtgtgcacgaaccccccgttcagcccgacc |
| | gctgcgccttatccggtaactatcgtcttgagtccaacccggtaagacacgacttatcgcca |
| | ctggcagcagccactggtaacaggattagcagagcgaggtatgtaggcggtgctacaga |
| | gttcttgaagtggtggcctaactacggctacactagaagaacagtatttggtatctgcgctc |
| | tgctgaagccagttaccttcggaaaaagagttggtagctcttgatccggcaaacaaaccac |
| | cgctggtagcggtggtttttttgtttgcaagcagcagattacgcgcagaaaaaaaggatctc |
| | aagaagatcctttgatcttttctacggggtctgacgctcagtggaacgaaaactcacgttaa |
| | gggattttggtcatgagattatcaaaaaggatcttcacctagatccttttaaattaaaaatga |
| | agttttaaatcaatctaaagtatatatgagtaaacttggtctgacagttaccaatgcttaatc |
| | agtgaggcacctatctcagcgatctgtctatttcgttcatccatagttgcctgactccccgtcg |
| | tgtagataactacgatacgggagggcttaccatctggccccagtgctgcaatgataccgcg |
| | agacccacgctcaccggctccagatttatcagcaataaaccagccagccggaagggccg |
| | agcgcagaagtggtcctgcaactttatccgcctccatccagtctattaattgttgccgggaa |
| | gctagagtaagtagttcgccagttaatagtttgcgcaacgttgttgccattgctacaggcat |
| | cgtggtgtcacgctcgtcgtttggtatggcttcattcagctccggttcccaacgatcaaggcg |
| | agttacatgatcccccatgttgtgcaaaaaagcggttagctccttcggtcctccgatcgttgt |
| | cagaagtaagttggccgcagtgttatcactcatggttatggcagcactgcataattctcttac |
| | tgtcatgccatccgtaagatgcttttctgtgactggtgagtactcaaccaagtcattctgaga |
| | atagtgtatgcggcgaccgagttgctcttgcccggcgtcaatacgggataataccgcgcca |
| | catagcagaactttaaaagtgctcatcattggaaaacgttcttcggggcgaaaactctcaa |
| | ggatcttaccgctgttgagatccagttcgatgtaacccactcgtgcacccaactgatcttca |
| | gcatcttttactttcaccagcgtttctgggtgagcaaaaacaggaaggcaaaatgccgcaa |
| | aaaagggaataagggcgacacggaaatgttgaatactcatactcttcctttttcaatattat |
| | tgaagcatttatcagggttattgtctcatgagcggatacatatttgaatgtatttagaaaaat |
| | aaacaaataggggttccgcgcacatttccccgaaaagtgccacctgacgtc |
| | (SEQ ID NO: 37) |
| HPV-1a L1 | MAVWLPAQNKFYLPPQPITRILSTDEYVTRTNLFYHATSE |
| amino acid | RLLLVGHPLFEISSNQTVTIPKVSPNAFRVFRVRFADPNRF |
| | AFGDKAIFNPETERLVWGLRGIEIGRGQPLGIGITGHPLL |
| | NKLDDAENPTNYINTHANGDSRQNTAFDAKQTQMFLVG |
| | CTPASGEHWTSSRCPGEQVKLGDCPRVQMIESVIEDGDM |
| | MDIGFGAMDFAALQQDKSDVPLDVVQATCKYPDYIRMN |
| | HEAYGNSMFFFARREQMYTRHFFTRGGSVGDKEAVPQS |
| | LYLTADAEPRTTLATTNYVGTPSGSMVSSDVQLFNRSYW |
| | LQRCQGQNNGICWRNQLFITVGDNTRGTSLSISMKNNAS |
| | TTYSNANFNDFLRHTEEFDLSFIVQLCKVKLTPENLAYIH |
| | TMDPNILEDWQLSVSQPPTNPLEDQYRFLGSSLAAKCPEQ |
| | APPEPQTDPYSQYKFWEVDLTERMSEQLDQFPLGRKFLY |
| | QSGMTQRTATSSTTKRKTVRVSTSAKRRRKA |
| | (SEQ ID NO: 38) |
| HPV-1a L2 | MYRLRRKRAAPKDIYPSCKISNTCPPDIQNKIEHTTIADKI |
| amino acid | LQYGSLGVFLGGLGIGTARGSGGRIGYTPLGEGGGVRVA |
| | TRPTPVRPTIPVETVGPSEIFPIDVVDPTGPAVIPLQDLGRD |
| | FPIPTVQVIAEIHPISDIPNIVASSTNEGESAILDVLQGSATIR |
| | TVSRTQYNNPSFTVASTSNISAGEASTSDIVFVSNGSGDRV |
| | VGEDIPLVELNLGLETDTSSVVQETAFSSSTPIAERPSFRPS |
| | RFYNRRLYEQVQVQDPRFVEQPQSMVTFDNPAFEPELDE |
| | VSIIFQRDLDALAQTPVPEFRDVVYLSKPTFSREPGGRLRV |
| | SRLGKSSTIRTRLGTAIGARTHFFYDLSSIAPEDSIELLPLG |
| | EHSQTTVISSNLGDTAFIQGETAEDDLEVISLETPQLYSEE |
| | ELLDTNESVGENLQLTITNSEGEVSILDLTQSRVRPPFGTE |
| | DTSLHVYYPNSSKGTPIINPEESFTPLVIIALNNSTGDFELH |
| | PSLRKRRKRAYV (SEQ ID NO: 39) |
| pDY0042HPV16 | gacggatcgggagatctcccgatcccctatggtgcactctcagtacaatctgctctgatgcc |
| SHELL L1-HCV | gcatagttaagccagtatctgctccctgcttgtgtgttggaggtcgctgagtagtgcgcgag |
| IRES- L2 | caaaatttaagctacaacaaggcaaggcttgaccgacaattgcatgaagaatctgcttag |
| (seq | ggttaggcgttttgcgctgcttcgcgatgtacgggccagatatacgcgttgacattgattatt |
| gqWJjOcE) | gactagttattaatagtaatcaattacggggtcattagttcatagcccatatatggagttccg |
| CMV | cgttacataacttacggtaaatggcccgcctggctgaccgcccaacgacccccgcccattg |
| promoter: | acgtcaataatgacgtatgttcccatagtaacgccaatagggactttccattgacgtcaatg |
| nucleotides | ggtggagtatttacggtaaactgcccacttggcagtacatcaagtgtatcatatgccaagta |
| 232 to 819 | cgccccctattgacgtcaatgacggtaaatggcccgcctggcattatgcccagtacatgac |
| T7 promoter: | cttatgggactttcctacttggcagtacatctacgtattagtcatcgctattaccatggtgatg |
| nucleotides | cggttttggcagtacatcaatgggcgtggatagcggtttgactcacggggatttccaagtct |
| 863 to 879 | ccaccccattgacgtcaatgggagtttgttttggcaccaaaatcaacgggactttccaaaat |
| HPV-16 L1 | gtcgtaacaactccgccccattgacgcaaatgggcggtaggcgtgtacggtgggaggtct |
| coding | atataagcagagctctctggctaactagagaacccactgcttactggcttatcgaaattaat |
| sequence: | acgactcactatagggagacccaagctggctagcgtttaaacttaagcttgccaccatgag |
| nucleotides | cctgtggctgcccagcgaggccaccgtgtacctgccccccgtgcccgtgagcaaggtggtg |
| 923 to 2440 | agcaccgacgagtacgtggccaggaccaacatctactaccacgccggcaccagcaggct |
| IRES: | gctggccgtgggccacccctacttccccatcaagaagcccaacaacaacaagatcctggt |
| nucleotides | gcccaaggtgagcggcctgcagtacagggtgttcaggatccacctgcccgaccccaacaa |
| 2441 to 2879 | gttcggcttccccgacaccagcttctacaaccccgacacccagaggctggtgtgggcctgc |
| HPV-16 L2 | gtgggcgtggaggtgggcaggggccagcccctgggcgtgggcatcagcggccaccccct |
| coding | gctgaacaagctggacgacaccgagaacgccagcgcctacgccgccaacgccggcgtg |
| sequence: | gacaacagggagtgcatcagcatggactacaagcagacccagctgtgcctgatcggctgc |
| nucleotides | aagccccccatcggcgagcactggggcaagggcagcccctgcaccaacgtggccgtgaa |
| 2880 to 4301 | ccccggcgactgcccccccctggagctgatcaacaccgtgatccaggacggcgacatggt |
| BGH polyA: | ggacaccggcttcggcgccatggacttcaccaccctgcaggccaacaagagcgaggtgc |
| nucleotides | ccctggacatctgcaccagcatctgcaagtaccccgactacatcaagatggtgagcgagc |
| 4352 to 4576 | cctacggcgacagcctgttcttctacctgaggagggagcagatgttcgtgaggcacctgttc |
| | aacagggccggcgccgtgggcgagaacgtgcccgacgacctgtacatcaagggcagcg |
| | gcagcaccgccaacctggccagcagcaactacttccccacccccagcggcagcatggtga |
| | ccagcgacgcccagatcttcaacaagccctactggctgcagagggcccagggccacaac |
| | aacggcatctgctggggcaaccagctgttcgtgaccgtggtggacaccaccaggagcacc |
| | aacatgagcctgtgcgccgccatcagcaccagcgagaccacctacaagaacaccaacttc |
| | aaggagtacctgaggcacggcgaggagtacgacctgcagttcatcttccagctgtgcaag |
| | atcaccctgaccgccgacgtgatgacctacatccacagcatgaacagcaccatcctggag |
| | gactggaacttcggcctgcagcccccccccggcggcaccctggaggacacctacaggttc |
| | gtgaccagccaggccatcgcctgccagaagcacaccccccccgcccccaaggaggaccc |
| | cctgaagaagtacaccttctgggaggtgaacctgaaggagaagttcagcgccgacctgga |
| | ccagttccccctgggcaggaagttcctgctgcaggccggcctgaaggccaagcccaagtt |
| | caccctgggcaagaggaaggccacccccaccaccagcagcaccagcaccaccgccaag |
| | aggaagaagaggaagctgtgattctagtgtacgtagccagcccccgattgggggcgaca |
| | ctccaccatagatcactcccctgtgaggaactactgtcttcacgcagaaagcgtctagccat |
| | ggcgttagtatgagagtcgtgcagcctccaggaccccccctcccgggagagccatagtgg |
| | tctgcggaaccggtgagtacaccggaattgccaggacgaccgggtcctttcttggatcaac |
| | ccgctcaatgcctggagatttgggcgtgcccccgcaagactgctagccgagtagtgttggg |
| | tcgcgaaaggccttgtggtactgcctgatagggtgcttgcgagtgccccgggaggtctcgt |
| | agaccgtgcaccatgagcacgaatcctaaacctcaaagaaaaaccaaacgtaacaccaa |
| | ccgccgcccacaggacgtcttcatatgtctagccaccatgaggcacaagaggagcgccaa |
| | gaggaccaagagggccagcgccacccagctgtacaagacctgcaagcaggccggcacc |
| | tgcccccccgacatcatccccaaggtggagggcaagaccatcgccgaccagatcctgcag |
| | tacggcagcatgggcgtgttcttcggcggcctgggcatcggcaccggcagcggcaccggc |
| | ggcaggaccggctacatccccctgggcaccaggccccccaccgccaccgacaccctggc |
| | ccccgtgaggccccccctgaccgtggaccccgtgggccccagcgaccccagcatcgtgag |
| | cctggtggaggagaccagcttcatcgacgccggcgcccccaccagcgtgcccagcatccc |
| | ccccgacgtgagcggcttcagcatcaccaccagcaccgacaccacccccgccatcctgga |
| | catcaacaacaccgtgaccaccgtgaccacccacaacaaccccaccttcaccgaccccag |
| | cgtgctgcagccccccacccccgccgagaccggcggccacttcaccctgagcagcagcac |
| | catcagcacccacaactacgaggagatccccatggacaccttcatcgtgagcaccaaccc |
| | caacaccgtgaccagcagcacccccatccccggcagcaggcccgtggccaggctgggcc |
| | tgtacagcaggaccacccagcaggtgaaggtggtggaccccgccttcgtgaccaccccca |
| | ccaagctgatcacctacgacaaccccgcctacgagggcatcgacgtggacaacaccctgt |
| | acttcagcagcaacgacaacagcatcaacatcgcccccgaccccgacttcctggacatcg |
| | tggccctgcacaggcccgccctgaccagcaggaggaccggcatcaggtacagcaggatc |
| | ggcaacaagcagaccctgaggaccaggagcggcaagagcatcggcgccaaggtgcact |
| | actactacgacctgagcaccatcgaccccgccgaggagatcgagctgcagaccatcaccc |
| | ccagcacctacaccaccaccagccacgccgccagccccaccagcatcaacaacggcctg |
| | tacgacatctacgccgacgacttcatcaccgacaccagcaccacccccgtgcccagcgtg |
| | cccagcaccagcctgagcggctacatccccgccaacaccaccatccccttcggtggcgcct |
| | acaacatccccctggtgagcggccccgacatccccatcaacatcaccgaccaggccccca |
| | gcctgatccccatcgtgcccggcagcccccagtacaccatcatcgccgacgccggcgactt |
| | ctacctgcaccccagctactacatgctgaggaagaggaggaagaggctgccctacttcttc |
| | agcgacgtgagcctggccgcctgagcggccgctcgagtctagagggcccgtttaaacccg |
| | ctgatcagcctcgactgtgccttctagttgccagccatctgttgtttgcccctcccccgtgcct |
| | tccttgaccctggaaggtgccactcccactgtcctttcctaataaaatgaggaaattgcatc |
| | gcattgtctgagtaggtgtcattctattctggggggtggggtggggcaggacagcaagggg |
| | gaggattgggaagacaatagcaggcatgctggggatgcggtgggctctatggcttctgag |
| | gcggaaagaaccagctggggctctagggggtatccccacgcgccctgtagcggcgcatta |
| | agcgcggcgggtgtggtggttacgcgcagcgtgaccgctacacttgccagcgccctagcg |
| | cccgctcctttcgctttcttcccttcctttctcgccacgttcgccggctttccccgtcaagctcta |
| | aatcgggggctccctttagggttccgatttagtgctttacggcacctcgaccccaaaaaact |
| | tgattagggtgatggttcacgtagtgggccatcgccctgatagacggtttttcgccctttgac |
| | gttggagtccacgttctttaatagtggactcttgttccaaactggaacaacactcaaccctat |
| | ctcggtctattcttttgatttataagggattttgccgatttcggcctattggttaaaaaatgag |
| | ctgatttaacaaaaatttaacgcgaattaattctgtggaatgtgtgtcagttagggtgtgga |
| | aagtccccaggctccccagcaggcagaagtatgcaaagcatgcatctcaattagtcagca |
| | accaggtgtggaaagtccccaggctccccagcaggcagaagtatgcaaagcatgcatctc |
| | aattagtcagcaaccatagtcccgcccctaactccgcccatcccgcccctaactccgccca |
| | gttccgcccattctccgccccatggctgactaattttttttatttatgcagaggccgaggccgc |
| | ctctgcctctgagctattccagaagtagtgaggaggcttttttggaggcctaggcttttgcaa |
| | aaagctcccgggagcttgtatatccattttcggatctgatcaagagacaggatgaggatcg |
| | tttcgcatgattgaacaagatggattgcacgcaggttctccggccgcttgggtggagaggc |
| | tattcggctatgactgggcacaacagacaatcggctgctctgatgccgccgtgttccggctg |
| | tcagcgcaggggcgcccggttctttttgtcaagaccgacctgtccggtgccctgaatgaact |
| | gcaggacgaggcagcgcggctatcgtggctggccacgacgggcgttccttgcgcagctgt |
| | gctcgacgttgtcactgaagcgggaagggactggctgctattgggcgaagtgccggggca |
| | ggatctcctgtcatctcaccttgctcctgccgagaaagtatccatcatggctgatgcaatgc |
| | ggcggctgcatacgcttgatccggctacctgcccattcgaccaccaagcgaaacatcgcat |
| | cgagcgagcacgtactcggatggaagccggtcttgtcgatcaggatgatctggacgaaga |
| | gcatcaggggctcgcgccagccgaactgttcgccaggctcaaggcgcgcatgcccgacg |
| | gcgaggatctcgtcgtgacccatggcgatgcctgcttgccgaatatcatggtggaaaatgg |
| | ccgcttttctggattcatcgactgtggccggctgggtgtggcggaccgctatcaggacatag |
| | cgttggctacccgtgatattgctgaagagcttggcggcgaatgggctgaccgcttcctcgtg |
| | ctttacggtatcgccgctcccgattcgcagcgcatcgccttctatcgccttcttgacgagttct |
| | tctgagcgggactctggggttcgaaatgaccgaccaagcgacgcccaacctgccatcacg |
| | agatttcgattccaccgccgccttctatgaaaggttgggcttcggaatcgttttccgggacgc |
| | cggctggatgatcctccagcgcggggatctcatgctggagttcttcgcccaccccaacttgt |
| | ttattgcagcttataatggttacaaataaagcaatagcatcacaaatttcacaaataaagc |
| | atttttttcactgcattctagttgtggtttgtccaaactcatcaatgtatcttatcatgtctgtat |
| | accgtcgacctctagctagagcttggcgtaatcatggtcatagctgtttcctgtgtgaaattg |
| | ttatccgctcacaattccacacaacatacgagccggaagcataaagtgtaaagcctgggg |
| | tgcctaatgagtgagctaactcacattaattgcgttgcgctcactgcccgctttccagtcggg |
| | aaacctgtcgtgccagctgcattaatgaatcggccaacgcgcggggagaggcggtttgcg |
| | tattgggcgctcttccgcttcctcgctcactgactcgctgcgctcggtcgttcggctgcggcg |
| | agcggtatcagctcactcaaaggcggtaatacggttatccacagaatcaggggataacgc |
| | aggaaagaacatgtgagcaaaaggccagcaaaaggccaggaaccgtaaaaaggccgc |
| | gttgctggcgtttttccataggctccgcccccctgacgagcatcacaaaaatcgacgctcaa |
| | gtcagaggtggcgaaacccgacaggactataaagataccaggcgtttccccctggaagct |
| | ccctcgtgcgctctcctgttccgaccctgccgcttaccggatacctgtccgcctttctcccttc |
| | gggaagcgtggcgctttctcatagctcacgctgtaggtatctcagttcggtgtaggtcgttcg |
| | ctccaagctgggctgtgtgcacgaaccccccgttcagcccgaccgctgcgccttatccggt |
| | aactatcgtcttgagtccaacccggtaagacacgacttatcgccactggcagcagccactg |
| | gtaacaggattagcagagcgaggtatgtaggcggtgctacagagttcttgaagtggtggc |
| | ctaactacggctacactagaagaacagtatttggtatctgcgctctgctgaagccagttacc |
| | ttcggaaaaagagttggtagctcttgatccggcaaacaaaccaccgctggtagcggtggtt |
| | tttttgtttgcaagcagcagattacgcgcagaaaaaaaggatctcaagaagatcctttgatc |
| | ttttctacggggtctgacgctcagtggaacgaaaactcacgttaagggattttggtcatgag |
| | attatcaaaaaggatcttcacctagatccttttaaattaaaaatgaagttttaaatcaatcta |
| | aagtatatatgagtaaacttggtctgacagttaccaatgcttaatcagtgaggcacctatct |
| | cagcgatctgtctatttcgttcatccatagttgcctgactccccgtcgtgtagataactacgat |
| | acgggagggcttaccatctggccccagtgctgcaatgataccgcgagacccacgctcacc |
| | ggctccagatttatcagcaataaaccagccagccggaagggccgagcgcagaagtggtc |
| | ctgcaactttatccgcctccatccagtctattaattgttgccgggaagctagagtaagtagtt |
| | cgccagttaatagtttgcgcaacgttgttgccattgctacaggcatcgtggtgtcacgctcgt |
| | cgtttggtatggcttcattcagctccggttcccaacgatcaaggcgagttacatgatccccc |
| | atgttgtgcaaaaaagcggttagctccttcggtcctccgatcgttgtcagaagtaagttggc |
| | cgcagtgttatcactcatggttatggcagcactgcataattctcttactgtcatgccatccgta |
| | agatgcttttctgtgactggtgagtactcaaccaagtcattctgagaatagtgtatgcggcg |
| | accgagttgctcttgcccggcgtcaatacgggataataccgcgccacatagcagaacttta |
| | aaagtgctcatcattggaaaacgttcttcggggcgaaaactctcaaggatcttaccgctgtt |
| | gagatccagttcgatgtaacccactcgtgcacccaactgatcttcagcatcttttactttcac |
| | cagcgtttctgggtgagcaaaaacaggaaggcaaaatgccgcaaaaaagggaataagg |
| | gcgacacggaaatgttgaatactcatactcttcctttttcaatattattgaagcatttatcagg |
| | gttattgtctcatgagcggatacatatttgaatgtatttagaaaaataaacaaataggggtt |
| | ccgcgcacatttccccgaaaagtgccacctgacgtc (SEQ ID NO: 40) |
| HPV-16 L1 | MSLWLPSEATVYLPPVPVSKVVSTDEYVARTNIYYHAGTS |
| amino acid | RLLAVGHPYFPIKKPNNNKILVPKVSGLQYRVFRIHLPDP |
| | NKFGFPDTSFYNPDTQRLVWACVGVEVGRGQPLGVGISG |
| | HPLLNKLDDTENASAYAANAGVDNRECISMDYKQTQLCL |
| | IGCKPPIGEHWGKGSPCTNVAVNPGDCPPLELINTVIQDG |
| | DMVDTGFGAMDFTTLQANKSEVPLDICTSICKYPDYIKM |
| | VSEPYGDSLFFYLRREQMFVRHLFNRAGAVGENVPDDLY |
| | IKGSGSTANLASSNYFPTPSGSMVTSDAQIFNKPYWLQRA |
| | QGHNNGICWGNQLFVTVVDTTRSTNMSLCAAISTSETTY |
| | KNTNFKEYLRHGEEYDLQFIFQLCKITLTADVMTYIHSM |
| | NSTILEDWNFGLQPPPGGTLEDTYRFVTSQAIACQKHTPP |
| | APKEDPLKKYTFWEVNLKEKFSADLDQFPLGRKFLLQAG |
| | LKAKPKFTLGKRKATPTTSSTSTTAKRKKRKL |
| | (SEQ ID NO: 17) |
| HPV-16 L2 | MRHKRSAKRTKRASATQLYKTCKQAGTCPPDIIPKVEGK |
| amino acid | TIADQILQYGSMGVFFGGLGIGTGSGTGGRTGYIPLGTRP |
| | PTATDTLAPVRPPLTVDPVGPSDPSIVSLVEETSFIDAGAPT |
| | SVPSIPPDVSGFSITTSTDTTPAILDINNTVTTVTTHNNPTFT |
| | DPSVLQPPTPAETGGHFTLSSSTISTHNYEEIPMDTFIVSTN |
| | PNTVTSSTPIPGSRPVARLGLYSRTTQQVKVVDPAFVTTPT |
| | KLITYDNPAYEGIDVDNTLYFSSNDNSINIAPDPDFLDIVAL |
| | HRPALTSRRTGIRYSRIGNKQTLRTRSGKSIGAKVHYYYD |
| | LSTIDPAEEIELQTITPSTYTTTSHAASPTSINNGLYDIYAD |
| | DFITDTSTTPVPSVPSTSLSGYIPANTTIPFGGAYNIPLVSGP |
| | DIPINITDQAPSLIPIVPGSPQYTIIADAGDFYLHPSYYMLR |
| | KRRKRLPYFFSDVSLAA (SEQ ID NO: 18) |
| pDY0067 | taatcagcatcatgatgtggtaccacatcatgatgctgattataagaatgcggccgccaca |
| Minicircle | ctctagtggatctcgagttaataattcagaagaactcgtcaagaaggcgatagaaggcga |
| U6-sgRNA | tgcgctgcgaatcgggagcggcgataccgtaaagcacgaggaagcggtcagcccattcg |
| EFS-SpCas9 | ccgccaagctcttcagcaatatcacgggtagccaacgctatgtcctgatagcggtccgcca |
| (with stop | cacccagccggccacagtcgatgaatccagaaaagcggccattttccaccatgatattcgg |
| codon)-bGH | caagcaggcatcgccatgggtcacgacgagatcctcgccgtcgggcatgctcgccttgag |
| poly A | cctggcgaacagttcggctggcgcgagcccctgatgctcttcgtccagatcatcctgatcga |
| (seq | caagaccggcttccatccgagtacgtgctcgctcgatgcgatgtttcgcttggtggtcgaat |
| j34j8UIJ) | gggcaggtagccggatcaagcgtatgcagccgccgcattgcatcagccatgatggatact |
| U6 promoter: | ttctcggcaggagcaaggtgtagatgacatggagatcctgccccggcacttcgcccaatag |
| nucleotides | cagccagtcccttcccgcttcagtgacaacgtcgagcacagctgcgcaaggaacgcccgt |
| 4044 to 4284 | cgtggccagccacgatagccgcgctgcctcgtcttgcagttcattcagggcaccggacagg |
| gRNA | tcggtcttgacaaaaagaaccgggcgcccctgcgctgacagccggaacacggcggcatc |
| scaffold: | agagcagccgattgtctgttgtgcccagtcatagccgaatagcctctccacccaagcggcc |
| nucleotides | ggagaacctgcgtgcaatccatcttgttcaatcatgcgaaacgatcctcatcctgtctcttga |
| 4311 to 4386 | tcagagcttgatcccctgcgccatcagatccttggcggcgagaaagccatccagtttacttt |
| EFS-NS | gcagggcttcccaaccttaccagagggcgccccagctggcaattccggttcgcttgctgtcc |
| promoter: | ataaaaccgcccagtctagctatcgccatgtaagcccactgcaagctacctgctttctctttg |
| nucleotides | cgcttgcgttttcccttgtccagatagcccagtagctgacattcatccggggtcagcaccgtt |
| 4405 to 4660 | tctgcggactggctttctacgtgctcgaggggggccaaacggtctccagcttggctgttttg |
| hSpCas9: | gcggatgagagaagattttcagcctgatacagattaaatcagaacgcagaagcggtctga |
| nucleotides | taaaacagaatttgcctggcggcagtagcgcggtggtcccacctgaccccatgccgaactc |
| 4684 to 8862 | agaagtgaaacgccgtagcgccgatggtagtgtggggtctccccatgcgagagtaggga |
| BGH polyA: | actgccaggcatcaaataaaacgaaaggctcagtcgaaagactgggcctttcgttttatct |
| nucleotides | gttgtttgtcggtgaacgctctcctgagtaggacaaatccgccgggagcggatttgaacgtt |
| 8887 to 9094 | gcgaagcaacggcccggagggtggcgggcaggacgcccgccataaactgccaggcatc |
| | aaattaagcagaaggccatcctgacggatggcctttttgcgtttctacaaactcttttgtttat |
| | ttttctaaatacattcaaatatgtatccgctcatgaccaaaatcccttaacgtgagttttcgtt |
| | ccactgagcgtcagaccccgtagaaaagatcaaaggatcttcttgagatcctttttttctgc |
| | gcgtaatctgctgcttgcaaacaaaaaaaccaccgctaccagcggtggtttgtttgccgga |
| | tcaagagctaccaactctttttccgaaggtaactggcttcagcagagcgcagataccaaat |
| | actgtccttctagtgtagccgtagttaggccaccacttcaagaactctgtagcaccgcctac |
| | atacctcgctctgctaatcctgttaccagtggctgctgccagtggcgataagtcgtgtcttac |
| | cgggttggactcaagacgatagttaccggataaggcgcagcggtcgggctgaacggggg |
| | gttcgtgcacacagcccagcttggagcgaacgacctacaccgaactgagatacctacagc |
| | gtgagctatgagaaagcgccacgcttcccgaagggagaaaggcggacaggtatccggta |
| | agcggcagggtcggaacaggagagcgcacgagggagcttccagggggaaacgcctggt |
| | atctttatagtcctgtcgggtttcgccacctctgacttgagcgtcgatttttgtgatgctcgtca |
| | ggggggcggagcctatggaaaaacgccagcaacgcggcctttttacggttcctggcctttt |
| | gctggccttttgctcacatgttctttcctgcgttatcccctgattctgtggataaccgtattacc |
| | gcctttgagtgagctgataccgctcgccgcagccgaacgaccgagcgcagcgagtcagtg |
| | agcgaggaagcggaagagcgcctgatgcggtattttctccttacgcatctgtgcggtatttc |
| | acaccgcatatggtgcactctcagtacaatctgctctgatgccgcatagttaagccagtata |
| | cactccgctatcgctacgtgactgggtcatggctgcgccccgacacccgccaacacccgct |
| | gacgcgccctgacgggcttgtctgctcccggcatccgcttacagacaagctgtgaccgtctc |
| | cgggagctgcatgtgtcagaggttttcaccgtcatcaccgaaacgcgcgaggcagcagat |
| | caattcgcgcgcgaaggcgaagcggcatgcataatgtgcctgtcaaatggacgaagcag |
| | ggattctgcaaaccctatgctactccgtcaagccgtcaattgtctgattcgttaccaattatg |
| | acaacttgacggctacatcattcactttttcttcacaaccggcacggaactcgctcgggctg |
| | gccccggtgcattttttaaatacccgcgagaaatagagttgatcgtcaaaaccaacattgc |
| | gaccgacggtggcgataggcatccgggtggtgctcaaaagcagcttcgcctggctgatac |
| | gttggtcctcgcgccagcttaagacgctaatccctaactgctggcggaaaagatgtgacag |
| | acgcgacggcgacaagcaaacatgctgtgcgacgctggcgatacattaccctgttatccct |
| | agatgacattaccctgttatcccagatgacattaccctgttatccctagatgacattaccctg |
| | ttatccctagatgacatttaccctgttatccctagatgacattaccctgttatcccagatgaca |
| | ttaccctgttatccctagatacattaccctgttatcccagatgacataccctgttatccctaga |
| | tgacattaccctgttatcccagatgacattaccctgttatccctagatacattaccctgttatc |
| | ccagatgacataccctgttatccctagatgacattaccctgttatcccagatgacattaccct |
| | gttatccctagatacattaccctgttatcccagatgacataccctgttatccctagatgacatt |
| | accctgttatcccagatgacattaccctgttatccctagatacattaccctgttatcccagatg |
| | acataccctgttatccctagatgacattaccctgttatcccagatgacattaccctgttatccc |
| | tagatacattaccctgttatcccagatgacataccctgttatccctagatgacattaccctgtt |
| | atcccagatgacattaccctgttatccctagatacattaccctgttatcccagatgacatacc |
| | ctgttatccctagatgacattaccctgttatcccagataaactcaatgatgatgatgatgatg |
| | gtcgagactcagcggccgcggtgccagggcgtgcccttgggctccccgggcgcgactata |
| | agctgcgagcaacttcacttgggtatgccggcggtagcgctgagggcctatttcccatgatt |
| | ccttcatatttgcatatacgatacaaggctgttagagagataattggaattaatttgactgta |
| | aacacaaagatattagtacaaaatacgtgacgtagaaagtaataatttcttgggtagtttg |
| | cagttttaaaattatgttttaaaatggactatcatatgcttaccgtaacttgaaagtatttcga |
| | tttcttggctttatatatcttgtggaaaggacgaaacaccgggtcttcgagaagacctgtttt |
| | agagctagaaatagcaagttaaaataaggctagtccgttatcaacttgaaaaagtggcac |
| | cgagtcggtgcttttttgaattcgctagctaggtcttgaaaggagtgggaattggctccggt |
| | gcccgtcagtgggcagagcgcacatcgcccacagtccccgagaagttggggggaggggt |
| | cggcaattgatccggtgcctagagaaggtggcgcggggtaaactgggaaagtgatgtcgt |
| | gtactggctccgcctttttcccgagggtgggggagaaccgtatataagtgcagtagtcgcc |
| | gtgaacgttctttttcgcaacgggtttgccgccagaacacaggaccggttctagagcgctgc |
| | caccatggacaagaagtacagcatcggcctggacatcggcaccaactctgtgggctgggc |
| | cgtgatcaccgacgagtacaaggtgcccagcaagaaattcaaggtgctgggcaacaccg |
| | accggcacagcatcaagaagaacctgatcggagccctgctgttcgacagcggcgaaaca |
| | gccgaggccacccggctgaagagaaccgccagaagaagatacaccagacggaagaac |
| | cggatctgctatctgcaagagatcttcagcaacgagatggccaaggtggacgacagcttct |
| | tccacagactggaagagtccttcctggtggaagaggataagaagcacgagcggcacccc |
| | atcttcggcaacatcgtggacgaggtggcctaccacgagaagtaccccaccatctaccac |
| | ctgagaaagaaactggtggacagcaccgacaaggccgacctgcggctgatctatctggcc |
| | ctggcccacatgatcaagttccggggccacttcctgatcgagggcgacctgaaccccgac |
| | aacagcgacgtggacaagctgttcatccagctggtgcagacctacaaccagctgttcgag |
| | gaaaaccccatcaacgccagcggcgtggacgccaaggccatcctgtctgccagactgag |
| | caagagcagacggctggaaaatctgatcgcccagctgcccggcgagaagaagaatggc |
| | ctgttcggaaacctgattgccctgagcctgggcctgacccccaacttcaagagcaacttcg |
| | acctggccgaggatgccaaactgcagctgagcaaggacacctacgacgacgacctggac |
| | aacctgctggcccagatcggcgaccagtacgccgacctgtttctggccgccaagaacctgt |
| | ccgacgccatcctgctgagcgacatcctgagagtgaacaccgagatcaccaaggcccccc |
| | tgagcgcctctatgatcaagagatacgacgagcaccaccaggacctgaccctgctgaaag |
| | ctctcgtgcggcagcagctgcctgagaagtacaaagagattttcttcgaccagagcaaga |
| | acggctacgccggctacattgacggcggagccagccaggaagagttctacaagttcatca |
| | agcccatcctggaaaagatggacggcaccgaggaactgctcgtgaagctgaacagagag |
| | gacctgctgcggaagcagcggaccttcgacaacggcagcatcccccaccagatccacctg |
| | ggagagctgcacgccattctgcggcggcaggaagatttttacccattcctgaaggacaacc |
| | gggaaaagatcgagaagatcctgaccttccgcatcccctactacgtgggccctctggccag |
| | gggaaacagcagattcgcctggatgaccagaaagagcgaggaaaccatcaccccctgg |
| | aacttcgaggaagtggtggacaagggcgcttccgcccagagcttcatcgagcggatgacc |
| | aacttcgataagaacctgcccaacgagaaggtgctgcccaagcacagcctgctgtacgag |
| | tacttcaccgtgtataacgagctgaccaaagtgaaatacgtgaccgagggaatgagaaag |
| | cccgccttcctgagcggcgagcagaaaaaggccatcgtggacctgctgttcaagaccaac |
| | cggaaagtgaccgtgaagcagctgaaagaggactacttcaagaaaatcgagtgcttcga |
| | ctccgtggaaatctccggcgtggaagatcggttcaacgcctccctgggcacataccacgat |
| | ctgctgaaaattatcaaggacaaggacttcctggacaatgaggaaaacgaggacattctg |
| | gaagatatcgtgctgaccctgacactgtttgaggacagagagatgatcgaggaacggctg |
| | aaaacctatgcccacctgttcgacgacaaagtgatgaagcagctgaagcggcggagata |
| | caccggctggggcaggctgagccggaagctgatcaacggcatccgggacaagcagtccg |
| | gcaagacaatcctggatttcctgaagtccgacggcttcgccaacagaaacttcatgcagct |
| | gatccacgacgacagcctgacctttaaagaggacatccagaaagcccaggtgtccggcc |
| | agggcgatagcctgcacgagcacattgccaatctggccggcagccccgccattaagaag |
| | ggcatcctgcagacagtgaaggtggtggacgagctcgtgaaagtgatgggccggcacaa |
| | gcccgagaacatcgtgatcgaaatggccagagagaaccagaccacccagaagggacag |
| | aagaacagccgcgagagaatgaagcggatcgaagagggcatcaaagagctgggcagc |
| | cagatcctgaaagaacaccccgtggaaaacacccagctgcagaacgagaagctgtacct |
| | gtactacctgcagaatgggcgggatatgtacgtggaccaggaactggacatcaaccggct |
| | gtccgactacgatgtggaccatatcgtgcctcagagctttctgaaggacgactccatcgac |
| | aacaaggtgctgaccagaagcgacaagaaccggggcaagagcgacaacgtgccctccg |
| | aagaggtcgtgaagaagatgaagaactactggcggcagctgctgaacgccaagctgatt |
| | acccagagaaagttcgacaatctgaccaaggccgagagaggcggcctgagcgaactgg |
| | ataaggccggcttcatcaagagacagctggtggaaacccggcagatcacaaagcacgtg |
| | gcacagatcctggactcccggatgaacactaagtacgacgagaatgacaagctgatccg |
| | ggaagtgaaagtgatcaccctgaagtccaagctggtgtccgatttccggaaggatttccag |
| | ttttacaaagtgcgcgagatcaacaactaccaccacgcccacgacgcctacctgaacgcc |
| | gtcgtgggaaccgccctgatcaaaaagtaccctaagctggaaagcgagttcgtgtacggc |
| | gactacaaggtgtacgacgtgcggaagatgatcgccaagagcgagcaggaaatcggca |
| | aggctaccgccaagtacttcttctacagcaacatcatgaactttttcaagaccgagattacc |
| | ctggccaacggcgagatccggaagcggcctctgatcgagacaaacggcgaaaccgggg |
| | agatcgtgtgggataagggccgggattttgccaccgtgcggaaagtgctgagcatgcccc |
| | aagtgaatatcgtgaaaaagaccgaggtgcagacaggcggcttcagcaaagagtctatc |
| | ctgcccaagaggaacagcgataagctgatcgccagaaagaaggactgggaccctaaga |
| | agtacggcggcttcgacagccccaccgtggcctattctgtgctggtggtggccaaagtgga |
| | aaagggcaagtccaagaaactgaagagtgtgaaagagctgctggggatcaccatcatgg |
| | aaagaagcagcttcgagaagaatcccatcgactttctggaagccaagggctacaaagaa |
| | gtgaaaaaggacctgatcatcaagctgcctaagtactccctgttcgagctggaaaacggc |
| | cggaagagaatgctggcctctgccggcgaactgcagaagggaaacgaactggccctgcc |
| | ctccaaatatgtgaacttcctgtacctggccagccactatgagaagctgaagggctccccc |
| | gaggataatgagcagaaacagctgtttgtggaacagcacaagcactacctggacgagat |
| | catcgagcagatcagcgagttctccaagagagtgatcctggccgacgctaatctggacaa |
| | agtgctgtccgcctacaacaagcaccgggataagcccatcagagagcaggccgagaata |
| | tcatccacctgtttaccctgaccaatctgggagcccctgccgccttcaagtactttgacacca |
| | ccatcgaccggaagaggtacaccagcaccaaagaggtgctggacgccaccctgatccac |
| | cagagcatcaccggcctgtacgagacacggatcgacctgtctcagctgggaggcgacaa |
| | gcgacctgccgccacaaagaaggctggacaggctaagaagaagaaagattacaaagac |
| | gatgacgataagtaactagagctcgctgatcagcctcgactgtgccttctagttgccagcca |
| | tctgttgtttgcccctcccccgtgccttccttgaccctggaaggtgccactcccactgtcctttc |
| | ctaataaaatgaggaaattgcatcgcattgtctgagtaggtgtcattctattctggggggtg |
| | gggtggggcaggacagcaagggggaggattgggaagagaatagcaggcatgctgggg |
| | actgaggcggaaagaaccagctgtggaatgtgtgtcagttagggtgtggaaagtccccag |
| | gctccccagcaggcagaagtatgcaaagcatgcatctcaattagtcagcaaccaggtgtg |
| | gaaagtccccaggctccccagcaggcagaagtatgcaaagcatgcatctcaattagtcag |
| | caaccatagtcccgcccctaactccgcccatcccgcccctaactccgcccagttccgcccat |
| | tctccgccccatggctgactaattttttttatttatgcagaggccgaggccgcctcggcctctg |
| | agctattccagaagtagtgaggaggcttttttggaggcctaggcttttgcaaaaagcttggg |
| | cccgccccaactggggtaacctttgagttctctcagttggggg (SEQ ID NO: 41) |
| pDY0070 | gatcgcgaaaagcgaacaggagataggcaaggctacagccaaatacttcttttattctaa |
| Minicircle | cattatgaatttctttaagacggaaatcactctggcaaacggagagatacgcaaacgacct |
| U6-sgRNA | ttaattgaaaccaatggggagacaggtgaaatcgtatgggataagggccgggacttcgcg |
| CMV- | acggtgagaaaagttttgtccatgccccaagtcaacatagtaaagaaaactgaggtgcag |
| ABE7.10- | accggagggttttcaaaggaatcgattcttccaaaaaggaatagtgataagctcatcgctc |
| TadA-SpCas9- | gtaaaaaggactgggacccgaaaaagtacggtggcttcgatagccctacagttgcctattc |
| bGH poly A | tgtcctagtagtggcaaaagttgagaagggaaaatccaagaaactgaagtcagtcaaag |
| with AmpR | aattattggggataacgattatggagcgctcgtcttttgaaaagaaccccatcgacttcctt |
| (seq | gaggcgaaaggttacaaggaagtaaaaaaggatctcataattaaactaccaaagtatag |
| r8zksrDI) | tctgtttgagttagaaaatggccgaaaacggatgttggctagcgccggagagcttcaaaa |
| U6 promoter: | ggggaacgaactcgcactaccgtctaaatacgtgaatttcctgtatttagcgtcccattacg |
| nucleotides | agaagttgaaaggttcacctgaagataacgaacagaagcaactttttgttgagcagcaca |
| 6019 to 6259 | aacattatctcgacgaaatcatagagcaaatttcggaattcagtaagagagtcatcctagc |
| gRNA | tgatgccaatctggacaaagtattaagcgcatacaacaagcacagggataaacccatacg |
| scaffold: | tgagcaggcggaaaatattatccatttgtttactcttaccaacctcggcgctccagccgcatt |
| nucleotides | caagtattttgacacaacgatagatcgcaaacgatacacttctaccaaggaggtgctagac |
| 6286 to 6361 | gcgacactgattcaccaatccatcacgggattatatgaaactcggatagatttgtcacagct |
| CMV | tgggggtgactctggtggttctcccaagaagaagaggaaagtctaaccggtcatcatcacc |
| enhancer: | atcaccattgagtttaaacccgctgatcagcctcgactgtgccttctagttgccagccatctg |
| nucleotides | ttgtttgcccctcccccgtgccttccttgaccctggaaggtgccactcccactgtcctttccta |
| 6392 to 6771 | ataaaatgaggaaattgcatcgcattgtctgagtaggtgtcattctattctggggggtgggg |
| CMV | tggggcaggacagcaagggggaggattgggaagagaatagcaggcatgctggggatgc |
| promoter: | ggtgggctctatggctgaggcggaaagaaccagctgtggaatgtgtgtcagttagggtgtg |
| nucleotides | gaaagtccccaggctccccagcaggcagaagtatgcaaagcatgcatctcaattagtcag |
| 6772 to 6975 | caaccaggtgtggaaagtccccaggctccccagcaggcagaagtatgcaaagcatgcat |
| T7 promoter: | ctcaattagtcagcaaccatagtcccgcccctaactccgcccatcccgcccctaactccgcc |
| nucleotides | cagttccgcccattctccgccccatggctgactaattttttttatttatgcagaggccgaggcc |
| 7017 to 7036 | gcctcggcctctgagctattccagaagtagtgaggaggcttttttggaggcctaggcttttgc |
| TadA E coli: | aaaaagcttgggcccgccccaactggggtaacctttgagttctctcagttgggggtaatca |
| nucleotides | gcatcatgatgtggtaccacatcatgatgctgattataagaatgcggccgccacactctagt |
| 7049 to 7537 | ggatctcgagttaataattcagaagaactcgtcaagaaggcgatagaaggcgatgcgctg |
| TadA mutant | cgaatcgggagcggcgataccgtaaagcacgaggaagcggtcagcccattcgccgccaa |
| E coli: | gctcttcagcaatatcacgggtagccaacgctatgtcctgatagcggtccgacttggtctga |
| nucleotides | cagttaccaatgcttaatcagtgaggcacctatctcagcgatctgtctatttcgttcatccata |
| 7652 to 8131 | gttgcctgactccccgtcgtgtagataactacgatacgggagggcttaccatctggcccca |
| Cas9(D10A): | gtgctgcaatgataccgcgagacccacgctcaccggctccagatttatcagcaataaacca |
| nucleotides | gccagccggaagggccgagcgcagaagtggtcctgcaactttatccgcctccatccagtct |
| 8240 to | attaattgttgccgggaagctagagtaagtagttcgccagttaatagtttgcgcaacgttgtt |
| 11298 | gccattgctacaggcatcgtggtgtcacgctcgtcgtttggtatggcttcattcagctccggtt |
| | cccaacgatcaaggcgagttacatgatcccccatgttgtgcaaaaaagcggttagctcctt |
| | cggtcctccgatcgttgtcagaagtaagttggccgcagtgttatcactcatggttatggcag |
| | cactgcataattctcttactgtcatgccatccgtaagatgcttttctgtgactggtgagtactc |
| | aaccaagtcattctgagaatagtgtatgcggcgaccgagttgctcttgcccggcgtcaata |
| | cgggataataccgcgccacatagcagaactttaaaagtgctcatcattggaaaacgttctt |
| | cggggcgaaaactctcaaggatcttaccgctgttgagatccagttcgatgtaacccactcgt |
| | gcacccaactgatcttcagcatcttttactttcaccagcgtttctgggtgagcaaaaacagg |
| | aaggcaaaatgccgcaaaaaagggaataagggcgacacggaaatgttgaatactcata |
| | ctcttcctttttcaatattattgaagcatttatcagggttattgtctcatgagcggatacatattt |
| | gaatgtatttagaaaaataaacaaataggggttccgcgcacatttccccgaaaaggcttgc |
| | tgtccataaaaccgcccagtctagctatcgccatgtaagcccactgcaagctacctgctttc |
| | tctttgcgcttgcgttttcccttgtccagatagcccagtagctgacattcatccggggtcagc |
| | accgtttctgcggactggctttctacgtgctcgaggggggccaaacggtctccagcttggct |
| | gttttggcggatgagagaagattttcagcctgatacagattaaatcagaacgcagaagcg |
| | gtctgataaaacagaatttgcctggcggcagtagcgcggtggtcccacctgaccccatgcc |
| | gaactcagaagtgaaacgccgtagcgccgatggtagtgtggggtctccccatgcgagagt |
| | agggaactgccaggcatcaaataaaacgaaaggctcagtcgaaagactgggcctttcgtt |
| | ttatctgttgtttgtcggtgaacgctctcctgagtaggacaaatccgccgggagcggatttg |
| | aacgttgcgaagcaacggcccggagggtggcgggcaggacgcccgccataaactgcca |
| | ggcatcaaattaagcagaaggccatcctgacggatggcctttttgcgtttctacaaactcttt |
| | tgtttatttttctaaatacattcaaatatgtatccgctcatgaccaaaatcccttaacgtgagt |
| | tttcgttccactgagcgtcagaccccgtagaaaagatcaaaggatcttcttgagatccttttt |
| | ttctgcgcgtaatctgctgcttgcaaacaaaaaaaccaccgctaccagcggtggtttgtttg |
| | ccggatcaagagctaccaactctttttccgaaggtaactggcttcagcagagcgcagatac |
| | caaatactgtccttctagtgtagccgtagttaggccaccacttcaagaactctgtagcaccg |
| | cctacatacctcgctctgctaatcctgttaccagtggctgctgccagtggcgataagtcgtgt |
| | cttaccgggttggactcaagacgatagttaccggataaggcgcagcggtcgggctgaacg |
| | gggggttcgtgcacacagcccagcttggagcgaacgacctacaccgaactgagataccta |
| | cagcgtgagctatgagaaagcgccacgcttcccgaagggagaaaggcggacaggtatcc |
| | ggtaagcggcagggtcggaacaggagagcgcacgagggagcttccagggggaaacgc |
| | ctggtatctttatagtcctgtcgggtttcgccacctctgacttgagcgtcgatttttgtgatgct |
| | cgtcaggggggcggagcctatggaaaaacgccagcaacgcggcctttttacggttcctgg |
| | ccttttgctggccttttgctcacatgttctttcctgcgttatcccctgattctgtggataaccgta |
| | ttaccgcctttgagtgagctgataccgctcgccgcagccgaacgaccgagcgcagcgagt |
| | cagtgagcgaggaagcggaagagcgcctgatgcggtattttctccttacgcatctgtgcgg |
| | tatttcacaccgcatatggtgcactctcagtacaatctgctctgatgccgcatagttaagcca |
| | gtatacactccgctatcgctacgtgactgggtcatggctgcgccccgacacccgccaacac |
| | ccgctgacgcgccctgacgggcttgtctgctcccggcatccgcttacagacaagctgtgac |
| | cgtctccgggagctgcatgtgtcagaggttttcaccgtcatcaccgaaacgcgcgaggcag |
| | cagatcaattcgcgcgcgaaggcgaagcggcatgcataatgtgcctgtcaaatggacgaa |
| | gcagggattctgcaaaccctatgctactccgtcaagccgtcaattgtctgattcgttaccaat |
| | tatgacaacttgacggctacatcattcactttttcttcacaaccggcacggaactcgctcgg |
| | gctggccccggtgcattttttaaatacccgcgagaaatagagttgatcgtcaaaaccaaca |
| | ttgcgaccgacggtggcgataggcatccgggtggtgctcaaaagcagcttcgcctggctg |
| | atacgttggtcctcgcgccagcttaagacgctaatccctaactgctggcggaaaagatgtg |
| | acagacgcgacggcgacaagcaaacatgctgtgcgacgctggcgatacattaccctgtta |
| | tccctagatgacattaccctgttatcccagatgacattaccctgttatccctagatgacatta |
| | ccctgttatccctagatgacatttaccctgttatccctagatgacattaccctgttatcccaga |
| | tgacattaccctgttatccctagatacattaccctgttatcccagatgacataccctgttatcc |
| | ctagatgacattaccctgttatcccagatgacattaccctgttatccctagatacattaccct |
| | gttatcccagatgacataccctgttatccctagatgacattaccctgttatcccagatgacat |
| | taccctgttatccctagatacattaccctgttatcccagatgacataccctgttatccctagat |
| | gacattaccctgttatcccagatgacattaccctgttatccctagatacattaccctgttatcc |
| | cagatgacataccctgttatccctagatgacattaccctgttatcccagatgacattaccctg |
| | ttatccctagatacattaccctgttatcccagatgacataccctgttatccctagatgacatta |
| | ccctgttatcccagatgacattaccctgttatccctagatacattaccctgttatcccagatga |
| | cataccctgttatccctagatgacattaccctgttatcccagataaactcaatgatgatgatg |
| | atgatggtcgagactcagcggccgcggtgccagggcgtgcccttgggctccccgggcgcg |
| | actataagctgcgagcaacttcacttgggtatgccggcggtagcgctgagggcctatttccc |
| | atgattccttcatatttgcatatacgatacaaggctgttagagagataattggaattaatttg |
| | actgtaaacacaaagatattagtacaaaatacgtgacgtagaaagtaataatttcttgggt |
| | agtttgcagttttaaaattatgttttaaaatggactatcatatgcttaccgtaacttgaaagta |
| | tttcgatttcttggctttatatatcttgtggaaaggacgaaacaccgggtcttcgagaagacc |
| | tgttttagagctagaaatagcaagttaaaataaggctagtccgttatcaacttgaaaaagt |
| | ggcaccgagtcggtgcttttttatgtacgggccagatatacgcgttgacattgattattgact |
| | agttattaatagtaatcaattacggggtcattagttcatagcccatatatggagttccgcgtt |
| | acataacttacggtaaatggcccgcctggctgaccgcccaacgacccccgcccattgacgt |
| | caataatgacgtatgttcccatagtaacgccaatagggactttccattgacgtcaatgggtg |
| | gagtatttacggtaaactgcccacttggcagtacatcaagtgtatcatatgccaagtacgcc |
| | ccctattgacgtcaatgacggtaaatggcccgcctggcattatgcccagtacatgaccttat |
| | gggactttcctacttggcagtacatctacgtattagtcatcgctattaccatggtgatgcggtt |
| | ttggcagtacatcaatgggcgtggatagcggtttgactcacggggatttccaagtctccacc |
| | ccattgacgtcaatgggagtttgttttggcaccaaaatcaacgggactttccaaaatgtcgt |
| | aacaactccgccccattgacgcaaatgggcggtaggcgtgtacggtgggaggtctatata |
| | agcagagctggtttagtgaaccgtcagatccgctagagatccgcggccgctaatacgactc |
| | actatagggagagccgccaccatgtccgaagtcgagttttcccatgagtactggatgagac |
| | acgcattgactctcgcaaagagggcttgggatgaacgcgaggtgcccgtgggggcagtac |
| | tcgtgcataacaatcgcgtaatcggcgaaggttggaataggccgatcggacgccacgacc |
| | ccactgcacatgcggaaatcatggcccttcgacagggagggcttgtgatgcagaattatcg |
| | acttatcgatgcgacgctgtacgtcacgcttgaaccttgcgtaatgtgcgcgggagctatga |
| | ttcactcccgcattggacgagttgtattcggtgcccgcgacgccaagacgggtgccgcagg |
| | ttcactgatggacgtgctgcatcacccaggcatgaaccaccgggtagaaatcacagaagg |
| | catattggcggacgaatgtgcggcgctgttgtccgacttttttcgcatgcggaggcaggag |
| | atcaaggcccagaaaaaagcacaatcctctactgactctggtggttcttctggtggttctag |
| | cggcagcgagactcccgggacctcagagtccgccacacccgaaagttctggtggttcttct |
| | ggtggttcttccgaagtcgagttttcccatgagtactggatgagacacgcattgactctcgc |
| | aaagagggctcgagatgaacgcgaggtgcccgtgggggcagtactcgtgctcaacaatc |
| | gcgtaatcggcgaaggttggaatagggcaatcggactccacgaccccactgcacatgcgg |
| | aaatcatggcccttcgacagggagggcttgtgatgcagaattatcgacttatcgatgcgac |
| | gctgtacgtcacgtttgaaccttgcgtaatgtgcgcgggagctatgattcactcccgcattg |
| | gacgagttgtattcggtgttcgcaacgccaagacgggtgccgcaggttcactgatggacgt |
| | gctgcattacccaggcatgaaccaccgggtagaaatcacagaaggcatattggcggacg |
| | aatgtgcggcgctgttgtgttacttttttcgcatgcccaggcaggtctttaacgcccagaaaa |
| | aagcacaatcctctactgactctggtggttcttctggtggttctagcggcagcgagactccc |
| | gggacctcagagtccgccacacccgaaagttctggtggttcttctggtggttctgataaaaa |
| | gtattctattggtttagccatcggcactaattccgttggatgggctgtcataaccgatgaata |
| | caaagtaccttcaaagaaatttaaggtgttggggaacacagaccgtcattcgattaaaaa |
| | gaatcttatcggtgccctcctattcgatagtggcgaaacggcagaggcgactcgcctgaaa |
| | cgaaccgctcggagaaggtatacacgtcgcaagaaccgaatatgttacttacaagaaatt |
| | tttagcaatgagatggccaaagttgacgattctttctttcaccgtttggaagagtccttccttg |
| | tcgaagaggacaagaaacatgaacggcaccccatctttggaaacatagtagatgaggtg |
| | gcatatcatgaaaagtacccaacgatttatcacctcagaaaaaagctagttgactcaactg |
| | ataaagcggacctgaggttaatctacttggctcttgcccatatgataaagttccgtgggcac |
| | tttctcattgagggtgatctaaatccggacaactcggatgtcgacaaactgttcatccagtta |
| | gtacaaacctataatcagttgtttgaagagaaccctataaatgcaagtggcgtggatgcga |
| | aggctattcttagcgcccgcctctctaaatcccgacggctagaaaacctgatcgcacaatta |
| | cccggagagaagaaaaatgggttgttcggtaaccttatagcgctctcactaggcctgacac |
| | caaattttaagtcgaacttcgacttagctgaagatgccaaattgcagcttagtaaggacac |
| | gtacgatgacgatctcgacaatctactggcacaaattggagatcagtatgcggacttatttt |
| | tggctgccaaaaaccttagcgatgcaatcctcctatctgacatactgagagttaatactgag |
| | attaccaaggcgccgttatccgcttcaatgatcaaaaggtacgatgaacatcaccaagact |
| | tgacacttctcaaggccctagtccgtcagcaactgcctgagaaatataaggaaatattcttt |
| | gatcagtcgaaaaacgggtacgcaggttatattgacggcggagcgagtcaagaggaatt |
| | ctacaagtttatcaaacccatattagagaagatggatgggacggaagagttgcttgtaaaa |
| | ctcaatcgcgaagatctactgcgaaagcagcggactttcgacaacggtagcattccacatc |
| | aaatccacttaggcgaattgcatgctatacttagaaggcaggaggatttttatccgttcctc |
| | aaagacaatcgtgaaaagattgagaaaatcctaacctttcgcataccttactatgtgggac |
| | ccctggcccgagggaactctcggttcgcatggatgacaagaaagtccgaagaaacgatta |
| | ctccatggaattttgaggaagttgtcgataaaggtgcgtcagctcaatcgttcatcgagagg |
| | atgaccaactttgacaagaatttaccgaacgaaaaagtattgcctaagcacagtttacttta |
| | cgagtatttcacagtgtacaatgaactcacgaaagttaagtatgtcactgagggcatgcgt |
| | aaacccgcctttctaagcggagaacagaagaaagcaatagtagatctgttattcaagacc |
| | aaccgcaaagtgacagttaagcaattgaaagaggactactttaagaaaattgaatgcttc |
| | gattctgtcgagatctccggggtagaagatcgatttaatgcgtcacttggtacgtatcatga |
| | cctcctaaagataattaaagataaggacttcctggataacgaagagaatgaagatatctta |
| | gaagatatagtgttgactcttaccctctttgaagatcgggaaatgattgaggaaagactaa |
| | aaacatacgctcacctgttcgacgataaggttatgaaacagttaaagaggcgtcgctatac |
| | gggctggggacgattgtcgcggaaacttatcaacgggataagagacaagcaaagtggta |
| | aaactattctcgattttctaaagagcgacggcttcgccaataggaactttatgcagctgatc |
| | catgatgactctttaaccttcaaagaggatatacaaaaggcacaggtttccggacaaggg |
| | gactcattgcacgaacatattgcgaatcttgctggttcgccagccatcaaaaagggcatac |
| | tccagacagtcaaagtagtggatgagctagttaaggtcatgggacgtcacaaaccggaaa |
| | acattgtaatcgagatggcacgcgaaaatcaaacgactcagaaggggcaaaaaaacagt |
| | cgagagcggatgaagagaatagaagagggtattaaagaactgggcagccagatcttaa |
| | aggagcatcctgtggaaaatacccaattgcagaacgagaaactttacctctattacctaca |
| | aaatggaagggacatgtatgttgatcaggaactggacataaaccgtttatctgattacgac |
| | gtcgatcacattgtaccccaatcctttttgaaggacgattcaatcgacaataaagtgcttac |
| | acgctcggataagaaccgagggaaaagtgacaatgttccaagcgaggaagtcgtaaag |
| | aaaatgaagaactattggcggcagctcctaaatgcgaaactgataacgcaaagaaagttc |
| | gataacttaactaaagctgagaggggtggcttgtctgaacttgacaaggccggatttatta |
| | aacgtcagctcgtggaaacccgccaaatcacaaagcatgttgcacagatactagattccc |
| | gaatgaatacgaaatacgacgagaacgataagctgattcgggaagtcaaagtaatcactt |
| | taaagtcaaaattggtgtcggacttcagaaaggattttcaattctataaagttagggagata |
| | aataactaccaccatgcgcacgacgcttatcttaatgccgtcgtagggaccgcactcatta |
| | agaaatacccgaagctagaaagtgagtttgtgtatggtgattacaaagtttatgacgtccgt |
| | aagat (SEQ ID NO: 42) |
| pDY0070 | accaacctgtctgacatcatcgagaaggagacaggcaagcagctggtcatccaggagag |
| Minicircle | catcctgatgctgcccgaagaagtcgaagaagtgatcggaaacaagcctgagagcgata |
| U6-sgRNA | tcctggtccataccgcctacgacgagagtaccgacgaaaatgtgatgctgctgacatccga |
| EFS- | cgccccagagtataagccctgggctctggtcatccaggattccaacggagagaacaaaat |
| AncBE4Max- | caaaatgctgtctggcggctcaaaaagaaccgccgacggcagcgaattcgagcccaaga |
| bGH | agaagaggaaagtcggaagcggaTAAgaattctaactagagctcgctgatcagcctcg |
| poly A | actgtgccttctagttgccagccatctgttgtttgcccctcccccgtgccttccttgaccctgg |
| (seq | aaggtgccactcccactgtcctttcctaataaaatgaggaaattgcatcgcattgtctgagt |
| XD7gRDHQ) | aggtgtcattctattctggggggtggggtggggcaggacagcaagggggaggattggga |
| U6 promoter: | agagaatagcaggcatgctggggagcctgaggcggaaagaaccagctgtggaatgtgtg |
| nucleotides | tcagttagggtgtggaaagtccccaggctccccagcaggcagaagtatgcaaagcatgca |
| 5021 to 5261 | tctcaattagtcagcaaccaggtgtggaaagtccccaggctccccagcaggcagaagtat |
| gRNA | gcaaagcatgcatctcaattagtcagcaaccatagtcccgcccctaactccgcccatcccg |
| scaffold: | cccctaactccgcccagttccgcccattctccgccccatggctgactaattttttttatttatgc |
| nucleotides | agaggccgaggccgcctcggcctctgagctattccagaagtagtgaggaggcttttttgga |
| 5288 to 5363 | ggcctaggcttttgcaaaaagcttgggcccgccccaactggggtaacctttgagttctctca |
| EFS-NS | gttgggggtaatcagcatcatgatgtggtaccacatcatgatgctgattataagaatgcgg |
| promoter: | ccgccacactctagtggatctcgagttaataattcagaagaactcgtcaagaaggcgatag |
| nucleotides | aaggcgatgcgctgcgaatcgggagcggcgataccgtaaagcacgaggaagcggtcag |
| 5394 to 5649 | cccattcgccgccaagctcttcagcaatatcacgggtagccaacgctatgtcctgatagcg |
| T7 promoter: | gtccgccacacccagccggccacagtcgatgaatccagaaaagcggccattttccaccat |
| nucleotides | gatattcggcaagcaggcatcgccatgggtcacgacgagatcctcgccgtcgggcatgct |
| 5660 to 5679 | cgccttgagcctggcgaacagttcggctggcgcgagcccctgatgctcttcgtccagatcat |
| Cas9(D10A): | cctgatcgacaagaccggcttccatccgagtacgtgctcgctcgatgcgatgtttcgcttgg |
| nucleotides | tggtcgaatgggcaggtagccggatcaagcgtatgcagccgccgcattgcatcagccatg |
| 4684 to 8862 | atggatactttctcggcaggagcaaggtgtagatgacatggagatcctgccccggcacttc |
| UGI element: | gcccaatagcagccagtcccttcccgcttcagtgacaacgtcgagcacagctgcgcaagg |
| Nucleotides | aacgcccgtcgtggccagccacgatagccgcgctgcctcgtcttgcagttcattcagggca |
| 10,660 to | ccggacaggtcggtcttgacaaaaagaaccgggcgcccctgcgctgacagccggaacac |
| 10,908 | ggcggcatcagagcagccgattgtctgttgtgcccagtcatagccgaatagcctctccacc |
| BGH polyA: | caagcggccggagaacctgcgtgcaatccatcttgttcaatcatgcgaaacgatcctcatc |
| nucleotides | ctgtctcttgatcagagcttgatcccctgcgccatcagatccttggcggcgagaaagccatc |
| 358 to 565 | cagtttactttgcagggcttcccaaccttaccagagggcgccccagctggcaattccggttc |
| | gcttgctgtccataaaaccgcccagtctagctatcgccatgtaagcccactgcaagctacct |
| | gctttctctttgcgcttgcgttttcccttgtccagatagcccagtagctgacattcatccgggg |
| | tcagcaccgtttctgcggactggctttctacgtgctcgaggggggccaaacggtctccagct |
| | tggctgttttggcggatgagagaagattttcagcctgatacagattaaatcagaacgcaga |
| | agcggtctgataaaacagaatttgcctggcggcagtagcgcggtggtcccacctgacccc |
| | atgccgaactcagaagtgaaacgccgtagcgccgatggtagtgtggggtctccccatgcg |
| | agagtagggaactgccaggcatcaaataaaacgaaaggctcagtcgaaagactgggcct |
| | ttcgttttatctgttgtttgtcggtgaacgctctcctgagtaggacaaatccgccgggagcgg |
| | atttgaacgttgcgaagcaacggcccggagggtggcgggcaggacgcccgccataaact |
| | gccaggcatcaaattaagcagaaggccatcctgacggatggcctttttgcgtttctacaaa |
| | ctcttttgtttatttttctaaatacattcaaatatgtatccgctcatgaccaaaatcccttaacg |
| | tgagttttcgttccactgagcgtcagaccccgtagaaaagatcaaaggatcttcttgagatc |
| | ctttttttctgcgcgtaatctgctgcttgcaaacaaaaaaaccaccgctaccagcggtggttt |
| | gtttgccggatcaagagctaccaactctttttccgaaggtaactggcttcagcagagcgca |
| | gataccaaatactgtccttctagtgtagccgtagttaggccaccacttcaagaactctgtag |
| | caccgcctacatacctcgctctgctaatcctgttaccagtggctgctgccagtggcgataag |
| | tcgtgtcttaccgggttggactcaagacgatagttaccggataaggcgcagcggtcgggct |
| | gaacggggggttcgtgcacacagcccagcttggagcgaacgacctacaccgaactgaga |
| | tacctacagcgtgagctatgagaaagcgccacgcttcccgaagggagaaaggcggacag |
| | gtatccggtaagcggcagggtcggaacaggagagcgcacgagggagcttccaggggga |
| | aacgcctggtatctttatagtcctgtcgggtttcgccacctctgacttgagcgtcgatttttgt |
| | gatgctcgtcaggggggcggagcctatggaaaaacgccagcaacgcggcctttttacggt |
| | tcctggccttttgctggccttttgctcacatgttctttcctgcgttatcccctgattctgtggata |
| | accgtattaccgcctttgagtgagctgataccgctcgccgcagccgaacgaccgagcgca |
| | gcgagtcagtgagcgaggaagcggaagagcgcctgatgcggtattttctccttacgcatct |
| | gtgcggtatttcacaccgcatatggtgcactctcagtacaatctgctctgatgccgcatagtt |
| | aagccagtatacactccgctatcgctacgtgactgggtcatggctgcgccccgacacccgc |
| | caacacccgctgacgcgccctgacgggcttgtctgctcccggcatccgcttacagacaagc |
| | tgtgaccgtctccgggagctgcatgtgtcagaggttttcaccgtcatcaccgaaacgcgcg |
| | aggcagcagatcaattcgcgcgcgaaggcgaagcggcatgcataatgtgcctgtcaaatg |
| | gacgaagcagggattctgcaaaccctatgctactccgtcaagccgtcaattgtctgattcgt |
| | taccaattatgacaacttgacggctacatcattcactttttcttcacaaccggcacggaactc |
| | gctcgggctggccccggtgcattttttaaatacccgcgagaaatagagttgatcgtcaaaa |
| | ccaacattgcgaccgacggtggcgataggcatccgggtggtgctcaaaagcagcttcgcc |
| | tggctgatacgttggtcctcgcgccagcttaagacgctaatccctaactgctggcggaaaa |
| | gatgtgacagacgcgacggcgacaagcaaacatgctgtgcgacgctggcgatacattac |
| | cctgttatccctagatgacattaccctgttatcccagatgacattaccctgttatccctagatg |
| | acattaccctgttatccctagatgacatttaccctgttatccctagatgacattaccctgttat |
| | cccagatgacattaccctgttatccctagatacattaccctgttatcccagatgacataccct |
| | gttatccctagatgacattaccctgttatcccagatgacattaccctgttatccctagatacat |
| | taccctgttatcccagatgacataccctgttatccctagatgacattaccctgttatcccagat |
| | gacattaccctgttatccctagatacattaccctgttatcccagatgacataccctgttatccc |
| | tagatgacattaccctgttatcccagatgacattaccctgttatccctagatacattaccctgt |
| | tatcccagatgacataccctgttatccctagatgacattaccctgttatcccagatgacatta |
| | ccctgttatccctagatacattaccctgttatcccagatgacataccctgttatccctagatga |
| | cattaccctgttatcccagatgacattaccctgttatccctagatacattaccctgttatccca |
| | gatgacataccctgttatccctagatgacattaccctgttatcccagataaactcaatgatg |
| | atgatgatgatggtcgagactcagcggccgcggtgccagggcgtgcccttgggctccccg |
| | ggcgcgactataagctgcgagcaacttcacttgggtatgccggcggtagcgctgagggcc |
| | tatttcccatgattccttcatatttgcatatacgatacaaggctgttagagagataattggaa |
| | ttaatttgactgtaaacacaaagatattagtacaaaatacgtgacgtagaaagtaataattt |
| | cttgggtagtttgcagttttaaaattatgttttaaaatggactatcatatgcttaccgtaacttg |
| | aaagtatttcgatttcttggctttatatatcttgtggaaaggacgaaacaccgggtcttcgag |
| | aagacctgttttagagctagaaatagcaagttaaaataaggctagtccgttatcaacttga |
| | aaaagtggcaccgagtcggtgcttttttatgtacgggccagatatacgcgtttaggtcttga |
| | aaggagtgggaattggctccggtgcccgtcagtgggcagagcgcacatcgcccacagtcc |
| | ccgagaagttggggggaggggtcggcaattgatccggtgcctagagaaggtggcgcggg |
| | gtaaactgggaaagtgatgtcgtgtactggctccgcctttttcccgagggtgggggagaac |
| | cgtatataagtgcagtagtcgccgtgaacgttctttttcgcaacgggtttgccgccagaaca |
| | caggccgcggccgctaatacgactcactatagggagagccgccaccatgaaacggacag |
| | ccgacggaagcgagttcgagtcaccaaagaagaagcggaaagtcagcagtgaaaccgg |
| | accagtggcagtggacccaaccctgaggagacggattgagccccatgaatttgaagtgtt |
| | ctttgacccaagggagctgaggaaggagacatgcctgctgtacgagatcaagtggggca |
| | caagccacaagatctggcgccacagctccaagaacaccacaaagcacgtggaagtgaat |
| | ttcatcgagaagtttacctccgagcggcacttctgcccctctaccagctgttccatcacatgg |
| | tttctgtcttggagcccttgcggcgagtgttccaaggccatcaccgagttcctgtctcagcac |
| | cctaacgtgaccctggtcatctacgtggcccggctgtatcaccacatggaccagcagaaca |
| | ggcagggcctgcgcgatctggtgaattctggcgtgaccatccagatcatgacagccccag |
| | agtacgactattgctggcggaacttcgtgaattatccacctggcaaggaggcacactggcc |
| | aagatacccacccctgtggatgaagctgtatgcactggagctgcacgcaggaatcctggg |
| | cctgcctccatgtctgaatatcctgcggagaaagcagccccagctgacatttttcaccattg |
| | ctctgcagtcttgtcactatcagcggctgcctcctcatattctgtgggctacaggcctgaagt |
| | ctggaggatctagcggaggatcctctggcagcgagacaccaggaacaagcgagtcagca |
| | acaccagagagcagtggcggcagcagcggcggcagcgacaagaagtacagcatcggcc |
| | tggccatcggcaccaactctgtgggctgggccgtgatcaccgacgagtacaaggtgccca |
| | gcaagaaattcaaggtgctgggcaacaccgaccggcacagcatcaagaagaacctgatc |
| | ggagccctgctgttcgacagcggcgaaacagccgaggccacccggctgaagagaaccgc |
| | cagaagaagatacaccagacggaagaaccggatctgctatctgcaagagatcttcagca |
| | acgagatggccaaggtggacgacagcttcttccacagactggaagagtccttcctggtgg |
| | aagaggataagaagcacgagcggcaccccatcttcggcaacatcgtggacgaggtggcc |
| | taccacgagaagtaccccaccatctaccacctgagaaagaaactggtggacagcaccga |
| | caaggccgacctgcggctgatctatctggccctggcccacatgatcaagttccggggccac |
| | ttcctgatcgagggcgacctgaaccccgacaacagcgacgtggacaagctgttcatccag |
| | ctggtgcagacctacaaccagctgttcgaggaaaaccccatcaacgccagcggcgtggac |
| | gccaaggccatcctgtctgccagactgagcaagagcagacggctggaaaatctgatcgcc |
| | cagctgcccggcgagaagaagaatggcctgttcggaaacctgattgccctgagcctgggc |
| | ctgacccccaacttcaagagcaacttcgacctggccgaggatgccaaactgcagctgagc |
| | aaggacacctacgacgacgacctggacaacctgctggcccagatcggcgaccagtacgc |
| | cgacctgtttctggccgccaagaacctgtccgacgccatcctgctgagcgacatcctgaga |
| | gtgaacaccgagatcaccaaggcccccctgagcgcctctatgatcaagagatacgacga |
| | gcaccaccaggacctgaccctgctgaaagctctcgtgcggcagcagctgcctgagaagta |
| | caaagagattttcttcgaccagagcaagaacggctacgccggctacattgacggcggagc |
| | cagccaggaagagttctacaagttcatcaagcccatcctggaaaagatggacggcaccg |
| | aggaactgctcgtgaagctgaacagagaggacctgctgcggaagcagcggaccttcgac |
| | aacggcagcatcccccaccagatccacctgggagagctgcacgccattctgcggcggcag |
| | gaagatttttacccattcctgaaggacaaccgggaaaagatcgagaagatcctgaccttcc |
| | gcatcccctactacgtgggccctctggccaggggaaacagcagattcgcctggatgacca |
| | gaaagagcgaggaaaccatcaccccctggaacttcgaggaagtggtggacaagggcgct |
| | tccgcccagagcttcatcgagcggatgaccaacttcgataagaacctgcccaacgagaag |
| | gtgctgcccaagcacagcctgctgtacgagtacttcaccgtgtataacgagctgaccaaag |
| | tgaaatacgtgaccgagggaatgagaaagcccgccttcctgagcggcgagcagaaaaa |
| | ggccatcgtggacctgctgttcaagaccaaccggaaagtgaccgtgaagcagctgaaag |
| | aggactacttcaagaaaatcgagtgcttcgactccgtggaaatctccggcgtggaagatcg |
| | gttcaacgcctccctgggcacataccacgatctgctgaaaattatcaaggacaaggacttc |
| | ctggacaatgaggaaaacgaggacattctggaagatatcgtgctgaccctgacactgtttg |
| | aggacagagagatgatcgaggaacggctgaaaacctatgcccacctgttcgacgacaaa |
| | gtgatgaagcagctgaagcggcggagatacaccggctggggcaggctgagccggaagc |
| | tgatcaacggcatccgggacaagcagtccggcaagacaatcctggatttcctgaagtccg |
| | acggcttcgccaacagaaacttcatgcagctgatccacgacgacagcctgacctttaaag |
| | aggacatccagaaagcccaggtgtccggccagggcgatagcctgcacgagcacattgcc |
| | aatctggccggcagccccgccattaagaagggcatcctgcagacagtgaaggtggtgga |
| | cgagctcgtgaaagtgatgggccggcacaagcccgagaacatcgtgatcgaaatggcca |
| | gagagaaccagaccacccagaagggacagaagaacagccgcgagagaatgaagcgg |
| | atcgaagagggcatcaaagagctgggcagccagatcctgaaagaacaccccgtggaaa |
| | acacccagctgcagaacgagaagctgtacctgtactacctgcagaatgggcgggatatgt |
| | acgtggaccaggaactggacatcaaccggctgtccgactacgatgtggaccatatcgtgc |
| | ctcagagctttctgaaggacgactccatcgacaacaaggtgctgaccagaagcgacaag |
| | aaccggggcaagagcgacaacgtgccctccgaagaggtcgtgaagaagatgaagaact |
| | actggcggcagctgctgaacgccaagctgattacccagagaaagttcgacaatctgacca |
| | aggccgagagaggcggcctgagcgaactggataaggccggcttcatcaagagacagctg |
| | gtggaaacccggcagatcacaaagcacgtggcacagatcctggactcccggatgaacac |
| | taagtacgacgagaatgacaagctgatccgggaagtgaaagtgatcaccctgaagtcca |
| | agctggtgtccgatttccggaaggatttccagttttacaaagtgcgcgagatcaacaactac |
| | caccacgcccacgacgcctacctaaacgccgtcgtgggaaccgccctgatcaaaaagtac |
| | cctaagctggaaagcgagttcgtgtacggcgactacaaggtgtacgacgtgcggaagatg |
| | atcgccaagagcgagcaggaaatcggcaaggctaccgccaagtacttcttctacagcaac |
| | atcatgaactttttcaagaccgagattaccctggccaacggcgagatccggaagcggcctc |
| | tgatcgagacaaacggcgaaaccggggagatcgtgtgggataagggccgggattttgcc |
| | accgtgcggaaagtgctgagcatgccccaagtgaatatcgtgaaaaagaccgaggtgca |
| | gacaggcggcttcagcaaagagtctatcctgcccaagaggaacagcgataagctgatcg |
| | ccagaaagaaggactgggaccctaagaagtacggcggcttcgacagccccaccgtggcc |
| | tattctgtgctggtggtggccaaagtggaaaagggcaagtccaagaaactgaagagtgtg |
| | aaagagctgctggggatcaccatcatggaaagaagcagcttcgagaagaatcccatcga |
| | ctttctggaagccaagggctacaaagaagtgaaaaaggacctgatcatcaagctgcctaa |
| | gtactccctgttcgagctggaaaacggccggaagagaatgctggcctctgccggcgaact |
| | gcagaagggaaacgaactggccctgccctccaaatatgtgaacttcctgtacctggccag |
| | ccactatgagaagctgaagggctcccccgaggataatgagcagaaacagctgtttgtgga |
| | acagcacaagcactacctggacgagatcatcgagcagatcagcgagttctccaagagag |
| | tgatcctggccgacgctaatctggacaaagtgctgtccgcctacaacaagcaccgggata |
| | agcccatcagagagcaggccgagaatatcatccacctgtttaccctgaccaatctgggag |
| | cccctgccgccttcaagtactttgacaccaccatcgaccggaagaggtacaccagcacca |
| | aagaggtgctggacgccaccctgatccaccagagcatcaccggcctgtacgagacacgg |
| | atcgacctgtctcagctgggaggtgacagcggcgggagcggcgggagcggggggagca |
| | ctaatctgagcgacatcattgagaaggagactgggaaacagctggtcattcaggagtcca |
| | tcctgatgctgcctgaggaggtggaggaagtgatcggcaacaagccagagtctgacatcc |
| | tggtgcacaccgcctacgacgagtccacagatgagaatgtgatgctgctgacctctgacgc |
| | ccccgagtataagccttgggccctggtcatccaggattctaacggcgagaataagatcaa |
| | gatgctgagcggaggatccggaggatctggaggcagc (SEQ ID NO: 43) |
| pDY0110 | ccagtggcgataagtcgtgtcttaccgggttggactcaagacgatagttaccggataaggc |
| pVITRO- | gcagcggtcgggctgaacggggggttcgtgcacacagcccagcttggagcgaacgacct |
| HPV39 L1L2 | acaccgaactgagatacctacagcgtgagctatgagaaagcgccacgcttcccgaaggg |
| (seq | agaaaggcggacaggtatccggtaagcggcagggtcggaacaggagagcgcacgagg |
| mnAcZxCM) | gagcttccagggggaaacgcctggtatctttatagtcctgtcgggtttcgccacctctgactt |
| CMV | gagcgtcgatttttgtgatgctcgtcaggggggcggagcctatggaaaaacgccagcaac |
| enhancer: | gcggcctttttacggttcctggccttttgctggccttttgctcacatgttcttaattaacctgca |
| nucleotides | ggcgttacataacttacggtaaatggcccgcctggctgaccgcccaacgacccccgcccat |
| 427 to 730 | tgacgtcaataatgacgtatgttcccatagtaacgccaatagggactttccattgacgtcaa |
| HPV-39 L2 | tgggtggagtatttacggtaaactgcccacttggcagtacatcaagtgtatcatatgccaag |
| coding | tacgccccctattgacgtcaatgacggtaaatggcccgcctggcattatgcccagtacatg |
| sequence: | accttatgggactttcctacttggcagtacatctacgtattagtcatcgctattaccatgatga |
| nucleotides | tgcggttttggcagtacatcaatgggcgtggatagcggtttgactcacggggatttccaagt |
| 2175 to 3587 | ctccaccccattgacgtcaatgggagtttgttttgactagtggagccgagagtaattcatac |
| FMDV IRES: | aaaaggagggatcgccttcgcaaggggagagcccagggaccgtccctaaattctcacag |
| nucleotides | acccaaatccctgtagccgccccacgacagcgcgaggagcatgcgcccagggctgagcg |
| 3597 to 4041 | cgggtagatcagagcacacaagctcacagtccccggcggtggggggaggggcgcgctg |
| EM7 | agcgggggccagggagctggcgcggggcaaactgggaaagtggtgtcgtgtgctggctc |
| promoter: | cgccctcttcccgagggtgggggagaacggtatataagtgcggtagtcgccttggacgttc |
| nucleotides | tttttcgcaacgggtttgccgtcagaacgcaggtgagtggcgggtgtggcttccgcgggcc |
| 4074 to 4120 | ccggagctggagccctgctctgagcgggccgggctgatatgcgagtgtcgtccgcagggtt |
| T7 promoter: | tagctgtgagcattcccacttcgagtggcgggcggtgcgggggtgagagtgcgaggccta |
| nucleotides | gcggcaaccccgtagcctcgcctcgtgtccggcttgaggcctagcgtggtgtccgccgccg |
| 4112 to 4130 | cgtgccactccggccgcactatgcgttttttgtccttgctgccctcgattgccttccagcagca |
| EF-1-alpha | tgggctaacaaagggagggtgtggggctcactcttaaggagcccatgaagcttacgttgg |
| polyA: | ataggaatggaagggcaggaggggcgactggggcccgcccgccttcggagcacatgtcc |
| nucleotides | gacgccacctggatggggcgaggcctgtggctttccgaagcaatcgggcgtgagtttagc |
| 4981 to 5553 | ctacctgggccatgtggccctagcactgggcacggtctggcctggcggtgccgcgttccctt |
| mEF-1-alpha | gcctcccaacaagggtgaggccgtcccgcccggcaccagttgcttgcgcggaaagatggc |
| intron: | cgctcccggggccctgttgcaaggagctcaaaatggaggacgcggcagcccggtggagc |
| nucleotides | gggcgggtgagtcacccacacaaaggaagagggccttgcccctcgccggccgctgcttcc |
| 6137 to 7084 | tgtgaccccgtggtctatcggccgcatagtcacctcgggcttctcttgagcaccgctcgtcgc |
| HPV39 L1 | ggcggggggaggggatctaatggcgttggagtttgttcacatttggtgggtggagactagt |
| coding | caggccagcctggcgctggaagtcattcttggaatttgcccctttgagtttggagcgaggct |
| sequence: | aattctcaagcctcttagcggttcaaaggtattttctaaacccgtttccaggtgttgtgaaag |
| nucleotides | ccaccgctaattcaaagcaatccggagtatacggatccgccaccatggtgtcccacagag |
| 7142 to 8659 | ccgccagacggaagcgggccagcgccaccgacctgtatcggacctgtaagcagagcggc |
| SV40 polyA | acctgcccccctgatgtggtcgacaaggtggagggcaccacactggccgacaagatcctg |
| signal: | cagtggaccagcctgggcatcttcctgggcggcctgggcattggcaccggcacaggcacc |
| nucleotides | ggcggcagaaccggctacatccccctcggcggcagacccaacaccgtggtggacgtgtcc |
| 8682 to 8803 | cccgccagaccccccgtggtcatcgagcccgtgggccccagcgagcccagcatcgtgcag |
| | ctggtcgaggacagcagcgtgatcaccagcggcacccccgtgcccaccttcaccggcacc |
| | agcggcttcgagattacctctagctccaccaccacccctgccgtgctggacatcaccccca |
| | gcagcggcagcgtgcagatcacctccacctcctacaccaaccccgccttcacagacccaa |
| | gcctgatcgaggtgccccagaccggcgagacaagcggcaacatcttcgtgagcaccccc |
| | acctccggcacacacggatacgaggaaatccccatggaagtgttcgccacccacggcacc |
| | gggaccgagcccatcagcagcacccctacccctggcatctctcgggtggcaggacctcgg |
| | ctgtactctagggctcaccagcaggtccgggtgtccaacttcgacttcgtgacccaccccag |
| | cagcttcgtgaccttcgacaaccctgccttcgagcctgtggacaccaccctgacctacgag |
| | gccgccgatatcgcccccgaccccgacttcctggacatcgtgcggctgcacagacccgccc |
| | tgaccagccggaagggcaccgtgcggttctctcggctcggcaagaaagccacaatggtca |
| | ccagacggggcacccagatcggcgcccaggtgcactactaccacgacatcagctctatcg |
| | cccctgccgagagcatcgagctgcagcccctggtgcacgccgagcccagcgacgcctccg |
| | acgccctgttcgacatctacgccgacgtggacaacaacacctacctggacaccgccttcaa |
| | caacacccgggacagcggcaccacctacaacaccggcagcctccccagcgtggccagca |
| | gcgccagcaccaagtacgccaacaccaccatccctttcagcaccagctggaacatgcccg |
| | tgaacaccggccctgatatcgctctgcccagcaccaccccccagctgcctctggtgcccag |
| | cggcccaatcgacacaacctacgccatcaccatccagggcagcaactactacctgctgcc |
| | cctgctgtacttcttcctgaagaagcggaagagaatcccctacttcttcagcgacggctacg |
| | tggccgtgtgatagtctaggagcaggtttccccaatgacacaaaacgtgcaacttgaaact |
| | ccgcctggtctttccaggtctagaggggtaacactttgtactgcgtttggctccacgctcgat |
| | ccactggcgagtgttagtaacagcactgttgcttcgtagcggagcatgacggccgtgggaa |
| | ctcctccttggtaacaaggacccacggggccaaaagccacgcccacacgggcccgtcatg |
| | tgtgcaaccccagcacggcgactttactgcgaaacccactttaaagtgacattgaaactgg |
| | tacccacacactggtgacaggctaaggatgcccttcaggtaccccgaggtaacacgcgac |
| | actcgggatctgagaaggggactggggcttctataaaagcgctcggtttaaaaagcttcta |
| | tgcctgaataggtgaccggaggtcggcacctttcctttgcaattactgaccctatgaataca |
| | ctgactgtttgacaattaatcatcggcatagtatatcggcatagtataatacgactcactata |
| | ggagggccaccatgattgaacaagatggattgcacgcaggttctccggccgcttgggtgg |
| | agaggctattcggctatgactgggcacaacagacaatcggctgctctgatgccgccgtgtt |
| | ccggctgtcagcgcaggggcgcccggttctttttgtcaagaccgacctgtccggtgccctga |
| | atgaactgcaagacgaggcagcgcggctatcgtggctggccacgacgggcgttccttgcg |
| | cagctgtgctcgacgttgtcactgaagcgggaagggactggctgctattgggcgaagtgcc |
| | ggggcaggatctcctgtcatctcaccttgctcctgccgagaaagtatccatcatggctgatg |
| | caatgcggcggctgcatacgcttgatccggctacctgcccattcgaccaccaagcgaaac |
| | atcgcatcgagcgagcacgtactcggatggaagccggtcttgtcgatcaggatgatctgga |
| | cgaagagcatcaggggctcgcgccagccgaactgttcgccaggctcaaggcgagcatgc |
| | ccgacggcgaggatctcgtcgtgacacatggcgatgcctgcttgccgaatatcatggtgga |
| | aaatggccgcttttctggattcatcgactgtggccggctgggtgtggcggaccgctatcagg |
| | acatagcgttggctacccgtgatattgctgaagagcttggcggcgaatgggctgaccgctt |
| | cctcgtgctttacggtatcgccgctcccgattcgcagcgcatcgccttctatcgccttcttgac |
| | gagttcttctgagcgggactctggggttcgaaatgaccgaccaagcgaattcgctaggatt |
| | atccctaatacctgccaccccactcttaatcagtggtggaagaacggtctcagaactgtttg |
| | tttcaattggccatttaagtttagtagtaaaagactggttaatgataacaatgcatcgtaaa |
| | accttcagaaggaaaggagaatgttttgtggaccactttggttttcttttttgcgtgtggcagt |
| | tttaagttattagtttttaaaatcagtactttttaatggaaacaacttgaccaaaaatttgtca |
| | cagaattttgagacccattaaaaaagttaaatgagaaacctgtgtgttcctttggtcaacac |
| | cgagacatttaggtgaaagacatctaattctggttttacgaatctggaaacttcttgaaaat |
| | gtaattcttgagttaacacttctgggtggagaatagggttgttttccccccacataattggaa |
| | ggggaaggaatatcatttaaagctatgggagggttgctttgattacaacactggagagaa |
| | atgcagcatgttgctgattgcctgtcactaaaacaggccaaaaactgagtccttgggttgca |
| | tagaaagctgcctgcagggcctgaaataacctctgaaagaggaacttggttaggtaccttc |
| | tgaggcggaaagaaccagctgtggaatgtgtgtcagttagggtgtggaaagtccccaggc |
| | tccccagcaggcagaagtatgcaaagcatgcatctcaattagtcagcaaccaggtgtgga |
| | aagtccccaggctccccagcaggcagaagtatgcaaagcatgcatctcaattagtcagca |
| | accatagtcccactagtggagccgagagtaattcatacaaaaggagggatcgccttcgca |
| | aggggagagcccagggaccgtccctaaattctcacagacccaaatccctgtagccgcccc |
| | acgacagcgcgaggagcatgcgctcagggctgagcgcggggagagcagagcacacaa |
| | gctcatagaccctggtcgtgggggggaggaccggggagctggcgcggggcaaactggg |
| | aaagcggtgtcgtgtgctggctccgccctcttcccgagggtgggggagaacggtatataag |
| | tgcggcagtcgccttggacgttctttttcgcaacgggtttgccgtcagaacgcaggtgaggg |
| | gcgggtgtggcttccgcgggccgccgagctggaggtcctgctccgagcgggccgggcccc |
| | gctgtcgtcggcggggattagctgcgagcattcccgcttcgagttgcgggcggcgcggga |
| | ggcagagtgcgaggcctagcggcaaccccgtagcctcgcctcgtgtccggcttgaggcct |
| | agcgtggtgtccgcgccgccgccgcgtgctactccggccgcactctggtcttttttttttttgtt |
| | gttgttgccctgctgccttcgattgccgttcagcaataggggctaacaaagggagggtgcg |
| | gggcttgctcgcccggagcccggagaggtcatggttggggaggaatggagggacaggag |
| | tggcggctggggcccgcccgccttcggagcacatgtccgacgccacctggatggggcgag |
| | gcctggggtttttcccgaagcaaccaggctggggttagcgtgccgaggccatgtggcccca |
| | gcacccggcacgatctggcttggcggcgccgcgttgccctgcctccctaactagggtgagg |
| | ccatcccgtccggcaccagttgcgtgcgtggaaagatggccgctcccgggccctgttgcaa |
| | ggagctcaaaatggaggacgcggcagcccggtggagcgggcgggtgagtcacccacac |
| | aaaggaagagggcctggtccctcaccggctgctgcttcctgtgaccccgtggtcctatcgg |
| | ccgcaatagtcacctcgggcttttgagcacggctagtcgcggcggggggaggggatgtaa |
| | tggcgttggagtttgttcacatttggtgggtggagactagtcaggccagcctggcgctggaa |
| | gtcatttttggaatttgtccccttgagttttgagcggagctaattctcgggcttcttagcggttc |
| | aaaggtatcttttaaacccttttttaggtgttgtgaaaaccaccgctaattcaaagcaaccg |
| | gtgatatcaaagatccgccaccatggcaatgtggagaagcagcgacagcatggtgtacct |
| | gccccctcccagcgtggccaaggtggtcaacaccgacgactacgtgacccggaccggcat |
| | ctactactacgccggcagctctcggctgctgaccgtgggccacccctacttcaaagtgggc |
| | atgaacggcggcagaaagcaggacatccccaaggtgtccgcctaccagtaccgggtgttc |
| | agagtgaccctgcccgaccccaacaagttcagcatccccgacgccagcctgtacaacccc |
| | gagacacagcggctggtctgggcctgcgtgggcgtggaagtgggcagaggccagcccct |
| | gggcgtgggcatcagcggccaccccctgtacaacagacaggacgacaccgagaacagc |
| | cccttcagcagcaccaccaacaaggacagccgggacaacgtgtccgtggactacaagca |
| | gacccagctgtgcatcatcggctgcgtgcctgccattggcgagcactggggcaagggcaa |
| | ggcctgcaagcccaacaatgtgtccaccggcgactgcccccctctggaactggtcaacac |
| | acccatcgaggacggcgacatgatcgacaccggctacggcgccatggacttcggcgccct |
| | gcaggaaaccaagagcgaggtccccctggacatctgccagagcatctgcaagtaccccg |
| | actacctgcagatgagcgccgacgtgtacggcgactccatgttcttttgcctgcggcggga |
| | gcagctgttcgcccggcacttctggaacagaggcggcatggtcggcgacgctatccctgcc |
| | cagctgtatatcaagggcaccgacatcagagccaaccccggcagctccgtgtactgcccc |
| | agccccagcggctccatggtcaccagcgacagccagctgttcaacaagccctactggctg |
| | cacaaggcccagggccacaacaacggcatctgctggcacaaccagctgtttctgaccgtg |
| | gtggacaccaccagaagcaccaacttcaccctgagcaccagcatcgagagcagcatccc |
| | cagcacctacgacccctccaagttcaaagagtacacccggcacgtcgaggaatacgacct |
| | gcagttcatcttccagctgtgtaccgtgaccctgaccaccgacgtgatgagctacatccaca |
| | ccatgaacagcagcatcctggacaactggaacttcgccgtggcccctccccctagcgcca |
| | gcctggtggatacctacagatacctgcagagcgccgccatcacctgccagaaggacgccc |
| | ctgcccccgagaagaaggacccctacgacggcctgaagttctggaacgtggacctgcgg |
| | gagaagttcagcctggaactcgaccagtttcccctgggccggaagttcctgctgcaagcca |
| | gagtcagacggaggcccaccatcggccccagaaagcggcctgccgctagcacctctagc |
| | agctccgccaccaagcacaagcggaagcgggtgtccaagtgatagtctagctggccaga |
| | catgataagatacattgatgagtttggacaaaccacaactagaatgcagtgaaaaaaatg |
| | ctttatttgtgaaatttgtgatgctattgctttatttgtaaccattataagctgcaataaacaag |
| | ttaacaacaacaattgcattcattttatgtttcaggttcagggggaggtgtgggaggtttttt |
| | aaagcaagtaaaacctctacaaatgtggtatggaaatgttaattaactagccatgaccaa |
| | aatcccttaacgtgagttttcgttccactgagcgtcagaccccgtagaaaagatcaaagga |
| | tcttcttgagatcctttttttctgcgcgtaatctgctgcttgcaaacaaaaaaaccaccgctac |
| | cagcggtggtttgtttgccggatcaagagctaccaactctttttccgaaggtaactggcttca |
| | gcagagcgcagataccaaatactgttcttctagtgtagccgtagttaggccaccacttcaa |
| | gaactctgtagcaccgcctacatacctcgctctgctaatcctgttaccagtggctgctg |
| | (SEQ ID NO: 44) |
| HPV-39 L1 | MAMWRSSDSMVYLPPPSVAKVVNTDDYVTRTGIYYYAGS |
| amino acids | SRLLTVGHPYFKVGMNGGRKQDIPKVSAYQYRVFRVTLP |
| | DPNKFSIPDASLYNPETQRLVWACVGVEVGRGQPLGVGIS |
| | GHPLYNRQDDTENSPFSSTTNKDSRDNVSVDYKQTQLCII |
| | GCVPAIGEHWGKGKACKPNNVSTGDCPPLELVNTPIEDG |
| | DMIDTGYGAMDFGALQETKSEVPLDICQSICKYPDYLQM |
| | SADVYGDSMFFCLRREQLFARHFWNRGGMVGDAIPAQL |
| | YIKGTDIRANPGSSVYCPSPSGSMVTSDSQLFNKPYWLHK |
| | AQGHNNGICWHNQLFLTVVDTTRSTNFTLSTSIESSIPSTY |
| | DPSKFKEYTRHVEEYDLQFIFQLCTVTLTTDVMSYIHTMN |
| | SSILDNWNFAVAPPPSASLVDTYRYLQSAAITCQKDAPAPE |
| | KKDPYDGLKFWNVDLREKFSLELDQFPLGRKFLLQARV |
| | RRRPTIGPRKRPAASTSSSSATKHKRKRVSK |
| | (SEQ ID NO: 45) |
| HPV-39 L2 | MVSHRAARRKRASATDLYRTCKQSGTCPPDVVDKVEGT |
| amino acids | TLADKILQWTSLGIFLGGLGIGTGTGTGGRTGYIPLGGRP |
| | NTVVDVSPARPPVVIEPVGPSEPSIVQLVEDSSVITSGTPVP |
| | TFTGTSGFEITSSSTTTPAVLDITPSSGSVQITSTSYTNPAFT |
| | DPSLIEVPQTGETSGNIFVSTPTSGTHGYEEIPMEVFATHG |
| | TGTEPISSTPTPGISRVAGPRLYSRAHQQVRVSNFDFVTHP |
| | SSFVTFDNPAFEPVDTTLTYEAADIAPDPDFLDIVRLHRPA |
| | LTSRKGTVRFSRLGKKATMVTRRGTQIGAQVHYYHDISSI |
| | APAESIELQPLVHAEPSDASDALFDIYADVDNNTYLDTAFN |
| | NTRDSGTTYNTGSLPSVASSASTKYANTTIPFSTSWNMPV |
| | NTGPDIALPSTTPQLPLVPSGPIDTTYAITIQGSNYYLLPLL |
| | YFFLKKRKRIPYFFSDGYVAV (SEQ ID NO: 46) |
| pDY0111 | aaggatcttaccgctgttgagatccagttcgatgtaacccactcgtgcacccaactgatctt |
| p45sheLL | cagcatcttttactttcaccagcgtttctgggtgagcaaaaacaggaaggcaaaatgccgc |
| (seq | aaaaaagggaataagggcgacacggaaatgttgaatactcatactcttcctttttcaatatt |
| IpPNYOUs) | attgaagcatttatcagggttattgtctcatgagcggatacatatttgaatgtatttagaaaa |
| CMV | ataaacaaataggggttccgcgcacatttccccgaaaagtgccacctgacgtcgacggat |
| enhancer: | cgggagatctcccgatcccctatggtgcactctcagtacaatctgctctgatgccgcatagtt |
| nucleotides | aagccagtatctgctccctgcttgtgtgttggaggtcgctgagtagtgcgcgagcaaaattt |
| 536 to 915 | aagctacaacaaggcaaggcttgaccgacaattgcatgaagaatctgcttagggttaggc |
| CMV | gttttgcgctgcttcgcgatgtacgggccagatatacgcgttgacattgattattgactagtt |
| promoter: | attaatagtaatcaattacggggtcattagttcatagcccatatatggagttccgcgttacat |
| nucleotides | aacttacggtaaatggcccgcctggctgaccgcccaacgacccccgcccattgacgtcaat |
| 916 to 1119 | aatgacgtatgttcccatagtaacgccaatagggactttccattgacgtcaatgggtggagt |
| HPV-45 L1 | atttacggtaaactgcccacttggcagtacatcaagtgtatcatatgccaagtacgccccct |
| coding | attgacgtcaatgacggtaaatggcccgcctggcattatgcccagtacatgaccttatggg |
| sequence: | actttcctacttggcagtacatctacgtattagtcatcgctattaccatggtgatgcggttttg |
| nucleotides | gcagtacatcaatgggcgtggatagcggtttgactcacggggatttccaagtctccacccc |
| 1280 to 2821 | attgacgtcaatgggagtttgttttggaaccaaaatcaacgggactttccaaaatgtcgtaa |
| HPV-45 L2 | caactccgccccattgacgcaaatgggcggtaggcgtgtacggtgggaggtctatataag |
| coding | cagagctctccctatcagtgatagagatctccctatcagtgatagagatcgtcgacgagctc |
| sequence: | gtttagtgaaccgtcagatcgcctggagacgccatccacgctgttttgacctccatagaaga |
| nucleotides | caccgggaccgatccagcctccgggggatccactagagccaccatggccctctggagacc |
| 3521 to 4912 | ctccgattccaccgtgtacttgcccccccccagcgtcgcacgcgtcgtgtctaccgacgact |
| WPRE | acgtcagcaggacctcaatcttctaccacgccgggtccagtaggctgctgaccgtgggga |
| element: | acccctacttccgcgtcgtgcccaacggcgccggcaacaagcaagccgtccccaaagtca |
| nucleotides | gtgcctaccagtaccgcgtcttccgcgtggccctgccagaccccaacaagttcggcctgcc |
| 50006 to | cgacagcaccatctacaaccccgagacccagaggctcgtctgggcctgcgtgggcatgga |
| 5594 | gatcggcaggggccaacccctgggcatcgggttgtccgggcaccccttctacaacaagct |
| BGH polvA: | cgacgacaccgagtccgcccacgccgccaccgccgtcatcacccaggacgtccgcgaca |
| nucleotides | acgtcagcgtcgactacaaacagacccaactctgcatcctgggctgcgtgcccgccatcgg |
| 5637 to 5861 | cgaacattgggcaaaggggaccttgtgcaagcccgcccagctccagcccggcgattgccc |
| | ccccctcgagttgaagaatacaatcatcgaggacggcgacatggtcgacaccggctacgg |
| | cgccatggacttctccaccctccaagacaccaaatgtgaagtccccctggatatctgccag |
| | agtatttgcaagtaccccgactacctccagatgagcgccgacccatacggcgacagcatgt |
| | tcttctgtttgaggagggagcagctcttcgcccgccacttctggaaccgcgccggcgtcatg |
| | ggcgataccgtgcccaccgatttgtacatcaaggggacctcagccaacatgagggagaca |
| | ccggggtcctgcgtctacagtcccagcccatccgggagcatcatcaccagcgacagccag |
| | ctgttcaacaagccctactggctgcacaaagcacaggggcacaataacggcatctgctgg |
| | cacaaccaactcttcgtcaccgtggtcgataccacaaggtccaccaacctgaccctgtgcg |
| | caagcacccagaaccccgtcccctccacctacgatcccaccaagttcaaacagtactcccg |
| | ccacgtcgaagagtacgacctgcagttcatcttccaactctgtaccatcaccctgaccgccg |
| | aggtcatgagctacattcactccatgaactcctccatcctggagaactggaacttcggcgtg |
| | ccccccccccccaccacctccctcgtcgacacctacaggttcgtccagagcgtcgccgtca |
| | catgccagaaggacaccaccccccccgagaaacaggacccctacgacaagctgaagttc |
| | tggaccgtcgatttgaaggagaagttcagtagtgacctcgaccagtacccattgggcagg |
| | aaattcctggtccaagccggcctgaggaggcgccccacaatcggccccaggaagaggcc |
| | cgccgccagtaccagcaccgccagcaccgccagccgccccgcaaagcgcgtcaggatca |
| | ggtccaagaaatgagcccggtggatcccaatcaagctttttgcaaaagcctagggctcga |
| | ggaagcttaaaacagctctggggttgtacccaccccagaggcccacgtggcggctagtac |
| | tccggtattgcggtacccttgtacgcctgttttatactcccttcccgtaacttagacgcacaaa |
| | accaagttcaatagaagggggtacaaaccagtaccaccacgaacaagcacttctgtttcc |
| | ccggtgatgtcgtatagactgcttgcgtggttgaaagcgacggatccgttatccgcttatgt |
| | acttcgagaagcccagtaccacctcggaatcttcgatgcgttgcgctcagcactcaacccc |
| | agagtgtagcttaggctgatgagtctggacatccctcaccggtgacggtggtccaggctgc |
| | gttggcggcctacctatggctaacgccatgggacgctagttgtgaacaaggtgtgaagag |
| | cctattgagctacataagaatcctccggcccctgaatgcggctaatcccaacctcggagca |
| | ggtggtcacaaaccagtgattggcctgtcgtaacgcgcaagtccgtggcggaaccgacta |
| | ctttgggtgtccgtgtttccttttattttattgtggctgcttatggtgacaatcacagattgttat |
| | cataaagcgaattggattgcggccgctctagagccaccatggtcagtcatagggccgcca |
| | ggaggaagagagcaagcgccaccgatctgtaccgcacctgcaaacagagtggcacctgt |
| | ccacccgacgtcatcaataaggtcgaggggaccacactggccgacaagatcctgcaatg |
| | gagctcattgggcatcttcctcggcgggttggggatcggcacagggtccggcagcggcgg |
| | gaggaccggatacgtgccactgggcgggcgcagcaacaccgtcgtcgacgtcgggccaa |
| | cccgcccccccgtcgtcatcgagcccgtgggccccaccgaccccagcatcgtcaccctcgt |
| | ggaagacagttccgtcgtcgcaagcggcgcccccgtcccaaccttcaccggcacaagcgg |
| | cttcgagatcaccagcagcggcaccacaacccccgccgtcctcgatattacccccaccgtc |
| | gatagcgtcagcatcagcagcacctccttcaccaacccagccttcagcgacccaagcatc |
| | atcgaggtcccacagaccggcgaagtcagcggcaacatcttcgtcggcacccccaccagc |
| | gggtctcacggctacgaagagatcccactgcagaccttcgccagcagcggcagcggcac |
| | cgagccaatctcctccacaccattgcccaccgtcagaagagtggccggcccaaggctcta |
| | ctcccgcgccaaccagcaagtcagggtcagtacaagccagttcctgacccacccaagcag |
| | cctcgtcaccttcgacaaccccgcctacgagccactcgatacaaccttgagtttcgaaccca |
| | catccaacgtccccgacagtgacttcatggacatcatcaggctccaccgccccgccctgag |
| | tagccgcagggggaccgtccgcttctcccgcctcggccagcgcgccacaatgttcaccag |
| | gtccggcaagcagatcggcggccgcgtgcacttctatcacgacatctctccaatcgccgcc |
| | accgaagagatcgagctccaacccctgatctccgccaccaacgactccgatctcttcgacg |
| | tgtacgccgattttccgccacccgccagtaccaccccctcaaccatccataagagcttcacc |
| | taccccaaatacagtctcacaatgcccagcaccgccgccagtagctattccaacgtcaccg |
| | tgcccctgaccagcgcctgggacgtgcccatctacaccgggcccgatatcatcctcccgag |
| | tcacacccccatgtggccctccaccagccccacaaacgccagtacaacaacatacatcgg |
| | catccacgggacccagtactacctgtggccctggtactactacttccccaagaagaggaag |
| | aggatcccatacttcttcgccgacgggttcgtcgccgcatgagcccgggacccagctttctt |
| | gtacaaagtggttcgatctagaatggctagtggatcccccgggctgcaggaattcgatatc |
| | aagcttatcgataatcaacctctggattacaaaatttgtgaaagattgactggtattcttaac |
| | tatgttgctccttttacgctatgtggatacgctgctttaatgcctttgtatcatgctattgcttcc |
| | cgtatggctttcattttctcctccttgtataaatcctggttgctgtctctttatgaggagttgtgg |
| | cccgttgtcaggcaacgtggcgtggtgtgcactgtgtttgctgacgcaacccccactggttg |
| | gggcattgccaccacctgtcagctcctttccgggactttcgctttccccctccctattgccacg |
| | gcggaactcatcgccgcctgccttgcccgctgctggacaggggctcggctgttgggcactg |
| | acaattccgtggtgttgtcggggaaatcatcgtcctttccttggctgctcgcctgtgttgccac |
| | ctggattctgcgcgggacgtccttctgctacgtcccttcggccctcaatccagcggaccttcc |
| | ttcccgcggcctgctgccggctctgcggcctcttccgcgtcttcgccttcgccctcagacgag |
| | tcggatctccctttgggccgcctccccgcatcgataccgtcggcccgtttaaacccgctgatc |
| | agcctcgactgtgccttctagttgccagccatctgttgtttgcccctcccccgtgccttccttg |
| | accctggaaggtgccactcccactgtcctttcctaataaaatgaggaaattgcatcgcattg |
| | tctgagtaggtgtcattctattctggggggtggggtggggcaggacagcaagggggagga |
| | ttgggaagacaatagcaggcatgctggggatgcggtgggctctatggcttctgaggcgga |
| | aagaaccagctggggctctagggggtatccccacgcgccctgtagcggcgcattaagcgc |
| | ggcgggtgtggtggttacgcgcagcgtgaccgctacacttgccagcgccctagcgcccgct |
| | cctttcgctttcttcccttcctttctcgccacgttcgccggctttccccgtcaagctctaaatcg |
| | ggggctccctttagggttccgatttagtgctttacggcacctcgaccccaaaaaacttgatta |
| | gggtgatggttcacgtagtgggccatcgccctgatagacggtttttcgccctttgacgttgga |
| | gtccacgttctttaatagtggactcttgttccaaactggaacaacactcaaccctatctcggt |
| | ctattcttttgatttataagggattttgccgatttcggcctattggttaaaaaatgagctgattt |
| | aacaaaaatttaacgcgaattaattctgtggaatgtgtgtcagttagggtgtggaaagtccc |
| | caggctccccagcaggcagaagtatgcaaagcatgcagaattctatcaaatatttaaaga |
| | aaaaaaaattgtatcaactttctacaatctctttcagaagacagaagcagagggaatactt |
| | cctaaatcattcaactaggccagcattaccttaataccggaactagaaaatgacattacaa |
| | gaaaagaaaacaacagaccaatatctctcatgaacaaagatacaaacattttcaacaaa |
| | atattagcaaaaagaatccaagaatgtatcaaaaaatatacaccacaaccaagtagaatt |
| | tattccagatatgtaagggtggttcaacgtttgaaaatcaattaacgtaatttgtcccatcaa |
| | caggttaaagaagaaaatcacatggtcatattgatagacacagaaaaagcatttgacaaa |
| | atttaacacccattcatgatgcaatctctcagtaaactaggaatagaggaaaacttcctcag |
| | cttgaatgtaccttcctctcaattttgctatgaacctgaaactcctcttaaaaaataaagttttt |
| | catttaaaaagaaaacaaaaaacatggaggagcgttgatgtatctcattttagaccaatca |
| | gctatggatagttaggcgacagcacagatagctgctgtacttctgtttctggcaatgttcca |
| | gactacatttaaaaaatttttaattatagacttgtacttaatgttcaagaaaaatatgaaaat |
| | ggctttgccgtgttaatgctactcttttttaaaaaaaactaaagttcaaactttatttatatttc |
| | attagttttttagctactgttctttttctgttctgggatctcattcagaatgccacattacatata |
| | attctcatgtctccttgggttcctcttagttttgacagttcctcagacttttcttatttttgatgac |
| | cttgacagttttgaggagtactggttagatatagggtaatggtttttaaagtatatttgtcatg |
| | atttatactggggtaagggtttggggaggaagcccatggggtaaagtactgttctcatcac |
| | atcatatcaaggttatataccatcaatattgccacagatgttacttagccttttaatatttctct |
| | aatttagtgtatatgcaatgatagttctctgatttctgagattgagtttctcatgtgtaatgatta |
| | tttagagtttctctttcatctgttcaaatttttgtctagttttattttttactgatttgtaagactt |
| | ctttttataatctgcatattacaattctctttactggggtgttgcaaatattttctgtcattctatg |
| | gcctgacttttcttaatggttttttaattttaaaaataagtcttaatattcatgcaatctaattaa |
| | caatcttttctttgtggttaggactttgagtcataagaaatttttctctacactgaagtcatgat |
| | ggcatgcttctatattattttctaaaagatttaaagttttgccttctccatttagacttataattc |
| | actggaatttttttgtgtgtatggtatgacatatgggttcccttttattttttacatataaatata |
| | tttccctgtttttctaaaaaagaaaaagatcatcattttcccattgtaaaatgccatattttttt |
| | cataggtcacttacatatatcaatgggtctgtttctgagctctactctattttatcagcctcact |
| | gtctatccccacacatctcatgctttgctctaaatcttgatatttagtggaacattctttcccat |
| | tttgttctacaagaatatttttgttattgtcttttgggcttctatatacattttagaatgaggttg |
| | gcaagttaacaaacagcttttttggggtgaacatattgactacaaatttatgtggaaagaa |
| | agtaccaagttgaccagtgccgttccggtgctcaccgcgcgcgacgtcgccggagcggtc |
| | gagttctggaccgaccggctcgggttctcccgggacttcgtggaggacgacttcgccggtg |
| | tggtccgggacgacgtgaccctgttcatcagcgcggtccaggaccaggtggtgccggaca |
| | acaccctggcctgggtgtgggtgcgcggcctggacgagctgtacgccgagtggtcggagg |
| | tcgtgtccacgaacttccgggacgcctccgggccggccatgaccgagatcggcgagcagc |
| | cgtgggggcgggagttcgccctgcgcgacccggccggcaactgcgtgcacttcgtggccg |
| | aggagcaggactgacacgtgctacgagatttcgattccaccgccgccttctatgaaaggtt |
| | gggcttcggaatcgttttccgggacgccggctggatgatcctccagcgcggggatctcatg |
| | ctggagttcttcgcccaccccaacttgtttattgcagcttataatggttacaaataaagcaat |
| | agcatcacaaatttcacaaataaagcatttttttcactgcattctagttgtggtttgtccaaac |
| | tcatcaatgtatcttatcatgtctgtataccgtcgacctctagctagagcttggcgtaatcatg |
| | gtcatagctgtttcctgtgtgaaattgttatccgctcacaattccacacaacatacgagccgg |
| | aagcataaagtgtaaagcctggggtgcctaatgagtgagctaactcacattaattgcgttg |
| | cgctcactgcccgctttccagtcgggaaacctgtcgtgccagctgcattaatgaatcggcca |
| | acgcgcggggagaggcggtttgcgtattgggcgctcttccgcttcctcgctcactgactcgc |
| | tgcgctcggtcgttcggctgcggcgagcggtatcagctcactcaaaggcggtaatacggtt |
| | atccacagaatcaggggataacgcaggaaagaacatgtgagcaaaaggccagcaaaag |
| | gccaggaaccgtaaaaaggccgcgttgctggcgtttttccataggctccgcccccctgacg |
| | agcatcacaaaaatcgacgctcaagtcagaggtggcgaaacccgacaggactataaaga |
| | taccaggcgtttccccctggaagctccctcgtgcgctctcctgttccgaccctgccgcttacc |
| | ggatacctgtccgcctttctcccttcgggaagcgtggcgctttctcatagctcacgctgtagg |
| | tatctcagttcggtgtaggtcgttcgctccaagctgggctgtgtgcacgaaccccccgttca |
| | gcccgaccgctgcgccttatccggtaactatcgtcttgagtccaacccggtaagacacgac |
| | ttatcgccactggcagcagccactggtaacaggattagcagagcgaggtatgtaggcggt |
| | gctacagagttcttgaagtggtggcctaactacggctacactagaagaacagtatttggtat |
| | ctgcgctctgctgaagccagttaccttcggaaaaagagttggtagctcttgatccggcaaa |
| | caaaccaccgctggtagcggtttttttgtttgcaagcagcagattacgcgcagaaaaaaag |
| | gatctcaagaagatcctttgatcttttctacggggtctgacgctcagtggaacgaaaactca |
| | cgttaagggattttggtcatgagattatcaaaaaggatcttcacctagatccttttaaattaa |
| | aaatgaagttttaaatcaatctaaagtatatatgagtaaacttggtctgacagttaccaatg |
| | cttaatcagtgaggcacctatctcagcgatctgtctatttcgttcatccatagttgcctgactc |
| | cccgtcgtgtagataactacgatacgggagggcttaccatctggccccagtgctgcaatga |
| | taccgcgagacccacgctcaccggctccagatttatcagcaataaaccagccagccggaa |
| | gggccgagcgcagaagtggtcctgcaactttatccgcctccatccagtctattaattgttgc |
| | cgggaagctagagtaagtagttcgccagttaatagtttgcgcaacgttgttgccattgctac |
| | aggcatcgtggtgtcacgctcgtcgtttggtatggcttcattcagctccggttcccaacgatc |
| | aaggcgagttacatgatcccccatgttgtgcaaaaaagcggttagctccttcggtcctccga |
| | tcgttgtcagaagtaagttggccgcagtgttatcactcatggttatggcagcactgcataatt |
| | ctcttactgtcatgccatccgtaagatgcttttctgtgactggtgagtactcaaccaagtcatt |
| | ctgagaatagtgtatgcggcgaccgagttgctcttgcccggcgtcaatacgggataatacc |
| | gcgccacatagcagaactttaaaagtgctcatcattggaaaacgttcttcggggcgaaaac |
| | tctc (SEQ ID NO: 47) |
| HPV-45 L1 | MALWRPSDSTVYLPPPSVARVVSTDDYVSRTSIFYHAGSS |
| amino acid | RLLTVGNPYFRVVPNGAGNKQAVPKVSAYQYRVFRVALP |
| | DPNKFGLPDSTIYNPETQRLVWACVGMEIGRGQPLGIGLS |
| | GHPFYNKLDDTESAHAATAVITQDVRDNVSVDYKQTQLC |
| | ILGCVPAIGEHWAKGTLCKPAQLQPGDCPPLELKNTIIED |
| | GDMVDTGYGAMDFSTLQDTKCEVPLDICQSICKYPDYLQ |
| | MSADPYGDSMFFCLRREQLFARHFWNRAGVMGDTVPTD |
| | LYIKGTSANMRETPGSCVYSPSPSGSIITSDSQLFNKPYWL |
| | HKAQGHNNGICWHNQLFVTVVDTTRSTNLTLCASTQNPV |
| | PSTYDPTKFKQYSRHVEEYDLQFIFQLCTITLTAEVMSYIH |
| | SMNSSILENWNFGVPPPPTTSLVDTYRFVQSVAVTCQKDT |
| | TPPEKQDPYDKLKFWTVDLKEKFSSDLDQYPLGRKFLVQ |
| | AGLRRRPTIGPRKRPAASTSTASTASRPAKRVRIRSKK |
| | (SEQ ID NO: 48) |
| HPV-45 L2 | MVSHRAARRKRASATDLYRTCKQSGTCPPDVINKVEGTT |
| amino acid | LADKILQWSSLGIFLGGLGIGTGSGSGGRTGYVPLGGRSN |
| | TVVDVGPTRPPVVIEPVGPTDPSIVTLVEDSSVVASGAPVP |
| | TFTGTSGFEITSSGTTTPAVLDITPTVDSVSISSTSFTNPAFS |
| | DPSIIEVPQTGEVSGNIFVGTPTSGSHGYEEIPLQTFASSGS |
| | GTEPISSTPLPTVRRVAGPRLYSRANQQVRVSTSQFLTHPS |
| | SLVTFDNPAYEPLDTTLSFEPTSNVPDSDFMDIIRLHRPAL |
| | SSRRGTVRFSRLGQRATMFTRSGKQIGGRVHFYHDISPIA |
| | ATEEIELQPLISATNDSDLFDVYADFPPPASTTPSTIHKSFT |
| | YPKYSLTMPSTAASSYSNVTVPLTSAWDVPIYTGPDIILPS |
| | HTPMWPSTSPTNASTTTYIGIHGTQYYLWPWYYYFPKKR |
| | KRIPYFFADGFVAA (SEQ ID NO: 49) |
| pDY0112 | gaagggcaggaggggcgactggggcccgcccgccttcggagcacatgtccgacgccacc |
| pVITRO- | tggatggggcgaggcctgtggctttccgaagcaatcgggcgtgagtttagcctacctgggc |
| HPV68 L1L2 | catgtggccctagcactgggcacggtctggcctggcggtgccgcgttcccttgcctcccaac |
| (seq | aagggtgaggccgtcccgcccggcaccagttgcttgcgcggaaagatggccgctcccggg |
| OavfqSEA) | gccctgttgcaaggagctcaaaatggaggacgcggcagcccggtggagcgggcgggtga |
| HPV-68 L2 | gtcacccacacaaaggaagagggccttgcccctcgccggccgctgcttcctgtgaccccgt |
| coding | ggtctatcggccgcatagtcacctcgggcttctcttgagcaccgctcgtcgcggcgggggg |
| sequence: | aggggatctaatggcgttggagtttgttcacatttggtgggtggagactagtcaggccagc |
| nucleotides | ctggcgctggaagtcattcttggaatttgcccctttgagtttggagcgaggctaattctcaag |
| 632 to 2030 | cctcttagcggttcaaaggtattttctaaacccgtttccaggtgttgtgaaagccaccgctaa |
| FMDV IRES: | ttcaaagcaatccggagtatacggatccgccaccatggtgtcccacagagccgccagacg |
| nucleotides | gaagcgggccagcgccaccgacctgtacaagacctgcaagcagagcggcacctgcccca |
| 2064 to 2508 | gcgacgtgatcaacaaggtggagggcaccacactggccgacaagatcctgcagtggacc |
| EM7 | agcctgggcatcttcctgggcggcctgggcattggcaccggcagcggcacaggcggcag |
| promoter: | agccggctacatccccctcggcggcaagcccaacaccgtggtggacgtgtcccccgccag |
| nucleotides | accccccgtggtcatcgagcccgtgggccccaccgagcccagcatcgtgcagctggtcga |
| 2541 to 2587 | ggacagcagcgtgatcacctctggcacacccgtccccaccttcaccggcaccagcggcttc |
| T7 promoter: | gagatcaccagcagctccaccaccacccctgccgtgctggacatcacccccagcagcggc |
| nucleotides | agcgtgcaggtgtccagcaccagcttcaccaaccccgccttcaccgaccccaccatcatcg |
| 2579 to 2597 | aggtgccccagaccggcgaggtgtccggcaacgtgttcgtgagcacccccacctccggca |
| EF-1 alpha | ctcacggctatgaggaaatccccatgcaggtgttcgccacccacggcacaggcacagaac |
| polyA: | ctatcagcagcacccccatccctggcgtgtctcgggtggcaggaccccggctctactctag |
| nucleotides | ggctcaccagcaggtccgggtgtccaacttcgacttcgtgacccacccctctagcttcgtca |
| 3448 to 4020 | ccttcgacaaccctgccttcgagcctgtggacaccactctgacctatgagcccgccgatatc |
| mEF-1-alpha | gcccccgaccccgacttcctggacatcgtgcggctgcacagacccgccctgaccagcaga |
| intron: | cggggcaccgtgcggttcagcagagtgggcaagaaagccaccatgttcaccaggcgggg |
| nucleotides | gacccagatcggcgcccaggtgcactactaccacgacatcagcaatatcacaccagccga |
| 4604 to 5551 | cagcatcgagctgcagcccctggtggcccccgagcaggccgaccccatggacaacctgta |
| HPV-68 L1 | cgacatctacgctcccgatactgacaacaccaccgtgctggataccgccttccacaacgcc |
| coding | acctttaccaccagatcccacatcagcgtgcccagcctggccagcgccgccagcaccacct |
| sequence: | acacaaacaccaccatccctctgggcaccgcctggaacacccccgtgaacaccggccctg |
| nucleotides | acgtggtcctgcccagcacaacaccccagctgcctctgaccccctccacccccatcgacac |
| 5609 to 7141 | caccttcgccatcaccatctacggcagcaattactacctcctgcccctgctgttcttcctgctg |
| SV40 polyA: | aagaagcggaagcacctgccctactttttcaccgacggcatcgtggccagctgatagtcta |
| nucleotides | ggagcaggtttccccaatgacacaaaacgtgcaacttgaaactccgcctggtctttccagg |
| 7149 to 7270 | tctagaggggtaacactttgtactgcgtttggctccacgctcgatccactggcgagtgttagt |
| | aacagcactgttgcttcgtagcggagcatgacggccgtgggaactcctccttggtaacaag |
| | gacccacggggccaaaagccacgcccacacgggcccgtcatgtgtgcaaccccagcacg |
| | gcgactttactgcgaaacccactttaaagtgacattgaaactggtacccacacactggtga |
| | caggctaaggatgcccttcaggtaccccgaggtaacacgcgacactcgggatctgagaag |
| | gggactggggcttctataaaagcgctcggtttaaaaagcttctatgcctgaataggtgacc |
| | ggaggtcggcacctttcctttgcaattactgaccctatgaatacactgactgtttgacaatta |
| | atcatcggcatagtatatcggcatagtataatacgactcactataggagggccaccatgat |
| | tgaacaagatggattgcacgcaggttctccggccgcttgggtggagaggctattcggctat |
| | gactgggcacaacagacaatcggctgctctgatgccgccgtgttccggctgtcagcgcag |
| | gggcgcccggttctttttgtcaagaccgacctgtccggtgccctgaatgaactgcaagacg |
| | aggcagcgcggctatcgtggctggccacgacgggcgttccttgcgcagctgtgctcgacgt |
| | tgtcactgaagcgggaagggactggctgctattgggcgaagtgccggggcaggatctcct |
| | gtcatctcaccttgctcctgccgagaaagtatccatcatggctgatgcaatgcggcggctgc |
| | atacgcttgatccggctacctgcccattcgaccaccaagcgaaacatcgcatcgagcgag |
| | cacgtactcggatggaagccggtcttgtcgatcaggatgatctggacgaagagcatcagg |
| | ggctcgcgccagccgaactgttcgccaggctcaaggcgagcatgcccgacggcgaggat |
| | ctcgtcgtgacacatggcgatgcctgcttgccgaatatcatggtggaaaatggccgcttttc |
| | tggattcatcgactgtggccggctgggtgtggcggaccgctatcaggacatagcgttggct |
| | acccgtgatattgctgaagagcttggcggcgaatgggctgaccgcttcctcgtgctttacgg |
| | tatcgccgctcccgattcgcagcgcatcgccttctatcgccttcttgacgagttcttctgagcg |
| | ggactctggggttcgaaatgaccgaccaagcgaattcgctaggattatccctaatacctgc |
| | caccccactcttaatcagtggtggaagaacggtctcagaactgtttgtttcaattggccattt |
| | aagtttagtagtaaaagactggttaatgataacaatgcatcgtaaaaccttcagaaggaa |
| | aggagaatgttttgtggaccactttggttttcttttttgcgtgtggcagttttaagttattagttt |
| | ttaaaatcagtactttttaatggaaacaacttgaccaaaaatttgtcacagaattttgagac |
| | ccattaaaaaagttaaatgagaaacctgtgtgttcctttggtcaacaccgagacatttaggt |
| | gaaagacatctaattctggttttacgaatctggaaacttcttgaaaatgtaattcttgagtta |
| | acacttctgggtggagaatagggttgttttccccccacataattggaaggggaaggaatat |
| | catttaaagctatgggagggttgctttgattacaacactggagagaaatgcagcatgttgct |
| | gattgcctgtcactaaaacaggccaaaaactgagtccttgggttgcatagaaagctgcctg |
| | cagggcctgaaataacctctgaaagaggaacttggttaggtaccttctgaggcggaaaga |
| | accagctgtggaatgtgtgtcagttagggtgtggaaagtccccaggctccccagcaggca |
| | gaagtatgcaaagcatgcatctcaattagtcagcaaccaggtgtggaaagtccccaggct |
| | ccccagcaggcagaagtatgcaaagcatgcatctcaattagtcagcaaccatagtcccac |
| | tagtggagccgagagtaattcatacaaaaggagggatcgccttcgcaaggggagagccc |
| | agggaccgtccctaaattctcacagacccaaatccctgtagccgccccacgacagcgcga |
| | ggagcatgcgctcagggctgagcgcggggagagcagagcacacaagctcatagaccct |
| | ggtcgtgggggggaggaccggggagctggcgcggggcaaactgggaaagcggtgtcgt |
| | gtgctggctccgccctcttcccgagggtgggggagaacggtatataagtgcggcagtcgcc |
| | ttggacgttctttttcgcaacgggtttgccgtcagaacgcaggtgaggggcgggtgtggctt |
| | ccgcgggccgccgagctggaggtcctgctccgagcgggccgggccccgctgtcgtcggcg |
| | gggattagctgcgagcattcccgcttcgagttgcgggcggcgcgggaggcagagtgcgag |
| | gcctagcggcaaccccgtagcctcgcctcgtgtccggcttgaggcctagcgtggtgtccgc |
| | gccgccgccgcgtgctactccggccgcactctggtcttttttttttttgttgttgttgccctgctg |
| | ccttcgattgccgttcagcaataggggctaacaaagggagggtgcggggcttgctcgccc |
| | ggagcccggagaggtcatggttggggaggaatggagggacaggagtggcggctggggc |
| | ccgcccgccttcggagcacatgtccgacgccacctggatggggcgaggcctggggtttttc |
| | ccgaagcaaccaggctggggttagcgtgccgaggccatgtggccccagcacccggcacg |
| | atctggcttggcggcgccgcgttgccctgcctccctaactagggtgaggccatcccgtccgg |
| | caccagttgcgtgcgtggaaagatggccgctcccgggccctgttgcaaggagctcaaaat |
| | ggaggacgcggcagcccggtggagcgggcgggtgagtcacccacacaaaggaagagg |
| | gcctggtccctcaccggctgctgcttcctgtgaccccgtggtcctatcggccgcaatagtca |
| | cctcgggcttttgagcacggctagtcgcggcggggggaggggatgtaatggcgttggagtt |
| | tgttcacatttggtgggtggagactagtcaggccagcctggcgctggaagtcatttttggaa |
| | tttgtccccttgagttttgagcggagctaattctcgggcttcttagcggttcaaaggtatctttt |
| | aaacccttttttaggtgttgtgaaaaccaccgctaattcaaagcaaccggtgatatcaaag |
| | atccgccaccatggcactgtggagagccagcgacaacatggtgtacctgccccctcccag |
| | cgtggccaaggtggtcaacaccgacgactacgtgacccggaccggcatgtactactacgc |
| | cggcacctctcggctcctgaccgtgggccacccctacttcaaggtgcccatgagcggcggc |
| | agaaagcagggcatccccaaggtgtccgcctaccagtaccgggtgttcagagtgaccctg |
| | cccgaccccaacaagttcagcgtgcccgagagcaccctgtacaaccccgacacccagcg |
| | gatggtctgggcctgcgtgggcgtggagatcggcagaggccagcccctgggcgtgggcct |
| | gagcggccaccccctgtacaatcggctggacgacaccgagaacagccccttcagcagca |
| | acaagaaccccaaggacagccgggacaacgtggccgtggactgcaagcagacccagct |
| | gtgcatcatcggctgcgtgcctgccattggcgagcactgggccaagggcaagagctgcaa |
| | gcccaccaacgtgcagcagggcgactgcccccctctggaactggtcaacacacccatcga |
| | ggacggcgacatgatcgacaccggctacggcgccatggacttcggcaccctgcaggaaa |
| | ccaagagcgaggtccccctggacatctgccagagcgtgtgcaagtaccccgactacctgc |
| | agatgagcgccgacgtgtacggcgacagcatgttcttttgcctgcggcgggagcagctgtt |
| | cgcccggcacttctggaacagaggcggcatggtcggcgacaccatccccaccgacatgta |
| | catcaagggcaccgacatcagagagacacccagcagctacgtgtacgcccccagcccca |
| | gcggcagcatggtgtccagcgacagccagctgttcaacaagccctactggctgcacaagg |
| | cccagggccacaacaacggcatctgctggcacaaccagctgtttctgaccgtggtggaca |
| | ccaccagaagcaccaacttcaccctgagcaccaccaccgacagcaccgtgcccgccgtgt |
| | acgacagcaataagttcaaagaatacgtgcggcacgtggaggaatacgacctgcagttc |
| | atcttccagctgtgtaccatcaccctgtccaccgacgtgatgagctacatccacaccatgaa |
| | ccccgccatcctggacgactggaacttcggcgtggcccctccccctagcgccagcctggtg |
| | gatacctacagatacctgcagagcgccgccatcacctgccagaaggacgcccctgccccc |
| | gtgaagaaggacccctacgacggcctgaacttctggaatgtggacctgaaagagaagttc |
| | agcagcgagctggaccagttccccctgggccggaagttcctgctgcaagccggcgtgcgg |
| | agaaggcccaccatcggccccagaaagcggaccgccaccgcagccacaacctccacctc |
| | caagcacaagcggaagcgggtgtccaagtgatagtctagctggccagacatgataagat |
| | acattgatgagtttggacaaaccacaactagaatgcagtgaaaaaaatgctttatttgtga |
| | aatttgtgatgctattgctttatttgtaaccattataagctgcaataaacaagttaacaacaa |
| | caattgcattcattttatgtttcaggttcagggggaggtgtgggaggttttttaaagcaagta |
| | aaacctctacaaatgtggtatggaaatgttaattaactagccatgaccaaaatcccttaac |
| | gtgagttttcgttccactgagcgtcagaccccgtagaaaagatcaaaggatcttcttgagat |
| | cctttttttctgcgcgtaatctgctgcttgcaaacaaaaaaaccaccgctaccagcggtggtt |
| | tgtttgccggatcaagagctaccaactctttttccgaaggtaactggcttcagcagagcgca |
| | gataccaaatactgttcttctagtgtagccgtagttaggccaccacttcaagaactctgtag |
| | caccgcctacatacctcgctctgctaatcctgttaccagtggctgctgccagtggcgataag |
| | tcgtgtcttaccgggttggactcaagacgatagttaccggataaggcgcagcggtcgggct |
| | gaacggggggttcgtgcacacagcccagcttggagcgaacgacctacaccgaactgaga |
| | tacctacagcgtgagctatgagaaagcgccacgcttcccgaagggagaaaggcggacag |
| | gtatccggtaagcggcagggtcggaacaggagagcgcacgagggagcttccaggggga |
| | aacgcctggtatctttatagtcctgtcgggtttcgccacctctgacttgagcgtcgatttttgt |
| | gatgctcgtcaggggggcggagcctatggaaaaacgccagcaacgcggcctttttacggt |
| | tcctggccttttgctggccttttgctcacatgttcttaattaacctgcaggcgttacataactta |
| | cggtaaatggcccgcctggctgaccgcccaacgacccccgcccattgacgtcaataatga |
| | cgtatgttcccatagtaacgccaatagggactttccattgacgtcaatgggtggagtattta |
| | cggtaaactgcccacttggcagtacatcaagtgtatcatatgccaagtacgccccctattga |
| | cgtcaatgacggtaaatggcccgcctggcattatgcccagtacatgaccttatgggactttc |
| | ctacttggcagtacatctacgtattagtcatcgctattaccatgatgatgcggttttggcagt |
| | acatcaatgggcgtggatagcggtttgactcacggggatttccaagtctccaccccattgac |
| | gtcaatgggagtttgttttgactagtggagccgagagtaattcatacaaaaggagggatcg |
| | ccttcgcaaggggagagcccagggaccgtccctaaattctcacagacccaaatccctgta |
| | gccgccccacgacagcgcgaggagcatgcgcccagggctgagcgcgggtagatcagag |
| | cacacaagctcacagtccccggcggtggggggaggggcgcgctgagcgggggccaggg |
| | agctggcgcggggcaaactgggaaagtggtgtcgtgtgctggctccgccctcttcccgagg |
| | gtgggggagaacggtatataagtgcggtagtcgccttggacgttctttttcgcaacgggttt |
| | gccgtcagaacgcaggtgagtggcgggtgtggcttccgcgggccccggagctggagccct |
| | gctctgagcgggccgggctgatatgcgagtgtcgtccgcagggtttagctgtgagcattcc |
| | cacttcgagtggcgggcggtgcgggggtgagagtgcgaggcctagcggcaaccccgtag |
| | cctcgcctcgtgtccggcttgaggcctagcgtggtgtccgccgccgcgtgccactccggccg |
| | cactatgcgttttttgtccttgctgccctcgattgccttccagcagcatgggctaacaaaggg |
| | agggtgtggggctcactcttaaggagcccatgaagcttacgttggataggaatg |
| | (SEQ ID NO: 50) |
| HPV-68 L1 | MALWRASDNMVYLPPPSVAKVVNTDDYVTRTGMYYYA |
| amino acid | GTSRLLTVGHPYFKVPMSGGRKQGIPKVSAYQYRVFRVT |
| | LPDPNKFSVPESTLYNPDTQRMVWACVGVEIGRGQPLGV |
| | GLSGHPLYNRLDDTENSPFSSNKNPKDSRDNVAVDCKQT |
| | QLCIIGCVPAIGEHWAKGKSCKPTNVQQGDCPPLELVNT |
| | PIEDGDMIDTGYGAMDFGTLQETKSEVPLDICQSVCKYPD |
| | YLQMSADVYGDSMFFCLRREQLFARHFWNRGGMVGDTI |
| | PTDMYIKGTDIRETPSSYVYAPSPSGSMVSSDSQLFNKPY |
| | WLHKAQGHNNGICWHNQLFLTVVDTTRSTNFTLSTTTDS |
| | TVPAVYDSNKFKEYVRHVEEYDLQFIFQLCTITLSTDVMS |
| | YIHTMNPAILDDWNFGVAPPPSASLVDTYRYLQSAAITCQ |
| | KDAPAPVKKDPYDGLNFWNVDLKEKFSSELDQFPLGRKF |
| | LLQAGVRRRPTIGPRKRTATAATTSTSKHKRKRVSK |
| | SSWP (SEQ ID NO: 51) |
| HPV-68 L2 | GSATMVSHRAARRKRASATDLYKTCKQSGTCPSDVINKV |
| amino acid | EGTTLADKILQWTSLGIFLGGLGIGTGSGTGGRAGYIPLG |
| | GKPNTVVDVSPARPPVVIEPVGPTEPSIVQLVEDSSVITSGT |
| | PVPTFTGTSGFEITSSSTTTPAVLDITPSSGSVQVSSTSFTNP |
| | AFTDPTIIEVPQTGEVSGNVFVSTPTSGTHGYEEIPMQVFA |
| | THGTGTEPISSTPIPGVSRVAGPRLYSRAHQQVRVSNFDFV |
| | THPSSFVTFDNPAFEPVDTTLTYEPADIAPDPDFLDIVRLH |
| | RPALTSRRGTVRFSRVGKKATMFTRRGTQIGAQVHYYH |
| | DISNITPADSIELQPLVAPEQADPMDNLYDIYAPDTDNTTV |
| | LDTAFHNATFTTRSHISVPSLASAASTTYTNTTIPLGTAWN |
| | TPVNTGPDVVLPSTTPQLPLTPSTPIDTTFAITIYGSNYYLL |
| | PLLFFLLKKRKHLPYFF (SEQ ID NO: 52) |

We claim:

1. A papillomaviral delivery vehicle, comprising:
a papillomavirus-derived capsid; and
DNA encoding a gene editing material encapsulated by the capsid.

2. The papillomaviral delivery vehicle of claim 1, wherein the capsid is derived from a mammalian papillomavirus.

3. The papillomaviral delivery vehicle of claim 2, wherein the capsid is derived from a human papillomavirus (HPV).

4. The papillomaviral delivery vehicle of claim 2, wherein the mammalian papillomavirus is selected from the group consisting of an HPV-1, an HPV-2, an HPV-3, an HPV-4, an HPV-5, an HPV-6, an HPV-7, an HPV-8, an HPV-9, an HPV-10, an HPV-11, an HPV-12, an HPV-13, an HPV-14, an HPV-15, an HPV-16, an HPV-17, an HPV-18, an HPV-19, an HPV-20, an HPV-21, an HPV-22, an HPV-23, an HPV-24, an HPV-25, an HPV-26, an HPV-27, an HPV-28, an HPV-29, an HPV-30, an HPV-31, an HPV-32, an HPV-33, an HPV-34, an HPV-35, an HPV-36, an HPV-37, an HPV-38, an HPV-39, an HPV-40, an HPV-41, an HPV-42, an HPV-43, an HPV-44, an HPV-45, an HPV-47, an HPV-48, an HPV-49, an HPV-50, an HPV-51, an HPV-52, an HPV-53, an HPV-54, an HPV-56, an HPV-57, an HPV-58, an HPV-59, an HPV-60, an HPV-61, an HPV-62, an HPV-63, an HPV-65, an HPV-66, an HPV-67, an HPV-68, an HPV-69, an HPV-70, an HPV-71, an HPV-72, an HPV-73, an HPV-74, an HPV-75, an HPV-76, an HPV-77, an HPV-78, an HPV-80, an HPV-81, an HPV-82, an HPV-83, an HPV-84, an HPV-85, an HPV-86, an HPV-87, an HPV-88, an HPV-89, an HPV-90, an HPV-91, an HPV-92, an HPV-93, an HPV-94, an HPV-95, an HPV-96, an HPV-97, an HPV-98, an HPV-99, an HPV-100, an HPV-101, an HPV-102, an HPV-103, an HPV-104, an HPV-105, an HPV-106, an HPV-107, an HPV-108, an HPV-109, an HPV-110, an HPV-111, an HPV-112, an HPV-113, an HPV-114, an HPV-115, an HPV-116, an HPV-117, an HPV-118, an HPV-119, an HPV-120, an HPV-121, an HPV-122, an HPV-123, an HPV-124, an HPV-125, an HPV-126, an HPV-127, an HPV-128, an HPV-129, an HPV-130, an HPV-131, an HPV-132, an HPV-133, an HPV-134, an HPV-135, an HPV-136, an HPV-137, an HPV-138, an HPV-139, an HPV-140, an HPV-141, an HPV-142, an HPV-143, an HPV-144, an HPV-145, an HPV-146, an HPV-147, an HPV-148, an HPV-149, an HPV-150, an HPV-151, an HPV-152, an HPV-153, an HPV-154, an HPV-155, an HPV-156, an HPV-157, an HPV-158, an HPV-159, an HPV-160, an HPV-161, an HPV-162, an HPV-163, an HPV-164, an HPV-165, an HPV-166, an HPV-167, an HPV-168, an HPV-169, an HPV-170, an HPV-171, an HPV-172, an HPV-173, an HPV-174, an HPV-175, an HPV-176, an HPV-177, an HPV-178, an HPV-179, an HPV-180, an HPV-181, an HPV-182, an HPV-183, an HPV-184, an HPV-185, an HPV-186, an HPV-187, an HPV-188, an HPV-189, an HPV-190, an HPV-191, an HPV-192, an HPV-193, an HPV-194, an HPV-195, an HPV-196, an HPV-197, an HPV-199, an HPV-200, an HPV-201, an HPV-202, an HPV-203, an HPV-204, an HPV-205, an HPV-206, an HPV-207, an HPV-208, an HPV-209, an HPV-210, an HPV-211, an HPV-212, an HPV-213, an HPV-214, an HPV-215, an HPV-216, an HPV-219, an HPV-220, an HPV-221, an HPV-222, an HPV-223, an HPV-224, an HPV-225, a MmuPV-1, and a variant thereof.

5. The papillomaviral delivery vehicle of claim 1, wherein the capsid comprises a L1 capsid protein.

6. The papillomaviral delivery vehicle of claim 5, wherein the L1 capsid protein comprises an amino acid sequence selected from the group consisting of: SEQ ID NO: 2, 5, 8, 11, 14, 17, 20, 23, 26, 29, 32, 35, 38, 45, 48, and 51.

7. The papillomaviral delivery vehicle of claim 1, wherein the capsid comprises a L2 capsid protein.

8. The papillomaviral delivery vehicle of claim 7, wherein the L2 capsid protein comprises an amino acid sequence selected from the group consisting of: SEQ ID NO: 3, 6, 9, 12, 15, 18, 21, 24, 27, 30, 33, 36, 39, 46, 49, and 52.

9. The papillomaviral delivery vehicle of any previous claims, wherein the DNA encoding the gene editing material comprises a minicircle.

10. The papillomaviral delivery vehicle of claim 9, wherein the minicircle does not comprise a sequence of a bacterial origin.

11. The papillomaviral delivery vehicle of any previous claims, wherein the gene editing material is selected from the group consisting of a nuclease, a nuclease coupled to a deaminase, a deaminase, a nickase, a transcriptase, a reverse transcriptase, an integration enzyme, an epigenetic modifier, a DNA methyltransferases, a guide RNA, a homology-directed repair (HDR) template, a reporter gene, a polynucleotide linked to a sequence complementary to an integration site, a split intein, a derivative thereof, and a combination thereof.

12. The papillomaviral delivery vehicle of claim 11, wherein the nuclease comprises a DNA-binding nuclease, a DNA-cleaving nuclease, a meganuclease, a zinc-finger nuclease (ZFN), a transcription activator-like effector nuclease (TALEN), a derivative thereof, or a combination thereof.

13. The papillomaviral delivery vehicle of claim 12, wherein the DNA binding nuclease comprises a clustered regularly interspaced short palindromic repeat (CRISPR)-associated (Cas) DNA-binding nuclease.

14. The papillomaviral delivery vehicle of claim 13, wherein the Cas DNA-binding nuclease comprises a Cascade (type I) nuclease, type III nuclease, a Cas9 nuclease, a Cas12 nuclease, a variant thereof, or a combination thereof.

15. The papillomaviral delivery vehicle of claim 11, wherein the nuclease comprises an RNA-targeting nuclease, an RNA-binding nuclease, an RNA-cleaving nuclease, a derivative thereof, or a combination thereof.

16. The papillomaviral delivery vehicle of claim 11, wherein the nuclease comprises a Cas13a nuclease, a Cas13b nuclease, a Cas13c nuclease, a Cas13d nuclease, a Cas13e nucleases, a Cas7-11 nuclease, a variant thereof, or a combination thereof.

17. The papillomaviral delivery vehicle of claim 11, wherein the guide RNA comprises a single-guide RNA (sgRNA), a dual-guide RNA (dgRNA), a prime-editing guide RNA (pegRNA), a nicking-guide RNA (ngRNA), a derivative thereof, or a combination thereof.

18. The papillomaviral delivery vehicle of claim 11, wherein the reporter gene encodes a fluorescent protein.

19. The papillomaviral delivery vehicle of claim 18, wherein the fluorescent protein comprises a green fluorescent protein (GFP), a tdTomato protein, DsRed protein, a derivative thereof, or a combination thereof.

20. The papillomaviral delivery vehicle of claim 11, wherein the deaminase comprises an AncBE4 deaminase, an ABE7.10 deaminase, a derivative thereof, or a combination thereof.

21. The papillomaviral delivery vehicle of claim 1, wherein the gene-editing material comprises a single-stranded DNA editing material.

22. The papillomaviral delivery vehicle of claim 1, wherein the gene-editing material comprises a double-stranded DNA editing material.

23. A cell comprising the papillomaviral delivery vehicle of any of claims 1-20.

24. The cell of claim 23, comprising a eukaryotic cell.

25. The cell of claim 23, comprising a mammalian cell.

26. The cell of claim 23, comprising a human cell.

27. The cell of claim 23, comprising a hematopoietic stem cell, a progenitor cell, a satellite cell, a mesenchymal progenitor cell, an astrocyte cell, a T-cell, a B cell, a hepatocyte cell, a heart cell, a muscle cell, a retinal cell, a renal cell, or a colon cell.

28. A method of synthesizing a papillomaviral delivery vehicle according to any one of claims 1-20, the method comprising:
(a) transfecting a cell with:
(i) a first vector encoding a papillomavirus-derived capsid under conditions conducive for the cell to synthesize the papillomavirus-derived capsid; and
(ii) a second vector encoding a DNA encoding a gene editing material under conditions conducive for the cell to replicate the second vector;
(b) allowing the cell to assemble the papillomaviral delivery vehicle.

29. The method of claim 28, wherein the papillomaviral delivery vehicle is isolated from the cells.

30. A method of editing a polynucleotide target in a cell, the method comprising:
(a) transducing the papillomaviral delivery vehicle of any of claims 1-20 into the cell comprising the polynucleotide target under conditions conducive for the cell to synthesize the gene editing material; and
(b) allowing the gene editing material to edit the polynucleotide target.

31. The method of claim 30, wherein the polynucleotide target is a DNA.

32. The method of claim 30, wherein the polynucleotide target is a RNA.

33. The method of claim 30, further comprising knocking down the polynucleotide target.

34. Use of a papillomaviral delivery vehicle of any of claims 1-22 to edit a polynucleotide target in a cell.

35. The use of claim 34, wherein the polynucleotide target is a DNA.

36. The use of claim 34, wherein the polynucleotide target is a RNA.
